(19) United States
(12) Patent Application Publication
(10) Pub. No.: US 2025/0034091 A1
(43) Pub. Date: Jan. 30, 2025

(54) Small molecule inhibitors of lanosterol synthase

(71) Applicant: Board of Regents, The University of Texas System, Austin, TX (US)

(72) Inventors: Jef De Brabander, Dallas, TX (US); Deepak Nijhawan, Dallas, TX (US); Huayu Wang, Dallas, TX (US); Christopher Carroll, Dallas, TX (US); Thu Nguyen, Dallas, TX (US)

(73) Assignee: Board of Regents, The University of Texas System, Austin, TX (US)

(21) Appl. No.: 18/892,318

(22) Filed: Sep. 20, 2024

Related U.S. Application Data
(63) Continuation of application No. PCT/US23/64677, filed on Mar. 18, 2023.
(60) Provisional application No. 63/324,080, filed on Mar. 27, 2022.

(51) Int. Cl. C07D 209/70; A61K 31/402; A61K 31/4035; A61K 31/4188; A61K 31/422; A61K 31/4439; A61K 31/444; A61K 31/506; C07D 207/12; C07D 401/04; C07D 401/12; C07D 401/14; C07D 403/04; C07D 405/04; C07D 405/10; C07D 405/12; C07D 405/14; C07D 413/12; C07D 487/04

(52) U.S. Cl. CPC C07D 209/70; A61K 31/402; A61K 31/4035; A61K 31/4188; A61K 31/422; A61K 31/4439; A61K 31/444; A61K 31/506; C07D 207/12; C07D 401/04; C07D 401/12; C07D 401/14; C07D 403/04; C07D 405/04; C07D 405/10; C07D 405/12; C07D 405/14; C07D 413/12; C07D 487/04

(57) ABSTRACT

Compositions for treating a neurological disorder comprise lanosterol synthase inhibitors.

### CROSS-REFERENCES TO RELATED APPLICATIONS

This application is a continuation of PCT/US23/64677, filed Mar. 18, 2023, 2022, which claims priority to U.S. Provisional Application No. 63/324,080, filed Mar. 27, 2022, the disclosures of which are hereby incorporated by reference in its entirety for all purposes.

### INTRODUCTION

Glioblastoma, or GBM, is the most common primary malignant brain tumor in the United States (Ostrom et al., 2019). GBM carries a dismal prognosis, with a median survival time of 12 months, which increases to 14 months with current standard therapy (Stupp et al., 2005). Standard treatment, entailing surgical resection followed by concurrent radiation and temozolomide therapies, displays guarded antitumor activity, but relapse and tumor recurrence are inevitable. One of the key factors underlying poor outcomes for patients with GBM is the paucity of effective therapies for this disease.

### SUMMARY OF THE INVENTION

The invention provides small molecule inhibitors of the cholesterol biosynthetic enzyme lanosterol synthase. By inhibiting lanosterol synthase, these compounds inhibit the biosynthesis of lanosterol and hence cholesterol. They divert biosynthetic flux to the synthesis of a non-canonical sterol: 24,25-epoxycholesterol (EPC). This up regulation of EPC with small molecule lanosterol synthase inhibitors leads to the potent specific killing of glioblastoma cell lines. The invention provides a representative panel of analogs of lanosterol synthase inhibitors that kill glioblastoma cell lines, particularly in the low, single digit nano molar range. In embodiments the inhibitors are orally available and/or brain-penetrant. The invention provides methods and compositions for treating a glioblastoma, and developing novel therapeutics for treating a glioblastoma.

In an aspect the invention provides a compound of formula I:

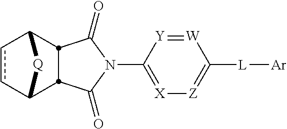

or a salt, hydrate or stereoisomer thereof, wherein.
Q is selected from cyclopropane-1,2-diyl, CH2, (CH2)2, O;
X, Y, Z and W are each independently selected from CR and N; R=H, halogen (e.g. F, Cl, Br); CH3, optionally fluorinated (e.g. CF3), optionally deuterated (e.g. CD3);
L is selected from: —OCHRC(O)— [R=H, Me, CF3, CHF2, CH2F], —NHCH2C(O)—, —NHCHMeC(O)—, —NMeCH2C(O)—, —NMeCHMeC(O)—, —SCH2C(O)—, —SCHMeC(O)—, —CH2OC(O)—, —CHMeOC(O)—, —CH2NHC(O)—, CHMeNHC(O)—, —OCH2NMeC(O)—, —CHMeNMeC(O)—, —CH2NHSO2-, —CHMeNHSO2-, —CH2NMeSO2-, —CMeNMeSO2-, —O(CH2)n- [n=0-2], —OCHMe-, —OCHMeCH2-, —OCH2CHR— [R=Me, OH, OMe, OCF3, OCHF2, OCH2F], —OCHMeCHR— [R=OH, OMe, OCF3, OCHF2, OCH2F], —(CH2)nC(O)— [n=0-2], —CHRCH2C(O)— [R=Me, OH, OMe, OCF3, OCHF2, OCH2F], —CH2CHRC(O)— [R=Me, OH, OMe, OCF3, OCHF2, OCH2F], —CHORCH2n- [n=0-2; R=H, Me, CF3, CHF2, CH2F], —(CH2)nCHOR— [n=1-2; R=H, Me, CF3, CHF2, CH2F], —CHORCH2CHOR′— [R, R′ independently selected from H, Me, CF3, CHF2, CH2F], —CHORCHMeCHOR′— [R, R′ independently selected from H, Me, CF3, CHF2, CH2F], —CHORCMeCH2- [R=H, Me, CF3, CHF2, CH2F], —CH(OR)CH═CH— [R=H, Me, CF3, CHF2, CH2F], —(CH2)n- [n=0-3], —CH2NR— [R=H, Me]-, —CH═CHC(O)—, CH═CHCHR— [R=OH, OMe, OCF3, OCHF2, OCH2F], —CMe=CHC(O)—, CH═CMeC(O)—, CMe=CHCHOR— [R=H, Me, CF3, CHF2, CH2F], CH═CMeCHOR [R=H, Me, CF3, CHF2, CH2F], —C≡CC(O)—, —C≡CCHR [R=H, Me, OH, OMe, OCF3, OCHF2, OCH2F], -(cyclopropane-1,2-diyl)C(O)—, -(cyclopropane-1,2-diyl)CHR— [R=H, Me, OH, OMe, OCF3, OCHF2, OCH2F], -(oxiran-2,3-diyl)C(O)—, -(oxiran-2,3-diyl)CHR— [R=H, Me, OH, OMe, OCF3, OCHF2, OCH2F], —C(Oxcyclopropane-1,2-diyl)-, —CHR(cyclopropane-1,2-diyl)- [R=H, Me, OH, OMe, OCF3, OCHF2, OCH2F], —C(O)(oxiran-2,3-diyl)-, —CHR(oxiran-2,3-diyl)- [R=H, Me, OH, OMe, OCF3, OCHF2, OCH2F]; and
Ar is selected from 2-, or 3- or, 4-mono- or, di- or trisubstituted phenyls (with preferred substituents selected from halogen (e.g. F, Cl, Br), Me or OMe, cycloPr, CN, —NHCHO, each optionally fluorinated and optionally deuterated (e.g. OCF3, CF3, CD3); and optionally substituted phenyls and heteroaryls), mono- and fused bicyclic heteroaryl, fused heteroaryl [e.g. pyridine; pyrimide, pyrazine, pyridazine, oxazole, benzoxazole, pyrrazole, quinoline, quinoxaline, isoquinoline], biaryls and fused aryls (naphtyls); each optionally fluorinated and optionally deuterated (e.g. OCF3, CF3, CD3); with optional substituents selected from halogen (e.g. F, Cl, Br), CF3, Me or OMe, cPr, CN, aryl, and heteroaryl, each optionally fluorinated and optionally deuterated (e.g. OCF3, CF3, CD3).

In embodiments:
Q is cyclopropane-1,2-diyl;
X is N and Y, Z and W are each CH; or X, Y, Z and W are each CH;
L is selected from: —OCHC(O)—, —NHCH2C(O)—, —NHCHMeC(O)—, —NMeCH2C(O)—, —NMeCHMeC(O)—, —CH2OC(O)—, —CHMeOC(O)—, —CH2NHC(O)—, CHMeNHC(O)—, —OCH2NMeC(O)—, —CHMeNMeC(O)—, —OCHMe-, —OCHMeCH2-, —OCH2CHMe-, —OCHMeCHOH—, —(CH2)C(O)—, —CHMeCH2C(O)—, —CH2CHMeC(O)—, —CHOHCH2-, —CH2CHOH—, —CHOHCH2CHOH′—, —CHOHCHMeCHOH—, —CHOHCMeCH2-, —CH(OH)CH═CH2-, —CH2NH—, -(cyclopropane-1,2-diyl)C(O)—, -(cyclopropane-1,2-diyl)CH2-, -(oxiran-2,3-diyl)C(O)—, -(oxiran-2,3-diyl)CH2-, —C(O)(cyclopropane-1,2-diyl)-, —CH2(cyclopropane-1,2-diyl)-, —C(O)(oxiran-2,3-diyl)-, —CH2(oxiran-2,3-diyl)-; and/or
Ar is 2-, or 3- or, 4-mono- or, di- or trisubstituted phenyls, with substituents selected from halogen, Me or OMe, cycloPr, CN, and —NHCHO, each optionally fluorinated and optionally deuterated.

In embodiments, the compound:
having a structure of Table 1, 2, 3, 4, 5, 6, 7, 8 or 9, herein.
inhibits lanosterol synthase (LSS);
is orally bioavailable; and/or
crosses the blood-brain barrier.

In an aspect the invention provides a pharmaceutical composition comprising a compound herein, or a pharmaceutically acceptable salt thereof, and a pharmaceutically acceptable carrier or excipient.

In an aspect the invention provides a method of inhibiting lanosterol synthase, comprising administering to a person in need thereof a small molecule lanosterol synthase inhibitor disclosed herein.

In an aspect the invention provides a method of method of upregulating 24,25-epoxycholesterol (EPC), comprising administering to a person in need thereof a small molecule lanosterol synthase inhibitor disclosed herein.

In an aspect the invention provides a method of treating a neurological disease or condition comprising administering to a person in need thereof a small molecule lanosterol synthase inhibitor disclosed herein.

In an aspect the invention provides a method of screening for candidate therapeutics for treating a neurological disease or condition, the method comprising identifying inhibitors of lanosterol synthase (p75).

In embodiments of the methods herein:
the neurological disease or condition is glioblastoma (GBM) or a neurodegenerative disease, such as amyotrophic lateral sclerosis, multiple sclerosis, Parkinson's disease, Alzheimer's disease, Huntington's disease; and/or
the method further comprising the antecedent step of detecting or diagnosing the disease or condition and/or the subsequent step of detecting a resultant improvement or delay of progression of the disease or condition.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We demonstrate that the tetracyclic dicarboximide MM0299 inhibits lanosterol synthase (denoted as Lss in mouse or LSS in human), the first enzyme in the post-squalene cholesterol biosynthetic pathway that synthesizes the tetracyclic sterol backbone. Like other LSS inhibitors, MM0299 blocks canonical cholesterol biosynthesis and diverts sterol synthetic flux towards the production of the shunt pathway product, 24(S),25-epoxycholesterol (EPC), which is responsible for blocking the growth of GSCs by depleting cellular cholesterol. In comparison to a known LSS inhibitor, MM0299 exhibits superior selectivity for LSS over other sterol biosynthetic enzymes. We disclose MM0299 derivatives that are orally bioavailable, penetrate the blood-brain barrier, and stimulate EPC production in tumors but not normal brain. The invention provides novel lanosterol synthase inhibitors as treatments for glioblastoma or other neurologic conditions.

Selective and Brain-Penetrant Lanosterol Synthase Inhibitors Target Glioma Stem-Like Cells by Activating a Shunt Pathway that Generates the Toxic Metabolite 24(S),25-Epoxycholesterol

We deployed a medicinal chemistry campaign to produce pharmacologically optimized preclinical LSSi leads and follow-up studies to assess their target engagement, safety, and anti-GBM efficacy profiles. We exploited the structural-activity relationship (SAR) embedded within a sizable collection of analogs produced through medicinal chemistry to correlate cellular phenotypic SAR data to cellular probe displacement EC50's in order to distinguish between on and off-target binding events. We used a high-throughput cell survival screen to identify chemicals that impair GBM cell fitness, using Mut 6, a murine GBM stem-like cell (GSC) line, from a genetically engineered mouse model of GBM wherein autochthonous tumor formation is driven by GFAP-Cre-mediated silencing of the tumor suppressors Trp53, Pten, and Nf1 in murine astrocytes.

We explored the mechanism of a tetracyclic dicarboximide (hereafter referred to as MM0299), which emerged from our screen. MM0299 inhibits lanosterol synthase (denoted as Lss in mouse or LSS in human), the first enzyme in the post-squalene cholesterol biosynthetic pathway that synthesizes the tetracyclic sterol backbone. Inhibition of LSS by MM0299 blocks canonical cholesterol biosynthesis and diverts sterol synthetic flux towards the production of the shunt pathway product, 24(S),25-epoxycholesterol (EPC), which in turn blocks the growth of GSCs.

Our invention provides a new approach to target tumor cell metabolism for brain tumor therapy. Metabolic reprogramming is a fundamental feature of the molecular pathogenesis of glioma and many strategies designed to exploit this process are undergoing preclinical and clinical testing (Zhou and Wahl, 2019). One such strategy includes targeting metabolic pathways that display higher flux in glioma cells relative to normal cells, as exemplified by studies evaluating the radiosensitizing effects of purine nucleotide synthesis inhibition in GBM (NCT04477200) (Zhou et al., 2020). Another strategy entails blocking the synthesis of the oncometabolite 2-hydroxyglutarate, which selectively accumulates in and drives the formation of isocitrate dehydrogenase (IDH) mutated gliomas (NCT02481154) (Mellinghoff et al., 2021). In contrast, our approach reveals potentially a new paradigm: activating, rather than inhibiting, a metabolic pathway in GBM cells to trigger production of a tumor-specific cytotoxic metabolite, EPC.

With regard to clinical translation, we also report a derivative of MM0299 that is both orally bioavailable and penetrates the blood-brain barrier. These findings demonstrate development of lanosterol synthase inhibitors as treatments for glioblastoma or other neurologic conditions.

### Results

### 1. MM0299 Inhibits Mut6 Cell Growth by Binding to Lanosterol Synthase

GFAP-Cre-mediated silencing of the tumor suppressors Trp53, Pten, and Nf1 in murine astrocytes leads to autochthonous tumors, which are histologically comparable to human GBM (Kwon et al., 2008). A glioma stem-like cell (GSC) line derived from GBM tumors arising in this model (hereafter referred to as Mut6) was subsequently used in a high-throughput chemical screen to identify small molecules that impair the proliferation of these GSCs (Shi et al., 2019). MM0299 (1) is a tetracyclic dicarboximide that emerged from this screen and reduces the viability of Mut6 cells in a dose-dependent manner (IC₅₀=0.0182 μM).

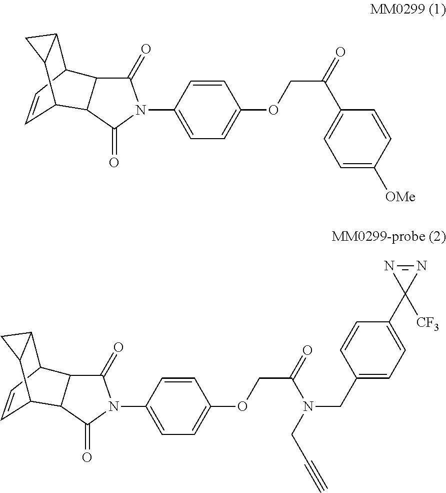 [SMILES: COc1ccc(C(=O)COc2ccc(N3C(=O)C4C5C=CC(C6CC56)C4C3=O)cc2)cc1 ; C#CCN(Cc1ccc(C2(C(F)(F)F)N=N2)cc1)C(=O)COc1ccc(N2C(=O)C3C4C=CC(C5CC45)C3C2=O)cc1]

We sought to understand the mechanism of action (MoA) of MM0299 by identifying which proteins it binds to in Mut6 cells. To accomplish this goal, we synthesized MM0299-probe (2), an analog of MM0299 that maintains anti-proliferative activity in Mut6 (IC₅₀=1.18 μM) and contains both diazirine and alkyne functional groups. Following ultraviolet (UV) light exposure, photoreactive diazirine groups form reactive carbenes that have the potential to react with nearby amino acid residues resulting in the covalent addition of the compound to a bound protein. We incubated Mut6 cells with increasing concentrations of probe 2 either with or without UV treatment (306 nm), and the resulting lysates were reacted with a fluorescent azide in a copper(I)-catalyzed azide-alkyne cycloaddition (commonly referred to as “click” chemistry). Analysis of clicked lysates by SDS-PAGE revealed multiple fluorescent protein bands, which represent proteins covalently bound to probe 2. Several protein bands increased in fluorescent intensity in a dose-dependent manner exclusively in the UV condition, the most prominent of which migrated at approximately 75 kDa (p75).

To determine whether binding to any of the observed bands was related to toxicity, we performed a series of probe-displacement assays by co-incubating cells with a fixed concentration of probe 2 and increasing concentrations of competitor 1. We hypothesized that 1 would cause a dose-dependent reduction in fluorescent band intensity for the putative target. Even though probe 2 bound to multiple proteins, we observed that only p75 fluorescence decreased in a dose-dependent manner. We repeated the probe-displacement assay on 12 structurally related MM0299 derivatives (analogs 3-14, Table 1, see SI for synthesis and structures) and quantified the p75 binding EC₅₀—the concentration of competitor required to displace 50% of probe-bound signal—to generate a structure-binding relationship. In parallel, we determined the anti-proliferative activity for each derivative in a dose-dependent manner to determine its IC₅₀, yielding a structure-activity relationship. After plotting the relationship between p75 probe displacement EC₅₀ and anti-proliferative activity IC₅₀ we noted that the slope of the best-fit line was 1.06, demonstrating that the EC₅₀ for probe competition is comparable to the IC₅₀ for anti-proliferative activity. In addition, there was a significant correlation (R²=0.864, p<0.0001) between p75 binding and cytotoxic activity over a 1000-fold range in potency, providing evidence that p75 is the functional target of MM0299.

To determine the identity of p75, we performed large-scale affinity purifications of probe-bound proteins. Mut6 cells were incubated with 0.1 μM probe 2 and either DMSO or 1.0 μM of an active competitor, analog 11 (IC₅₀=0.000791 μM), followed by UV crosslinking. Probe-bound proteins were biotinylated by performing a click reaction on lysates using a biotin-azide and then subjected to affinity purification with immobilized streptavidin. Proteins eluted from streptavidin beads were digested with trypsin, and the resulting peptides were identified using liquid chromatography-mass spectrometry (LC-MS). Analysis of LC-MS data from the above purifications revealed that lanosterol synthase (Lss) was the most enriched protein in the probe-only purification, displaying approximately 15.5-fold enrichment relative to the probe+competitor purification. These data indicate that p75 is Lss (MW=83.1 kDa). Remarkably, no other proteins were enriched more than 2-fold suggesting that MM0299 has few alternative targets.

We used a combination of pharmacology and genetics to further test whether p75 is indeed Lss. First, we asked if Ro 48-8071, a structurally distinct LSS inhibitor, was able to displace MM0299-probe (2) in a fashion similar to the probe-displacement assays described above for the MM0299 series. Ro 48-8071 displaced MM0299-probe in a dose-dependent fashion, indicated by a decrease in p75 band intensity with increasing concentrations of Ro 48-8071. In addition, Ro 48-8071 impaired Mut6 cell proliferation with a similar concentration (IC₅₀=0.0112 μM) to that observed for p75 binding (EC₅₀=0.00248 μM). To further investigate if p75 is Lss, we evaluated probe binding in HEK293T cells that ectopically express FLAG epitope-tagged human LSS. In these cells, we observed an increased p75 band intensity following incubation and crosslinking with probe 2 that was diminished by the addition of excess competitor 1, demonstrating specificity. Taken together, these results provide evidence that p75, implicated as the direct target of MM0299, is Lss.

### 2. MM0299 (1) Inhibits Lss Activity Leading to Canonical Pathway Repression and Shunt Pathway Induction.

Inhibitors of LSS block the canonical cholesterol biosynthetic pathway (referred to as the Bloch and Kandutsch-Russell pathways) and concomitantly stimulate the production of 24(S),25-epoxycholesterol (EPC) through a “shunt” pathway (Mark et al., 1996; Morand et al., 1997). For sterol synthesis, squalene, a symmetric polyene hydrocarbon intermediate in the cholesterol biosynthetic pathway, is oxidized by squalene epoxidase (SQLE) in the presence of NADPH and oxygen to generate (S)-2,3-oxidosqualene (OS), which in turn is converted by LSS into lanosterol. LSS inhibition shunts OS back to SQLE, which catalyzes a second oxidation event yielding the C2-symmetric (S,S)-2,3:22,23-dioxidosqualene (DOS). DOS is a preferred substrate for LSS and, even in the presence of an LSS inhibitor, DOS is converted by LSS into 24(S),25-epoxylanosterol (EPL) (Boutaud et al., 1992). EPL is further converted into EPC by the same enzymes that convert lanosterol into cholesterol (Nelson et al., 1981a; Nelson et al., 1981b). The overall consequence of LSS inhibition is reduced levels of canonical pathway intermediates and increased levels of shunt pathway intermediates. Therefore, to test the hypothesis that 1 is an LSS inhibitor, we quantified key intermediates in both the canonical and shunt pathways after treatment with varying doses of compound 1. Treatment of Mut6 cells with compound 1 led to a dose-dependent increase (up to 6.48-fold) in the primary LSS substrate, OS, and a dose-dependent decrease (up to 24.5-fold) in the product lanosterol (IC₅₀=0.0455 μM). Treatment with compound 1 also led to a dose-dependent increase in the shunt pathway intermediates DOS and EPL (up to 17.9-fold and 25.6-fold, respectively). Levels of EPC, the terminal product of the shunt pathway, peaked at 390 ng/mg protein (13.9-fold increase) when cells were treated with 0.1 μM compound 1, but steadily decreased at higher concentrations of 1. We considered that the decrease in EPC at higher concentrations might be the result of complete inhibition of LSS; however, the levels of DOS and EPL continued to increase at doses where EPC was decreasing. These data suggest that the reduction of EPC levels at higher doses is more likely the consequence of catabolism or efflux rather than reduced synthesis. Pertinent to this hypothesis, in astrocytes, EPC stimulates the expression of sterol transporters including ABCA1 and is itself readily effluxed (Wong et al., 2007). We hypothesize that a threshold level of EPC, reached at higher concentrations of 1, activates transporter expression, leading to EPC efflux and the observed decrease in EPC levels. Taken together, we conclude that inhibition of Lss by MM0299 (1) leads to a dose-dependent decrease in canonical intermediates and an increase in shunt pathway intermediates.

We next evaluated whether MM0299 (1) and related analogs could directly inhibit recombinant human LSS in a cell-free in vitro reaction. Recombinant LSS purified from E. coli converted OS to lanosterol in a time- and dose-dependent manner. Of note, lanosterol production was saturated over time, which may be the consequence of substrate depletion or product inhibition. Regardless, we evaluated enzymatic activity over 30 minutes with 64 μM of substrate, conditions under which the reaction remained linear. Under these conditions, compound 1 inhibits LSS in vitro activity in a dose-dependent manner with an IC₅₀=2.22 μM. Similar experiments with three different analogs (3, 6, 14) support the notion that inhibition of Lss enzyme activity correlates with anti-GBM activity in Mut6 cells. It was notable that the concentration of compound required to inhibit LSS activity in vitro is substantially higher than the concentration required to block cell growth. One possible explanation for this difference is that the conditions of the in vitro reaction, which includes non-ionic detergents in an aqueous buffer, influence substrate or compound solubility or binding.

An outstanding question regarding the shunt pathway is how LSS can still convert DOS into EPL in the presence of high concentrations of an inhibitor. Kinetic in vitro experiments have shown that the conversion of DOS into EPL is faster than OS into lanosterol, suggesting that DOS is the preferred substrate (Boutaud et al., 1992). To address this question, we assessed the efficacy of in vitro conversion of DOS to EPL by LSS. Consistent with prior results, the rate of DOS conversion to EPL was 2.67-fold faster than conversion of OS to lanosterol. Nonetheless, MM0299 (1) inhibited DOS conversion to EPL at a potency comparable to that of OS to lanosterol (IC₅₀=1.60 and 2.22 μM). Therefore, the conversion of DOS to EPL by LSS in the presence of MM0299 is not simply explained by an increased affinity LSS for DOS. A more likely explanation emerges from our observation that in cells treated with 1, DOS levels are 375-fold higher than OS. The effective concentration of DOS in the ER membrane may even be higher, thus limiting access of the inhibitor to the enzyme active site.

### 3. Induction of the Shunt Pathway by MM0299 Contributes to Toxicity in Mut6 Cells.

Next, we asked whether the increased EPC levels that result from LSS inhibition by MM0299 contribute to the anti-proliferative activity observed in Mut6 cells. EPC is a potent ligand for the LXR nuclear hormone transcription factors that promote cholesterol efflux by upregulating cholesterol transporters such as ABCA1 and ABCG1 (Janowski et al., 1999; Janowski et al., 1996; Lehmann et al., 1997; Willy et al., 1995). In addition, EPC blocks activation of SREBPs, master transcription factors that promote expression of enzymes involved in cholesterol biosynthesis and uptake (Horton et al., 2003; Radhakrishnan et al., 2007). Using mRNA sequencing, we found that treatment of Mut6 cells with 1 caused an upregulation of LXR target genes including Abca1, Abcg1, and Srebf1 and a concomitant downregulation of SREBP target genes, indicating that the levels of EPC produced following 1 treatment were sufficient to influence these pathways (Horton et al., 2003; Willy et al., 1995).

To better gauge the role of EPC in the anti-cancer activity of 1, we sought to determine whether the levels of EPC achieved in cells treated with 1 are sufficient for toxicity. When cells were incubated with a poly-deuterated derivative of 24(S),25-epoxylanosterol (d6-S-EPL), d6-EPC was produced in a time-dependent manner, and d6-S-EPL inhibited Mut6 growth with an IC₅₀ of 0.483 μM. Notably, the levels of d6-EPC produced when cells were incubated with an efficacious dose of d6-S-EPL (1 μM) were comparable to the EPC levels detected after treatment with a lethal dose of 1 (0.1 μM) (390 ng d6-EPC/mg protein and 342 ng EPC/mg protein, respectively). These data demonstrate that the EPC synthesized following treatment with 1 is sufficient to block cell growth.

Taken together, we concluded that the levels of EPC resulting from 1 treatment are sufficient to downregulate SREBP target genes, upregulate LXR target genes, and block cell proliferation. Through its coordinated action on the critical transcriptional regulators of cholesterol homeostasis, LXRs and SREBPs, EPC can cause lethal reductions in cellular cholesterol levels. To test this hypothesis, we evaluated cell proliferation following treatment with increasing doses of either S-EPL or 1 in the presence of exogenous lanosterol or cholesterol conjugated to methyl beta-cyclodextrin (MCD), which aids sterol solubility and delivery. Supplementing cells with either lanosterol or cholesterol rescued Mut6 cells from the toxic effects of either S-EPL or 1. These observations provide evidence that the anti-proliferative activity of 1 is due to the production of EPC and the resulting depletion of cellular cholesterol.

To assess whether EPC was necessary for MM0299 activity, we considered pharmacologic tools that could selectively block the shunt pathway. In the context of LSS inhibition, the production of DOS requires two cycles of oxidation by squalene epoxidase (SQLE), whereas OS only requires one. Therefore, we reasoned that co-administration of an SQLE inhibitor might have a greater effect on DOS levels and impede flux through the shunt pathway. Consistent with this prediction, we found that co-treatment with sublethal doses of the SQLE inhibitor, NB-598, completely blocked the accumulation of shunt pathway metabolites following 1 treatment but did not lower canonical sterol levels (Horie et al., 1990). Taken together, we concluded that NB-598 is a pharmacologic tool to selectively block the shunt pathway and assess the requirement for EPC for MM0299 activity. We found that in a dose-dependent manner, NB-598 rescued 1 toxicity, reflected by an 11.8-fold increase in the IC₅₀. By contrast, NB-598 did not rescue the activity of bortezomib, a compound with a mechanism unrelated to cholesterol synthesis or TASIN-30, an inhibitor of emopamil binding protein (EBP), an enzyme downstream of LSS in cholesterol biosynthesis (Theodoropoulos et al., 2020). These observations are consistent with the hypothesis that inhibition of LSS and the consequent increase in EPC are responsible for toxicity at low nanomolar range. We noted that 1 remained toxic to Mut6 cells at higher concentrations despite the addition of NB-598. Toxicity at these concentrations, which are not likely to be pharmacologically achievable, is unlikely to be a consequence of EPC production, and more likely to be the result of sterol depletion through direct inhibition of cholesterol biosynthesis. Notwithstanding, our combined observations led us to conclude that the specific induction of the shunt metabolite EPC is both necessary and sufficient for the most potent cytotoxic effects of MM0299 in Mut6 cells.

### 4. An Evaluation of LSS Selectivity for MM0299 and a Known LSS Inhibitor

Enzyme selectivity is a major challenge in the development of inhibitors for the post-squalene cholesterol biosynthetic pathway (Korade et al., 2016; Moebius et al., 1998; Wages et al., 2018). This poly-pharmacology may be particularly problematic for the use of LSS inhibitors as anti-GBM agents because inhibition of downstream biosynthetic enzymes would block production of the key metabolite, EPC (Rabelo et al., 2017). Hence, we systematically compared the off-target profiles for 1 to Ro 48-8071, an LSS inhibitor that has undergone extensive pharmacologic and biophysical evaluation, including structural determination in complex with LSS (Morand el al., 1997; Thoma et al., 2004).

Like 1, Ro 48-8071 treatment led to a dose-dependent increase in shunt pathway intermediates, and the peak levels of EPC resulting from Ro 48-8071 treatment correlated with toxicity in Mut6 cells (IC₅₀=0.0112 μM). Notably, the peak levels of EPC following Ro 48-8071 treatment were only 62% of those found after 1 treatment. This latter observation raised the hypothesis that Ro 48-8071 inhibited one or more enzymes downstream of LSS thereby blunting EPC synthesis. To evaluate whether Ro 48-8071 inhibits the synthesis of EPC downstream of EPL, we cultured cells with isotopically labeled EPL (d6-EPL) and measured d6-EPC production following compound treatment. Ro 48-8071 inhibited the production of d6-EPC while 1 had no effect on d6-EPC production. These data suggest that Ro 48-8071 inhibits LSS but may also have off-targets in the sterol synthesis pathway downstream of LSS thereby blunting EPC synthesis. For small molecules that inhibit enzymes downstream of LSS, our prediction is that lanosterol would not be efficiently converted to cholesterol and therefore would not rescue toxicity as effectively as cholesterol. Consistent with our hypothesis, cholesterol provided a greater degree of rescue for Ro-48-8071 (479.4-fold increase in the IC₅₀) than exogenous lanosterol (21.7-fold increase in the IC₅₀). By comparison, exogenous lanosterol and cholesterol provided a complete and equivalent degree of rescue in cells treated with 1. Taken together, these findings demonstrate that 1 does not inhibit enzymes downstream of LSS in the cholesterol synthesis pathway, and as a result, more efficiently produces the toxic metabolite, EPC.

To enable the unbiased identification of potential Ro 48-8071 off-targets, we synthesized a click chemistry-amenable derivative of Ro 48-8071. Ro 48-8071 contains a photoreactive benzophenone that, following UV exposure, is predicted to form a reactive triplet diradical capable of covalently modifying nearby amino acids in a bound protein. We replaced the terminal alkene group on Ro 48-8071 with a terminal alkyne (hereafter referred to as Ro-alkyne) that served as a functional handle for click chemistry reactions. After determining that Ro-alkyne maintained anti-proliferative activity against Mut6 (IC₅₀=0.396 μM), we analyzed cellular binding partners for Ro-alkyne. We identified multiple probe- and UV-dependent bands that were displaced with co-incubation of 10 μM Ro 48-8071; however, a band corresponding to LSS was only visible after ectopic expression of LSS in HEK293T cells.

To determine the identity of putative Ro 48-8071 off-targets, we performed affinity purifications of Ro-alkyne-bound proteins in the presence or absence of 10 μM Ro 48-8071. In striking contrast to MM0299, 130 proteins displayed greater enrichment than Lss (#131, 1.3-fold enrichment) in the Ro-alkyne-only condition, implicating numerous off-targets. Among the most enriched Ro-alkyne-bound proteins were three enzymes in the cholesterol biosynthetic pathway: Ebp (#2, 8.4-fold enrichment), Lbr (#5, 3.1-fold enrichment), and Dhcr7 (#7, 2.5-fold enrichment). Taken together, these results demonstrate that, unlike M0299, Ro-48-8071 has numerous off-targets including multiple enzymes involved in cholesterol biosynthesis.

Inhibition of these sterol biosynthetic enzymes might provide an explanation for our observation that Ro-48-8071 has a blunted increase in EPC following LSS inhibition. To evaluate how 1 and Ro 48-8071 influence these enzymes, we used a suite of small molecule tools that explore the cellular binding profiles for small molecules against several cholesterol biosynthetic enzymes (Theodoropoulos et al., 2020). We first tested 1 and Ro 48-8071 binding to EBP by incubating HCT116 cells with 1 μM TASIN-2 (an EBP-specific photoaffinity probe) in the presence or absence of increasing doses of TASIN-30 (an EBP-specific inhibitor), 1, or Ro 48-8071, followed by UV treatment and fluorescent dye conjugation. Consistent with the proteomics data, Ro 48-8071 and TASIN-30, but not 1, displaced TASIN-2 (band migrating at approximately 20 kDa). Additionally, Ro 48-8071 treatment caused the accumulation of 8,9-dehydrocholesterol (8,9-DHC), a non-canonical sterol that accumulates following genetic or pharmacologic EBP inhibition (Braverman et al., 1999; Theodoropoulos et al., 2020), while treatment with 1 had no effect on cellular 8,9-DHC levels. Taken together, these data provide evidence that Ro 48-8071 binds to and inhibits Ebp in cells.

Next, we performed analogous experiments using a probe (known as 4C12) specific for 7-dehydrocholesterol reductase (Dhcr7 in mouse or DHCR7 in human) (Theodoropoulos et al., 2020). The crosslinked Dhcr7 band, which migrates at 40 kDa, was displaced by 4C12 and Ro 48-8071; however, 1 did not displace Dhcr7-probe at any tested dose. Consistent with inhibition, the cellular levels of 7-dehydrodesmosterol, a Dhcr7 substrate, accumulated following treatment with Ro 48-8071 but not 1. Compounds that target EBP and DHCR7 can also inhibit DHCR24, which converts desmosterol into cholesterol (Theodoropoulos et al., 2020) (Wages et al., 2018). Although we do not have chemical tools to evaluate direct DHCR24 binding, we analyzed the levels of desmosterol in cells treated with 1 and Ro 48-8071. We observed accumulation of desmosterol in cells treated with high doses of Ro 48-8071, but not 1, supporting the hypothesis that Ro 48-8071 also inhibits Dhcr24. Taken together, these data demonstrate that while 1 and Ro 48-8071 both inhibit Lss, Ro 48-8071 also inhibits, in increasing order of potency, the activity of Ebp, Dhcr7, and Dhcr24.

Our observations demonstrate that Ro-48-8071 has substantially more off-targets than 1, and some of these off-targets blunt the production of EPC, the anti-proliferative metabolite. As a consequence, even though Ro-48-8071 inhibits LSS, its anti-proliferative effects are more likely to be multifactorial. To evaluate the contribution of EPC for Ro 48-8071-induced toxicity, we co-incubated Ro 48-8071 with increasing concentrations of NB-598. NB-598 was modestly protective against Ro 48-8071-induced toxicity (3.2-fold increase in Ro 48-8071 IC₅₀); however, this protective effect was not dose-dependent and was lost at higher concentrations of NB-598. By comparison, toxicity from 1, was rescued by NB-598 in a dose-dependent fashion, resulting in a maximal 11.8-fold increase in the IC₅₀.

### 5. MM0299 is Efficacious in Human GSC Lines.

Given that our studies of MM0299 have primarily utilized Mut6 cells—a GSC line from a single genetically-defined mouse model—we next sought to evaluate the anti-proliferative activity and mechanism of action of MM0299 in human GSC lines with different genetic drivers. We developed two GSC lines from patients at UT Southwestern Medical Center, denoted as UTSW63 and UTSW71. UTSW63 is derived from the resected tumor of a 71-year-old male patient with malignant neoplasm of the temporal lobe. Genomic alterations in bonafide GBM cancer drivers were found in a genomic analysis of the resected tumor. These included the expected loss of the tumor suppressors TP53 (deletion of 17p13.3-p11.1) and RBI (deletion of 13q13.11-14.3 and c.1215+1G>A, a canonical splice donor site mutation predicted to result in a frameshift) as well as an amplification of EGFR (93 copies of 7p11.2). UTSW71, derived from a 52-year-old male patient with glioblastoma, harbors alterations in distinct cancer drivers including PTEN (deletion of 10q21.1-q26.3 and P95S mutation) along with amplifications of regions that contain the oncogenes CDK4 (34 copies of 12q13.3-q14.1), MDM2 (89 copies of 12q15), and c-MYC (51 copies of 8q24.13-q24.21).

Like Mut6 cells, both UTSW63 and UTSW71 grow as neurospheres in serum-free medium (data not shown). MM0299 impedes proliferation of both UTSW63 and UTSW71, with an IC₅₀ of 0.0222 μM and 0.0212 μM, respectively. MM0299 toxicity in the two human GSC lines is completely rescued by exogenous MCD-conjugated lanosterol or cholesterol. A naturally occurring mechanism for the import of extracellular cholesterol is the uptake of circulating low-density lipoprotein (LDL). Therefore, we investigated whether human LDL could rescue MM0299 toxicity in human GBM cells. Addition of 20 μg/ml human LDL completely rescued the toxicity of 1 in both UTSW63 and UTSW71 cells. To investigate the role of the shunt pathway in MM0299 toxicity, we again turned to the tool compound NB-598. NB-598 is highly toxic in UTSW63 and UTSW71 with an IC₅₀ of 5.56 nM and 8.26 nM, respectively. Lower doses of NB-598, which do not affect cell growth, rescued toxicity of 1 as evidenced by a 15.0- and 21.9-fold increase in IC₅₀ for UTSW63 and UTSW71, respectively. Together, the data indicate that 1 is efficacious in cultured human GSC lines through the upregulation of EPC via the shunt pathway and consequent depletion of the cellular cholesterol pool.

### 6. Analog 13 is Bioavailable and Capable of Crossing the Blood-Brain Barrier

Having established the mechanism and mode of action for MM0299, we sought to identify and evaluate an analog for bioavailability in the brain, a prerequisite for in vivo studies in glioblastoma. in vitro and in vivo pharmacological evaluation of a series of analogs (Table 1) led us to pursue analog 13 as a candidate molecule to activate the shunt pathway in mouse brain.

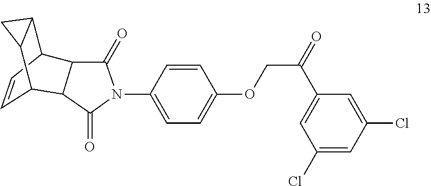 [SMILES: O=C(COc1ccc(N2C(=O)C3C4C=CC(C5CC45)C3C2=O)cc1)c1cc(Cl)cc(Cl)c1]

Analog 13 competed p75/LSS (EC₅₀=0.0287 μM), has drug-like physicochemical properties (MW=468 Da, C Log P=4.3, Log P=3.8, tPSA=64), induced the upregulation of EPC, and maintained anti-proliferative activity in both Mut6 cells (IC₅₀=0.0443 μM) and the human GSC line UTSW63 (IC₅₀=0.0293 μM). Analog 13 was stable in plasma (>24 h) and displayed superior metabolic stability in comparison to other analogs when incubated with murine S9 fractions (Table 1). After dosing mice with 13 via intravenous injection (IV) at 5 mg/kg or orally (PO) at 20 mg/kg, pharmacokinetic (PK) analysis indicated oral bioavailability, defined by AUCₒᵣₐₗ/AUCᵢᵥ×Doseᵢᵥ/Doseₒᵣₐₗ, of 39% in plasma and 58% in the brain. Crucially, 20 mg/kg of 13 dosed PO also displayed brain exposure with a total Cₘₐₓ of 5 μM (2381 ng/ml), free Cₘₐₓ in brain of 14 nM, and a brain to blood ratio of 1.8 (defined as free AUC_{brain}/free AUCₚₗₐₛₘₐ). This compares favorably to free IC₅₀ values calculated for anti-proliferative activity against human GSC lines (3 nM) using a measured fraction unbound (fu) for culture media of 0.11. Next, we used mass spectrometry to quantify the levels of EPC following treatment with 13 in plasma and brain. Plasma levels of EPC increased following administration of a single dose of 20 mg/kg in a time dependent manner. In brain, we readily detected desmosterol but did not detect EPC, even at time points where plasma EPC was markedly elevated. Cholesterol biosynthetic flux in the adult brain is expected to be low, which might explain the absence of detectable EPC. Alternatively, the levels of 13 in the brain, despite our predictions, may be inadequate to inhibit LSS. To distinguish between these possibilities, we asked whether we could detect EPC in orthotopic xenograft tumors derived from UTSW63 cells. 25 days following intracranial injection of UTSW63 cells, mice were dosed orally with vehicle or 20 mg/kg 13 for 3 days on a once or twice daily dosing schedule. Following treatment, we analyzed EPC levels and observed a dose-dependent increase in EPC levels in both serum and GBM tumors. These findings demonstrate that 13 administration leads to a selective EPC induction in glioblastoma cells versus normal brain and that EPC can be used as a pharmacodynamic marker to guide the optimization of MM0299 for further pre-clinical evaluation.

### DISCUSSION

Our investigation into MM0299 mechanism of action reveals a new approach to target tumor cell metabolism for brain tumor therapy. Metabolic reprogramming is a fundamental feature of the molecular pathogenesis of glioma and many strategies designed to exploit this process are undergoing preclinical and clinical testing (Zhou and Wahl, 2019). One such strategy includes targeting metabolic pathways that display higher flux in glioma cells relative to normal cells, as exemplified by studies evaluating the radiosensitizing effects of purine nucleotide synthesis inhibition in GBM (NCT04477200) (Zhou et al., 2020). Another strategy entails blocking the synthesis of the oncometabolite 2-hydroxyglutarate, which selectively accumulates in and drives the formation of isocitrate dehydrogenase (IDH) mutated gliomas (NCT02481154) (Mellinghoff et al., 2021). In contrast, our approach relies on a new paradigm: activating, rather than inhibiting, a metabolic pathway in GBM cells to trigger production of a tumor-specific cytotoxic metabolite, EPC.

Like most cells, cancer cells regulate their unesterified cholesterol pool through a balance of peripheral uptake, synthesis, and catabolism. As such, blocking cholesterol synthesis triggers well-described negative feedback pathways that maintain homeostasis via increased uptake of low-density lipoprotein (LDL). LDL particles, however, do not cross the blood brain barrier, and therefore, the brain must synthesize its own cholesterol. The bulk of cholesterol synthesis in the brain occurs during embryonic development and rates of cholesterol synthesis are dramatically lowered in the adult brain (Dietschy, 2009). Dividing GBM cells, however, are expected to have increased cholesterol demand relative to adult neurons and correspondingly rely on both de novo cholesterol synthesis and uptake of exogenous cholesterol (synthesized by astrocytes) to meet the demands for proliferation (Sassi et al., 2021). LSS inhibition has the potential to exploit a difference in cholesterol synthetic flux between normal brain and tumors. LSS inhibition shifts sterol flux from cholesterol synthesis to EPC synthesis, which ultimately blocks cell growth, and the prediction is that tumors will synthesize more EPC than normal brain. Consistent with this hypothesis, we found that in vivo administration of 13 led to no detectable EPC production in normal brain but dose-dependent increases in EPC in orthotopic glioblastoma tumors. Hence, inhibiting cholesterol biosynthesis via LSS inhibition may selectively target rapidly dividing GBMs with elevated sterol synthesis.

Currently, there are multiple lines of evidence that reducing cellular cholesterol pools would impede the progression of glioblastoma. The synthetic LXR agonists GW3965 and LXR-623 upregulate expression of the cholesterol transporter ABCA1, which promotes cholesterol efflux and reduces cellular cholesterol pools in GBM cells (Guo et al., 2011; Villa et al., 2016). Accordingly, LXR-623, which is brain-penetrant, delays progression of orthotopic glioblastoma tumors and extends the overall survival of mice harboring these tumors (Villa et al., 2016). There are, however, mechanism-related adverse events that have impeded the clinical development of LXR agonists for the treatment of glioblastoma or any other indication. For example, LXR agonists stimulate fatty acid synthesis, which leads to clinically unacceptable elevations in circulating triglycerides and accelerates the development of fatty liver disease (Schultz et al., 2000; Joseph et al., 2002, Bradley et al., 2007).

Alternative strategies to reduce cholesterol levels in GBM include inhibitors of critical enzymes in the sterol biosynthetic pathway. Cellular cholesterol levels are regulated within a narrow concentration range at the level of synthesis, uptake of LDL, and catabolism (or efflux). Reduced cholesterol levels are sensed by the SREBP cleavage activating protein (SCAP), which promotes the proteolytic cleavage and activation of SREBP-2 (Brown et al., 2018). Activated SREBP-2 translocates to the nucleus and promotes the transcription of genes required for cholesterol synthesis and cholesterol uptake. As such, these feedback pathways counterbalance the cholesterol-lowering effects of most enzymatic inhibitors of cholesterol biosynthesis (Brown et al., 1978).

Lanosterol synthase inhibition, however, is unique because it reduces cholesterol synthesis without stimulating these negative feedback pathways. LSS converts linear oxidosqualene into lanosterol, the first sterol ring-containing intermediate in the biosynthesis of cholesterol. Unlike other post-squalene enzymes, inhibition of LSS blocks the synthesis of cholesterol without causing the accumulation of sterol intermediates. Instead, partial inhibition of LSS diverts sterol flux into a “shunt” pathway that culminates in the synthesis of the non-canonical sterol EPC. EPC accumulation triggers a feedback mechanism that blocks the activation of SREBPs, thus preventing cells from upregulating the genes for de novo cholesterol biosynthesis and cholesterol uptake (Radhakrishnan et al., 2007). Additionally, EPC is a potent modulator of LXRs—nuclear hormone transcription factors whose activation induces cholesterol metabolism and efflux (Janowski et al., 1996; Janowski et al., 1999). As an endogenous LXR ligand, EPC does not drive fatty acid synthesis (Rowe et al., 2003) and LSS inhibitors do not manifest the adverse events characteristic of synthetic LXR agonists in animals. Thus, LSS inhibition has the potential to reduce the cellular cholesterol pool in multiple ways, including the direct inhibition of de novo cholesterol synthesis, inhibition of feedback pathways that promote cholesterol biosynthesis, and promotion of efflux through activation of LXR. Here, we show that synthesis of EPC through the shunt pathway was both necessary and sufficient to inhibit the proliferation of glioblastoma cells by the LSS inhibitor, MM0299.

Recent evidence also suggests that LSS is a potential glioblastoma target. Allis and colleagues reported that MI-2, a small molecule LSS inhibitor that increases EPC levels, displayed anti-proliferative activity in glioma. Previously, MI-2 was reported to inhibit the protein-protein interaction between Menin and MLL, which are important epigenetic regulators in leukemia (Grembecka et al., 2012; Shi et al., 2012). The activity of MI-2 in glioma, however, did not involve Menin or MLL but rather was the consequence of direct inhibition of LSS. Their findings not only nominated LSS as a potential target in GBM but also reinforced the importance of ongoing target deconvolution of compounds evaluated in preclinical cancer studies (Phillips et al., 2019).

Most LSS inhibitors have been developed primarily with the intent of reducing LDL levels to treat atherosclerosis and data supporting their efficacy in cancers is sparse. One limitation to their development has been the challenge in evaluating LSS selectivity. In fact, most reported LSS small molecule inhibitors are amphiphilic amine drugs that bind to many other proteins including multiple enzymes in the post-squalene cholesterol biosynthetic pathway (Rabelo et al., 2017). This polypharmacology is most prominent amongst LSS, EBP, and the sterol reductases DHCR7, DHCR14, and DHCR24 (Wages et al., 2018; Korade et al., 2016; Moebius et al., 1998). These enzymes share the need to stabilize a carbocationic charge generated during the transformation of highly lipophilic sterols and may explain why amphiphilic amines, which are protonated at physiological pH, have the capacity to bind either or all of these enzymes. Given the similarities in substrate structure, and the close physical proximity of these enzymes, photochemical probes whose binding and activity can be assigned to specific cholesterol biosynthetic enzymes have proven to be useful tools for studying these enzymes (Theodoropoulos et al., 2020). Using a combination of these photochemical probes and sterol mass spectrometry, we compared the selectivity of MM0299 to the well-characterized LSS inhibitor Ro 48-8071. While Ro 48-8071 bound to and inhibited LSS, our studies indicate that it also binds to numerous off-targets and inhibits multiple enzymes in the cholesterol pathway including EBP and DHCR7, which in turn blunts the production of EPC. A similar off-target survey for MM0299 revealed no off-targets and no evidence for binding to or inhibition of other cholesterol biosynthetic enzymes.

Our studies reinforce LSS as a target in glioblastoma and identify MM0299 as a highly selective LSS inhibitor. In addition to the aforementioned cholesterol lowering effects of EPC, EPC has also been shown to stimulate the formation of myelinating oligodendrocytes via an unknown mechanism (Hubler et al., 2021), and promote midbrain dopaminergic neurogenesis via LXR activation (Theofilopoulos et al., 2013; Theofilopoulos et al., 2019). Neurodegenerative conditions including multiple sclerosis (MS) and Parkinson's disease (PD) are the consequence of demyelination of neurons and loss of midbrain dopaminergic neurons, respectively. New therapies for these diseases are needed and brain penetrant MM0299 derivatives that induce EPC production have clinically utility in these diseases as well.

A major challenge in the development of therapies for GBM or other neurologic conditions is to identify small molecule drugs that can cross the blood-brain barrier. Here, we described derivatives of MM0299 that are orally bioavailable and blood-brain barrier penetrant. These derivatives lead to an increase in EPC in orthotopic xenograft tumors but not normal brain.

### CITATIONS

Boutaud, O., Dolis, D., and Schuber, F. (1992). Preferential cyclization of 2,3(S):22(S),23-dioxidosqualene by mammalian 2,3-oxidosqualene-lanosterol cyclase. Biochem Biophys Res Commun 188, 898-904. 10.1016/0006-291x(92)91140-1.
Bradley, M. N., Hong, C., Chen, M., Joseph, S. B., Wilpitz, D. C., Wang, X., Lusis, A. J., Collins, A., Hseuh, W. A., Collins, J. L., et al. (2007). Ligand activation of LXR beta reverses atherosclerosis and cellular cholesterol overload in mice lacking LXR alpha and apoE. J Clin Invest 117, 2337-2346. 10.1172/JC131909.
Braverman, N., Lin, P., Moebius, F. F., Obie, C., Moser, A., Glossmann, H., Wilcox, W. R., Rimoin, D. L., Smith, M., Kratz, L., et al. (1999). Mutations in the gene encoding 3 beta-hydroxysteroid-delta 8, delta 7-isomerase cause X-linked dominant Conradi-Hunermann syndrome. Nat Genet 22, 291-294. 10.1038/10357.
Brown, M. S., Faust, J. R., Goldstein, J. L., Kaneko, I., and Endo, A. (1978). Induction of 3-hydroxy-3-methylglutaryl coenzyme A reductase activity in human fibroblasts incubated with compactin (ML-236B), a competitive inhibitor of the reductase. J Biol Chem 253, 1121-1128.
Brown, M. S., Radhakrishnan, A., and Goldstein, J. L. (2018). Retrospective on Cholesterol Homeostasis: The Central Role of Scap. Annu Rev Biochem 87, 783-807. 10.1146/annurev-biochem-062917-011852.
Dietschy, J. M. (2009). Central nervous system: cholesterol turnover, brain development and neurodegeneration. Biol Chem 390, 287-293. 10.1515/BC.2009.035.
Grembecka, J., He, S., Shi, A., Purohit, T., Muntean, A. G., Sorenson, R. J., Showalter, H. D., Murai, M. J., Belcher, A. M., Hartley, T., et al. (2012). Menin-MLL inhibitors reverse oncogenic activity of MLL fusion proteins in leukemia. Nat Chem Biol 8, 277-284. 10.1038/nchembio.773.
Guo, D., Reinitz, F., Youssef, M., Hong, C., Nathanson, D., Akhavan, D., Kuga, D., Amzajerdi, A. N., Soto, H., Zhu, S., et al. (2011). An LXR agonist promotes glioblastoma cell death through inhibition of an EGFR/AKT/SREBP-1/LDLR-dependent pathway. Cancer Discov 1, 442-456. 10.1158/2159-8290.CD-11-0102.
Horie, M., Tsuchiya, Y., Hayashi, M., Iida, Y., lwasawa, Y., Nagata, Y., Sawasaki, Y., Fukuzumi, H., Kitani, K., and Kamei, T. (1990). NB-598: a potent competitive inhibitor of squalene epoxidase. J Biol Chem 265, 18075-18078.
Horton, J. D., Shah, N. A., Warrington, J. A., Anderson, N. N., Park, S. W., Brown, M. S., and Goldstein, J. L. (2003). Combined analysis of oligonucleotide microarray data from transgenic and knockout mice identifies direct SREBP target genes. Proc Natl Acad Sci USA 100, 12027-12032. 10.1073/pnas.1534923100.
Hubler, Z., Friedrich, R. M., Sax, J. L., Allimuthu, D., Gao, F., Rivera-Le6n, A. M., Pleshinger, M. J., Bederman, I., and Adams, D. J. (2021). Modulation of lanosterol synthase drives 24,25-epoxysterol synthesis and oligodendrocyte formation. Cell Chemical Biology 28, 866-875.e865. 10.1016/j.chembiol.2021.01.025.
Janowski, B. A., Grogan, M. J., Jones, S. A., Wisely, G. B., Kliewer, S. A., Corey, E. J., and Mangelsdorf, D. J. (1999). Structural requirements of ligands for the oxysterol liver X receptors LXRalpha and LXRbeta. Proc Natl Acad Sci USA 96, 266-271. 10.1073/pnas.96.1.266.
Janowski, B. A., Willy, P. J., Devi, T. R., Falck, J. R., and Mangelsdorf, D. J. (1996). An oxysterol signalling pathway mediated by the nuclear receptor LXR alpha. Nature 383, 728-731. 10.1038/383728a0.
Joseph, S. B., Laffitte, B. A., Patel, P. H., Watson, M. A., Matsukuma, K. E., Walczak, R., Collins, J. L., Osborne, T. F., and Tontonoz, P. (2002). Direct and indirect mechanisms for regulation of fatty acid synthase gene expression by liver X receptors. J Biol Chem 277, 11019-11025. 10.1074/jbc.M111041200.
Korade, Z., Kim, H. Y., Tallman, K. A., Liu, W., Koczok, K., Balogh, I., Xu, L., Mimics, K., and Porter, N. A. (2016). The Effect of Small Molecules on Sterol Homeostasis: Measuring 7-Dehydrocholesterol in Dhcr7-Deficient Neuro2a Cells and Human Fibroblasts. J Med Chem 59, 1102-1115. 10.1021/acs.jmedchem.5b01696.
Kwon, C. H., Zhao, D., Chen, J., Alcantara, S., Li, Y., Burns, D. K., Mason, R. P., Lee, E. Y., Wu, H., and Parada, L. F. (2008). Pten haploinsufficiency accelerates formation of high-grade astrocytomas. Cancer Res 68, 3286-3294. 10.1158/0008-5472.CAN-07-6867.
Lehmann, J. M., Kliewer, S. A., Moore, L. B., Smith-Oliver, T. A., Oliver, B. B., Su, J. L., Sundseth, S. S., Winegar, D. A., Blanchard, D. E., Spencer, T. A., and Willson, T. M. (1997). Activation of the nuclear receptor LXR by oxysterols defines a new hormone response pathway. J Biol Chem 272, 3137-3140. 10.1074/jbc.272.6.3137.
Madhusudhan, N., Hu, B., Mishra, P., Calva-Moreno, J. F., Patel, K., Boriack, R., Ready, J. M., and Nijhawan, D. (2020). Target Discovery of Selective Non-Small-Cell Lung Cancer Toxins Reveals Inhibitors of Mitochondrial Complex I. ACS Chem Biol 15, 158-170. 10.1021/acschembio.9b00734.
Mark, M., Muller, P., Maier, R., and Eisele, B. (1996). Effects of a novel 2,3-oxidosqualene cyclase inhibitor on the regulation of cholesterol biosynthesis in HepG2 cells. J Lipid Res 37, 148-158.
Mellinghoff, I. K., Penas-Prado, M., Peters, K. B., Burris, H. A., 3rd, Maher, E. A., Janku, F., Cote, G. M., de la Fuente, M. I., Clarke, J. L., Ellingson, B. M., et al. (2021). Vorasidenib, a Dual Inhibitor of Mutant IDH1/2, in Recurrent or Progressive Glioma; Results of a First-in-Human Phase I Trial. Clin Cancer Res 27, 4491-4499. 10.1158/1078-0432.CCR-21-0611.
Moebius, F. F., Reiter, R. J., Bermoser, K., Glossmann, H., Cho, S. Y., and Paik, Y. K. (1998). Pharmacological analysis of sterol delta8-delta7 isomerase proteins with [3H]ifenprodil. Mol Pharmacol 54, 591-598. 10.1124/mol.54.3.591.
Morand, O. H., Aebi, J. D., Dehmlow, H., Ji, Y. H., Gains, N., Lengsfeld, H., and Himber, J. (1997). Ro 48-8.071, a new 2,3-oxidosqualene:lanosterol cyclase inhibitor lowering plasma cholesterol in hamsters, squirrel monkeys, and minipigs: comparison to simvastatin. J Lipid Res 38, 373-390.
Nelson, J. A., Steckbeck, S. R., and Spencer, T. A. (1981a). 24(S),25-Epoxycholesterol is a natural product of mammalian steroid biosynthesis. Journal of the American Chemical Society 103, 6974-6975. 10.1021/ja00413a040.
Nelson, J. A., Steckbeck, S. R., and Spencer, T. A. (1981b). Biosynthesis of 24,25-epoxycholesterol from squalene 2,3;22,23-dioxide. J Biol Chem 256, 1067-1068.
Ostrom. Q. T., Cioffi, G., Gittleman, H., Patil, N., Waite, K., Kruchko, C., and Barnholtz-Sloan, J. S. (2019). CBTRUS Statistical Report: Primary Brain and Other Central Nervous System Tumors Diagnosed in the United States in 2012-2016. Neuro Oncol 21, vI-v100. 10.1093/neuonc/noz150.
Phillips, R. E., Yang, Y., Smith, R. C., Thompson, B. M., Yamasaki, T., Soto-Feliciano, Y. M., Funato, K., Liang, Y., Garcia-Bermudez, J., Wang, X., et al. (2019). Target identification reveals lanosterol synthase as a vulnerability in glioma. Proc Natl Acad Sci USA 116, 7957-7962. 10.1073/pnas.1820989116.
Rabelo, V. W., Romeiro, N.C., and Abreu, P. A. (2017). Design strategies of oxidosqualene cyclase inhibitors: Targeting the sterol biosynthetic pathway. J Steroid Biochem Mol Biol 171, 305-317. 10.1016/j.jsbmb.2017.05.002.
Radhakrishnan, A., Ikeda, Y., Kwon, H. J., Brown, M. S., and Goldstein, J. L. (2007). Sterol-regulated transport of SREBPs from endoplasmic reticulum to Golgi: oxysterols block transport by binding to Insig. Proc Natl Acad Sci USA 104, 6511-6518. 10.1073/pnas.0700899104.
Rowe, A. H., Argmann, C. A., Edwards, J. Y., Sawyez, C. G., Morand, O. H., Hegele, R. A., and Huff, M. W. (2003). Enhanced Synthesis of the Oxysterol 24(S),25-Epoxycholesterol in Macrophages by Inhibitors of 2,3-Oxidosqualene:Lanosterol Cyclase. Circulation Research 93, 717-725. 10.1161/01.res.0000097606.43659.f4.
Sassi, K., Nury, T., Samadi, M., Fennira, F. B. A., Vejux, A., and Lizard, G. (2021). Cholesterol Derivatives as Promising Anticancer Agents in Glioblastoma Metabolic Therapy. In Gliomas, W. Debinski, ed. 10.36255/exonpublications.gliomas.2021.chapter6.
Schultz, J. R., Tu, H., Luk, A., Repa, J. J., Medina, J. C., Li, L., Schwendner, S., Wang, S., Thoolen, M., Mangelsdorf, D. J., et al. (2000). Role of LXRs in control of lipogenesis. Genes Dev 14, 2831-2838. 10.1101/gad.850400.
Shi, A., Murai, M. J., He, S., Lund, G., Hartley, T., Purohit, T., Reddy, G., Chruszcz, M., Grembecka, J., and Cierpicki, T. (2012). Structural insights into inhibition of the bivalent menin-MLL interaction by small molecules in leukemia. Blood 120, 4461-4469. 10.1182/blood-2012-05-429274.
Shi, Y., Lim, S. K., Liang, Q., Iyer, S. V., Wang, H. Y., Wang, Z., Xie, X., Sun, D., Chen, Y. J., Tabar, V., et al. (2019). Gboxin is an oxidative phosphorylation inhibitor that targets glioblastoma. Nature 567, 341-346. 10.1038/s41586-019-0993-x.
Stupp, R., Mason, W. P., van den Bent, M. J., Weller, M., Fisher, B., Taphoorn, M. J., Belanger, K., Brandes, A. A., Marosi, C., Bogdahn, U., et al. (2005). Radiotherapy plus concomitant and adjuvant temozolomide for glioblastoma. N Engl J Med 352, 987-996. 10.1056/NEJMoa043330.
Theodoropoulos, P. C., Wang, W., Budhipramono, A., Thompson, B. M., Madhusudhan, N., Mitsche, M. A., McDonald, J. G., De Brabander, J. K., and Nijhawan, D. (2020). A Medicinal Chemistry-Driven Approach Identified the Sterol Isomerase EBP as the Molecular Target of TASIN Colorectal Cancer Toxins. J Am Chem Soc 142, 6128-6138. 10.1021/jacs.9b13407.
Theofilopoulos, S., Abreu de Oliveira, W. A., Yang, S., Yutuc, E., Saeed, A., Abdel-Khalik, J., Ullgren, A., Cedazo-Minguez, A., Bjorkhem, I., Wang, Y., et al. (2019). 24(S),25-Epoxycholesterol and cholesterol 24S-hydroxylase (CYP46A1) overexpression promote midbrain dopaminergic neurogenesis in vivo. J Biol Chem 294, 4169-4176. 10.1074/jbc.RA118.005639.
Theofilopoulos, S., Wang, Y., Kitambi, S. S., Sacchetti, P., Sousa, K. M., Bodin, K., Kirk, J., Salto, C., Gustafsson, M., Toledo, E. M., et al. (2013). Brain endogenous liver X receptor ligands selectively promote midbrain neurogenesis. Nat Chem Biol 9, 126-133. 10.1038/nchembio.1156.
Thoma, R., Schulz-Gasch, T., D'Arcy, B., Benz, J., Aebi, J., Dehmlow, H., Hennig, M., Stihle, M., and Ruf, A. (2004). Insight into steroid scaffold formation from the structure of human oxidosqualene cyclase. Nature 432, 118-122. 10.1038/nature02993.
Villa, G. R., Hulce, J. J., Zanca, C., Bi, J., Ikegami, S., Cahill, G. L., Gu, Y., Lum, K. M., Masui, K., Yang, H., et al. (2016). An LXR-Cholesterol Axis Creates a Metabolic Co-Dependency for Brain Cancers. Cancer Cell 30, 683-693. 10.1016/j.ccell.2016.09.008.
Wages, P. A., Kim, H. H., Korade, Z., and Porter, N. A. (2018). Identification and characterization of prescription drugs that change levels of 7-dehydrocholesterol and desmosterol. J Lipid Res 59, 1916-1926. 10.1194/jlr.M086991.
Willy, P. J., Umesono, K., Ong, E. S., Evans, R. M., Heyman, R. A., and Mangelsdorf, D. J. (1995). LXR, a nuclear receptor that defines a distinct retinoid response pathway. Genes Dev 9, 1033-1045. 10.1101/gad.9.9.1033.
Wong, J., Quinn, C. M., Guillemin, G., and Brown, A. J. (2007). Primary human astrocytes produce 24(S),25-epoxycholesterol with implications for brain cholesterol homeostasis. J Neurochem 103, 1764-1773. 10.1111/j.1471-4159.2007.04913.x.
Zhou, W., and Wahl, D. R. (2019). Metabolic Abnormalities in Glioblastoma and Metabolic Strategies to Overcome Treatment Resistance. Cancers (Basel) 11. 10.3390/cancersi1091231.
Zhou, W., Yao, Y., Scott, A. J., Wilder-Romans, K., Dresser, J. J., Werner, C. K., Sun, H., Pratt, D., Sajjakulnukit, P., Zhao, S. G., et al. (2020). Purine metabolism regulates DNA repair and therapy resistance in glioblastoma. Nat Commun 11, 3811. 10.1038/s41467-020-17512-x.

### METHODS REFERENCES

1. Theodoropoulos, P. C. et al. Discovery of tumor-specific irreversible inhibitors of stearoyl CoA desaturase. Nat Chem Biol 12, 218-225, doi:10.1038/nchembio.2016 (2016).
2. McDonald, J. G., Smith, D. D., Stiles, A. R. & Russell, D. W. A comprehensive method for extraction and quantitative analysis of sterols and secosteroids from human plasma. J Lipid Res 53, 1399-1409, doi:10.1194fjIr.D022285 (2012).
3. Kurten, C., Uhlen, M. & Syren, P. O. Overexpression of functional human oxidosqualene cyclase in Escherichia coli. Protein Expr Purif 115, 46-53, doi:10.1016/j.pep.2015.04.015 (2015).
4. McNaney, C. A. et al. An automated liquid chromatography-mass spectrometry process to determine metabolic stability half-life and intrinsic clearance of drug candidates by substrate depletion. Assay Drug Dev Technol 6, 121-129, doi:10.1089/adt.2007.103 (2008).
5. Kalvass, J. C. & Maurer, T. S. Influence of nonspecific brain and plasma binding on CNS exposure: implications for rational drug discovery. Biopharm Drug Dispos 23, 327-338, doi:10.1002/bdd.325 (2002).
6. Louis, D. N. et al. The 2021 WHO Classification of Tumors of the Central Nervous System: a summary. Neuro Oncol 23, 1231-1251, doi:10.1093/neuonc/noab106 (2021).

### Synthesis

All reactions were carried out under nitrogen atmosphere with dry solvents under anhydrous conditions, unless otherwise noted. Anhydrous solvents were obtained by passing them through commercially available alumina columns (Innovative technology, Inc., MA). All reagents were commercial compounds of the highest purity available. Analytical thin layer chromatography (TLC) was performed on aluminium plates with Merck Kieselgel 60F254 and visualized by UV irradiation (254 nm) or by staining with a solution of potassium permanganate. Flash column chromatography was carried out using Merck Kieselgel 60 (230-400 mesh) under pressure. Infrared spectra were obtained on a Perkin-Elmer I1000 FTIR series, from a thin film deposited onto a NaCl glass. Optical rotations were measured on a Rudolph Research Analytical Autopol® IV polarimeter at 20° C. 1H NMR spectra were recorded in CDCl₃, CD₃OD, DMSOd₆ and (CD₃)₂CO at ambient temperature on a Varian Inova-400 spectrometer at 400 MHz with residual protic solvent as the internal reference (CDCl₃, d_{H}=7.26 ppm; (CD₃)₂CO, d_{H}=2.05 ppm; CD₃OD, d_{H}=3.31 ppm; DMSO-d₆, d_{H}=2.50 ppm); chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities. app=apparent, b=broad, d=doublet, dd=doublet of doublets, ddd=doublet of doublet of doublets, dddd=doublet of doublet of doublet of doublets, m=multiplet, s=singlet, t=triplet. ¹³C NMR spectra were recorded in CDCl₃, CD₃OD, DMSO-d₆ and (CD₃)₂CO at ambient temperature on the same spectrometer at 100 MHz with the central peak of CDCl₃ (d_{C}=77.0 ppm), CD₃OD (d_{C}=49.0 ppm), DMSO-d6 (d_{C}=39.4 ppm) or (CD₃)₂CO (d_{C}=30.8 ppm) as the internal reference. Electrospray ionization mass spectra (ESI-MS) were recorded on a Shimadzu 2010-LCMS. HRMS were performed on a Shimadzu IT-TOF. Microwave reactions were carried out on a Biotage® Initiator Classic. DMAP=4-(dimethylamino)pyridine, DMF=N,N-dimethylformamide, DIPEA=N,N-diisopropylethylamine, DHP=tetrahydro-2H-pyran, EDC=N-ethylcarbodiimide hydrochloride, HATU=1-[bis(dimethylamino)methylene]-1H-1,2,3-triazolo[4,5-b]pyridinium 3-oxid hexafluorophosphate, MW=microwave, NMP=1-methyl-2-pyrrolidinone, py=pyridine, TBDMS=tert-butyldimethylsilyl, THF=tetrahydrofuran, THP=3,4-dihydro-2H-pyran. Unless otherwise noted, commercially available materials were used without further purification. All solvents were of HPLC or ACS grade. Solvents used for moisture sensitive operations were distilled from drying reagents under a nitrogen atmosphere: Et₂O and THF from sodium benzophenone ketyl; benzene and toluene from sodium; CH₂Cl₂ from CaH₂, pyridine over solid KOH, anhydrous N,N-dimethylformamide, and CH₃CN were purchased from commercial sources. Reactions were performed under an atmosphere of argon with magnetic stirring unless noted otherwise. Flash chromatography (FC) was performed using E Merck silica gel 60 (240-400 mesh) according to the protocol of Still, Kahn, and Mitral.

General Procedure A for the Condensation of anhydride 97 with (Hetero)aromatic Amines (Schemes 1-2). A mixture of anhydride 97 (1.0 equiv.) and the corresponding (hetero)aromatic amine (1.0 equiv.) in pyridine (0.1 M) was heated at 140° C. in a pressure-sealed tube. The reaction was monitored by TLC analysis. The reaction was cooled to rt and diluted with CH₂Cl₂ (10 mL) and washed with excessive amount of 10% aq. CuSO₄. The organic phase was washed with H₂O and dried over anhydrous MgSO₄, and filtered. The solvent was removed under reduced pressure and the residue was purified by flash chromatography on silica gel as indicated to give the corresponding dicarboximides.

General Procedure B for the Condensation of anhydride 97 with (Hetero)aromatic Amines (Schemes 1-2). A mixture of anhydride 97 (1.0 equiv.) and the corresponding (hetero)aromatic amine (1.0 equiv.) in AcOH (0.5 M) was heated to 90° C. using a conventional heating method or to 100° C. for 45 min using a microwave reactor. The reaction was monitored by TLC analysis. The reaction was cooled to rt and crystallization occurred. The crystals were collected through filtration and the residue was washed with Et₂O/hexanes (1:1) to remove excess AcOH, and no further purification is needed. When no crystallization occurred, the mixture was diluted with EtOAc and neutralized with saturated aq. NaHCO₃. The organic phase was washed with brine, dried over anhydrous Na₂SO₄, and filtered. The solvent was removed under reduced pressure and the residue was purified by flash chromatography on silica gel as indicated to give the corresponding dicarboximides.

General Procedure C for the Synthesis of Compounds 92-95, 10, 11, 15, 16, 20, 29, 30a-45a, 47a-64a, 52b, 55b-58b, 60b-63b, and 71-86 via Alkylation (Scheme 1). A mixture of (optionally substituted) nitrophenol or intermediates 98-106 (1.0 equiv.), the corresponding bromide (1.5 equiv.), K₂CO₃ (1.5 equiv.) and NaI (0.5 equiv.) in acetone (0.1 M) was heated to 80° C. and stirred overnight. The solvent was removed under reduced pressure and the residue was purified by flash chromatography on silica gel as indicated to give the alkylated products.

General Procedure D for the One-Pot Reduction-Cycloaddition for the Synthesis of Compounds 7-9 from Nitroarenes 93-95 (Scheme 1). To a suspension of Fe (5 equiv.) in AcOH (1.0 M) was added the corresponding nitroarene 93-95 (1.0 equiv.), maleic anhydride (1.2 equiv.) and 1,3,5-cycloheptatriene (3.0 equiv.). The mixture was heated to 120° C. and stirred for 24 h. The mixture was cooled to rt and filtered through a Celite pad, and the residue was washed with EtOAc (2×). The combined organic phases were washed with a saturated aq. NaHCO₃ solution, brine and dried over anhydrous MgSO₄, and filtered. The solvent was removed under reduced pressure and the residue was purified by flash chromatography on silica gel as indicated to give the cycloadducts 7-9.

General Procedure E for the Synthesis of Enones 110, 111a-f via Wittig Olefination (Scheme 2). A mixture of the corresponding benzaldehyde 107 or 108 (1 equiv.) and the corresponding ylide (1.5 equiv.), in CH₂Cl₂ (0.2 M) was refluxed at 70° C. TLC monitored the reaction. The solvent was removed under reduced pressure and the residue was purified by flash chromatography on silica gel as indicated to give the enones 110 or 111a-f.

General Procedure F for the Synthesis of Cyclopropane Analogs via Corey-Chaykovsky cyclopropanation (Scheme 2). A flamed-dried flask was charged with trimethylsulfoxonium iodide (1.5 equiv.) and NaH (1.5 equiv., 60% dispersion in oil). The mixture was dissolved in anhydrous DMSO (0.5 M) with stirring at rt for 30 min. Noted that the reaction mixture became homogenous after 30 min. The corresponding enone (1.0 equiv.) was added to the reaction mixture in one portion. The resulting mixture was heated to 85° C. and stirred overnight. After cooling to rt, the reaction was quenched with cold H₂O. The mixture was extracted with EtOAc, and the combined organic layers were washed with brine and dried over anhydrous Na₂SO₄, and filtered. The solvent was removed under reduced pressure and the residue was purified by flash chromatography on silica gel as indicated to give the cyclopropyl derivatives.

### 1-(4-Methoxyphenyl)-2-(4-nitrophenoxy)ethan-1-one (92)

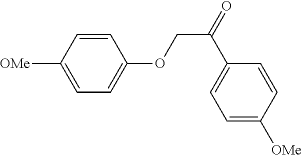 [SMILES: COc1ccc(OCC(=O)c2ccc(OC)cc2)cc1]

General Procedure C. Purification by flash chromatography on silica gel (gradient elution, 0→30% EtOAc in hexanes) followed by recrystallization in CH₂Cl₂/hexanes afforded 92 (3.8 g, 13.2 mmol, 92%) as a pale yellow solid. IR (cm⁻¹) 2914, 2842, 1690, 1600, 1509, 1341, 1231, 1172, 1112, 970, 836; ¹H NMR (400 MHz, CDCl₃) δ 8.19 (d, J=9.2 Hz, 2H), 7.97 (d, J=8.8 Hz, 2H), 6.99 (d, J=9.2 Hz, 2H), 6.98 (d, J=9.2 Hz, 2H), 5.36 (s, 2H), 3.90 (s, 3H); ¹³C NMR (100 MHz, CDCl₃) δ 191.5, 164.6, 163.2, 130.6 (2C), 127.2, 126.1 (2C), 115.0 (2C), 114.5 (2C), 110.2, 70.7, 55.8; ES-API MS: m/z calcd for C₁₅H₁₄NO₅ 288.1, found 288.1 [M+H]⁺.

### 2-(2-Fluoro-4-nitrophenoxy)-1-(4-methoxyphenyl)ethan-1-one (93)

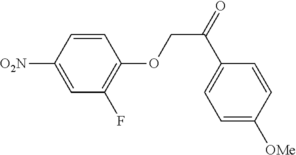 [SMILES: COc1ccc(C(=O)COc2ccc([N+](=O)[O-])cc2F)cc1]

General Procedure C. Purification by flash chromatography on silica gel (gradient elution, 0425% EtOAc in hexanes) afforded 93 (45 mg, 0.14 mmol, 96%) as a yellow solid. IR (cm⁻¹) 1686, 1600, 1508, 1346, 1293, 1239, 1171, 970; ¹H NMR (400 MHz, DMSO-d₆) δ 8.20 (dd, J=11.1, 2.6 Hz, 1H), 8.06 (d, J=9.2 Hz, 1H), 7.99 (d, J=8.8 Hz, 2H), 7.34 (t, J=8.9 Hz, 1H), 7.11 (d, J=8.8 Hz, 2H), 5.88 (s, 2H), 3.87 (s, 3H); ¹³C NMR (100 MHz, DMSO-d₆) δ 191.3, 163.8, 152.0 (d, J=10.0 Hz, 1C), 150.2 (d, J=246.9 Hz, 1C), 140.3 (d, J=7.0 Hz, 1C), 130.3 (2C), 126.8, 121.1 (d, J=2.7 Hz, 1C), 114.7, 114.1 (2C), 112.1 (d, J=21.3 Hz, 1C), 70.9, 55.7; ES-API MS: m/z calcd for C₁₅H₁₃FNO₅ 306.1, found 306.1 [M+H]⁺.

### 2-(3-Fluoro-4-nitrophenoxy)-1-(4-methoxyphenyl)ethan-1-one (94)

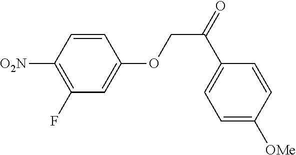 [SMILES: COc1ccc(C(=O)COc2ccc([N+](=O)[O-])c(F)c2)cc1]

General Procedure C. Purification by recrystallization in acetone/H₂O afforded 94 (376 mg, 1.23 mmol, 83%) as a brownish solid. IR (cm⁻¹) 1678, 1600, 1511, 1332, 1243, 1171, 1098, 829; ¹H NMR (400 MHz, DMSO-d₆) δ 8.15 (dd, J=9.2, 9.2 Hz, 1H), 8.00 (dd, J=8.8 Hz, 2H), 7.29 (d, J=14.0 Hz, 1H), 7.11 (d, J=8.4 Hz, 2H), 7.02 (d, J=9.2 Hz, 1H), 5.78 (s, 2H), 3.87 (s, 3H); ¹³C NMR (100 MHz, DMSO-d₆) δ 192.0, 164.9 (d, J=11.3 Hz, 1C), 164.4, 157.3 (d, J=260.0 Hz, 1C), 131.0 (2C), 128.6 (2C), 127.5, 114.8 (2C), 112.5 (d, J=2.0 Hz, 1C), 104.8 (d, J=24.6 Hz, 1C), 71.5, 56.3 (d, J=2.3 Hz, 1C); ES-API MS: m/z calcd for C₁₅H₁₃FNO₅ 306.1, found 306.1 [M+H]⁺.

### 1-(4-Methoxyphenyl)-2-(4-nitro-3-(trifluoromethyl)phenoxy)ethan-1-one (95)

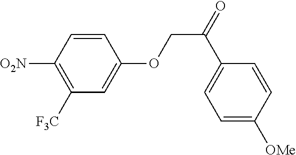 [SMILES: COc1ccc(C(=O)COc2ccc([N+](=O)[O-])c(C(F)(F)F)c2)cc1]

General Procedure C. Purification by flash chromatography on silica gel (gradient elution, 0→30% EtOAc in hexanes) followed by recrystallization in CH₂Cl₂/hexanes afforded 95 (802 mg, 2.26 mmol, 94%) as a yellow-orange solid. IR (cm⁻¹) 2940, 1690, 1601, 1534, 1314, 1229, 1174, 1149, 974, 834; ¹H NMR (400 MHz, CDCl₃) δ 7.98 (d, J=8.4 Hz, 1H), 7.96 (d, J=8.4 Hz, 2H), 7.36 (d, J=2.8 Hz, 1H), 7.08 (dd, J=9.2, 2.4 Hz, 1H), 7.00 (d, J=8.4 Hz, 2H), 5.41 (s, 2H), 3.90 (s, 3H); ¹³C NMR (100 MHz, CDCl₃) δ 190.8, 164.8, 161.4, 130.6 (2C), 130.2, 128.3, 126.9, 126.6, 121.9 (q, J=272.1 Hz, 1C), 117.0, 115.6 (q, J=5.9 Hz, 1C), 114.6 (2C), 70.7, 55.9; ES-API MS: m/z calcd for C₁₆H₁₃F₃NO₅ 356.1, found 356.1 [M+H]⁺.

### 2-(4-Aminophenoxy)-1-(4-methoxyphenyl)ethan-1-one (96)

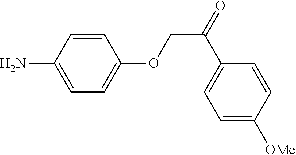 [SMILES: COc1ccc(C(=O)COc2ccc(N)cc2)cc1]

A mixture of nitroarene 92 (1.0 equiv.) and iron powder (5.0 equiv.) in saturated NH₄Cl solution (1.0 M) and EtOH (1.0 M) was heated to 70° C. and stirred until TLC showed complete conversion. The mixture was cooled to rt and filtered through a Celite pad, and the residue was washed with EtOAc (2×). The combined organic layers were washed with saturated aq. NaHCO₃, brine and dried over anhydrous MgSO₄, and filtered. The solvent was removed under reduced pressure and the residue was purified by flash chromatography on silica gel (gradient elution, 0→70% EtOAc in hexanes) to give aniline 96 (78 mg, 0.30 mmol, 90%) as a yellow solid. IR (cm⁻¹) 3408, 3334, 1600, 1506, 1258, 1213, 1172, 828; ¹H NMR (400 MHz, CDCl₃) δ 7.96 (d, J=8.8 Hz, 2H), 6.92 (d, J=8.8 Hz, 2H), 6.76 (d, J=8.8 Hz, 2H), 6.59 (d, J=8.8 Hz, 2H), 5.10 (s, 2H), 3.84 (s, 3H), 3.42 (br, 2H); ¹³C NMR (100 MHz, CDCl₃) δ 193.8, 164.1, 151.4, 141.0, 130.7 (2C), 127.9, 116.4 (2C), 116.3 (2C), 114.1 (2C), 71.9, 55.7; ES-API MS: m/z calcd for C₁₅H₁₆NO₃ 258.1, found 258.1 [M+H]⁺.

### (3aR,4R,4aR,5aS,6S,6aS)-4,4a,5,5a,6,6a-Hexahydro-1H-4,6-ethenocyclopropa[f]isobenzofuran-1,3(3aH)-Dione; (3aR,4S,4a,5aS,6R,6aS)-4,4a,5,5a,6,6a-hexahydro-1H-4,6-ethenocyclopropa[f]isobenzofuran-1,3(3aH)-dione (97)

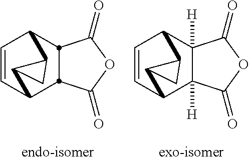

A mixture of 1,3,5-cycloheptatriene (10 g, 0.108 mol) and maleic anhydride (12.0 g, 0.122 mol) in xylene (50 mL) was heated to 150° C. for 12 h. The reaction was cooled to rt and concentrated under reduced pressure to remove the solvent. The residue was purified by flash chromatography on silica gel (gradient elution, 0→10% EtOAc in hexanes and 10430% EtOAc in hexanes) to afford the exo-isomer 97-exo (1 g, 5.25 mmol, 5%) and endo-isomer 97-endo (14 g, 73.6 mmol, 70%) both as white solids. 97-endo: IR (cm⁻¹) 3008, 2945, 1856, 1823, 1774, 1376, 1227, 1093, 1078, 952, 915; ¹H NMR (500 MHz, CDCl₃) δ 5.87 (m, 2H), 3.45 (m, 2H), 3.23 (dd, J=7.5, 1.5 Hz, 2H), 1.10 (m, 2H), 0.35 (m, 1H), 0.25 (m, 1H); ¹³C NMR (100 MHz, CDCl₃) δ 172.5 (2C), 128.6 (2C), 46.0 (2C), 33.8 (2C), 9.7 (2C), 5.3; ES-API MS: m/z calcd for C₁₁H₁₀O₃, found 191.1 [M+H]⁺. 97-exo: IR (cm⁻¹) 3016, 2983, 1848, 1780, 1305, 1221, 1086, 1072, 1042, 960, 915, 851; ¹H NMR (500 MHz, CDCl₃) δ 5.94 (dd, J=4.0, 4.0 Hz, 2H), 3.41 (m, 2H), 3.08 (m, 2H), 1.12 (m, 2H), 0.21 (dd, J=13.5, 7.0 Hz, 1H), 0.13 (m, 1H); ¹³C NMR (100 MHz, CDCl₃) δ 172.5 (2C), 129.4 (2C), 46.7 (2C), 32.7 (2C), 5.6 (2C), 1.9; ES-API MS: m/z calcd for C₁₁H₁₁O₃ 191.1, found 191.1 [M+H]⁺.

### (3aR,4R,4aR,5aS,6S,6aS)-2-(4-(2-(4-Methoxyphenyl)-2-oxoethoxy)phenyl)-4,4a,5,5a,6,6a-hexahydro-4,6-ethenocyclopropa[f]isoindole-1,3(2H,3aH)-dione (1)

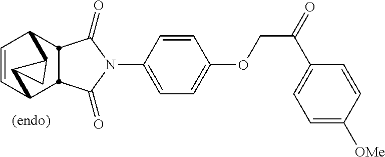 [SMILES: COc1ccc(C(=O)COc2ccc(N3C(=O)[C@@H]4[C@H](C3=O)[C@@H]3C=C[C@H]4C4CC43)cc2)cc1]

A mixture of anhydride 97-endo (1.0 equiv.) and the corresponding aniline (1.0 equiv.) in AcOH (0.2 M) was heated to 120° C. for 30 min. The mixture was cooled to rt and quenched with a saturated aq. NaHCO₃ solution, and extracted with EtOAc (3×). The combined organic layer was washed with brine and dried over anhydrous MgSO₄, and filtered. The solvent was removed under reduced pressure and the residue was purified by crystallization in EtOAc/hexanes to afford 1 (40 mg, 0.093 mmol, 35%) as a white solid. IR (cm⁻¹) 1704, 1601, 1511, 1389, 1224, 1172; ¹H NMR (400 MHz, DMSO-d₆) δ 8.00 (d, J=8.0 Hz, 2H), 7.09 (d, J=8.0 Hz, 2H), 6.98 (s, 4H), 5.82 (m, 2H), 5.54 (s, 2H), 3.85 (s, 3H), 3.27 (m, 2H), 3.16 (m, 2H), 1.17 (m, 2H), 0.25 (m, 1H), 0.07 (m, 1H); ¹³C NMR (100 MHz, DMSO-d₆) δ 192.8, 177.9 (2C), 163.8, 157.8, 130.4 (2C), 128.1 (2C), 127.7 (2C), 127.4, 125.2, 115.0 (2C), 114.3 (2C), 70.2, 55.8, 45.0 (2C), 33.4 (2C), 9.7 (2C), 4.5; ES-API MS: m/z calcd for C₂₆H₂₄NO₅ 430.2, found 430.2 [M+H]⁺.

### (3aR,4S,4aS,5aR,6R,6aS)-2-(4-(2-(4-Methoxyphenyl)-2-oxoethoxy)phenyl)-4,4a,5,5a,6,6a-hexahydro-4,6-ethenocyclopropa[f]isoindole-1,3(2H,3aH)-dione (2)

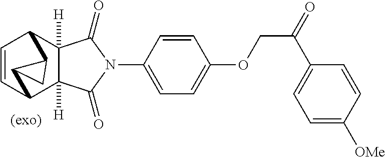 [SMILES: COc1ccc(C(=O)COc2ccc(N3C(=O)[C@@H]4[C@H](C3=O)[C@H]3C=C[C@@H]4C4CC43)cc2)cc1]

A mixture of anhydride 97-exo (1.0 equiv.) and the corresponding aniline (1.0 equiv.) in AcOH (0.2 M) was heated to 120° C. for 30 min. The mixture was cooled to rt and quenched with saturated aq. NaHCO₃, and extracted with EtOAc (3×). The combined organic layer was washed with brine and dried over anhydrous MgSO₄, and filtered. The solvent was removed under reduced pressure and the residue was purified by crystallization in EtOAc/hexanes afforded 2 (35 mg, 0.081 mmol, 33%) as a white solid. IR (cm⁻¹) 1702, 1599, 1510, 1387, 1308, 1228, 1172; ¹H NMR (500 MHz, DMSO-d₆) δ 8.00 (d, J=9.0 Hz, 2H), 7.12 (d, J=9.5 Hz, 2H), 7.09 (d, J=9.5 Hz, 2H), 7.02 (d, J=8.5 Hz, 2H), 5.95 (m, 2H), 5.56 (s, 2H), 3.86 (s, 3H), 3.24 (m, 2H), 2.89 (m, 2H), 0.95 (m, 2H), 0.12 (m, 1H), −0.02 (m, 1H); ¹³C NMR (100 MHz, DMSO-d₆) δ 192.3, 177.3 (2C), 163.2, 157.3, 129.9 (2C), 128.8 (2C), 127.7 (2C), 126.8, 124.8, 114.5 (2C), 113.7 (2C), 69.5, 55.2, 44.7 (2C), 31.5 (2C), 4.7 (2C), 0.86; ES-API MS: m/z calcd for C₂₆H₂₄NO₅ 430.2, found 430.2 [M+H]⁺.

### 1-(4-(2-(4-Methoxyphenyl)-2-oxoethoxy)phenyl)-1H-pyrrole-2,5-dione (5)

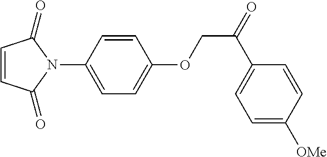 [SMILES: COc1ccc(C(=O)COc2ccc(N3C(=O)C=CC3=O)cc2)cc1]

A mixture of aniline 96 (219 mg, 0.851 mmol) and maleic anhydride (100 mg, 1.02 mmol) in AcOH (8.5 mL) was heated to 120° C. and stirred for 12 h. The mixture was cooled to rt and quenched with cold aq. saturated NaHCO₃, and extracted with EtOAc (3×). The combined organic layer was washed with brine and dried over anhydrous MgSO₄, and filtered. The solvent was removed under reduced pressure and the residue was purified by flash chromatography on silica gel (gradient elution, 0→40% EtOAc in hexanes) to afford compound 5 (148 mg, 0.44 mmol, 52%) as a yellow solid. IR (cm⁻¹) 1714, 1601, 1511, 1397, 1224, 1172; ¹H NMR (400 MHz, CDCl₃) δ 8.00 (d, J=9.2 Hz, 2H), 7.24 (d, J=8.8 Hz, 2H), 7.02 (d, J=8.8 Hz, 2H), 6.97 (d, J=8.8 Hz, 2H), 6.83 (s, 2H), 5.23 (s, 2H), 3.89 (s, 3H); ¹³C NMR (100 MHz, CDCl₃) δ 192.8, 169.9 (2C), 164.4, 157.8, 134.4 (2C), 130.8 (2C), 127.8 (2C), 127.7, 124.9, 115.6 (2C), 114.3 (2C), 71.1, 55.8; ES-API MS: m/z calcd for C₁₉H₁₆NO₅ 338.1, found 338.1 [M+H]⁺.

### (3aR,4S,7R,7aS)-2-(4-(2-(4-Methoxyphenyl)-2-oxoethoxy)phenyl)-3a,4,7,7a-tetrahydro-1H-4,7-ethanoisoindole-1,3(2H)-dione (3)

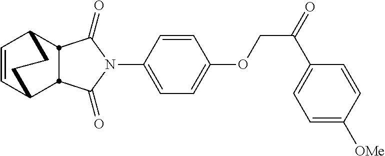 [SMILES: COc1ccc(C(=O)COc2ccc(N3C(=O)[C@@H]4[C@H](C3=O)[C@@H]3C=C[C@H]4CC3)cc2)cc1]

To a solution of maleimide 5 (1 equiv.) in CH₂Cl₂ (0.1 M) was added the cyclohexadiene (5.0 equiv.). The resulting mixture was heated to 60° C. until TLC showed complete consumption starting material. The solvent was removed under reduced pressure and the residue was purified by flash chromatography on silica gel (gradient elution, 0→40% EtOAc in hexanes) to afford cycloadduct 3 (9.0 mg, 0.021 mmol, 36%) as a white solid. IR (cm⁻¹) 2954, 1707, 1601, 1512, 1389, 1299, 1233, 1172; ¹H NMR (500 MHz, CDCl₃) δ 7.98 (d, J=8.8 Hz, 2H), 7.09 (d, J=9.0 Hz, 2H), 6.98 (d, J=9.0 Hz, 2H), 6.96 (d, J=9.0 Hz, 2H), 6.27 (dd, J=4.0, 3.5 Hz, 2H), 5.19 (s, 2H), 3.89 (s, 3H), 3.24 (m, 2H), 2.99 (m, 2H), 1.65 (d, J=7.5 Hz, 2H), 1.43 (d, J=8.0 Hz, 2H); ¹³C NMR (100 MHz, CDCl₃) δ 192.8, 178.4 (2C), 164.3, 158.1, 132.6 (2C), 130.8 (2C), 128.0 (2C), 125.6, 115.5 (2C), 114.3 (2C), 105.0, 71.2, 55.8, 44.4 (2C), 32.2 (2C), 23.9 (2C); ES-API MS: m/z calcd for C₂₅H₂₄NO₅ 418.2, found 418.2 [M+H]⁺.

### (3aR,4S,7R,7aS)-2-(4-(2-(4-Methoxyphenyl)-2-oxoethoxy)phenyl)-3a,4,7,7a-tetrahydro-1H-4,7-methanoisoindole-1,3(2H)-done (4)

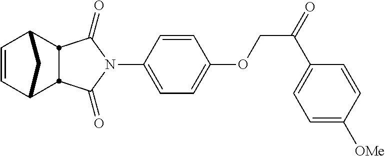

To a solution of maleimide 5 (1 equiv.) in CH₂Cl₂ (0.1 M) was added the cyclopentadiene (5.0 equiv.). The resulting mixture was heated to 60° C. until TLC showed complete consumption starting material. The solvent was removed under reduced pressure and the residue was purified by flash chromatography on silica gel (gradient elution, 0→70% EtOAc in hexanes) to afford cycloadduct 4 (30 mg, 0.074 mmol, 63%) as a pale yellow solid. IR (cm⁻¹) 2843, 1706, 1601, 1512, 1386, 1263, 1227, 1173; ¹H NMR (500 MHz, CDCl₃) δ 7.98 (d, J=8.5 Hz, 2H), 7.05 (d, J=9.0 Hz, 2H), 6.97 (d, J=9.0 Hz, 2H), 6.96 (d, J=8.5 Hz, 2H), 6.24 (s, 2H), 5.18 (s, 2H), 3.89 (s, 3H), 3.49 (m, 2H), 3.41 (m, 2H), 1.78 (d, J=9.0 Hz, 1H), 1.60 (d, J=9.0 Hz, 1H); ¹³C NMR (100 MHz, CDCl₃) δ 192.8, 177.2 (2C), 164.3, 158.1, 134.8 (2C), 130.8 (2C), 128.1 (2C), 127.6, 125.5, 115.5 (2C), 114.2 (2C), 71.1, 55.7, 52.4, 45.8 (2C), 45.6 (2C); ES-API MS: m/z calcd for C₂₄H₂₂NO₅, found 404.1 [M+H]⁺.

### (3aR,4R,4aR,5aS,6S,6aS)-2-(2-Fluoro-4-(2-(4-methoxyphenyl)-2-oxoethoxy)phenyl)-4,4a,5,5a,6,6a-hexahydro-4,6-ethenocyclopropa[f]isoindole-1,3(2H,3aH)-dione (7)

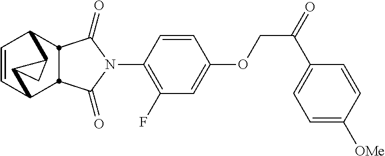 [SMILES: COc1ccc(C(=O)COc2ccc(N3C(=O)[C@@H]4[C@H](C3=O)[C@@H]3C=C[C@H]4C4CC43)c(F)c2)cc1]

Procedure D. Purification by flash chromatography on silica gel (gradient elution, 0→50% EtOAc in hexanes) afforded 7 (30 mg, 0.067 mmol, 14%) as a white solid. IR (cm⁻¹) 1707, 1693, 1600, 1518, 1385, 1291, 1243, 1166; ¹H NMR (400 MHz, CDCl₃) δ 7.97 (d, J=8.8 Hz, 2H), 7.06 (m, 1H), 6.97 (d, J=8.8 Hz, 2H), 6.78 (m, 1H), 6.75 (m, 1H), 5.85 (dd, J=4.4, 3.6 Hz, 2H), 5.19 (s, 2H), 3.89 (s, 3H), 3.48 (m, 2H), 3.15 (m, 2H), 1.14 (m, 2H), 0.35-0.26 (m, 2H); ¹³C NMR (100 MHz, DMSO-d₆) δ 192.6, 177.5 (2C), 164.1, 160.0, 159.1, 156.6, 130.7 (2C), 128.0, 127.8, 127.6 (2C), 114.5 (2C), 110.0, 103.4, 70.7, 56.1, 45.7, 45.2, 33.7, 33.6, 9.9 (2C), 4.9; ES-API MS: m/z calcd for C₂₆H₂₃FNO₅ 448.2, found 448.2 [M+H]⁺.

### (3aR,4R,4aR,5aS,6S,6aS)-2-(3-Fluoro-4-(2-(4-methoxyphenyl)-2-oxoethoxy)phenyl)-4,4a,5,5a,6,6a-hexahydro-4,6-ethenocyclopropa[f]isoindole-1,3(2H,3aH)-dione (8)

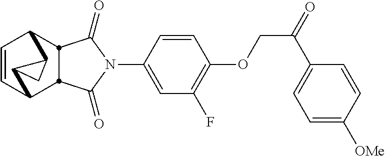 [SMILES: COc1ccc(C(=O)COc2ccc(N3C(=O)[C@@H]4[C@H](C3=O)[C@@H]3C=C[C@H]4C4CC43)cc2F)cc1]

Procedure D. Purification by flash chromatography on silica gel (gradient elution, 0→50% EtOAc in hexanes) afforded 8 (183 mg, 0.40 mmol, 59%) as a white solid. IR (cm⁻¹) 1774, 1709, 1600, 1515, 1441, 1388, 1268, 1240, 1174; ¹H NMR (400 MHz, DMSO-d₆) δ 7.99 (d, J=8.8 Hz, 2H), 7.15 (dd, J=9.2, 9.2 Hz, 1H), 7.10 (d, J=8.8 Hz, 2H), 7.04 (dd, J=11.6, 2.0 Hz, 1H), 6.84 (ddd, J=8.8, 1.6, 1.6 Hz, 1H), 5.82 (dd, J=4.4, 3.6 Hz, 2H), 5.68 (s, 2H), 3.86 (s, 3H), 3.28 (m, 2H), 3.18 (m, 2H), 1.18 (m, 2H), 0.26 (dd, J=12.8, 7.3 Hz, 1H), 0.08 (dd, J=8.8, 3.7 Hz, 1H); ¹³C NMR (100 MHz, DMSO-d₆) δ 192.2, 177.4 (2C), 163.7, 150.7 (d, J=243.5 Hz, 1C), 145.8 (d, J=10.1 Hz, 1C), 130.2 (2C), 127.5 (2C), 127.1, 124.8 (d, J=9.1 Hz, 1C), 123.1 (d, J=3.3 Hz, 1C), 115.0, 114.8, 114.1 (2C), 70.5, 55.7, 44.8 (2C), 33.3 (2C), 9.5 (2C), 4.4; ES-API MS: m/z calcd for C₂₆H₂₃FNO₅ 448.2, found 448.1 [M+H]⁺.

### (3aR,4R,4aR,5aS,6S,6aS)-2-(4-(2-(4-Methoxyphenyl)-2-oxoethoxy)-2-(trifluoromethyl)phenyl)-4,4a,5,5a,6,6a-hexahydro-4,6-ethenocyclopropa[f]isoindole-1,3(2H,3aH)-dione (9)

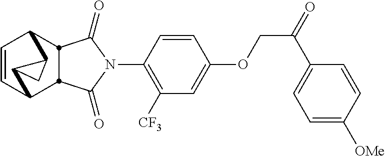 [SMILES: COc1ccc(C(=O)COc2ccc(N3C(=O)[C@@H]4[C@H](C3=O)[C@@H]3C=C[C@H]4C4CC43)c(C(F)(F)F)c2)cc1]

Procedure D. Purification by flash chromatography on silica gel (gradient elution, 0→30% EtOAc in hexanes) afforded 9 (432 mg, 0.87 mmol, 72%) as a white solid. IR (cm⁻¹) 3062, 3016, 1715, 1694, 1600, 1506, 1316, 1174; ¹H NMR (500 MHz, CDCl₃) δ 7.97 (d, J=9.2 Hz, 2H), 7.31 (d, J=2.8 Hz, 1H), 7.12 (dd, J=8.4, 2.8 Hz, 1H), 7.03 (d, J=8.4 Hz, 1H), 6.98 (d, J=8.8 Hz, 2H), 5.86 (m, 2H), 5.25 (s, 2H), 3.89 (s, 3H), 3.47 (m, 2H), 3.16 (m, 2H), 1.12 (m, 2H), 0.35-0.26 (m, 2H); ¹³C NMR (100 MHz, CDCl₃) δ 191.8, 177.8 (2C), 164.5, 158.9, 132.5, 132.1, 130.8 (2C), 128.2, 127.8, 127.4, 118.8, 118.6, 114.7 (q, J=5.3 Hz, 1C), 114.4 (2C), 71.0, 55.8, 46.3, 45.6, 33.8, 33.6, 10.3, 10.1, 5.05, 4.96; ES-API MS: m/z calcd for C₂₇H₂₃F₃NO₅ 498.2, found 498.1 [M+H]⁺.

### (3aR,4R,4aR,5aS,6S,6aS)-2-(3-Chloro-4-hydroxyphenyl)-4,4a,5,5a,6,6a-hexahydro-4,6-ethenocyclopropa[f]isoindole-1,3(2H,3aH)-dione (98)

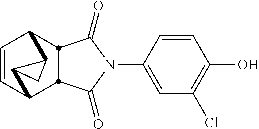 [SMILES: O=C1[C@@H]2[C@H](C(=O)N1c1ccc(O)c(Cl)c1)[C@@H]1C=C[C@H]2C2CC21]

Procedure B (microwave). Compound 98 was obtained by crystallization as a white solid (466 mg, 1.48 mmol, 94%). IR (cm⁻¹) 3396, 1694, 1496, 1291, 1195, 1166, 736; ¹H NMR (400 MHz, CDCl₃) δ 7.08 (d, J=2.4 Hz, 1H), 6.93 (d, J=8.8 Hz, 1H), 6.87 (dd, J=8.8, 2.4 Hz, 1H), 5.81 (dd, J=4.8, 3.2 Hz, 2H), 3.43 (m, 2H), 3.08 (dd, J=1.6, 1.6 Hz, 2H), 2.66 (br, 1H), 1.11 (m, 2H), 0.31-0.21 (m, 2H); ¹³C NMR (100 MHz, CDCl₃) δ 178.2 (2C), 152.8, 128.0, 127.9 (2C), 126.4, 124.1, 120.7, 116.7, 45.4 (2C), 33.9 (2C), 10.0 (2C), 4.8; ES-API MS: m/z calcd for C₁₇H₁₅ClNO₃ 316.1, found 316.1 [M+H]⁺.

### (3aR,4R,4aR,5aS,6S,6aS)-2-(3,5-Dichloro-4-hydroxyphenyl)-4,4a,5,5a,6,6a-hexahydro-4,6-ethenocyclopropa[f]isoindole-1,3(2H,3aH)-dione (99)

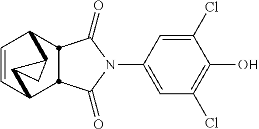 [SMILES: O=C1[C@@H]2[C@H](C(=O)N1c1cc(Cl)c(O)c(Cl)c1)[C@@H]1C=C[C@H]2C2CC21]

Procedure B (microwave). Compound 99 was obtained by crystallization as a light brown solid (423 mg, 1.21 mmol, 77%). IR (cm⁻¹) 3234, 1699, 1489, 1416, 1184, 1162, 732, ¹H NMR (400 MHz, CDCl₃) δ 7.13 (s, 2H), 6.08 (br, 1H), 5.86 (dd, J=4.4, 3.2 Hz, 2H), 3.49 (m, 2H), 3.13 (dd, J=1.6, 1.6 Hz, 2H), 1.15 (m, 2H), 0.36-0.26 (m, 2H); ¹³C NMR (100 MHz, CDCl₃) δ 177.5 (2C), 148.3, 128.1 (2C), 126.8 (2C), 124.7, 121.4 (2C), 45.5 (2C), 34.0 (2C), 10.1 (2C), 4.9, ES-API MS: m/z calcd for C₁₇H₁₄Cl₂NO₃ 350.0 found 350.2 [M+H]⁺.

### (3aR,4R,4aR,5aS,6S,6aS)-2-(4-Mercaptophenyl)-4,4a,5,5a,6,6a-hexahydro-4,6-ethenocyclopropa[f]isoindole-1,3(2H,3aH)-dione (100)

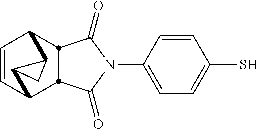 [SMILES: O=C1[C@@H]2[C@H](C(=O)N1c1ccc(S)cc1)[C@@H]1C=C[C@H]2C2CC21]

Procedure B (microwave). Compound 100 was obtained by crystallization as a yellow crystal (80%). IR (cm⁻¹) 3003, 2942, 1704, 1496, 1389, 1193, 1172, 812, 733; ¹H NMR (400 MHz, CDCl₃) δ 7.31 (d, J=8.4 Hz, 2H), 7.04 (d, J=8.8 Hz, 2H), 5.84 (dd, J=4.8, 3.2 Hz, 2H), 3.50 (s, 1H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 177.7 (2C), 132.1, 129.9 (2C), 129.6, 128.0 (2C), 127.2 (2C), 45.5 (2C), 34.0 (2C), 10.1 (2C), 4.9; ES-API MS: m/z calcd for C₁₇H₁₆NO₂S 298.1, found 298.1 [M+H]⁺.

### (3aR,4R,4aR,5aS,6S,6aS)-2-(4-Aminophenyl)-4,4a,5,5a,6,6a-hexahydro-4,6-ethenocyclopropa[f]isoindole-1,3(2H,3aH)-dione (101)

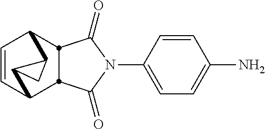 [SMILES: Nc1ccc(N2C(=O)[C@@H]3[C@H](C2=O)[C@@H]2C=C[C@H]3C3CC32)cc1]

Procedure A. Purification by flash chromatography on silica gel (gradient elution, 0→75% EtOAc in hexanes) afforded 101 (97 mg, 0.35 mmol, 66%) as a yellow solid. IR (cm⁻¹) 3336, 3060, 3029, 2948, 1699, 1517, 1404, 1199, 1165, 916, 734; ¹H NMR (400 MHz, CDCl₃) δ 6.90 (d, J=8.8 Hz, 2H), 6.67 (d, J=8.8 Hz, 2H), 5.83 (dd, J=4.8, 3.2 Hz, 2H), 3.76 (br, 2H), 3.47 (m, 2H), 3.09 (dd, J=2.0, 1.6 Hz, 2H), 1.13 (m, 1H), 0.29 (m, 2H); ¹³C NMR (100 MHz, CDCl₃) δ 178.1 (2C), 146.7, 127.8 (2C), 127.5 (2C), 122.2, 115.1 (2C), 45.2 (2C), 33.8 (2C), 9.9 (2C), 4.6; ES-API MS: m/z calcd for C₁₇H₁₇N₂O₂ 281.1, found 281.1 [M+H]⁺.

### (3aR,4R,4aR,5aS,6S,6aS)-2-(4-Hydroxyphennyl)-4,4a,5,5a,6,6a-hexahydro-4,6-ethenocyclopropa[f]isoindole-1,3(2H,3aH)-dione (102a)

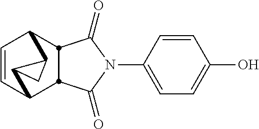 [SMILES: O=C1[C@@H]2[C@H](C(=O)N1c1ccc(O)cc1)[C@@H]1C=C[C@H]2C2CC21]

Procedure B (microwave). Compound 102a was obtained by crystallization as a white solid (145 mg, 0.52 mmol, 98%). IR (cm⁻¹) 3398, 1772, 1692, 1517, 1188; ¹H NMR (500 MHz, DMSO-d₆) δ 9.71 (s, 1H), 6.87 (d, J=8.5 Hz, 2H), 6.79 (d, J=9.0 Hz, 2H), 5.81 (dd, J=4.0, 4.0 Hz, 2H), 3.27 (m, 2H), 3.15 (m, 2H), 1.17 (m, 2H), 0.26 (dd, J=13.0, 7.0 Hz, 1H), 0.08 (m, 1H); ¹³C NMR (100 MHz, CDCl₃) δ 177.8 (2C), 157.2, 128.0 (2C), 127.5 (2C), 123.3, 115.3 (2C), 44.7 (2C), 33.2 (2C), 9.5 (2C), 4.3; ES-API MS: m/z calcd for C₁₇H₁₆NO₃ 282.1, found 282.1 [M+H]⁺.

### (3aR,4R,4aR,5aS,6S,6aS)-2-(2-Fluoro-4-hydroxyphenyl)-4,4a,5,5a,6.6a-hexahydro-4,6-ethenocyclopropa[f]isoindole-1,3(2H,3aH)-dione (102b)

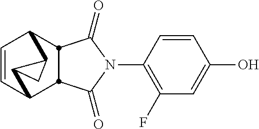 [SMILES: O=C1[C@@H]2[C@H](C(=O)N1c1ccc(O)cc1F)[C@@H]1C=C[C@H]2C2CC21]

Procedure B. Purification by flash chromatography on silica gel (gradient elution, 0→55% EtOAc in hexanes) followed by recrystallization in CH₂Cl₂/hexanes afforded 102b (95%) as a brown solid. IR (cm⁻¹) 3372, 1698, 1518, 1401, 1311, 1186; ¹H NMR (500 MHz, CDCl₃) δ 6.86 (ddd, J=67.5, 8.0, 7.5 Hz, 1H), 6.51 (m, 2H), 5.94 (br, 1H), 5.86 (m, 2H), 3.48 (m, 2H), 3.17 (d, J=18.0 Hz, 1H), 1.15 (m, 2H), 0.35-0.27 (m, 2H); ¹³C NMR (100 MHz, DMSO-d₆) δ 177.6 (2C), 159.5 (d, J=63.0 Hz, 1C), 156.7, 135.5, 130.8, 128.0, 127.7, 112.1 (d, J=20.8 Hz, 1C), 103.5 (d, J=9.7 Hz, 1C), 45.7, 45.1, 33.7, 33.5, 9.8 (2C), 4.8; ES-API MS: m/z calcd for C₁₇H₁₅FNO₃ 300.1, found 300.1 [M+H]⁺.

### (3aR,4R,4aR,5aS,6S,6aS)-2-(5-Hydroxypyridin-2-yl)-4,4a,5,5a,6,6a-hexahydro-4,6-ethenocyclopropa[f]isoindole-1,3(2H,3aH)-dione (103)

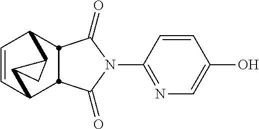 [SMILES: O=C1[C@@H]2[C@H](C(=O)N1c1ccc(O)cn1)[C@@H]1C=C[C@H]2C2CC21]

Procedure B (microwave). Purification by flash chromatography on silica gel (gradient elution, 0→70% EtOAc in CH₂Cl₂) followed by recrystallization in CH₂Cl₂/hexanes afforded 103 (1.64 g, 5.81 mmol, 72%) as an orange solid. IR (cm⁻¹) 3290, 3010, 2360, 1710, 1580, 1488, 1288, 1181; ¹H NMR (400 MHz, CDCl₃) δ 7.93 (d, J=2.8 Hz, 1H), 7.08 (dd, J=8.4, 2.8 Hz, 1H), 6.81 (d, J=8.8 Hz, 1H), 5.68 (dd, J=4.0, 4.0 Hz, 2H), 3.26 (m, 2H), 2.96 (m, 2H), 0.96 (m, 2H), 0.15-0.06 (m, 2H); ¹³C NMR (100 MHz, CDCl₃) δ 177.8 (2C), 154.1, 137.1, 136.7, 127.5 (2C), 124.8, 122.8, 45.2 (2C), 33.4 (2C), 9.5 (2C), 4.4; ES-API MS: m/z calcd for C₁₆H₁₅N₂O₃ 283.1, found 283.1 [M+H]⁺.

### (3aR,4R,4aR,5aS,6S,6aS)-2-(5-Hydroxypyrimidin-2-yl)-4,4a,5,5a,6,6a-hexahydro-4,6-ethenocyclopropa[f]isoindole-1,3(2H,3aH)-dione (104)

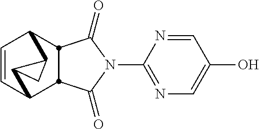 [SMILES: O=C1[C@@H]2[C@H](C(=O)N1c1ncc(O)cn1)[C@@H]1C=C[C@H]2C2CC21]

Procedure A. Purification by flash chromatography on silica gel (gradient elution, 0→5% MeOH in CH₂Cl₂) followed by recrystallization in MeOH/CH₂Cl₂/hexanes afforded 104 (1.08 g, 3.81 mmol, 48%) as an orange solid. IR (cm⁻¹) 3008, 1710, 1567, 1422, 1291, 1183, 731; ¹H NMR (400 MHz, CD₃OD) δ 8.38 (s, 1H), 5.86 (dd, J=4.8, 3.6 Hz, 2H), 3.39 (m, 2H), 3.26 (dd, J=1.6, 1.6 Hz, 2H), 1.21 (m, 2H), 0.36-0.26 (m, 2H); ¹³C NMR (100 MHz, CD₃OD) δ 177.5 (2C), 152.2, 145.7 (2C), 144.3, 127.4 (2C), 45.6 (2C), 33.5 (2C), 9.3 (2C), 3.7; ES-API MS: m/z calcd for C₁₅H₁₄N₃O₃ 284.1, found 284.1 [M+H]⁺.

### (3aR,4R,4aR,5aS,6S,6aS)-2-(3-Chloro-4-(2-(4-methoxyphenyl)-2-oxoethoxy)phenyl)-4,4a,5,5a,6,6a-hexahydro-4,6-ethenocyclopropa[f]isoindole-1,3(2H,3aH)-dione (10)

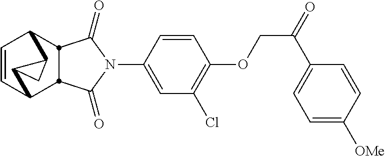 [SMILES: COc1ccc(C(=O)COc2ccc(N3C(=O)[C@@H]4[C@H](C3=O)[C@@H]3C=C[C@H]4C4CC43)cc2Cl)cc1]

Method C. Purification by flash chromatography on silica gel (gradient elution, 0→50% EtOAc in hexanes) followed by recrystallization in CH₂Cl₂/hexanes afforded 10 (112 mg, 0.24 mmol, 77%) as a white solid. IR (cm⁻¹) 2957, 1711, 1601, 1505, 1233, 1174, 972, 733; ¹H NMR (400 MHz, CDCl₃) δ 8.01 (d, J=8.8 Hz, 2H), 7.23 (d, J=2.4 Hz, 1H), 7.00 (dd, J=8.8, 2.4 Hz, 1H), 6.96 (d, J=9.2 Hz, 2H), 6.88 (d, J=8.8 Hz, 1H), 5.84 (dd, J=4.8, 3.2 Hz, 2H), 5.26 (s, 2H), 3.88 (s, 3H), 3.47 (m, 2H), 3.11 (dd, J=1.6, 1.6 Hz, 2H), 1.14 (m, 2H), 0.35-0.25 (m, 2H); ¹³C NMR (100 MHz, CDCl₃) δ 192.4, 177.6 (2C), 164.4, 153.9, 131.0 (2C), 128.8, 128.0 (2C), 127.5, 126.1, 125.9, 123.7, 114.3 (2C), 114.0, 72.1, 55.8, 45.4 (2C), 34.0 (2C), 10.1 (2C), 4.9; ES-API MS: m/z calcd for C₂₆H₂₃ClNO₅ 264.1 found 464.1 [M+H]⁺.

### (3aR,4R,4aR,5aS,6S,6aS)-2-(3,5-Dichloro-4-(2-(4-methoxyphenyl)-2-oxoethoxy)phenyl)-4,4a,5,5a,6,6a-hexahydro-4,6-ethenocyclopropa[f]isoindole-1,3(2H,3aH)-dione (11)

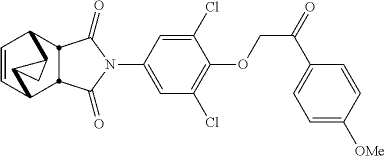 [SMILES: COc1ccc(C(=O)COc2c(Cl)cc(N3C(=O)[C@@H]4[C@H](C3=O)[C@@H]3C=C[C@H]4C4CC43)cc2Cl)cc1]

Method C. Purification by flash chromatography on silica gel (gradient elution, 0→35% EtOAc in hexanes) followed by recrystallization in CH₂Cl₂/hexanes afforded 11 (121 mg, 0.24 mmol, 85%) as a white solid. IR (cm⁻¹) 3077, 3010, 2954, 1714, 1601, 1471, 1172, 971, 733; ¹H NMR (400 MHz, CDCl₃) δ 7.98 (d, J=9.2 Hz, 2H), 7.22 (s, 2H), 6.96 (d, J=8.8 Hz, 2H), 5.86 (dd, J=4.4, 3.6 Hz, 2H), 5.20 (s, 2H), 3.88 (s, 3H), 3.50 (m, 2H), 3.14 (dd, J=1.6, 1.6 Hz, 2H), 1.16 (m, 2H), 0.37-0.26 (m, 2H); ¹³C NMR (100 MHz, CDCl₃) δ 191.2, 177.1 (2C), 164.2, 151.1, 130.7 (2C), 129.8 (2C), 128.9, 128.1 (2C), 127.6, 127.3 (2C), 114.2 (2C), 74.7, 55.8, 45.5 (2C), 34.1 (2C), 10.1 (2C), 4.9, ES-API MS: m/z calcd for C₂₆H₂₂Cl₂NO₅ 498.1, found 498.1 [M+H]⁺.

### (3aR,4R,4aR,5aS,6S,6aS)-2-(4-((2-(4-Methoxyphenyl)-2-oxoethyl)thio)phenyl)-4,4a,5,5a,6,6a-hexahydro-4,6-ethenocyclopropa[f]isoindole-1,3(2H,3aH)-dione (15)

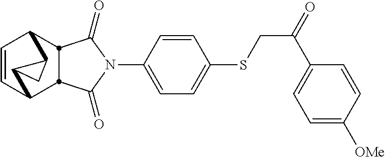 [SMILES: COc1ccc(C(=O)CSc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→55% EtOAc in hexanes) followed by recrystallization in CH₂Cl₂/hexanes afforded 15 (88 mg, 0.20 mmol, 59%) as a pale yellow solid. IR (cm⁻¹) 2955, 2358, 1706, 1669, 1599, 1497, 1379, 1261, 1175, 733; ¹H NMR (400 MHz, CDCl₃) δ 7.92 (d, J=9.2 Hz, 2H), 7.43 (d, J=8.8 Hz, 2H), 7.10 (d, J=8.8 Hz, 2H), 6.93 (d, J=8.8 Hz, 2H), 5.84 (dd, J=4.8, 3.6 Hz, 2H), 4.26 (s, 2H), 3.87 (s, 3H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 192.6, 177.6 (2C), 164.1, 136.3, 131.2 (2C), 130.5, 130.3 (2C), 128.5, 128.0 (2C), 127.1 (2C), 114.1 (2C), 55.7, 45.5 (2C), 41.0, 34.0 (2C), 10.1 (2C), 4.9; ES-API MS: m/z calcd for C₂₆H₂₃NO₄SNa 468.1, found 468.1 [M+Na]⁺.

### (3aR,4R,4aR,5aS,6S,6aS)-2-(4-((2-(4-Methoxyphenyl)-2-oxoethyl)amino)phenyl)-4,4a,5,5a,6,6a-hexahydro-4,6-ethenocyclopropa[f]isoindole-1,3(2H,3aH)-dione (16)

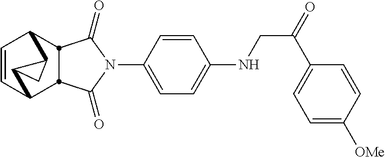 [SMILES: COc1ccc(C(=O)CNc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→45% EtOAc in hexanes) followed by recrystallization in CH₂Cl₂/hexanes afforded 16 (32 mg, 0.07 mmol, 71%) as a white solid. IR (cm⁻¹) 3394, 2959, 2924, 2852, 1702, 1602, 1524, 1260, 1172, 818, 735; ¹H NMR (400 MHz, CDCl₃) δ 7.99 (d, J=8.8 Hz, 2H), 6.98 (d, J=8.8 Hz, 4H), 6.70 (d, J=8.8 Hz, 2H), 5.85 (dd, J=4.8, 3.6 Hz, 2H), 5.12 (br, 1H), 4.52 (s, 2H), 3.89 (s, 3H), 3.48 (m, 2H), 3.10 (dd, J=1.6, 1.6 Hz, 2H), 1.13 (m, 2H), 0.33-0.25 (m, 2H); ¹³C NMR (100 MHz, CDCl₃) δ 193.0, 178.2 (2C), 164.1, 147.2, 130.1 (2C), 127.7 (3C), 127.5 (2C), 121.4, 114.1 (2C), 113.0 (2C), 55.6, 49.6, 45.2 (2C), 33.8 (2C), 9.9 (2C), 4.6; ES-API MS: m/z calcd for C₂₆H₂₃N₂O₄ 429.2 found 429.1 [M+H]⁺.

### (3aR,4R,4aR,5aS,6S,6aS)-2-(4-(2-Oxo-2-phenylethoxy)phenyl)-4,4a,5,5a,6,6a-hexahydro-4,6-ethenocyclopropa[f]isoindole-1,3(2H,3aH)-dione (20)

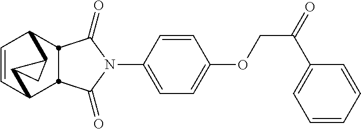 [SMILES: O=C(COc1ccc(N2C(=O)[C@@H]3[C@H](C2=O)[C@@H]2C=C[C@H]3C3CC32)cc1)c1ccccc1]

Procedure C. Purification by flash chromatography on silica gel (gradient elution, 0→45% EtOAc in hexanes) followed by recrystallization in CH₂Cl₂/hexanes afforded 20 (32 mg, 0.08 mmol, 45%) as a pale yellow solid. IR (cm⁻¹) 1704, 1513, 1218, 1184, 734; ¹H NMR (400 MHz, CDCl₃) δ 7.99 (d, J=7.2 Hz, 2H), 7.62 (t, J=7.6 Hz, 1H), 7.50 (dd, J=8.0, 7.6 Hz, 2H), 7.09 (d, J=8.8 Hz, 2H), 6.98 (d, J=8.8 Hz, 2H), 5.84 (dd, J=4.0, 4.0 Hz, 2H), 5.25 (s, 2H), 3.48 (m, 2H), 3.12 (m, 2H), 1.14 (m, 2H), 0.34-0.25 (m, 2H); ¹³C NMR (100 MHz, CDCl₃) δ 194.3, 178.0 (2C), 158.0, 134.6, 134.2, 129.1 (2C), 128.4 (2C), 128.00 (2C), 127.98 (2C), 125.6, 115.5 (2C), 71.2, 45.5 (2C), 34.0 (2C), 10.1 (2C), 4.9; ES-API MS: m/z calcd for C₂₅H₂₁NO₄Na 422.1 found 422.1 [M+Na]⁺.

### (3aR,4R,4aR,5aS,6S,6aS)-2-(4-(4-Methoxyphenethoxy)phenyl)-4,4a,5,5a,6,6a-hexahydro-4,6-ethenocyclopropa[f]isoindole-1,3(2H,3aH)-dione (29)

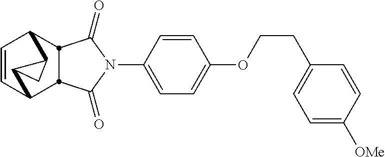 [SMILES: COc1ccc(CCOc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→35% EtOAc in hexanes) afforded 29 (77 mg, 0.19 mmol, 52%) as a white solid. IR (cm⁻¹) 3007, 2953, 1705, 1513, 1245, 1180, 1031, 826; ¹H NMR (400 MHz, CDCl₃) δ 7.19 (d, J=8.8 Hz, 2H), 7.05 (d, J=8.8 Hz, 2H), 6.92 (d, J=9.2 Hz, 2H), 6.85 (d, J=8.8 Hz, 2H), 5.85 (dd, J=4.8, 3.6 Hz, 2H), 4.12 (t, J=7.2 Hz, 2H), 3.79 (s, 3H), 3.48 (m, 2H), 3.11 (dd, J=1.6, 1.6 Hz, 2H), 3.03 (t, J=7.2 Hz, 2H), 1.14 (m, 2H), 0.34-0.25 (m, 2H); ¹³C NMR (100 MHz, CDCl₃) δ 178.1 (2C), 158.9, 158.5, 130.2, 130.1 (2C), 127.94 (2C), 127.86 (2C), 124.6, 115.2 (2C), 114.1 (2C), 69.3, 55.5, 45.4 (2C), 34.9, 34.0 (2C), 10.1 (2C), 4.9; ES-API MS: m/z calcd for C₂₆H₂₆NO₄ 416.2, found 416.2 [M+H]⁺.

### (3aR,4R,4aR,5aS,6S,6aS)-2-(4-(2-(3-Methoxyphenyl)-2-oxoethoxy)phenyl)-4,4a,5,5a,6,6a-hexahydro-4,6-ethenocyclopropa[f]isoindole-1,3(2H,3aH)-dione (30a)

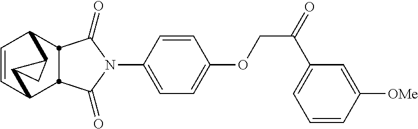 [SMILES: COc1cccc(C(=O)COc2ccc(N3C(=O)[C@@H]4[C@H](C3=O)[C@@H]3C=C[C@H]4C4CC43)cc2)c1]

Procedure C. Purification by flash chromatography on silica gel (gradient elution, 0→50% EtOAc in hexanes) followed by recrystallization in CH₂Cl₂/hexanes afforded 30a (140 mg, 0.33 mmol, 92%) as pale yellow foam. IR (cm⁻¹) 2954, 2359, 1704, 1512, 1392, 1236, 1185, 1041, 735; ¹H NMR (400 MHz, CDCl₃) δ 7.52 (d, J=8.0 Hz, 1H), 7.47 (dd, J=2.4, 1.2 Hz, 1H), 7.36 (dd, J=8.4, 7.6 Hz, 1H), 7.13 (dd, J=8.0, 2.4 Hz, 1H), 7.06 (d, J=8.8 Hz, 2H), 6.94 (d, J=8.8 Hz, 2H), 5.81 (dd, J=4.8, 3.6 Hz, 2H), 5.21 (s, 2H), 3.82 (s, 3H), 3.43 (m, 2H), 3.08 (dd, J=1.6, 1.6 Hz, 2H), 1.11 (m, 2H), 0.31-0.22 (m, 2H); ¹³C NMR (100 MHz, CDCl₃) δ 193.9, 177.9 (2C), 160.0, 157.9, 135.7, 129.9, 127.9 (2C), 127.8 (2C), 125.5, 120.6, 120.5, 115.3 (2C), 112.4, 71.0, 55.6, 45.3 (2C), 33.9 (2C), 10.0 (2C), 4.7; ES-API MS: m/z calcd for C₂₆H₂₃NO₅Na 452.1, found 452.1 [M+Na]⁺.

### (3aR,4R,4aR,5aS,6S,6aS)-2-(4-(2-(2-Methoxyphenyl)-2-oxoethoxy)phenyl)-4,4a,5,5a,6,6a-hexahydro-4,6-ethenocyclopropa[f]isoindole-1,3(2H,3aH)-dione (31a)

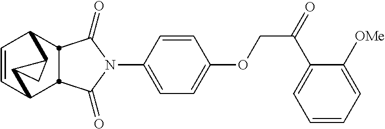 [SMILES: COc1ccccc1C(=O)COc1ccc(N2C(=O)[C@@H]3[C@H](C2=O)[C@@H]2C=C[C@H]3C3CC32)cc1]

Procedure C. Purification by flash chromatography on silica gel (gradient elution, 0→60% EtOAc in hexanes) to obtain a mixture of starting material phenol and the product, which was further re-purified via HPLC (reverse phase, MeCN/H₂O/0.1% TFA) to the product 31a obtain pale yellow foam (33 mg, 0.077 mmol, 43%). IR (cm⁻¹) 3053, 3008, 2952, 1706, 1597, 1512, 1184, 973, 735; ¹H NMR (400 MHz, CDCl₃) δ 7.93 (dd, J=8.0, 1.6 Hz, 1H), 7.54 (ddd, J=8.0, 8.0, 1.6 Hz, 1H), 7.06 (d, J=8.8 Hz, 2H), 7.06 (m, 1H), 7.01 (d, J=8.4 Hz, 2H), 6.94 (d, J=9.2 Hz, 2H), 5.84 (dd, J=4.8, 3.6 Hz, 2H), 5.22 (s, 2H), 3.95 (s, 3H), 3.47 (m, 2H), 3.12 (m, 2H), 1.13 (m, 2H), 0.34-0.25 (m, 2H); ¹³C NMR (00 MHz, CDCl₃) δ 195.1, 178.1 (2C), 159.3, 158.2, 135.0, 131.1, 127.7 (2C), 127.6 (2C), 124.9, 124.6, 121.2, 115.4 (2C), 111.5, 74.4, 55.7, 45.2 (2C), 33.8 (2C), 9.9 (2C), 4.7; ES-API MS: m/z calcd for C₂₆H₂₃NO₅Na 452.1, found 452.2 [M+Na]⁺.

### (3aR,4R,4aR,5aS,6S,6aS)-2-(4-(2-(4-Ethoxyphenyl)-2-oxoethoxy)phenyl)-4,4a,5,5a,6,6a-hexahydro-4,6-ethenocyclopropa[f]isoindole-1,3(2H,3aH)-dione (32a)

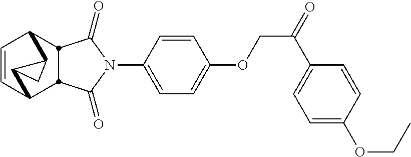 [SMILES: CCOc1ccc(C(=O)COc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→45% EtOAc in hexanes) followed by recrystallization in CH₂Cl₂/hexanes afforded 32a (93 mg, 0.21 mmol, 73%) as a yellow solid. IR (cm⁻¹) 1705, 1600, 1511, 1393, 1226, 1172; ¹H NMR (400 MHz, CDCl₃) δ 7.96 (d, J=8.8 Hz, 2H), 7.08 (d, J=8.8 Hz, 2H), 6.97 (d, J=9.2 Hz, 2H), 6.94 (d, J=8.8 Hz, 2H), 5.84 (dd, J=4.4, 3.2 Hz, 2H), 5.18 (s, 2H), 4.11 (q, J=6.8 Hz, 2H), 3.48 (m, 2H), 3.11 (m, 2H), 1.45 (t, J=7.2 Hz, 3H), 1.14 (m, 2H), 0.34-0.25 (m, 2H); ¹³C NMR (100 MHz, CDCl₃) δ 192.8, 178.0 (2C), 163.8, 158.1, 130.8 (2C), 128.1, 128.0 (4C), 127.5, 125.5, 116.1, 115.5 (2C), 114.7 (2C), 71.1, 64.1, 45.5 (2C), 34.0 (2C), 14.9, 10.1 (2C), 4.9; ES-API MS: m/z calcd for C₂₇H₂₅NO₅Na 466.2, found 466.1 [M+Na]⁺.

### (3aR,4R,4aR,5aS,6S,6aS)-2-(4-(2-(Oxo-2-(4-(prop-2-yn-1-yloxy)phenyl)ethoxy)phenyl)-4,4a,5,5a,6,6a-hexahydro-4,6-ethenocyclopropa[f]isoindole-1,3(2H,3aH)-dione (33a)

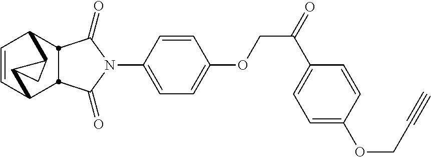 [SMILES: C#CCOc1ccc(C(=O)COc2ccc(N3C(=O)[C@@H]4[C@H](C3=O)[C@@H]3C=C[C@H]4C4CC43)cc2)cc1]

Procedure C. Purification flash chromatography on silica gel (gradient elution, 0→50% EtOAc in hexanes) followed by recrystallization in CH₂Cl₂/hexanes afforded 33a (32 mg, 0.07 mmol, 86%) as a colorless solid. IR (cm⁻¹) 3298, 3052, 3011, 2956, 1772, 1704, 1600, 1511, 1223, 1173, 734; ¹H NMR (400 MHz, CDCl₃) δ 7.98 (d, J=8.8 Hz, 2H), 7.06 (d, J=8.8 Hz, 2H), 7.03 (d, J=9.2 Hz, 2H), 6.96 (d, J=9.2 Hz, 2H), 5.82 (dd, J=4.8, 3.2 Hz, 2H), 5.17 (s, 2H), 4.76 (d, J=2.4 Hz, 2H), 3.46 (m, 2H), 3.10 (dd, J=2.0, 2.0 Hz, 2H), 2.55 (t, J=2.4 Hz, 1H), 1.12 (m, 2H), 0.32-0.23 (m, 2H); ¹³C NMR (100 MHz, CDCl₃) δ 192.9, 178.0 (2C), 162.1, 158.1, 130.8 (2C), 128.4, 128.1, 127.99 (2C), 127.98, 125.6, 115.5 (2C), 115.1 (2C), 76.6, 71.2, 56.1, 45.5 (2C), 34.0 (2C), 10.1 (2C); ES-API MS: m/z calcd for C₂₈H₂₄NO₅ 454.2, found 454.1 [M+H]⁺.

### (3aR,4R,4aR,5aS,6S,6aS)-2-(4-(2-(4-Butoxyphenyl)-2-oxoethoxy)phenyl)-4,4a,5,5a,6,6a-hexahydro-4,6-ethenocyclopropa[f]isoindole-1,3(2H,3aH)-dione (34a)

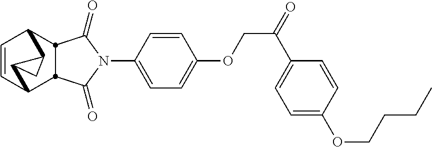 [SMILES: CCCCOc1ccc(C(=O)COc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→45% EtOAc in hexanes) followed by recrystallization in CH₂Cl₂/hexanes afforded 34a (110 mg, 0.23 mmol, 56%) as a colorless needle. IR (cm⁻¹) 2957, 2874, 1705, 1601, 1512, 1224, 1173, 965, 830, 734; ¹H NMR (400 MHz, CDCl₃) δ 7.96 (d, J=8.8 Hz, 2H), 7.08 (d, J=9.2 Hz, 2H), 6.97 (d, J=9.2 Hz, 2H), 6.94 (d, J=8.8 Hz, 2H), 5.84 (dd, J=4.4, 3.2 Hz, 2H), 5.18 (s, 2H), 4.04 (t, J=6.8 Hz, 2H), 3.48 (m, 2H), 3.11 (dd, J=2.0, 2.0 Hz, 2H), 1.79 (pent, J=7.2 Hz, 2H), 1.50 (hex, J=7.6 Hz, 2H), 1.14 (m, 2H), 0.98 (t, J=7.2 Hz, 3H), 0.34-0.25 (m, 2H); ¹³C NMR (100 MHz, CDCl₃) δ 192.8, 178.0 (2C), 164.0, 158.1, 130.8 (2C), 128.0 (4C), 127.4, 125.5, 115.5 (2C), 114.7 (2C), 71.1, 68.2, 45.5 (2C), 34.0 (2C), 31.3, 19.4, 14.0, 10.1 (2C), 4.9; ES-API MS: m/z calcd for C₂₉H₂₉NO₅Na 494.2, found 494.1 [M+Na]⁺.

### (3aR,4R,4aR,5aS,6S,6aS)-2-(4-(2-(4-fluorophenyl)-2-oxoethoxy)phenyl)-4,4a,5,5a,6,6a-hexahydro-4,6-ethenocyclopropa[f]isoindole-1,3(2H,3aH)-dione (35a)

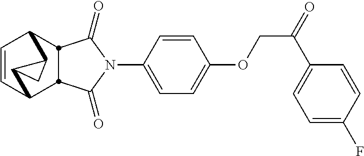 [SMILES: O=C(COc1ccc(N2C(=O)[C@@H]3[C@H](C2=O)[C@@H]2C=C[C@H]3C3CC32)cc1)c1ccc(F)cc1]

Procedure C. Purification by flash chromatography on silica gel (gradient elution, 0→45% EtOAc in hexanes) followed by recrystallization in CH₂Cl₂/hexanes afforded 35a (150 mg, 0.36 mmol, 44%) as a colorless solid. IR (cm⁻¹) 1704, 1509, 1225, 1186, 971, 835, 736; ¹H NMR (400 MHz, CDCl₃) δ 8.03 (dd, J=8.8, 5.2 Hz, 2H), 7.17 (dd, J=8.4, 8.8 Hz, 2H), 7.09 (d, J=8.8 Hz, 2H), 6.97 (d, J=9.2 Hz, 2H), 5.84 (dd, J=4.8, 3.6 Hz, 2H), 5.19 (s, 2H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 193.0, 178.0 (2C), 166.4 (d, J=255 Hz, 1C), 157.9, 131.3 (d, J=9.5 Hz, 2C), 131.1 (d, J=3.0 Hz, 1C), 128.04 (2C), 127.97 (2C), 125.7, 116.3 (d, J=21.8 Hz, 2C), 115.5 (2C), 71.3, 45.5 (2C), 34.0 (2C), 10.1 (2C), 4.9; ES-API MS: m/z calcd for C₂₅H₂₀FNO₄Na 440.1, found 440.1 [M+Na]⁺.

### (3aR,4R,4aR,5aS,6S,6aS)-2-(4-(2-(4-Chlorophenyl)-2-oxoethoxy)phenyl)-4,4a,5,5a,6,6a-hexahydro-4,6-ethenocyclopropa[f]isoindole-1,3(2H,3aH)-dione (36a)

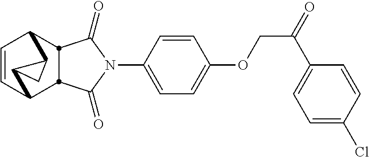 [SMILES: O=C(COc1ccc(N2C(=O)[C@@H]3[C@H](C2=O)[C@@H]2C=C[C@H]3C3CC32)cc1)c1ccc(Cl)cc1]

Procedure C. Purification by flash chromatography on silica gel (gradient elution, 0→45% EtOAc in hexanes) followed by recrystallization in CH₂Cl₂/hexanes afforded 36a (223 mg, 0.51 mmol, 51%) as a colorless solid. IR (cm⁻¹) 1704, 1515, 1397, 1219, 1174, 1087, 966, 736; ¹H NMR (400 MHz, CDCl₃) δ 7.94 (d, J=8.4 Hz, 2H), 7.47 (d, J=8.4 Hz, 2H), 7.09 (d, J=8.8 Hz, 2H), 6.96 (d, J=9.2 Hz, 2H), 5.84 (dd, J=4.8, 3.6 Hz, 2H), 5.18 (s, 2H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 193.2, 177.8 (2C), 157.6, 140.5, 132.7, 129.7 (2C), 129.2 (2C), 127.84 (2C), 127.76 (2C), 125.6, 115.2 (2C), 71.1, 45.2 (2C), 33.8 (2C), 9.9 (2C), 4.7; ES-API MS: m/z calcd for C₂₅H₂₁ClNO₄ 434.1, found 434.1 [M+H]⁺.

### (3aR,4R,4aR,5aS,6S,6aS)-2-(4-(2-(4-Bromophenyl)-2-oxoethoxy)phenyl)-4,4a,5,5a,6,6a-hexahydro-4,6-ethenocyclopropa[f]isoindole-1,3(2H,3aH)-dione (37a)

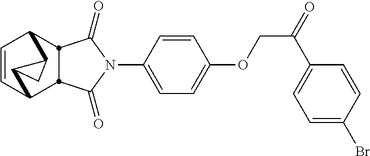 [SMILES: O=C(COc1ccc(N2C(=O)[C@@H]3[C@H](C2=O)[C@@H]2C=C[C@H]3C3CC32)cc1)c1ccc(Br)cc1]

Procedure C. Purification by flash chromatography on silica gel (gradient elution, 0→40% EtOAc in hexanes) followed by recrystallization in CH₂Cl₂/hexanes afforded 37a (35 mg, 0.073 mmol, 69%) as a pale yellow solid. IR (cm⁻¹) 1703, 1585, 1514, 1399, 1229, 1194, 984, 822; ¹H NMR (400 MHz, CDCl₃) δ 7.85 (d, J=8.4 Hz, 2H), 7.63 (d, J=8.4 Hz, 2H), 7.09 (d, J=8.8 Hz, 2H), 6.96 (d, J=9.2 Hz, 2H), 5.84 (dd, J=4.0, 3.6 Hz, 2H), 5.18 (s, 2H), 3.47 (m, 2H), 3.11 (m, 2H), 1.14 (m, 2H), 0.34-0.25 (m, 2H); ¹³C NMR (100 MHz, CDCl₃) δ 193.6, 178.0 (2C), 157.8, 133.3, 132.4 (2C), 130.0 (2C), 129.5, 128.04 (2C), 127.96 (2C), 125.8, 115.4 (2C), 71.2, 45.4 (2C), 34.0 (2C), 10.1 (2C), 4.9; ES-API MS: m/z calcd for C₂₅H₂₀BrNO₄Na 500.0, found 500.0 [M+Na]⁺.

### (3aR,4R,4aR,5aS,6S,6aS)-2-(4-(2-oxo-2-(p-Tolyl)ethoxy)phenyl)-4,4a,5,5a,6,6a-hexahydro-4,6-ethenocyclopropa[f]isoindole-1,3(2H,3aH)-dione (38a)

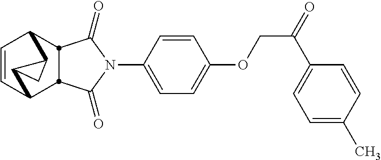 [SMILES: Cc1ccc(C(=O)COc2ccc(N3C(=O)[C@@H]4[C@H](C3=O)[C@@H]3C=C[C@H]4C4CC43)cc2)cc1]

Procedure C. Purification by flash chromatography on silica gel (gradient elution, 0→40% EtOAc in hexanes) followed by recrystallization in CH₂Cl₂/hexanes afforded 38a (100 mg, 0.24 mmol, 65%) as a white solid. IR (cm⁻¹) 3055, 3008, 2952, 1705, 1609, 1514, 1386, 1310, 1228, 1183, 964, 818, 734; ¹H NMR (400 MHz, CDCl₃) δ 7.89 (d, J=8.0 Hz, 2H), 7.29 (d, J=8.0 Hz, 2H), 7.08 (d, J=8.8 Hz, 2H), 6.97 (d, J=8.8 Hz, 2H), 5.84 (dd, J=4.8, 3.6 Hz, 2H), 5.22 (s, 2H), 3.48 (m, 2H), 3.11 (dd, J=1.6, 1.2 Hz, 2H), 2.43 (s, 3H), 1.14 (m, 2H), 0.34-0.25 (m, 2H); ¹³C NMR (100 MHz, CDCl₃) δ 193.9, 178.0 (2C), 158.1, 145.2, 132.2, 129.7 (2C), 128.5 (2C), 128.0 (4C), 125.6, 115.5 (2C), 71.2, 45.5 (2C), 34.0 (2C), 22.0, 10.1 (2C), 4.9; ES-API MS: m/z calcd for C₂₆H₂₄NO₄ 414.2, found 414.1 [M+H]⁺.

### (3aR,4R,4aR,5aS,6S,6aS)-2-(4-(2-Oxo-2-(4-(trifluoromethyl)phenyl)ethoxy)phenyl)-4,4a,5,5a,6,6a-hexahydro-4,6-ethenocyclopropa[f]isoindole-1,3(2H,3aH)-dione (39a)

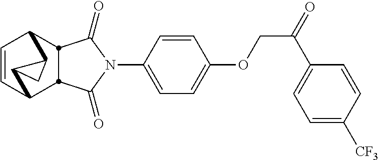 [SMILES: O=C(COc1ccc(N2C(=O)[C@@H]3[C@H](C2=O)[C@@H]2C=C[C@H]3C3CC32)cc1)c1ccc(C(F)(F)F)cc1]

Procedure C. Purification by flash chromatography on silica gel (gradient elution, 0→50% EtOAc in hexanes) followed by recrystallization in CH₂Cl₂/hexanes afforded 39a (25 mg, 0.053 mmol, 30%) as a colorless solid. IR (cm⁻¹) 1704, 1516, 1326, 1176, 1123, 1067, 718; ¹H NMR (400 MHz, CDCl₃) δ 8.10 (d, J=8.4 Hz, 2H), 7.77 (d, J=8.0 Hz, 2H), 7.10 (d, J=8.8 Hz, 2H), 6.97 (d, J=9.2 Hz, 2H), 5.84 (d, J=4.4, 3.2 Hz, 2H), 5.23 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 193.9, 178.0 (2C), 157.7, 129.0 (2C), 128.1 (2C), 128.0 (2C), 126.19, 126.15, 126.11, 126.07, 125.9, 115.5 (2C), 71.5, 45.5 (2C), 34.0 (2C), 10.1 (2C), 4.9, ES-API MS: m/z calcd for C₂₆H₂₀F₃NO₄Na 490.1, found 490.1 [M+Na]⁺.

### (3aR,4R,4aR,5aS,6S,6aS)-2-(4-(2-(Oxo-2-(4-(trifluoromethoxy)phenyl)ethoxy)phenyl)-4,4a,5,5a,6,6a-hexahydro-4,6-ethenocyclopropa[f]isoindole-1,3(2H,3aH)-dione (40a)

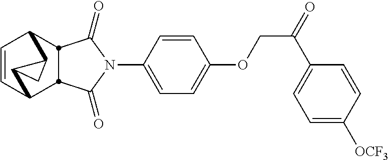 [SMILES: O=C(COc1ccc(N2C(=O)[C@@H]3[C@H](C2=O)[C@@H]2C=C[C@H]3C3CC32)cc1)c1ccc(OC(F)(F)F)cc1]

Procedure C. Purification by flash chromatography on silica gel (gradient elution, 0→40% EtOAc in hexanes) followed by recrystallization in CH₂Cl₂/hexanes afforded 40a (35 mg, 0.072 mmol, 51%) as a colorless solid. IR (cm⁻¹) 1704, 1515, 1398, 1260, 1230, 1194, 1174, 989, 734; ¹H NMR (400 MHz, CDCl₃) δ 8.06 (d, J=8.8 Hz, 2H), 7.32 (d, J=8.4 Hz, 2H), 7.09 (d, J=8.8 Hz, 2H), 6.97 (d, J=8.8 Hz, 2H), 5.84 (dd, J=4.8, 3.6 Hz, 2H), 5.19 (s, 2H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 193.2, 178.0 (2C), 157.8, 153.4, 132.8, 130.7 (2C), 128.08 (2C), 127.98 (2C), 125.8, 121.7, 120.8 (2C), 115.5 (2C), 71.3, 45.5 (2C), 34.0 (2C), 10.1 (2C), 4.9; ES-API MS: m/z calcd for C₂₆H₂₀F₃NO₅Na 506.1, found 506.1 [M+Na]⁺.

### (3aR,4R,4aR,5aS,6S,6aS)-2-(4-(2-(4-Hydroxyphenyl)-2-oxoethoxy)phenyl)-4,4a5,5a,6,6a-hexahydro-4,6-ethenocyclopropa[f]isoindole-1,3(2H,3aH)-dione (41a)

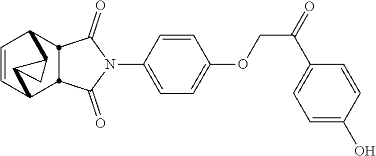 [SMILES: O=C(COc1ccc(N2C(=O)[C@@H]3[C@H](C2=O)C2C=C[C@H]3C3C[C@@H]23)cc1)c1ccc(O)cc1]

The acetate 42a (530 mg, 1.16 mmol) was dissolved in MeOH (4.0 mL) and added K₂CO₃ (320 mg, 2.31 mmol). The resulting mixture was stirred at rt for 2 h. The solvent was removed under reduced pressure and the residue was purified by flash chromatography on silica gel (gradient elution, 0470% EtOAc in hexanes) to give phenol 41a (375 mg, 0.90 mmol, 78%) as a white solid. IR (cm⁻¹) 3334, 1704, 1700, 1602, 1512, 1392, 1186, 1173, 735; ¹H NMR (400 MHz, CDCl₃) δ 7.92 (d, J=8.0 Hz, 2H), 7.07 (d, J=9.2 Hz, 2H), 6.97 (d, J=8.8 Hz, 2H), 6.86 (d, J=8.8 Hz, 2H), 5.84 (dd, J=4.8, 3.6 Hz, 2H), 5.18 (s, 2H), 3.48 (m, 2H), 3.12 (dd, J=1.6, 1.6 Hz, 2H), 1.15 (m, 2H), 0.34-0.25 (m, 2H); ¹³C NMR (100 MHz, CDCl₃) δ 192.8, 178.2 (2C), 161.0, 158.2, 131.1 (2C), 128.03 (2C), 127.99 (2C), 127.7, 125.5, 115.9 (2C), 115.5 (2C), 71.0, 45.5 (2C), 34.0 (2C), 10.1 (2C), 4.9; ES-API MS: m/z calcd for C₂₅H₂₂NO₅ 416.1, found 416.1 [M+H]⁺.

### 4-(2-(4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)phenoxy)acetyl)phenyl acetate (42a)

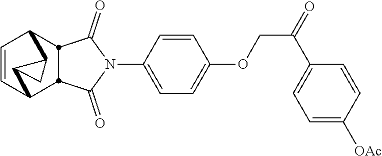 [SMILES: CC(=O)Oc1ccc(C(=O)COc2ccc(N3C(=O)[C@@H]4[C@H](C3=O)C3C=C[C@H]4C4C[C@@H]34)cc2)cc1]

Procedure C. Purification by flash chromatography on silica gel (gradient elution, 0→50% EtOAc in hexanes) followed by recrystallization in CH₂Cl₂/hexanes afforded 42a (45 mg, 0.098 mmol, 55%) as a white solid. IR (cm⁻¹) 1760, 1705, 1600, 1513, 1193, 1166; ¹H NMR (400 MHz, CDCl₃) δ 8.03 (d, J=8.8 Hz, 2H), 7.24 (d, J=8.8 Hz, 2H), 7.09 (d, J=9.2 Hz, 2H), 6.97 (d, J=8.8 Hz, 2H), 5.84 (dd, J=4.4, 3.6 Hz, 2H), 5.21 (s, 2H), 3.48 (m, 2H), 3.12 (dd, J=1.6, 1.2 Hz, 2H), 2.33 (s, 3H), 1.14 (m, 2H), 0.34-0.25 (m, 2H); ¹³C NMR (100 MHz, CDCl₃) δ 193.1, 178.0 (2C), 168.9, 157.9, 155.2, 132.2, 130.2 (2C), 128.03 (2C), 127.98 (2C), 125.7, 122.3 (2C), 115.5 (2C), 71.2, 45.5 (2C), 34.0 (2C), 21.4, 10.1 (2C), 4.9; ES-API MS: m/z calcd for C₂₇H₂₃NO₆Na 480.1, found 480.1 [M+Na]⁺.

### 4-(2-(4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)phenoxy)acetyl)phenyl methyl carbonate (43a)

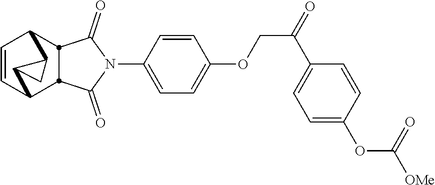 [SMILES: COC(=O)Oc1ccc(C(=O)COc2ccc(N3C(=O)[C@@H]4[C@H](C3=O)C3C=C[C@H]4C4C[C@@H]34)cc2)cc1]

Procedure C. Purification by flash chromatography on silica gel (gradient elution, 0→55% EtOAc in hexanes) followed by recrystallization in CH₂Cl₂/hexanes afforded 43a (47 mg, 0.099 mmol, 56%) as a white solid. IR (cm⁻¹) 1770, 1704, 1508, 1262, 1216, 734; ¹H NMR (400 MHz, CDCl₃) δ 8.04 (d, J=8.8 Hz, 2H), 7.33 (d, J=8.8 Hz, 2H), 7.09 (d, J=9.2 Hz, 2H), 6.97 (d, J=8.8 Hz, 2H), 5.84 (dd, J=4.8, 3.6 Hz, 2H), 5.21 (s, 2H), 3.93 (s, 3H), 3.48 (m, 2H), 3.12 (dd, J=1.6, 2.0 Hz, 2H), 1.15 (m, 2H), 0.34-0.25 (m, 2H); ¹³C NMR (100 MHz, CDCl₃) δ 193.1, 178.0 (2C), 157.9, 155.3, 153.6, 132.3, 130.3 (2C), 128.04 (2C), 127.98 (2C), 125.7, 121.7 (2C), 115.5 (2C), 71.2, 55.9, 45.5 (2C), 34.0 (2C), 10.1 (2C), 4.9; ES-API MS: m/z calcd for C₂₇H₂₃NO₇Na 496.1, found 496.1 [M+Na]⁺.

### Methyl(4-(2-(4-((3aR,4R,4aR,5aS,6S,6aS)-1,3-dioxo-3,3a,4,4a,5,5a,6,6a-octahydro-4,6-ethenocyclopropa[f]isoindol-2(1H)-yl)phenoxy)acetyl)phenyl)carbamate (44a)

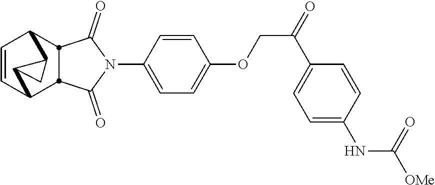 [SMILES: COC(=O)Nc1ccc(C(=O)COc2ccc(N3C(=O)[C@@H]4[C@H](C3=O)C3C=C[C@H]4C4C[C@@H]34)cc2)cc1]

Procedure C. Purification by flash chromatography on silica gel (gradient elution, 0→60% EtOAc in hexanes) followed by recrystallization in CH₂Cl₂/hexanes afforded 44a (55 mg, 0.11 mmol, 68%) as a pale yellow solid. IR (cm⁻¹) 3315, 2954, 1706, 1683, 1516, 1393, 1224, 1190, 1084, 972; ¹H NMR (400 MHz, CDCl₃/CD₃OD) δ 7.83 (d, J=8.8 Hz, 2H), 7.46 (d, J=8.4 Hz, 2H), 6.95 (d, J=9.2 Hz, 2H), 6.88 (d, J=9.2 Hz, 2H), 5.73 (dd, J=4.4, 3.6 Hz, 2H), 5.15 (s, 2H), 3.68 (s, 3H), 3.35 (m, 2H), 3.03 (m, 2H), 1.05 (m, 2H), 0.24-0.14 (m, 2H); ¹³C NMR (100 MHz, CDCl₃/CD₃OD) δ 193.1, 178.3 (2C), 157.9, 154.3, 144.2, 129.5 (2C), 128.6, 127.8 (2C), 127.7 (2C), 125.1, 117.7 (2C), 115.2 (2C), 70.5, 52.3, 45.2 (2C), 33.7 (2C), 9.8 (2C), 4.5; ES-API MS: m/z calcd for C₂₇H₂₅N₂O₆ 473.2, found 473.2 [M+H]⁺.

### Methyl4-(2-(4-((3aR,4R,4aR,5aS,6S,6aS)-1,3-dioxo-3,3a,4,4a,5,5a,6,6a-octahydro-4,6-ethenocyclopropa[f]isoindol-2(1H)-yl)phenoxy)acetyl)benzoate (45a)

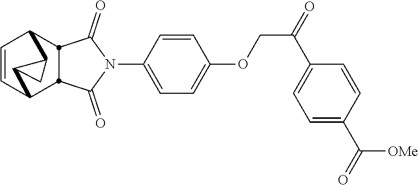 [SMILES: COC(=O)c1ccc(C(=O)COc2ccc(N3C(=O)[C@@H]4[C@H](C3=O)C3C=C[C@H]4C4C[C@@H]34)cc2)cc1]

Procedure C. Purification by flash chromatography on silica gel (gradient elution, 0→50% EtOAc in hexanes) followed by recrystallization in CH₂Cl₂/hexanes afforded 45a (27 mg, 0.060 mmol, 33%) as a yellow solid. IR (cm⁻¹) 1705, 1513, 1282, 1218, 1186, 1110, 734; ¹H NMR (400 MHz, CDCl₃) δ 8.15 (d, J=8.4 Hz, 2H), 8.04 (d, J=8.4 Hz, 2H), 7.09 (d, J=8.8 Hz, 2H), 6.98 (d, J=8.8 Hz, 2H), 5.84 (dd, J=4.8, 3.6 Hz, 2H), 5.24 (s, 2H), 3.96 (s, 3H), 3.48 (m, 2H), 3.12 (m, 2H), 1.15 (m, 2H), 0.34-0.25 (m, 2H); ¹³C NMR (100 MHz, CDCl₃) δ 194.1, 178.0 (2C), 166.2, 157.8, 137.8, 134.8, 130.2 (2C), 128.4 (2C), 128.1 (2C), 128.0 (2C), 125.8, 115.5 (2C), 71.4, 52.8, 45.5 (2C), 34.0 (2C), 10.1 (2C), 4.9; ES-API MS: m/z calcd for C₂₇H₂₃NO₆Na 480.1, found 480.1 [M+Na]⁺.

### (4R,4aR,5aS,6S,6aS)-2-(S-(2-(6-Methoxypyridin-3-yl)-2-oxoethoxy)pyridin-2-yl)-4,4a,5,5a,6,6a-hexahydro-4,6-ethenocyclopropa[f]isoindole-1,3(2H,3aH)-dione (46a)

Procedure C. Purification via flash chromatography on silicagel (0-45% EtOAc/hexanes) and recrystallization from CH₂Cl₂/hexanes delivered 46a a white solid (63 mg, 0.15 mmol, 42%). ¹H NMR (400 MHz, CDCl₃) δ 8.10 (d, J=8.4 Hz, 2H), 7.89 (d, J=8.4 Hz, 2H), 7.81 (dd, J=8.4, 1.2 Hz, 2H), 7.63 (dddd, J=7.2, 7.6, 1.2, 1.2 Hz, 1H), 7.51 (dd, J=7.6, 7.6 Hz, 2H), 7.10 (d, J=9.2 Hz, 2H), 6.99 (d, J=8.8 Hz, 2H), 5.84 (dd, J=4.8, 3.6 Hz, 2H), 5.27 (s, 2H), 3.48 (m, 2H), 3.12 (dd, J=1.6, 1.6 Hz, 2H), 1.15 (m, 2H), 0.35-0.25 (m, 2H); ¹³C NMR (100 MHz, CDCl₃) δ 196.0, 194.2, 178.0 (2C), 157.8, 142.3, 137.1, 136.9, 133.3, 130.4 (2C), 130.3 (2C), 128.7 (2C), 128.4 (2C), 128.1 (2C), 128.0 (2C), 125.8, 115.5 (2C), 71.5, 45.5 (2C), 34.0 (2C), 10.1 (2C), 4.9.

### (3aR,4R,4aR,5aS,6S,6aS)-2-(4-(2-(Naphthalen-2-yl)-2-oxoethoxy)phenyl)-4,4a,5,5a,6,6a-hexahydro-4,6-ethenocyclopropa[f]isoindole-1,3(2H,3aH)-dione (47a)

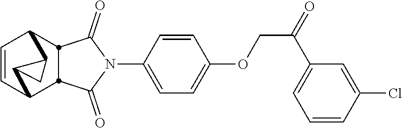 [SMILES: O=C(COc1ccc(N2C(=O)[C@@H]3[C@H](C2=O)C2C=C[C@H]3C3C[C@@H]23)cc1)c1cccc(Cl)c1]

Procedure C. Purification by flash chromatography on silica gel (gradient elution, 0→45% EtOAc in hexanes) followed by recrystallization in CH₂Cl₂/hexanes afforded 47a (33 mg, 0.076 mmol, 43%) as a white solid. IR (cm⁻¹) 3410, 2964, 1710, 1261, 1029, 801; ¹H NMR (400 MHz, CDCl₃) δ 7.97 (dd, J=2.0, 1.6 Hz, 1H), 7.86 (ddd, J=7.6, 1.6, 1.2 Hz, 1H), 7.59 (ddd, J=8.0, 1.2, 1.2 Hz, 1H), 7.44 (dd, J=8.0, 8.0 Hz, 1H), 7.09 (d, J=9.2 Hz, 2H), 6.97 (d, J=9.2 Hz, 2H), 5.84 (dd, J=4.8, 3.2 Hz, 2H), 5.20 (s, 2H), 3.48 (m, 2H), 3.12 (dd, J=1.6, 1.2 Hz, 2H), 1.14 (m, 2H), 0.34-0.25 (m, 2H); ¹³C NMR (100 MHz, CDCl₃) δ 193.3, 178.0 (2C), 157.8, 136.1, 135.4, 134.1, 130.4, 128.6, 128.1 (2C), 128.0 (2C), 126.5, 125.8, 115.5 (2C), 71.3, 45.5 (2C), 34.0 (2C), 10.1 (2C), 4.9; ES-API MS: m/z calcd for C₂₅H₂₁ClNO₄ 434.1, found 434.1 [M+H]⁺.

### (3aR,4R,4aR,5aS,6S,6aS)-2-(4-(2-(2,3-Dichlorophenyl)-2-oxoethoxy)phenyl)-4,4a,5,5a,6,6a-hexahydro-4,6-ethenocyclopropa[f]isoindole-1,3(2H,3aH)-dione (48a)

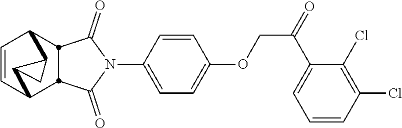 [SMILES: O=C(COc1ccc(N2C(=O)[C@@H]3[C@H](C2=O)C2C=C[C@H]3C3C[C@@H]23)cc1)c1cccc(Cl)c1Cl]

Procedure C. Purification by flash chromatography on silica gel (gradient elution, 0→40% EtOAc in hexanes) followed by recrystallization in CH₂Cl₂/hexanes afforded 48a (140 mg, 0.30 mmol, 42%) as a white solid. IR (cm⁻¹) 3056, 3010, 2955, 1705, 1512, 1187, 735; ¹H NMR (400 MHz, CDCl₃) δ 7.60 (dd, J=8.0, 2.0 Hz, 1H), 7.37 (dd, J=7.6, 1.6 Hz, 1H), 7.30 (dd, J=8.0, 7.6 Hz, 1H), 7.09 (d, J=9.2 Hz, 2H), 6.92 (d, J=8.8 Hz, 2H), 5.84 (dd, J=4.8, 3.6 Hz, 2H), 5.10 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 197.8, 178.0 (2C), 157.7, 138.9, 134.4, 133.3, 128.06, 128.04 (2C), 127.99 (2C), 127.7, 125.9, 115.5 (2C), 73.0, 45.5 (2C), 34.0 (2C), 10.1 (2C), 4.9; ES-API MS: m/z calcd for C₂₅H₂₀Cl₂NO₄ 468.1, found 468.1 [M+H]⁺.

### (3aR,4R,4aR,5aS,6S,6aS)-2-(4-(2-(2,5-Dichlorophenyl)-2-oxoethoxy)phenyl)-4,4a,5,5a,6,6a-hexahydro-4,6-ethenocyclopropa[f]isoindole-1,3(2H,3aH)-dione (49a)

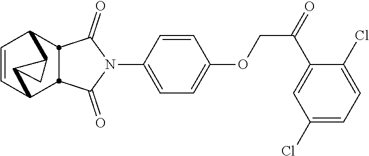 [SMILES: O=C(COc1ccc(N2C(=O)[C@@H]3[C@H](C2=O)C2C=C[C@H]3C3C[C@@H]23)cc1)c1cc(Cl)ccc1Cl]

Procedure C. Purification by flash chromatography on silica gel (gradient elution, 0→40% EtOAc in hexanes) followed by recrystallization in CH₂Cl₂/hexanes afforded 49a (37 mg, 0.080 mmol, 17%) as a white solid. IR (cm⁻¹) 2962, 1710, 1261, 1048, 803; ¹H NMR (400 MHz, CDCl₃) δ 7.55 (d, J=2.4 Hz, 1H), 7.42 (d, J=8.8, 2.4 Hz, 1H), 7.38 (d, J=8.8 Hz, 1H), 7.10 (d, J=8.8 Hz, 2H), 6.93 (d, J=8.8 Hz, 2H), 5.84 (dd, J=4.8, 3.6 Hz, 2H), 5.13 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 196.6, 178.0 (2C), 157.7, 137.6, 133.6, 132.9, 132.9, 131.9, 130.1, 128.1 (2C), 128.0 (2C), 125.9, 115.5 (2C), 73.1, 45.5 (2C), 34.0 (2C), 10.1 (2C), 4.9; ES-API MS: m/z calcd for C₂₅H₂₀Cl₂NO₄ 468.1, found 468.1 [M+H]⁺.

### (3aR,4R,4aR,5aS,6S,6aS)-2-(4-(2-(2,6-Dichlorophenyl)-2-oxoethoxy)phenyl)-4,4a,5,5a,6,6a-hexahydro-4,6-ethenocyclopropa[f]isoindole-1,3(2H,3aH)-dione (50a)

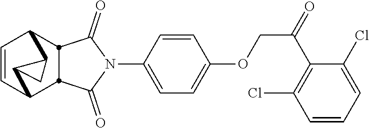 [SMILES: O=C(COc1ccc(N2C(=O)[C@@H]3[C@H](C2=O)C2C=C[C@H]3C3C[C@@H]23)cc1)c1c(Cl)cccc1Cl]

Procedure C. Purification by flash chromatography on silica gel (gradient elution, 0→40% EtOAc in hexanes) followed by recrystallization in CH₂Cl₂/hexanes afforded 50a (155 mg, 0.33 mmol, 47%) as a white solid. IR (cm⁻¹) 2961, 1709, 1513, 1184, 1045, 785; ¹H NMR (400 MHz, CDCl₃) δ 7.35 (m, 3H), 7.10 (d, J=8.8 Hz, 2H), 6.99 (d, J=9.2 Hz, 2H), 5.85 (dd, J=4.8, 3.6 Hz, 2H), 5.02 (s, 2H), 3.48 (m, 2H), 3.12 (dd, J=1.6, 2.0 Hz, 2H), 1.14 (m, 2H), 0.35-0.25 (m, 2H); ¹³C NMR (100 MHz, CDCl₃) δ 197.2, 178.0 (2C), 157.8, 136.7, 131.7, 131.6, 128.4 (2C), 128.0 (5C), 125.9, 115.7 (2C), 73.1, 45.5 (2C), 34.0 (2C), 10.1 (2C), 4.9; ES-API MS: m/z calcd for C₂₅H₂₀Cl₂NO₄ 468.1, found 468.1 [M+H]⁺.

### (3aR,4R,4aR,5aS,6S,6aS)-2-(4-(2-oxo-2-(2,3,4-Trichlorophenyl)ethoxy)phenyl)-4,4a,5,5a,6,6a-hexahydro-4,6-ethenocyclopropa[f]isoindole-1,3(2H,3aH)-dione (51a)

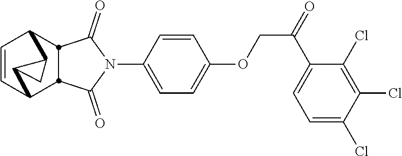 [SMILES: O=C(COc1ccc(N2C(=O)[C@@H]3[C@H](C2=O)C2C=C[C@H]3C3C[C@@H]23)cc1)c1ccc(Cl)c(Cl)c1Cl]

Procedure C. Purification by flash chromatography on silica gel (gradient elution, 0→40% EtOAc in hexanes) followed by recrystallization in CH₂Cl₂/hexanes afforded 51a (133 mg, 0.26 mmol, 41%) as a white solid. IR (cm⁻¹) 2964, 1709, 1512, 1261, 1026, 801; ¹H NMR (400 MHz, CDCl₃) δ 7.48 (d, J=8.4 Hz, 1H), 7.33 (d, J=8.4 Hz, 1H), 7.09 (d, J=9.2 Hz, 2H), 6.90 (d, J=9.2 Hz, 2H), 5.84 (dd, J=4.8, 3.6 Hz, 2H), 5.08 (s, 2H), 3.48 (m, 2H), 3.12 (dd, J=1.6, 2.0 Hz, 2H), 1.14 (m, 2H), 0.35-0.25 (m, 2H); ¹³C NMR (100 MHz, CDCl₃) δ 197.1, 178.0 (2C), 157.6, 137.8, 136.9, 133.3, 131.8, 129.1, 128.08 (2C), 127.99 (2C), 127.7, 126.0, 115.4 (2C), 73.0, 45.5 (2C), 34.0 (2C), 10.1 (2C), 4.9; ES-API MS: m/z calcd for C₂₅H₁₉Cl₃NO₄ 502.0, found 502.1 [M+H]⁺.

### (3aR,4R,4aR,5aS,6S,6aS)-2-(4-(2-(3,5-Dichlorophenyl)-2-oxoethoxy)phenyl)-4,4a,5,5a,6,6a-hexahydro-4,6-ethenocyclopropa[f]isoindole-1,3(2H,3aH)-dione (52a)

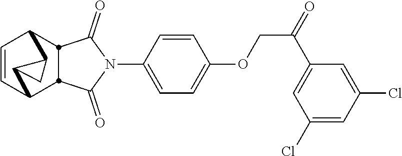 [SMILES: O=C(COc1ccc(N2C(=O)[C@@H]3[C@H](C2=O)C2C=C[C@H]3C3C[C@@H]23)cc1)c1cc(Cl)cc(Cl)c1]

Procedure C. Purification of by flash chromatography on silica gel (gradient elution, 0→40% EtOAc in hexanes) followed by recrystallization in CH₂Cl₂/hexanes afforded 52a (142 mg, 0.30 mmol, 43%) as a white solid. IR (cm⁻¹) 2960, 1708, 1513, 1214, 801; ¹H NMR (400 MHz, CDCl₃) δ 7.85 (d, J=2.0 Hz, 2H), 7.60 (t, J=2.0 Hz, 1H), 7.10 (d, J=9.2 Hz, 2H), 6.97 (d, J=9.2 Hz, 2H), 5.84 (dd, J=4.8, 3.2 Hz, 2H), 5.17 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 192.4, 178.0 (2C), 157.6, 136.9, 136.1, 133.9, 128.1 (2C), 128.0 (3C), 127.0 (2C), 126.0, 115.5 (2C), 71.3, 45.5 (2C), 34.0 (2C), 10.1 (2C), 4.9; ES-API MS: m/z calcd for C₂₅H₂₀Cl₂NO₄ 468.1, found 468.1 [M+H]⁺.

### (3aR,4R,4aR,5aS,6S,6aS)-2-(4-(2-(3,5-Dichlorophenyl)-2-oxoethoxy)-2-fluorophenyl)-4,4a,5,5a,6,6a-hexahydro-4,6-ethenocyclopropa[f]isoindole-1,3(2H,3aH)-dione (52b)

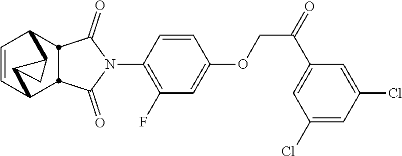 [SMILES: O=C(COc1ccc(N2C(=O)[C@@H]3[C@H](C2=O)C2C=C[C@H]3C3C[C@@H]23)c(F)c1)c1cc(Cl)cc(Cl)c1]

Procedure C. Purification by flash chromatography on silica gel (gradient elution, 0→30% EtOAc in hexanes) followed by recrystallization in CH₂Cl₂/hexanes afforded 52b (133 mg, 0.27 mmol, 32%) as a pale yellow solid. IR (cm⁻¹) 3085, 3005, 1716, 1516, 1221, 1171, 734; ¹H NMR (400 MHz, CDCl₃) δ 7.83 (d, J=1.6 Hz, 2H), 7.61 (m, 1H), 7.02 (br, 1H), 6.76 (s, 1H), 6.74 (s, 1H), 5.85 (dd, J=4.0, 3.6 Hz, 2H), 5.16 (s, 2H), 3.47 (m, 2H), 3.15 (m, 2H), 1.14 (m, 2H), 0.35-0.26 (m, 2H); ¹³C NMR (100 MHz, CDCl₃) δ 191.7 (2C), 177.2, 159.4 (d, J=4.8 Hz, 1C), 158.1 (d, J=237.1 Hz, 1C), 136.7, 136.2 (2C), 134.0 (2C), 130.1 (d, J=11.3 Hz, 1C), 127.9 (d, J=15.4 Hz, 1C), 126.9 (2C), 113.7 (d, J=13.9 Hz, 1C), 111.0 (d, J=17.3 Hz, 1C), 104.0 (d, J=26.0 Hz, 1C), 71.3, 45.9, 45.6, 33.9 (2C), 10.0 (2C), 4.9; ES-API MS: m/z calcd for C₂₅H₁₉Cl₂FNO₄ 486.1, found 486.1 [M+H]⁺.

### (3aR,4R,4aR,5aS,6S,6aS)-2-(4-(2-(3-Chloro-5-fluorophenyl)-2-oxoethoxy)phenyl)-4,4a,5,5a,6,6a-hexahydro-4,6-ethenocyclopropa[f]isoindole-1,3(2H,3aH)-dione (53a)

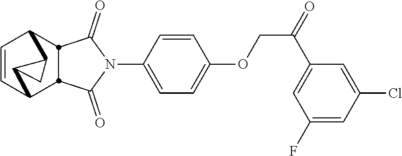 [SMILES: O=C(COc1ccc(N2C(=O)[C@@H]3[C@H](C2=O)C2C=C[C@H]3C3C[C@@H]23)cc1)c1cc(F)cc(Cl)c1]

Procedure C. Purification by flash chromatography on silica gel (gradient elution, 0→35% EtOAc in hexanes) followed by recrystallization in CH₂Cl₂/hexanes afforded 53a (37 mg, 0.082 mmol, 22%) as a white solid. IR (cm⁻¹) 2964, 1711, 1261, 1029, 802; ¹H NMR (400 MHz, CDCl₃) δ 7.77 (dd, J=1.2, 1.2 Hz, 1H), 7.59 (ddd, J=8.4, 10.2, 1.2 Hz, 1H), 7.34 (ddd, J=8.0, 2.0, 2.0 Hz, 1H), 7.10 (d, J=9.2 Hz, 2H), 6.97 (d, J=8.8 Hz, 2H), 5.84 (dd, J=4.8, 3.2 Hz, 2H), 5.16 (s, 2H), 3.48 (m, 2H), 3.12 (dd, J=1.6, 2.0 Hz, 2H), 1.14 (m, 2H), 0.35-0.25 (m, 2H); ¹³C NMR (100 MHz, CDCl₃) δ 192.4, 178.0 (2C), 162.9 (d, J=251.2 Hz, 1C), 157.6, 137.2 (d, J=7.1 Hz, 1C), 136.3 (d, J=9.5 Hz, 1C), 128.1 (2C), 128.0 (2C), 126.0, 124.7 (d, J=3.3 Hz, 1C), 121.7 (d, J=24.7 Hz, 1C), 115.5 (2C), 114.0 (d, J=22.6 Hz, 1C), 71.4, 45.5 (2C), 34.0 (2C), 10.1 (2C), 4.9; ES-API MS: m/z calcd for C₂₅H₂₀FClNO₄ 452.1, found 452.1 [M+H]⁺.

### (3aR,4R,4aR,5aS,6S,6aS)-2-(4-(2-(3-Chloro-5-(trifluoromethyl)phenyl)-2-oxoethoxy)phenyl)-4,4a,5,5a,6,6a-hexahydro-4,6-ethenocyclopropa[f]isoindole-1,3(2H,3aH)-dione (54a)

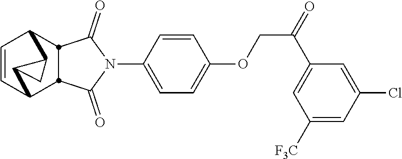 [SMILES: O=C(COc1ccc(N2C(=O)[C@@H]3[C@H](C2=O)C2C=C[C@H]3C3C[C@@H]23)cc1)c1cc(Cl)cc(C(F)(F)F)c1]

Procedure C. Purification by flash chromatography on silica gel (gradient elution, 0→35% EtOAc in hexanes) followed by recrystallization in CH₂Cl₂/hexanes afforded 54a (133 mg, 0.26 mmol, 28%) as a white solid. IR (cm⁻¹) 3079, 2963, 2857, 1710, 1513, 1326, 1175, 803; ¹H NMR (400 MHz, CDCl₃) δ 8.14 (m, 2H), 7.84 (m, 1H), 7.10 (d, J=8.8 Hz, 2H), 6.96 (d, J=8.8 Hz, 2H), 5.84 (dd, J=4.8, 3.6 Hz, 2H), 5.19 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 192.6, 178.0 (2C), 157.5, 136.4 (d, J=37.6 Hz, 1C), 133.2 (d, J=33.6 Hz, 1C), 132.0, 130.6 (d, J=3.6 Hz, 1C), 128.2 (2C), 128.0 (2C), 126.1, 123.7 (d, J=4.6 Hz, 1C), 122.9 (d, J=271.7 Hz, 1C), 115.4 (2C), 71.5, 45.5 (2C), 34.0 (2C), 10.1 (2C), 4.9; ES-API MS: m/z calcd for C₂₆H₂₀F₃ClNO₄ 502.1, found 502.1 [M+H]⁺.

### (3aR,4R,4aR,5aS,6S,6aS)-2-(4-(2-(3, 4-Dimethoxyphenyl)-2-oxoethoxy)phenyl)-4,4a,5,5a,6,6a-hexahydro-4,6-ethenocyclopropa[f]isoindole-1,3(2H,3aH)-dione (55a)

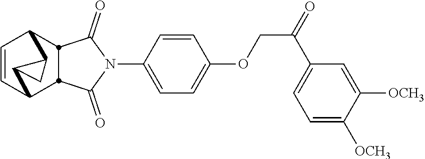 [SMILES: COc1ccc(C(=O)COc2ccc(N3C(=O)[C@@H]4[C@H](C3=O)C3C=C[C@H]4C4C[C@@H]34)cc2)cc1OC]

Procedure C. Purification by flash chromatography on silica gel (gradient elution, 0→60% EtOAc in hexanes) followed by recrystallization in CH₂Cl₂/hexanes afforded 55a (100 mg, 0.22 mmol, 61%) as a white solid. IR (cm⁻¹) 3056, 3008, 2957, 1705, 1513, 1267, 1169, 1021, 734; ¹H NMR (400 MHz, CDCl₃) δ 7.63 (dd, J=8.4, 2.0 Hz, 1H), 7.55 (d, J=2.0 Hz, 1H), 7.08 (d, J=8.8 Hz, 2H), 6.97 (d, J=8.8 Hz, 2H), 6.90 (d, J=8.4 Hz, 1H), 5.84 (dd, J=4.8, 3.2 Hz, 2H), 5.20 (s, 2H), 3.96 (s, 3H), 3.94 (s, 3H), 3.48 (m, 2H), 3.11 (dd, J=1.6, 1.6 Hz, 2H), 1.14 (m, 2H), 0.34-0.25 (m, 2H); ¹³C NMR (100 MHz, CDCl₃) δ 192.9, 178.0 (2C), 158.1, 154.2, 149.5, 127.99 (2C), 127.97 (2C), 127.8, 125.6, 123.1, 115.5 (2C), 110.5, 110.3, 71.1, 56.4, 56.3, 45.5 (2C), 34.0 (2C), 10.1 (2C), 4.9; ES-API MS: m/z calcd for C₂₇H₂₆NO₆ 460.2, found 460.1 [M+H]⁺.

### (3aR,4R,4aR,5aS,6S,6aS)-2-(4-(2-(3,4-Dimethoxyphenyl)-2-oxoethoxy)-2-fluorophenyl)-4,4a,5,5a,6,6a-hexahydro-4,6-ethenocyclopropa[f]isoindole-1,3(2H,3aH)-dione (55b)

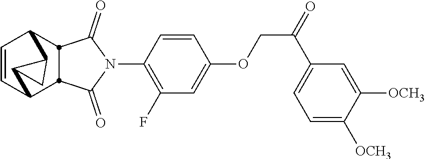 [SMILES: COc1ccc(C(=O)COc2ccc(N3C(=O)[C@@H]4[C@H](C3=O)[C@@H]3C=C[C@H]4C4CC43)c(F)c2)cc1OC]

Procedure C. Purification by flash chromatography on silica gel (gradient elution, 0→65% EtOAc in hexanes) followed by recrystallization in CH₂Cl₂/hexanes afforded 55b (130 mg, 0.27 mmol, 82%) as a orange solid. IR (cm⁻¹) 3009, 2957, 1715, 1595, 1515, 1265, 1165, 733; ¹H NMR (400 MHz, CDCl₃) δ 7.61 (dd, J=8.4, 2.0 Hz, 1H), 7.53 (d, J=2.0 Hz, 1H), 7.00 (m, 1H), 6.91 (d, J=8.4 Hz, 1H), 6.77 (m, 1H), 6.75 (m, 1H), 5.84 (dd, J=4.0, 4.0 Hz, 2H), 5.20 (s, 2H), 3.97 (s, 3H), 3.94 (s, 3H), 3.47 (m, 2H), 3.15 (m, 2H), 1.14 (m, 2H), 0.34-0.25 (m, 2H); ¹³C NMR (100 MHz, CDCl₃) δ 192.2, 177.3 (2C), 159.9 (d, J=10.4 Hz, 1C), 158.1 (d, J=251.8 Hz, 1C), 154.3, 149.6, 129.9 (d, J=19.1 Hz, 1C), 127.9 (d, J=19.5 Hz, 1C), 127.6, 123.1, 113.2 (d, J=13.0 Hz, 1C), 111.1 (d, J=10.3 Hz, 1C), 110.5, 110.4, 110.2, 104.0, 71.1, 56.4, 56.3, 45.9, 45.6, 33.9 (2C), 10.1 (2C), 4.9; ES-API MS: m/z calcd for C₂₇H₂₅FNO₆ 478.2, found 478.1 [M+H]⁺.

### (3aR,4R,4aR,5aS,6S,6aS)-2-(4-(2-(Benzo[d][1,3]dioxol-5-yl)-2-oxoethoxy)phenyl)-4,4a,5,5a,6,6a-hexahydro-4,6-ethenocyclopropa[f]isoindole-1,3(2H,3aH)-dione (56a)

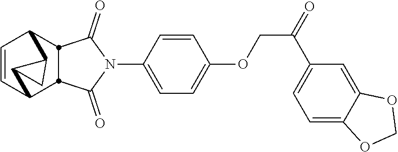 [SMILES: O=C(COc1ccc(N2C(=O)[C@@H]3[C@H](C2=O)[C@@H]2C=C[C@H]3C3CC32)cc1)c1ccc2c(c1)OCO2]

Procedure C. Purification by flash chromatography on silica gel (gradient elution, 0→50% EtOAc in hexanes) followed by recrystallization in CH₂Cl₂/hexanes afforded 56a (95 mg, 0.21 mmol, 61%) as a white solid. IR (cm⁻¹) 3009, 2955, 2909, 1704, 1512, 1252, 1037, 734; ¹H NMR (400 MHz, CDCl₃) δ 7.60 (dd, J=8.4, 1.6 Hz, 11H), 7.46 (d, J=1.6 Hz, 1H), 7.08 (d, J=8.8 Hz, 2H), 6.97 (d, J=8.8 Hz, 2H), 6.87 (d, J=8.4 Hz, 1H), 6.06 (s, 2H), 5.84 (dd, J=4.8, 3.6 Hz, 2H), 5.15 (s, 2H), 3.48 (m, 2H), 3.11 (dd, J=1.6, 1.6 Hz, 2H), 1.14 (m, 2H), 0.34-0.25 (m, 2H); ¹³C NMR (100 MHz, CDCl₃) δ 192.4, 178.0 (2C), 158.0, 152.7, 148.6, 129.4, 127.99 (2C), 127.98 (2C), 125.6, 124.9, 115.5 (2C), 108.4, 108.2, 102.2, 71.1, 45.5 (2C), 34.0 (2C), 10.1 (2C), 4.9; ES-API MS: m/z calcd for C₂₆H₂₂NO₆ 444.1, found 444.1 [M+H]⁺.

### (3aR,4R,4aR,5aS,6S,6aS)-2-(4-(2-(Benzo[d][1,3]dioxol-5-yl)-2-oxoethoxy)-2-fluorophenyl)-4,4a,5,5a,6,6a-hexahydro-4,6-ethenocyclopropa[f]isoindole-1,3(2H,3aH)-dione (56b)

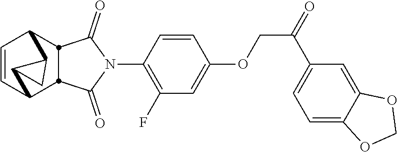 [SMILES: O=C(COc1ccc(N2C(=O)[C@@H]3[C@H](C2=O)[C@@H]2C=C[C@H]3C3CC32)c(F)c1)c1ccc2c(c1)OCO2]

Procedure C. Purification by flash chromatography on silica gel (gradient elution, 0→45% EtOAc in hexanes) followed by recrystallization in CH₂Cl₂/hexanes afforded 56b (88 mg, 0.19 mmol, 58%) as a milky solid. IR (cm⁻¹) 3010, 2955, 2913, 1715, 1516, 1254, 1170, 1038, 733; ¹H NMR (400 MHz, CDCl₃) δ 7.58 (dd, J=8.4, 1.6 Hz, 1H), 7.45 (d, J=1.6 Hz, 1H), 7.01 (m, 1H), 6.88 (d, J=8.0 Hz, 1H), 6.76 (m, 1H), 6.74 (m, 1H), 6.07 (s, 2H), 5.84 (dd, J=4.0, 4.0 Hz, 2H), 5.15 (s, 2H), 3.47 (m, 2H), 3.14 (m, 2H), 1.14 (m, 2H), 0.34-0.25 (m, 2H); ¹³C NMR (100 MHz, CDCl₃) δ 191.7, 177.3 (2C), 159.8 (d, J=10.3 Hz, 1C), 158.1 (d, J=251.7 Hz, 1C), 152.8, 148.7, 129.9 (d, J=18.3 Hz, 1C), 129.1, 127.9 (d, J=12.3 Hz, 1C), 124.8, 113.3, 113.2, 111.1 (d, J=18.2 Hz, 1C), 108.4, 108.2, 104.0 (d, J=28.3 Hz, 1C), 102.3, 71.2, 45.9, 45.6, 33.9 (2C), 10.0 (2C), 4.9; ES-API MS: m/z calcd for C₂₆H₂₁FNO₆ 462.1, found 462.1 [M+H]⁺.

### (3aR,4R,4aR,5aS,6S,6aS)-2-(4-(2-(3,4-Dimethylphenyl)-2-oxoethoxy)phenyl)-4,4a,5,5a,6,6a-hexahydro-4,6-ethenocyclopropa[f]isoindole-1,3(2H,3aH)-dione (57a)

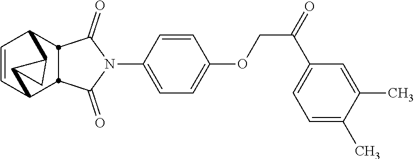 [SMILES: Cc1ccc(C(=O)COc2ccc(N3C(=O)[C@@H]4[C@H](C3=O)[C@@H]3C=C[C@H]4C4CC43)cc2)cc1C]

Procedure C. Purification by flash chromatography on silica gel (gradient elution, 0→45% EtOAc in hexanes) followed by recrystallization in CH₂Cl₂/hexanes afforded 57a (124 mg, 0.29 mmol, 82%) as a white solid. IR (cm⁻¹) 3052, 3008, 2953, 2921, 1705, 1513, 1236, 1185, 735; ¹H NMR (400 MHz, CDCl₃) δ 7.75 (d, J=1.6 Hz, 1H), 7.71 (dd, J=8.0, 1.6 Hz, 1H), 7.24 (d, J=8.0 Hz, 1H), 7.08 (d, J=9.2 Hz, 2H), 6.97 (d, J=9.2 Hz, 2H), 5.84 (dd, J=4.8, 3.2 Hz, 2H), 5.22 (s, 2H), 3.48 (m, 2H), 3.11 (dd, J=1.6, 1.6 Hz, 2H), 2.33 (s, 3H), 2.32 (s, 3H), 1.14 (m, 2H), 0.34-0.25 (m, 2H); ¹³C NMR (100 MHz, CDCl₃) δ 194.0, 178.0 (2C), 158.1, 144.0, 137.6, 132.5, 130.2, 129.4, 127.97 (2C), 127.96 (2C), 126.1, 125.5, 115.5 (2C), 71.1, 45.5 (2C), 34.0 (2C), 20.4, 20.0, 10.1 (2C), 4.9; ES-API MS: m/z calcd for C₂₇H₂₆NO₄ 428.2, found 428.1 [M+H]⁺.

### (3aR,4R,4aR,5aS,6S,6aS)-2-(4-(2-(3,4-Dimethylphenyl)-2-oxoethoxy)-2-fluorophenyl)-4,4a,5,5a,6,6a-hexahydro-4,6-ethenocyclopropa[f]isoindole-1,3(2H,3aH)-dione (57b)

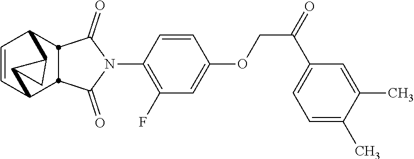 [SMILES: Cc1ccc(C(=O)COc2ccc(N3C(=O)[C@@H]4[C@H](C3=O)[C@@H]3C=C[C@H]4C4CC43)c(F)c2)cc1C]

Procedure C. Purification by flash chromatography on silica gel (gradient elution, 0→45% EtOAc in hexanes) followed by recrystallization in CH₂Cl₂/hexanes afforded 57b (105 mg, 0.24 mmol, 71%) as a pale pinky solid. IR (cm⁻¹) 3014, 2952, 1715, 1516, 1166, 1124, 733; ¹H NMR (400 MHz, CDCl₃) δ 7.74 (m, 1H), 7.70 (dd, J=7.6, 1.2 Hz, 1H), 7.25 (d, J=8.0 Hz, 1H), 7.00 (m, 1H), 6.77 (m, 1H), 6.74 (m, 1H), 5.84 (dd, J=4.0, 3.6 Hz, 2H), 5.22 (s, 2H), 3.47 (m, 2H), 3.14 (m, 2H), 2.33 (s, 3H), 2.32 (s, 3H), 1.14 (m, 2H), 0.34-0.25 (m, 2H); ¹³C NMR (100 MHz, CDCl₃) δ 193.3, 177.3 (2C), 159.9 (d, J=10.3 Hz, 1C), 158.1 (d, J=251.5 Hz, 1C), 144.1, 137.6, 132.3, 130.3, 129.9 (d, J=14.0 Hz, 1C), 129.3, 127.9 (d, J=16.3 Hz, 1C), 126.0, 113.2, 113.1, 111.2 (d, J=17.2 Hz, 1C), 103.9 (d, J=33.2 Hz, 1C), 71.1, 45.9, 45.6, 33.9 (2C), 20.4, 20.0, 10.0 (2C), 4.9; ES-API MS: m/z calcd for C₂₇H₂₅FNO₄ 446.2, found 446.1 [M+H]⁺.

### (3aR,4R,4aR,5aS,6S,6aS)-2-(4-(2-(3, 4-Dichlorophenyl)-2-oxoethoxy)phenyl)-4,4a,5,5a,6,6a-hexahydro-4,6-ethenocyclopropa[f]isoindole-1,3(2H,3aH)-dione (58a)

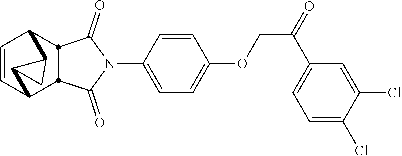 [SMILES: O=C(COc1ccc(N2C(=O)[C@@H]3[C@H](C2=O)[C@@H]2C=C[C@H]3C3CC32)cc1)c1ccc(Cl)c(Cl)c1]

Procedure C. Purification by flash chromatography on silica gel (gradient elution, 0→50% EtOAc in hexanes) followed by recrystallization in CH₂Cl₂/hexanes afforded 58a (33 mg, 0.070 mmol, 40%) as a white solid. IR (cm⁻¹) 2955, 1704, 1512, 1393, 1212, 1185, 995, 734; ¹H NMR (400 MHz, CDCl₃) δ 8.08 (d, J=2.0 Hz, 1H), 7.82 (dd, J=8.4, 2.0 Hz, 1H), 7.58 (d, J=8.4 Hz, 1H), 7.10 (d, J=8.8 Hz, 2H), 6.96 (d, J=9.2 Hz, 2H), 5.84 (dd, J=4.8, 3.6 Hz, 2H), 5.16 (s, 2H), 3.48 (m, 2H), 3.12 (dd, J=2.0, 1.6 Hz, 2H), 1.14 (m, 2H), 0.34-0.25 (m, 2H); ¹³C NMR (100 MHz, CDCl₃) δ 192.7, 178.0 (2C), 157.7, 138.9, 134.1, 133.9, 131.2, 130.6, 128.1 (2C), 128.0 (2C), 127.6, 125.9, 115.5 (2C), 71.4, 45.5 (2C), 34.0 (2C), 10.1 (2C), 4.9; ES-API MS: m/z calcd for C₂₅H₁₉Cl₂NO₄Na 490.1 found 490.5 [M+Na]⁺.

### (3aR,4R,4aR,5aS,6S,6aS)-2-(4-(2-(3,4-Dichlorophenyl)-2-oxoethoxy)-2-fluorophenyl)-4,4a,5,5a,6,6a-hexahydro-4,6-ethenocyclopropa[f]isoindole-1,3(2H,3aH)-dione (58b)

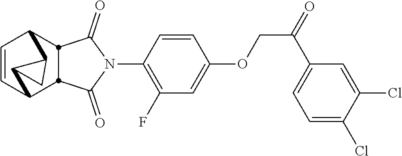 [SMILES: O=C(COc1ccc(N2C(=O)[C@@H]3[C@H](C2=O)[C@@H]2C=C[C@H]3C3CC32)c(F)c1)c1ccc(Cl)c(Cl)c1]

Procedure C. Purification by flash chromatography on silica gel (gradient elution, 0→35% EtOAc in hexanes) followed by recrystallization in CH₂Cl₂/hexanes afforded 58b (122 mg, 0.25 mmol, 75%) as a pale brown solid. IR (cm⁻¹) 2956, 1716, 1517, 1396, 1170, 735; ¹H NMR (400 MHz, CDCl₃) δ 8.07 (d, J=2.0 Hz, 1H), 7.81 (dd, J=8.4, 2.0 Hz, 1H), 7.59 (d, J=8.4 Hz, 1H), 7.02 (br, 1H), 6.76 (m, 1H), 6.74 (m, 1H), 5.85 (dd, J=4.0, 3.6 Hz, 2H), 5.16 (s, 2H), 3.47 (m, 2H), 3.15 (m, 2H), 1.13 (m, 2H), 0.35-0.26 (m, 2H); ¹³C NMR (100 MHz, CDCl₃) δ 192.0 (2C), 177.3, 159.4 (d, J=2.4 Hz, 1C), 158.1 (d, J=244.4 Hz, 1C), 139.1, 134.0, 133.9, 131.3, 130.5, 130.1, (d, J=15.8 Hz, 1C), 127.9 (d, J=15.1 Hz, 1C), 127.5 (2C), 113.6 (d, J=13.8 Hz, 1C), 111.0 (d, J=11.6 Hz, 1C), 104.0 (d, J=25.8 Hz, 1C), 71.3, 45.9, 45.6, 33.9 (2C), 10.0 (2C), 4.9; ES-API MS: m/z calcd for C₂₅H₁₉Cl₂FNO₄ 486.1 found 486.1 [M+H]⁺.

### (3aR,4R,4aR,5aS,6S,6aS)-2-(4-(2-(3-Fluoro-4-methoxyphenyl)-2-oxoethoxy)phenyl)-4,4a,5,5a,6,6a-hexahydro-4,6-ethenocyclopropa[f]isoindole-1,3(2H,3aH)-dione (59a)

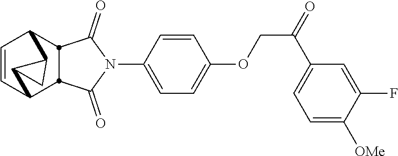 [SMILES: COc1ccc(C(=O)COc2ccc(N3C(=O)[C@@H]4[C@H](C3=O)[C@@H]3C=C[C@H]4C4CC43)cc2)cc1F]

Procedure C. Purification by flash chromatography on silica gel (gradient elution, 0→65% EtOAc in hexanes) followed by recrystallization in CH₂Cl₂/hexanes afforded 59a (32 mg, 0.072 mmol, 62%) as a pale yellow solid. IR (cm⁻¹) 3010, 2956, 1705, 1613, 1513, 1434, 1392, 1286, 1227, 1186, 734; ¹H NMR (400 MHz, CDCl₃) δ 7.80 (ddd, J=8.4, 2.0, 0.8 Hz, 1H), 7.75 (dd, J=11.6, 2.0 Hz, 1H), 7.08 (d, J=9.2 Hz, 2H), 7.01 (dd, J=8.4, 8.4 Hz, 1H), 6.97 (d, J=9.2 Hz, 2H), 5.84 (dd, J=4.8, 3.6 Hz, 2H), 5.15 (s, 2H), 3.97 (s, 3H), 3.48 (m, 2H), 3.11 (dd, J=1.6, 1.6 Hz, 2H), 1.14 (m, 2H), 0.34-0.25 (m, 2H); ¹³C NMR (100 MHz, CDCl₃) δ 192.3 (d, J=1.8, 1C), 178.0 (2C), 157.9, 153.1 (d, J=75.5, 1C), 152.0 (d, J=182.6, 1C), 128.03 (2C), 127.98 (2C), 127.8 (d, J=5.2, 1C), 126.0 (d, J=3.3, 1C), 125.7, 116.2 (d, J=19.1, 1C), 115.5 (2C), 112.7 (d, J=1.9, 1C), 71.2, 56.6, 45.5 (2C), 34.0 (2C), 10.1 (2C), 4.9; ES-API MS: m/z calcd for C₂₆H₂₂FNO₅Na 470.1, found 470.1 [M+Na]⁺.

### (3aR,4R,4aR,5aS,6S,6aS)-2-(4-(2-(3-Chloro-4-methoxyphenyl)-2-oxoethoxy)phenyl)-4,4a,5,5a,6,6a-hexahydro-4,6-ethenocyclopropa[f]isoindole-1,3(2H,3aH)-dione (60a)

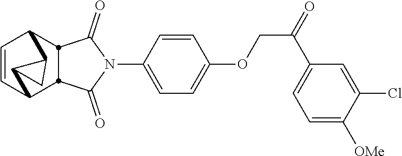 [SMILES: COc1ccc(C(=O)COc2ccc(N3C(=O)[C@@H]4[C@H](C3=O)[C@@H]3C=C[C@H]4C4CC43)cc2)cc1Cl]

Procedure C. Purification by flash chromatography on silica gel (gradient elution, 0→65% EtOAc in hexanes) followed by recrystallization in CH₂Cl₂/hexanes afforded 60a (57 mg, 0.12 mmol, 57%) as a pale yellow solid. IR (cm⁻¹) 1705, 1594, 1508, 1207, 1061, 734; ¹H NMR (400 MHz, CDCl₃) δ 8.04 (d, J=2.0 Hz, 1H), 7.92 (dd, J=8.8, 2.4 Hz, 1H), 7.08 (d, J=9.2 Hz, 2H), 6.99 (d, J=8.8 Hz, 1H), 6.97 (d, J=8.8 Hz, 2H), 5.84 (dd, J=4.4, 3.6 Hz, 2H), 5.15 (s, 2H), 3.98 (s, 3H), 3.48 (m, 2H), 3.11 (dd, J=1.2, 1.6 Hz, 2H), 1.13 (m, 2H), 0.34-0.25 (m, 2H); ¹³C NMR (100 MHz, CDCl₃) δ 192.2, 178.0 (2C), 159.6, 157.9, 130.8, 129.1, 128.1, 128.03 (2C), 127.97 (2C), 125.7, 123.4, 115.5 (2C), 111.7, 71.2, 56.7, 45.5 (2C), 34.0 (2C), 10.1 (2C), 4.9; ES-API MS: m/z calcd for C₂₆H₂₂ClNO₅Na 486.1, found 486.1 [M+Na]⁺.

### (3aR,4R,4aR,5aS,6S,6aS)-2-(4-(2-(3-Chloro-4-methoxyphenyl)-2-oxoethoxy)-2-fluorophenyl)-4,4a,5,5a,6,6a-hexahydro-4,6-ethenocyclopropa[f]isoindole-1,3(2H,3aH)-dione (60b)

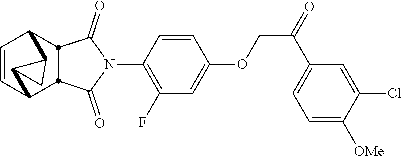 [SMILES: COc1ccc(C(=O)COc2ccc(N3C(=O)[C@@H]4[C@H](C3=O)[C@@H]3C=C[C@H]4C4CC43)c(F)c2)cc1Cl]

Procedure C. Purification by flash chromatography on silica gel (gradient elution, 0→55% EtOAc in hexanes) followed by recrystallization in CH₂Cl₂/hexanes afforded 60b (94 mg, 0.20 mmol, 58%) as a white solid. IR (cm⁻¹) 3057, 3011, 2954, 1716, 1595, 1516, 1260, 1169, 1061, 1008, 735; ¹H NMR (400 MHz, CDCl₃) δ 8.02 (d, J=2.4 Hz, 1H), 7.91 (dd, J=8.8, 2.0 Hz, 1H), 7.01 (br, 1H), 6.99 (d, J=8.8 Hz, 1H), 6.77 (m, 1H), 6.74 (m, 1H), 5.84 (dd, J=4.0, 4.0 Hz, 2H), 5.15 (s, 2H), 3.99 (s, 3H), 3.47 (m, 2H), 3.14 (m, 2H), 1.13 (m, 2H), 0.34-0.26 (m, 2H); ¹³C NMR (100 MHz, CDCl₃) δ 191.5 (2C), 177.3, 159.7, 159.6 (d, J=10.3 Hz, 1C), 158.1 (d, J=251.7 Hz, 1C), 130.7, 130.0 (d, J=12.8 Hz, 1C), 129.1, 127.9 (d, J=14.3 Hz, 1C), 127.9, 123.5, 113.4 (d, J=13.8 Hz, 1C), 111.7 (2C), 111.1 (d, J=14.3 Hz, 1C), 103.9 (d, J=23.2 Hz, 1C), 71.2, 56.7, 45.9, 45.6, 33.9 (2C), 10.0 (2C), 4.9; ES-API MS: m/z calcd for C₂₆H₂₂ClFNO₅ 482.1, found 482.1 [M+H]⁺.

### (3aR,4R,4aR,5aS,6S,6aS)-2-(4-(2-(4-Chloro-3-methylphenyl)-2-oxoethoxy)phenyl)-4,4a,5,5a,6,6a-hexahydro-4,6-ethenocyclopropa[f]isoindole-1,3(2H,3aH)-dione (61a)

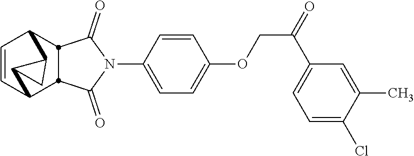 [SMILES: Cc1cc(C(=O)COc2ccc(N3C(=O)[C@@H]4[C@H](C3=O)[C@@H]3C=C[C@H]4C4CC43)cc2)ccc1Cl]

Procedure C. Purification by flash chromatography on silica gel (gradient elution, 0→40% EtOAc in hexanes) followed by recrystallization in CH₂Cl₂/hexanes afforded 61a (42 mg, 0.094 mmol, 53%) as a white solid. IR (cm⁻¹) 2964, 1710, 1261, 1046, 801; ¹H NMR (400 MHz, CDCl₃) δ 7.86 (d, J=1.6 Hz, 1H), 7.75 (dd, J=8.4, 2.0 Hz, 1H), 7.45 (d, J=8.4 Hz, 1H), 7.09 (d, J=8.8 Hz, 2H), 6.97 (d, J=9.2 Hz, 2H), 5.84 (dd, J=4.8, 3.6 Hz, 2H), 5.19 (s, 2H), 3.48 (m, 2H), 3.12 (dd, J=2.0, 1.6 Hz, 2H), 2.44 (s, 3H), 1.14 (m, 2H), 0.34-0.25 (m, 2H); ¹³C NMR (100 MHz, CDCl₃) δ 193.6, 178.0 (2C), 157.9, 140.9, 137.3, 133.0, 130.8, 129.8, 128.04 (2C), 127.98 (2C), 127.2, 125.7, 115.5 (2C), 71.2, 45.5 (2C), 34.0 (2C), 20.4, 10.1 (2C), 4.9; ES-API MS: m/z calcd for C₂₆H₂₃ClNO₄ 482.1, found 448.1 [M+H]⁺.

### (3aR,4R,4aR,5aS,6S,6aS)-2-(4-(2-(4-Chloro-3-methylphenyl)-2-oxoethoxy)-2-fluorophenyl)-4,4a,5,5a,6,6a-hexahydro-4,6-ethenocyclopropa[f]isoindole-1,3(2H,3aH)-dione (61b)

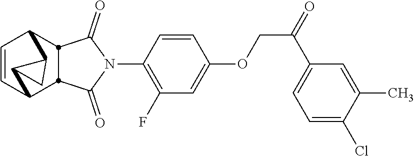 [SMILES: Cc1cc(C(=O)COc2ccc(N3C(=O)[C@@H]4[C@H](C3=O)[C@@H]3C=C[C@H]4C4CC43)c(F)c2)ccc1Cl]

Procedure C. Purification by flash chromatography on silica gel (gradient elution, 0→35% EtOAc in hexanes) followed by recrystallization in CH₂Cl₂/hexanes afforded 61b (80 mg, 0.17 mmol, 52%) as a pale pinky solid. IR (cm⁻¹) 1716, 1519, 1508, 1168, 1049, 734; ¹H NMR (400 MHz, CDCl₃) δ 7.84 (d, J=1.6 Hz, 1H), 7.75 (ddd, J=8.4, 8.4, 1.6 Hz, 1H), 7.46 (d, J=8.4 Hz, 1H), 7.02 (br, 1H), 6.77 (m, 1H), 6.74 (m, 1H), 5.85 (dd, J=4.0, 3.6 Hz, 2H), 5.19 (s, 2H), 3.47 (m, 2H), 3.15 (m, 2H), 1.13 (m, 2H), 0.35-0.26 (m, 2H); ¹³C NMR (100 MHz, CDCl₃) δ 192.9 (2C), 177.3, 159.6 (d, J=10.3 Hz, 1C), 158.1 (d, J=251.9 Hz, 1C), 143.3, 141.1, 137.4, 132.8, 131.6, 130.8, 129.9 (2C), 129.1, 127.9 (d, J=13.8 Hz, 1C), 127.1, 126.5, 71.2, 45.9, 45.6, 33.9 (2C), 20.4, 10.1 (2C), 4.9; ES-API MS: m/z calcd for C₂₆H₂₂ClFNO₄ 466.1, found 466.1 [M+H]⁺.

### (3aR,4R,4aR,5aS,6S,6aS)-2-(4-(2-(3-Chloro-4-fluorophenyl)-2-oxoethoxy)phenyl)-4,4a,5,5a,6,6a-hexahydro-4,6-ethenocyclopropa[f]isoindole-1,3(2H,3aH)-dione (62a)

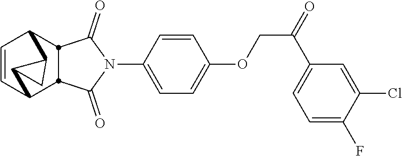 [SMILES: O=C(COc1ccc(N2C(=O)[C@@H]3[C@H](C2=O)[C@@H]2C=C[C@H]3C3CC32)cc1)c1ccc(F)c(Cl)c1]

Procedure C. Purification by flash chromatography on silica gel (gradient elution, 0→45% EtOAc in hexanes) followed by recrystallization in CH₂Cl₂/hexanes afforded 62a (25 mg, 0.055 mmol, 31%) as a white solid. IR (cm⁻¹) 2964, 1710, 1261, 1045, 802; ¹H NMR (400 MHz, CDCl₃) δ 8.09 (dd, J=7.2, 2.0 Hz, 1H), 7.92 (ddd, J=8.4, 4.4, 2.0 Hz, 1H), 7.26 (dd, J=8.4, 8.4 Hz, 1H), 7.09 (d, J=9.2 Hz, 2H), 6.96 (d, J=9.2 Hz, 2H), 5.84 (dd, J=4.8, 3.6 Hz, 2H), 5.15 (s, 2H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 192.3, 178.0 (2C), 161.7 (d, J=257.1 Hz, 1C), 157.7, 131.8 (d, J=3.7 Hz, 1C), 131.6 (d, J=1.2 Hz, 1C), 129.1 (d, J=8.6 Hz, 1C), 128.1 (2C), 128.0 (2C), 125.9, 122.5 (d, J=18.3 Hz, 1C), 117.4 (d, J=21.8 Hz, 1C), 115.5 (2C), 71.3, 45.5 (2C), 34.0 (2C), 10.1 (2C), 4.9; ES-API MS: m/z calcd for C₂₅H₂₀FClNO₄ 452.1, found 452.1 [M+H]⁺.

### (3aR,4R,4aR,5aS,6S,6aS)-2-(4-(2-(3-Chloro-4-fluorophenyl)-2-oxoethoxy)-2-fluorophenyl)-4,4a,5,5a,6,6a-hexahydro-4,6-ethenocyclopropa[f]isoindole-1,3(2H,3aH)-dione (62b)

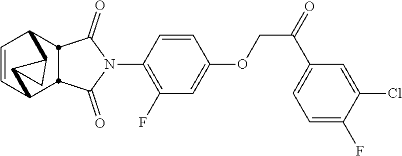 [SMILES: O=C(COc1ccc(N2C(=O)[C@@H]3[C@H](C2=O)[C@@H]2C=C[C@H]3C3CC32)c(F)c1)c1ccc(F)c(Cl)c1]

Procedure C. Purification by flash chromatography on silica gel (gradient elution, 0→45% EtOAc in hexanes) followed by recrystallization in CH₂Cl₂/hexanes afforded 62b (86 mg, 0.18 mmol, 55%) as a pale yellow solid. IR (cm⁻¹) 2956, 1716, 1517, 1253, 1170, 734; ¹H NMR (400 MHz, CDCl₃) δ 8.07 (d, J=8.8 Hz, 1H), 7.90 (m, 1H), 7.27 (dd, J=8.4, 8.4 Hz, 1H), 7.02 (br, 1H), 6.76 (m, 1H), 6.74 (m, 1H), 5.84 (dd, J=4.0, 3.6 Hz, 2H), 5.16 (s, 2H), 3.47 (m, 2H), 3.15 (m, 2H), 1.14 (m, 2H), 0.34-0.27 (m, 2H); ¹³C NMR (100 MHz, CDCl₃) δ 191.3 (2C), 177.0, 161.6 (d, J=257.6 Hz, 1C), 159.2 (d, J=11.0 Hz, 1C), 157.9 (d, J=252.0 Hz, 1C), 131.4 (2C), 131.4 (d, J=1.2 Hz, 1C), 129.9 (d, J=20.6 Hz, 1C), 128.8 (d, J=8.6 Hz, 1C), 127.7 (d, J=12.9 Hz, 1C), 122.5, 117.2 (d, J=21.7 Hz, 1C), 113.4 (d, J=13.8 Hz, 1C), 110.8 (d, J=12.6 Hz, 1C), 103.7 (d, J=24.7 Hz, 1C), 71.1, 45.7, 45.4, 33.7 (2C), 9.8 (2C), 4.7; ES-API MS: m/z calcd for C₂₅H₁₉ClF₂NO₄ 470.1, found 470.1 [M+H]⁺.

### (3aR,4R,4aR,5aS,6S,6aS)-2-(4-(2-(4-Chloro-3-(trifluoromethyl)phenyl)-2-oxoethoxy)phenyl)-4,4a,5,5a,6,6a-hexahydro-4,6-ethenocyclopropa[f]isoindole-1,3(2H,3aH)-dione (63a)

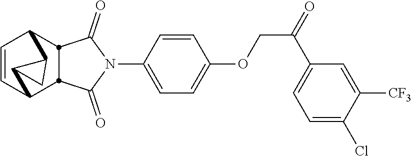 [SMILES: O=C(COc1ccc(N2C(=O)[C@@H]3[C@H](C2=O)[C@@H]2C=C[C@H]3C3CC32)cc1)c1ccc(Cl)c(C(F)(F)F)c1]

Procedure C. Purification by flash chromatography on silica gel (gradient elution, 0→35% EtOAc in hexanes) followed by recrystallization in CH₂Cl₂/hexanes afforded 63a (97 mg, 0.19 mmol, 34%) as a white solid. IR (cm⁻¹) 2961, 1710, 1604, 1513, 1261, 1180, 1036, 803, 735; ¹H NMR (400 MHz, CDCl₃) δ 8.33 (d, J=2.0 Hz, 1H), 8.10 (d, J=8.4, 2.0 Hz, 1H), 7.64 (d, J=8.4 Hz, 1H), 7.09 (d, J=8.8 Hz, 2H), 6.95 (d, J=8.8 Hz, 2H), 5.83 (dd, J=4.8, 3.6 Hz, 2H), 5.17 (s, 2H), 3.47 (m, 2H), 3.11 (dd, J=1.6, 1.6 Hz, 2H), 1.14 (m, 2H), 0.35-0.25 (m, 2H); ¹³C NMR (100 MHz, CDCl₃) δ 192.9, 178.0 (2C), 157.5, 138.5 (d, J=1.6 Hz, 1C), 133.0, 132.8, 123.4, 129.4 (d, J=3.2 Hz, 1C), 128.1 (2C), 128.0 (2C), 127.9, 127.8 (d, J=3.0 Hz, 1C), 126.0, 122.5 (d, J=272.4 Hz, 1C), 115.4 (2C), 71.5, 45.5 (2C), 34.0 (2C), 10.1 (2C), 4.9; ES-API MS: m/z calcd for C₂₆H₂₀F₃ClNO₄ 502.1, found 502.1 [M+H]⁺.

### (3aR,4R,4aR,5aS,6S,6aS)-2-(4-(2-(4-Chloro-3-(trifluoromethyl)phenyl)-2-oxoethoxy)-2-fluorophenyl)-4,4a,5,5a,6,6a-hexahydro-4,6-ethenocyclopropa[f]isoindole-1,3(2H,3aH)-dione (63b)

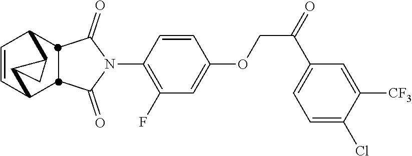 [SMILES: O=C(COc1ccc(N2C(=O)C3C(C2=O)[C@H]2C=C[C@@H]3C3CC32)c(F)c1)c1ccc(Cl)c(C(F)(F)F)c1]

Procedure C. Purification by flash chromatography on silica gel (gradient elution, 0→45% EtOAc in hexanes) followed by recrystallization in CH₂Cl₂/hexanes afforded 63b (31 mg, 0.06 mmol, 18%) as a pale yellow solid. IR (cm⁻¹) 2957, 1716, 1519, 1319, 1172, 1141, 1114, 1036, 735; ¹H NMR (400 MHz, CDCl₃) δ 8.32 (d, J=1.6 Hz, 1H), 8.09 (dd, J=8.4, 2.0 Hz, 1H), 7.66 (d, J=8.4 Hz, 1H), 7.03 (br, 1H), 6.77 (m, 1H), 6.74 (m, 1H), 5.85 (dd, J=4.0, 3.6 Hz, 2H), 5.18 (s, 2H), 3.47 (m, 2H), 3.15 (m, 2H), 1.14 (m, 2H), 0.35-0.26 (m, 2H); ¹³C NMR (100 MHz, CDCl₃) δ 191.9 (2C), 177.0, 157.9 (d, J=252.3 Hz, 1C), 159.0 (d, J=10.3 Hz, 1C), 138.5 (d, J=1.7 Hz, 1C), 132.6, 132.5, 132.3, 129.9 (d, J=12.1 Hz, 1C), 129.4 (d, J=32.0 Hz, 1C), 127.8, 127.6 (q, J=5.2 Hz, 1C), 123.6, 120.9, 113.6 (d, J=13.8 Hz, 1C), 110.7, 103.7 (d, J=23.0 Hz, 1C), 71.3, 45.7, 45.4, 33.7 (2C), 9.8 (2C), 4.7; ES-API MS: m/z calcd for C₂₆H₁₉ClF₄NO₄ 520.1, found 520.1 [M+H]⁺.

### (3aR,4R,4aR,5aS,6S,6aS)-2-(4-(2-(Naphthalen-2-yl)-2-oxoethoxy)phenyl)-4,4a,5,5a,6,6a-hexahydro-4,6-ethenocyclopropa[f]isoindole-1,3(2H,3aH)-dione (64a)

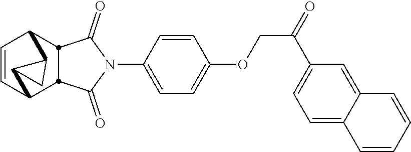 [SMILES: O=C(COc1ccc(N2C(=O)[C@@H]3[C@H](C2=O)[C@@H]2C=C[C@H]3C3CC32)cc1)c1ccc2ccccc2c1]

Procedure C. Purification by flash chromatography on silica gel (gradient elution, 0→40% EtOAc in hexanes) followed by recrystallization in CH₂Cl₂/hexanes afforded 64a (116 mg, 0.25 mmol, 73%) as a pale yellow solid. IR (cm⁻¹) 3055, 3010, 2954, 1704, 1512, 1188, 734; ¹H NMR (400 MHz, CDCl₃) δ 8.53 (s, 1H), 8.03 (dd, J=8.8, 1.6 Hz, 1H), 7.98 (d, J=8.0 Hz, 1H), 7.93 (d, J=8.8 Hz, 1H), 7.90 (d, J=8.0 Hz, 1H), 7.64 (dd, J=6.8, 7.2 Hz, 1H), 7.58 (dd, J=7.2, 7.2 Hz, 1H), 7.10 (d, J=8.8 Hz, 2H), 7.02 (d, J=8.8 Hz, 2H), 5.84 (dd, J=4.4, 3.6 Hz, 1H), 5.38 (s, 2H), 3.48 (m, 2H), 3.12 (m, 2H), 1.14 (m, 2H), 0.34-0.25 (m, 2H); ¹³C NMR (100 MHz, CDCl₃) δ 194.2, 178.0 (2C), 158.1, 136.2, 132.6, 132.0, 130.3, 129.9, 129.2, 129.0, 128.1, 128.03 (2C), 127.98 (2C), 127.3, 125.6, 123.8, 115.6 (2C), 71.3, 45.5 (2C), 34.0 (2C), 10.1 (2C), 4.9; ES-API MS: m/z calcd for C₂₉H₃₄NO₄ 460.2, found 460.1 [M+H]⁺.

### (3aR,4R,4aR,5aS,6S,6aS)-2-(5-(2-(4-Methoxyphenyl)-2-oxoethoxy)pyridin-2-yl)-4,4a,5,5a,6,6a-hexahydro-4,6-ethenocyclopropa[f]isoindole-1,3(2H,3aH)-dione (71)

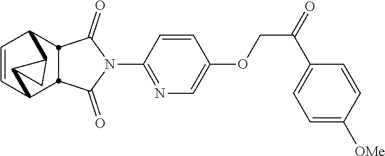 [SMILES: COc1ccc(C(=O)COc2ccc(N3C(=O)[C@@H]4[C@H](C3=O)[C@@H]3C=C[C@H]4C4CC43)nc2)cc1]

Procedure C. Purification by flash chromatography on silica gel (gradient elution, 0→70% EtOAc in hexanes) followed by recrystallization in CH₂Cl₂/hexanes afforded 71 (86 mg, 0.20 mmol, 57%) as a pale yellow solid. IR (cm⁻¹) 3063, 2940, 2840, 1710, 1601, 1401, 1232, 1173, 972; ¹H NMR (400 MHz, CDCl₃) δ 8.31 (d, J=2.8 Hz, 1H), 7.96 (d, J=8.8 Hz, 2H), 7.32 (dd, J=8.8, 3.2 Hz, 1H), 7.06 (d, J=8.8 Hz, 1H), 6.97 (d, J=8.8 Hz, 2H), 5.88 (dd, J=4.8, 3.2 Hz, 2H), 5.27 (s, 2H), 3.89 (s, 3H), 3.49 (m, 2H), 3.14 (dd, J=1.6, 1.6 Hz, 2H), 1.14 (m, 2H), 0.34-0.25 (m, 2H); ¹³C NMR (100 MHz, CDCl₃) δ 191.8, 177.5 (2C), 164.5, 154.6, 139.6, 137.9, 130.7 (2C), 128.0 (2C), 127.3, 123.8, 122.7, 114.4 (2C), 71.1, 55.8, 45.8 (2C), 33.9 (2C), 10.1 (2C), 5.0; ES-API MS: m/z calcd for C₂₅H₂₃N₂O₅ 431.2, found 431.2 [M+H]⁺.

### (3aR,4R,4aR,5aS,6S,6aS)-2-(4-(2-(6-Methoxypyridin-3-yl)-2-oxoethoxy)phenyl)-4,4a,5,5a,6,6a-hexahydro-4,6-ethenocyclopropa[f]isoindole-1,3(2H,3aH)-dione (72)

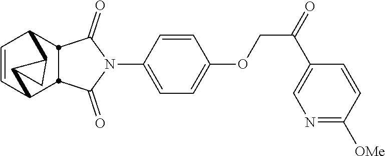 [SMILES: COc1ccc(C(=O)COc2ccc(N3C(=O)[C@@H]4[C@H](C3=O)[C@@H]3C=C[C@H]4C4CC43)cc2)cn1]

Procedure C. Purification by flash chromatography on silica gel (gradient elution, 0→50% EtOAc in hexanes) followed by recrystallization in CH₂Cl₂/hexanes afforded 72 (40 mg, 0.093 mmol, 26%) as a white solid. IR (cm⁻¹) 3053, 3009, 2953, 1705, 1602, 1513, 1377, 1298, 1226, 1186, 735; ¹H NMR (400 MHz, CDCl₃) δ 8.86 (d, J=2.4 Hz, 1H), 8.16 (dd, J=8.4, 2.4 Hz, 1H), 7.08 (d, J=8.8 Hz, 2H), 6.96 (d, J=9.2 Hz, 2H), 6.81 (d, J=8.8 Hz, 1H), 5.83 (dd, J=3.6, 4.4 Hz, 2H), 5.13 (s, 2H), 4.01 (s, 3H), 3.47 (m, 2H), 3.11 (m, 2H), 1.13 (m, 2H), 0.33-0.24 (m, 2H); ¹³C NMR (100 MHz, CDCl₃) δ 192.8, 178.0 (2C), 167.3, 157.8, 149.6, 138.6, 128.0 (2C), 127.9 (2C), 125.7, 124.7, 115.4 (2C), 111.8, 71.3, 54.4, 45.4 (2C), 34.0 (2C), 10.1 (2C), 4.9; ES-API MS: m/z calcd for C₂₅H₂₃N₂O₅ 431.2, found 431.2 [M+H]⁺.

### (3aR,4R,4aR,5aS,6S,6aS)-2-(S-(2-(6-Methoxypyridin-3-yl)-2-oxoethoxy)pyridin-2-yl)-4,4a,5,5a,6,6a-hexahydro-4,6-ethenocyclopropa[f]isoindole-1,3(2H,3aH)-dione (73)

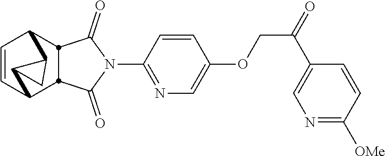 [SMILES: COc1ccc(C(=O)COc2ccc(N3C(=O)[C@@H]4[C@H](C3=O)[C@@H]3C=C[C@H]4C4CC43)nc2)cn1]

Procedure C. Purification by flash chromatography on silica gel (gradient elution, 0→70% EtOAc in hexanes) followed by recrystallization in CH₂Cl₂/hexanes afforded 73 (63 mg, 0.15 mmol, 42%) as a pale yellow solid. IR (cm⁻¹) 3054, 3011, 2953, 1710, 1602, 1490, 1376, 1298, 1231, 969, 730; ¹H NMR (400 MHz, CDCl₃) δ 8.84 (d, J=2.4 Hz, 1H), 8.32 (d, J=2.8 Hz, 1H), 8.15 (dd, J=8.8, 2.4 Hz, 1H), 7.33 (dd, J=8.8, 2.8 Hz, 1H), 7.07 (d, J=8.8 Hz, 1H), 6.83 (d, J=8.8 Hz, 1H), 5.88 (dd, J=4.4, 3.6 Hz, 2H), 5.22 (s, 2H), 4.02 (s, 3H), 3.49 (m, 2H), 3.14 (dd, J=1.6, 1.6 Hz, 2H), 1.13 (m, 2H), 0.35-0.25 (m, 2H); ¹³C NMR (100 MHz, CDCl₃) δ 191.6, 177.5 (2C), 167.5, 154.4, 149.5, 139.8, 138.5, 137.8, 128.1 (2C), 124.4, 123.9, 122.8, 112.0, 71.2, 54.5, 45.8 (2C), 33.9 (2C), 10.1 (2C), 5.0; ES-API MS: m/z calcd for C₂₄H₂₂N₃O₅ 432.2, found 432.2 [M+H]⁺.

### (3aR,4R,4aR,5aS,6S,6aS)-2-(5-(2-(4-Methoxyphenyl)-2-oxoethoxy)pyrimidin-2-yl)-4,4a,5,5a,6,6a-hexahydro-4,6-ethenocyclopropa[f]isoindole-1,3(2H,3aH)-<dione (74)

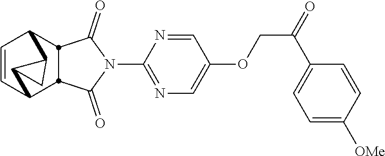 [SMILES: COc1ccc(C(=O)COc2cnc(N3C(=O)[C@@H]4[C@H](C3=O)[C@@H]3C=C[C@H]4C4CC43)nc2)cc1]

Procedure C. Purification by flash chromatography on silica gel (gradient elution, 0→25% EtOAc in hexanes) followed by recrystallization in CH₂Cl₂/hexanes afforded 74 (58 mg, 0.13 mmol, 79%) as a white solid. IR (cm⁻¹) 2975, 1714, 1601, 1428, 1237, 1177, 973, 730; ¹H NMR (400 MHz, CDCl₃) δ 8.45 (s, 2H), 7.93 (d, J=8.8 Hz, 2H), 6.98 (d, J=8.8 Hz, 2H), 5.91 (dd, J=4.4, 3.6 Hz, 2H), 5.36 (s, 2H), 3.90 (s, 3H), 3.50 (m, 2H), 3.17 (dd, J=1.6, 1.6 Hz, 2H), 1.13 (m, 2H), 0.35-0.26 (m, 2H); ¹³C NMR (100 MHz, CDCl₃) δ 190.7, 176.8 (2C), 164.7, 152.1, 146.7, 146.1 (2C), 130.6 (2C), 128.0 (2C), 126.9, 114.5 (2C), 71.0, 55.8, 46.0 (2C), 33.9 (2C), 10.1 (2C), 5.0; ES-API MS: m/z calcd for C₂₄H₂₂N₃O₅ 432.2, found 432.2 [M+H]⁺.

### (3aR,4R,4aR,5aS,6S,6aS)-2-(5-(2-(3-Chloro-4-methoxyphenyl)-2-oxoethoxy)pyridin-2-yl)-4,4a,5,5a,6,6a-hexahydro-4,6-ethenocyclopropa[f]isoindole-1,3(2H,3aH)-dione (75)

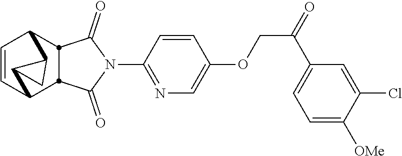 [SMILES: COc1ccc(C(=O)COc2ccc(N3C(=O)[C@@H]4[C@H](C3=O)[C@@H]3C=C[C@H]4C4CC43)nc2)cc1Cl]

Procedure C. Purification by flash chromatography on silica gel (gradient elution, 0→40% EtOAc in CH₂Cl₂) followed by recrystallization in CH₂Cl₂/hexanes afforded 75 (86 mg, 0.18 mmol, 52%) as a yellow solid. IR (cm⁻¹) 3055, 3009, 2954, 2846, 1775, 1711, 1595, 1478, 1400, 1263, 1209, 1061, 730; ¹H NMR (400 MHz, CDCl₃) δ 8.31 (d, J=3.2 Hz, 1H), 8.02 (d, J=2.4 Hz, 1H), 7.90 (dd, J=8.4, 2.4 Hz, 1H), 7.32 (dd, J=8.8, 2.8 Hz, 1H), 7.07 (d, J=8.8 Hz, 1H), 7.00 (d, J=8.8 Hz, 1H), 5.88 (dd, J=4.8, 3.6 Hz, 2H), 5.24 (s, 2H), 3.99 (s, 3H), 3.49 (m, 2H), 3.14 (dd, J=2.0, 2.0 Hz, 2H), 1.14 (m, 2H), 0.35-0.26 (m, 2H); ¹³C NMR (100 MHz, CDCl₃) δ 191.1, 177.5 (2C), 159.8, 154.4, 139.7, 137.8, 130.7, 129.0, 128.1 (2C), 127.8, 123.8, 123.6, 122.8, 111.8, 71.1, 56.7, 45.8 (2C), 33.9 (2C), 10.1 (2C), 5.0; ES-API MS: m/z calcd for C₂₅H₂₂ClN₂O₅ 465.1, found 465.1 [M+H]⁺.

### (3aR,4R,4aR,5aS,6S,6aS)-2-(5-(2-(4-Chloro-3-methylphenyl)-2-oxoethoxy)pyridin-2-yl)-4,4a,5,5a,6,6a-hexahydro-4,6-ethenocyclopropa[f]isoindole-1,3(2H,3aH)-dione (76)

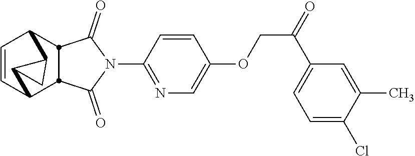 [SMILES: Cc1cc(C(=O)COc2ccc(N3C(=O)[C@@H]4[C@H](C3=O)[C@@H]3C=C[C@H]4C4CC43)nc2)ccc1Cl]

Procedure C. Purification by flash chromatography on silica gel (gradient elution, 0→65% EtOAc in hexanes) followed by recrystallization in CH₂Cl₂/hexanes afforded 76 (94 mg, 0.21 mmol, 59%) as a yellow solid. IR (cm⁻¹) 2956, 1710, 1580, 1478, 1400, 1291, 1232, 1186, 1050, 912, 730; ¹H NMR (400 MHz, CDCl₃) δ 8.31 (d, J=2.8 Hz, 1H), 7.83 (d, J=1.6 Hz, 1H), 7.72 (dd, J=8.4, 2.4 Hz, 1H), 7.47 (d, J=8.4 Hz, 1H), 7.31 (dd, J=8.8, 3.2 Hz, 1H), 7.07 (d, J=8.8 Hz, 1H), 5.88 (dd, J=4.8, 3.6 Hz, 2H), 5.27 (s, 2H), 3.49 (m, 2H), 3.14 (dd, J=2.0, 2.0 Hz, 2H), 2.45 (s, 3H), 1.14 (m, 2H), 0.35-0.26 (m, 2H); ¹³C NMR (100 MHz, CDCl₃) δ 192.6, 177.5 (2C), 154.4, 141.2, 139.8, 137.8, 132.7, 131.7, 130.7, 129.9, 128.1 (2C), 127.0, 123.9, 122.8, 71.2, 45.8 (2C), 33.9 (2C), 20.4, 10.1 (2C), 5.0; ES-API MS: m/z calcd for C₂₅H₂₂ClN₂O₄ 449.1, found 449.1 [M+H]⁺.

### (3aR,4R,4aR,5aS,6S,6aS)-2-(5-(2-(3-Chloro-4-fluorophenyl)-2-oxoethoxy)pyridin-2-yl)-4,4a,5,5a,6,6a-hexahydro-4,6-ethenocyclopropa[f]isoindole-1,3(2H,3aH)-dione (77)

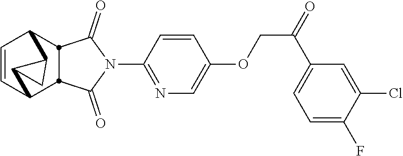 [SMILES: O=C(COc1ccc(N2C(=O)[C@@H]3[C@H](C2=O)[C@@H]2C=C[C@H]3C3CC32)nc1)c1ccc(F)c(Cl)c1]

Procedure C. Purification by flash chromatography on silica gel (gradient elution, 0→65% EtOAc in hexanes) followed by recrystallization in CH₂Cl₂/hexanes afforded 77 (71 mg, 0.16 mmol, 71%) as a yellow solid. IR (cm⁻¹) 3055, 3010, 2957, 1776, 1714, 1581, 1488, 1402, 1292, 1253, 1206, 1059, 831, 731; ¹H NMR (400 MHz, CDCl₃) δ 8.31 (d, J=2.8 Hz, 1H), 8.07 (dd, J=6.8, 2.4 Hz, 1H), 7.90 (ddd, J=8.4, 4.4, 2.0 Hz, 1H), 7.32 (dd, J=8.8, 3.2 Hz, 1H), 7.28 (d, J=9.2 Hz, 1H), 7.08 (d, J=8.8 Hz, 1H), 5.88 (dd, J=4.8, 3.6 Hz, 2H), 5.25 (s, 2H), 3.49 (m, 2H), 3.15 (dd, J=1.6, 1.6 Hz, 2H), 1.14 (m, 2H), 0.35-0.26 (m, 2H); ¹³C NMR (100 MHz, CDCl₃) δ 191.2, 177.5 (2C), 161.9 (d, J=257.9 Hz, 1C), 154.2, 140.0, 137.7, 131.5, 131.4 (d, J=3.8 Hz, 1C), 128.9 (d, J=8.6 Hz, 1C), 128.1 (2C), 123.4 (d, J=110.2 Hz, 1C), 122.7 (d, J=18.7 Hz, 1C), 117.5 (d, J=21.7 Hz, 1C), 71.2, 45.8 (2C), 33.9 (2C), 10.1 (2C), 5.0; ES-API MS: m/z calcd for C₂₄H₁₉ClFN₂O₄ 453.1, found 453.1 [M+H]⁺.

### (3aR,4R,4aR,5aS,6S,6aS)-2-(5-(2-(4-Chloro-3-(trifluoromethyl)phenyl)-2-oxoethoxy)pyridin-2-yl)-4,4a,5,5a,6,6a-hexahydro-4,6-ethenocyclopropa[f]isoindole-1,3(2H,3aH)-dione (78)

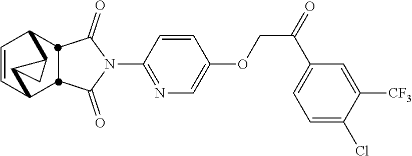 [SMILES: O=C(COc1ccc(N2C(=O)C3C(C2=O)[C@H]2C=C[C@@H]3C3CC32)nc1)c1ccc(Cl)c(C(F)(F)F)c1]

Procedure C. Purification by flash chromatography on silica gel (gradient elution, 0→65% EtOAc in hexanes) followed by recrystallization in CH₂Cl₂/hexanes afforded 78 (37 mg, 0.074 mmol, 44%) as a white solid. IR (cm⁻¹) 3055, 3012, 2957, 1774, 1710, 1604, 1478, 1401, 1320, 1209, 1181, 1141, 730; ¹H NMR (400 MHz, CDCl₃) δ 8.32 (d, J=3.6 Hz, 1H), 8.31 (d, J=2.4 Hz, 1H), 8.09 (dd, J=8.4, 2.0 Hz, 1H), 7.68 (d, J=8.4 Hz, 1H), 7.33 (dd, J=8.8, 2.8 Hz, 1H), 7.10 (d, J=8.8 Hz, 1H), 5.88 (dd, J=4.8, 3.6 Hz, 2H), 5.28 (s, 2H), 3.50 (m, 2H), 3.15 (dd, J=2.0, 2.0 Hz, 2H), 1.14 (m, 2H), 0.35-0.26 (m, 2H); ¹³C NMR (100 MHz, CDCl₃) δ 191.7, 177.5 (2C), 154.1, 140.1, 138.9 (q, J=2.0 Hz, 1C), 137.7, 132.7, 132.6, 129.9, 129.5, 128.1 (2C), 127.8 (q, J=5.0 Hz, 1C), 124.0, 122.9, 122.4 (q, J=273.0 Hz, 1C), 71.5, 45.8 (2C), 33.9 (2C), 10.1 (2C), 5.0; ES-API MS: m/z calcd for C₂₅H₁₉ClF₃N₂O₄ 503.1, found 503.1 [M+H]⁺.

### (3aR,4R,4aR,5aS,6S,6aS)-2-(5-(2-(3, 4-Dichlorophenyl)-2-oxoethoxy)pyridin-2-yl)-4,4a,5,5a,6,6a-hexahydro-4,6-ethenocyclopropa[f]isoindole-1,3(2H,3aH)-dione (79)

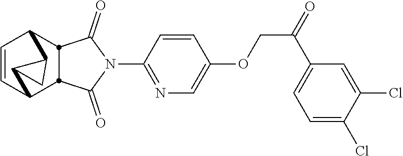 [SMILES: O=C(COc1ccc(N2C(=O)[C@@H]3[C@H](C2=O)[C@@H]2C=C[C@H]3C3CC32)nc1)c1ccc(Cl)c(Cl)c1]

Procedure C. Purification by flash chromatography on silica gel (gradient elution, 0→60% EtOAc in hexanes) followed by recrystallization in CH₂Cl₂/hexanes afforded 79 (133 mg, 0.28 mmol, 80%) as a yellow solid. IR (cm⁻¹) 3060, 3038, 3010, 2956, 1776, 1709, 1582, 1479, 1399, 1211, 1187, 1032, 730; ¹H NMR (400 MHz, CDCl₃) δ 8.31 (d, J=2.8 Hz, 1H), 8.06 (d, J=2.0 Hz, 1H), 7.80 (dd, J=8.4, 2.0 Hz, 1H), 7.60 (d, J=8.4 Hz, 1H), 7.32 (dd, J=8.8, 3.2 Hz, 1H), 7.08 (d, J=8.8 Hz, 1H), 5.88 (dd, J=4.8, 3.6 Hz, 2H), 5.25 (s, 2H), 3.49 (m, 2H), 3.15 (dd, J=1.6, 1.6 Hz, 2H), 1.14 (m, 2H), 0.35-0.26 (m, 2H); ¹³C NMR (100 MHz, CDCl₃) δ 191.6, 177.5 (2C), 154.2, 140.0, 139.2, 137.7, 134.1, 133.7, 131.4, 130.4, 128.1 (2C), 127.4, 123.9, 122.8, 71.3, 45.8 (2C), 33.9 (2C), 10.1 (2C), 5.0; ES-API MS: m/z calcd for C₂₄H₁₉Cl₂N₂O₄ 469.1, found 469.1 [M+H]⁺.

### (3aR,4R,4aR,5aS,6S,6aS)-2-(5-(2-(3,5-Dichlorophenyl)-2-oxoethoxy)pyridin-2-yl)-4,4a,5,5a,6,6a-hexahydro-4,6-ethenocyclopropa[f]isoindole-1,3(2H,3aH)-dione (80)

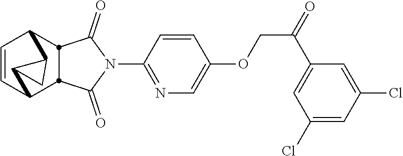 [SMILES: O=C(COc1ccc(N2C(=O)[C@@H]3[C@H](C2=O)[C@@H]2C=C[C@H]3C3CC32)nc1)c1cc(Cl)cc(Cl)c1]

Procedure C. Purification by flash chromatography on silica gel (gradient elution, 0→55% EtOAc in hexanes) followed by recrystallization in CH₂Cl₂/hexanes afforded 80 (44 mg, 0.094 mmol, 27%) as a white solid. IR (cm⁻¹) 3076, 2955, 1710, 1568, 1488, 1399, 1291, 1216, 1187, 730; ¹H NMR (400 MHz, CDCl₃) δ 8.31 (d, J=2.8 Hz, 1H), 7.83 (d, J=2.0 Hz, 2H), 7.62 (t, J=2.0 Hz, 1H), 7.32 (dd, J=8.4, 3.2 Hz, 1H), 7.09 (d, J=8.8 Hz, 1H), 5.88 (dd, J=4.4, 3.6 Hz, 2H), 5.25 (s, 2H), 3.49 (m, 2H), 3.15 (dd, J=1.5, 1.5 Hz, 2H), 1.14 (m, 2H), 0.35-0.26 (m, 2H); ¹³C NMR (100 MHz, CDCl₃) δ 191.3, 177.5 (2C), 154.1, 140.0, 137.8, 136.5, 136.3, 134.1 (2C), 128.1 (2C), 126.8 (2C), 123.9, 122.8, 71.3, 45.8 (2C), 33.9 (2C), 10.1 (2C), 5.0; ES-API MS: m/z calcd for C₂₄H₁₉Cl₂N₂O₄ 469.1, found 469.1 [M+H]⁺.

### (3aR,4R,4aR,5aS,6S,6aS)-2-(4-(2-(5,6-Dichloropyridin-3-yl)-2-oxoethoxy)phenyl)-4,4a,5,5a,6,6a-hexahydro-4,6-ethenocyclopropa[f]isoindole-1,3(2H,3aH)-<dione (81)

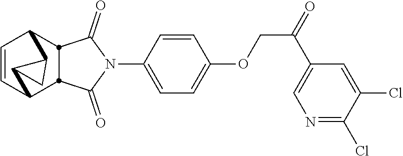 [SMILES: O=C(COc1ccc(N2C(=O)[C@@H]3[C@H](C2=O)[C@@H]2C=C[C@H]3C3CC32)cc1)c1cnc(Cl)c(Cl)c1]

Procedure C. Purification by flash chromatography on silica gel (gradient elution, 0→45% EtOAc in hexanes) followed by recrystallization in CH₂Cl₂/hexanes afforded 81 (50 mg, 0.10 mmol, 30%) as a pale yellow solid. IR (cm⁻¹) 3009, 1704, 1512, 1187, 734; ¹H NMR (400 MHz, CDCl₃) δ 8.91 (d, J=2.0 Hz, 1H), 8.35 (d, J=2.0 Hz, 1H), 7.11 (d, J=9.2 Hz, 2H), 6.96 (d, J=9.2 Hz, 2H), 5.84 (dd, J=4.8, 3.6 Hz, 2H), 5.13 (s, 2H), 3.48 (m, 2H), 3.12 (dd, J=1.6, 1.6 Hz, 2H), 1.14 (m, 2H), 0.35-0.25 (m, 2H); ¹³C NMR (100 MHz, CDCl₃) δ 192.6, 178.0 (2C), 157.3, 154.2, 147.4, 138.5, 137.7, 131.8, 130.1, 128.3 (2C), 128.0 (2C), 126.2, 115.4 (2C), 71.8, 45.5 (2C), 34.0 (2C), 10.1 (2C), 4.9; ES-API MS: m/z calcd for C₂₄H₁₉Cl₂N₂O₄ 469.1, found 469.1 [M+H]⁺.

### (3aR,4R,4aR,5aS,6S,6aS)-2-(5-(2-(3-Chloro-4-methoxyphenyl)-2-oxoethoxy)pyrimidin-2-yl)-4,4a,5,5a,6,6a-hexahydro-4,6-ethenocyclopropa[f]isoindole-1,3(2H,3aH)-dione (82)

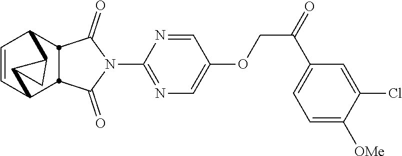 [SMILES: COc1ccc(C(=O)COc2cnc(N3C(=O)[C@@H]4[C@H](C3=O)[C@@H]3C=C[C@H]4C4CC43)nc2)cc1Cl]

Procedure C. Purification by flash chromatography on silica gel (gradient elution, 0→30% EtOAc in CH₂Cl₂) followed by recrystallization in CH₂Cl₂/hexanes afforded 82 (106 mg, 0.23 mmol, 65%) as a pale yellow solid. IR (cm⁻¹) 2952, 1714, 1595, 1428, 1263, 1212, 731; ¹H NMR (400 MHz, CDCl₃) δ 8.49 (s, 2H), 7.99 (d, J=2.0 Hz, 1H), 7.88 (dd, J=8.8, 2.0 Hz, 1H), 7.02 (d, J=8.8 Hz, 1H), 5.91 (dd, J=4.4, 3.6 Hz, 2H), 5.33 (s, 2H), 4.08 (s, 3H), 3.50 (m, 2H), 3.17 (dd, J=1.6, 1.6 Hz, 2H), 1.14 (m, 2H), 0.36-0.27 (m, 2H); ¹³C NMR (100 MHz, CDCl₃) δ 190.0, 176.8 (2C), 160.0, 152.0, 146.9, 146.1 (2C), 130.5, 128.9, 128.0 (2C), 127.4, 123.8, 111.9, 110.2, 71.0, 56.8, 46.0 (2C), 33.9 (2C), 10.1 (2C), 5.0; ES-API MS: m/z calcd for C₂₄H₂₁ClN₃O₅ 466.1, found 466.1 [M+H]⁺.

### (3aR,4R,4aR,5aS,6S,6aS)-2-(5-(2-(4-Chloro-3-methylphenyl)-2-oxoethoxy)pyrimidin-2-yl)-4,4a,5,5a,6,6a-hexahydro-4,6-ethenocyclopropa[f]isoindole-1,3(2H,3aH)-dione (83)

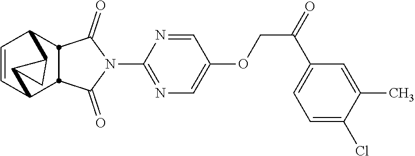 [SMILES: Cc1cc(C(=O)COc2cnc(N3C(=O)[C@@H]4[C@H](C3=O)[C@@H]3C=C[C@H]4C4CC43)nc2)ccc1Cl]

Procedure C. Purification by flash chromatography on silica gel (gradient elution, 0→15% EtOAc in CH₂Cl₂) followed by recrystallization in CH₂Cl₂/hexanes afforded 83 (67 mg, 0.15 mmol, 43%) as a white solid. IR (cm⁻¹) 2953, 1714, 1428, 1290, 1236, 1050, 731; ¹H NMR (400 MHz, CDCl₃) δ 8.48 (s, 2H), 7.82 (d, J=2.0 Hz, 1H), 7.70 (dd, J=8.4, 2.4 Hz, 1H), 7.49 (d, J=8.4 Hz, 1H), 5.91 (dd, J=4.8, 3.6 Hz, 2H), 5.36 (s, 2H), 3.50 (m, 2H), 3.17 (dd, J=1.6, 1.6 Hz, 2H), 2.46 (s, 3H), 1.14 (m, 2H), 0.36-0.27 (m, 2H); ¹³C NMR (100 MHz, CDCl₃) δ 191.5, 176.8 (2C), 152.0, 146.9, 146.1 (2C), 141.5, 137.7, 132.3, 130.6, 130.1, 128.0 (2C), 126.8, 71.1, 46.0 (2C), 33.9 (2C), 20.4, 10.1 (2C), 5.0; ES-API MS: m/z calcd for C₂₄H₂₁ClN₃O₄ 450.1, found 450.1 [M+H]⁺.

### (3aR,4R,4aR,5aS,6S,6aS)-2-(S-(2-(3-Chloro-4-fluorophenyl)-2-oxoethoxy)pyrimidin-2-yl)-4,4a,5,5a,6,6a-hexahydro-4,6-ethenocyclopropa[f]isoindole-1,3(2H,3aH)-dione (84)

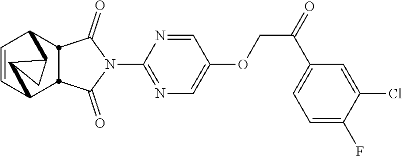 [SMILES: O=C(COc1cnc(N2C(=O)[C@@H]3[C@H](C2=O)C2C=C[C@H]3C3C[C@@H]23)nc1)c1ccc(F)c(Cl)c1]

Procedure C. Purification by flash chromatography on silica gel (gradient elution, 0→25% EtOAc in CH₂Cl₂) followed by recrystallization in CH₂Cl₂/hexanes afforded 84 (35 mg, 0.077 mmol, 45%) as a pale yellow solid. IR (cm⁻¹) 3010, 1713, 1428, 1251, 1187, 731; ¹H NMR (400 MHz, CDCl₃) δ 8.49 (s, 2H), 8.05 (dd, J=6.8, 2.0 Hz, 1H), 7.89 (ddd, J=8.8, 4.4, 2.0 Hz, 1H), 7.30 (dd, J=8.4, 8.4 Hz, 1H), 5.91 (dd, J=4.8, 3.6 Hz, 2H), 5.35 (s, 2H), 3.50 (m, 2H), 3.17 (dd, J=1.6, 1.6 Hz, 2H), 1.14 (m, 2H), 0.35-0.27 (m, 2H); ¹³C NMR (100 MHz, CDCl₃) δ 190.1, 176.8 (2C), 162.0 (d, J=258.3 Hz, 1C), 151.8, 147.1, 146.1 (2C), 131.4 (d, J=1.3 Hz, 1C), 131.1 (d, J=3.7 Hz, 1C), 128.8 (d, J=8.6 Hz, 1C), 128.1 (2C), 117.7 (d, J=21.7 Hz, 1C), 110.2, 71.1, 46.0 (2C), 33.9 (2C), 10.1 (2C), 5.0; ES-API MS: m/z calcd for C₂₃H₁₇ClFN₃O₄ 453.1 found 453.1 [M]⁺.

### (3aR,4R,4aR,5aS,6S,6aS)-2-(5-(2-(3,4-Dichlorophenyl)-2-oxoethoxy)pyrimidin-2-yl)-4,4a,5,5a,6,6a-hexahydro-4,6-ethenocyclopropa[f]isoindole-1,3(2H,3aH)-dione (85)

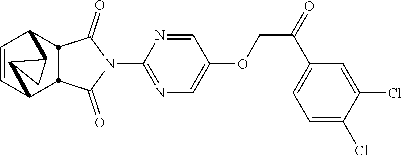 [SMILES: O=C(COc1cnc(N2C(=O)[C@@H]3[C@H](C2=O)C2C=C[C@H]3C3C[C@@H]23)nc1)c1ccc(Cl)c(Cl)c1]

Procedure C. Purification by flash chromatography on silica gel (gradient elution, 0→35% EtOAc in CH₂Cl₂) followed by recrystallization in CH₂Cl₂/hexanes afforded 85 (65 mg, 0.14 mmol, 40%) as a white solid. IR (cm⁻¹) 3009, 1712, 1429, 1397, 1216, 1184, 732; ¹H NMR (400 MHz, CDCl₃) δ 8.49 (s, 2H), 8.04 (d, J=2.0 Hz, 1H), 7.78 (dd, J=8.4, 2.0 Hz, 1H), 7.62 (d, J=8.4 Hz, 1H), 5.92 (dd, J=4.8, 3.2 Hz, 2H), 5.35 (s, 2H), 3.50 (m, 2H), 3.18 (dd, J=1.6, 2.0 Hz, 2H), 1.14 (m, 2H), 0.35-0.27 (m, 2H); ¹³C NMR (100 MHz, CDCl₃) δ 190.5, 176.8 (2C), 151.8, 147.1, 146.1 (2C), 139.6, 134.2, 133.4, 131.5, 130.3, 128.1 (2C), 127.2, 71.1, 46.0 (2C), 33.9 (2C), 10.1 (2C), 5.0; ES-API MS: m/z calcd for C₂₃H₁₈Cl₂N₃O₄ 470.1, found 470.1 [M+H]⁺.

### (3aR,4R,4aR,5aS,6S,6aS)-2-(5-(2-(3,5-Dichlorophenyl)-2-oxoethoxy)pyrimidin-2-yl)-4,4a,5,5a,6,6a-hexahydro-4,6-ethenocyclopropa[f]isoindole-1,3(2H,3aH)-dione (86)

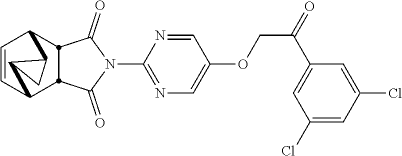 [SMILES: O=C(COc1cnc(N2C(=O)[C@@H]3[C@H](C2=O)C2C=C[C@H]3C3C[C@@H]23)nc1)c1cc(Cl)cc(Cl)c1]

Procedure C. Purification by flash chromatography on silica gel (gradient elution, 0→20% EtOAc in hexanes) followed by recrystallization in CH₂Cl₂/hexanes afforded 86 (33 mg, 0.07 mmol, 42%) as a pale yellow solid. IR (cm⁻¹) 3008, 1712, 1428, 1220, 731; ¹H NMR (400 MHz, CDCl₃) δ 8.50 (s, 2H), 7.81 (d, J=2.0 Hz, 2H), 7.65 (t, J=2.0 Hz, 1H), 5.92 (dd, J=4.8, 3.6 Hz, 2H), 5.34 (s, 2H), 3.51 (m, 2H), 3.18 (dd, J=1.6, 1.6 Hz, 2H), 1.14 (m, 2H), 0.36-0.27 (m, 2H); ¹³C NMR (100 MHz, CDCl₃) δ 190.3, 176.8 (2C), 151.8, 147.1, 146.1 (2C), 136.5, 136.2, 134.4 (2C), 128.1 (2C), 126.7 (2C), 71.1, 46.0 (2C), 33.9 (2C), 10.1 (2C), 5.0; ES-API MS: m/z calcd for C₂₃H₁₈Cl₂N₃O₄ 470.1, found 470.1 [M+H]⁺.

### (3aR,4R,4aR,5aS,6S,6aS)-2-(4-(Hydroxymethyl)phenyl)-4,4a,5,5a,6,6a-hexahydro-4,6-ethenocyclopropa[f]isoindole-1,3(2H,3aH)-dione (105)

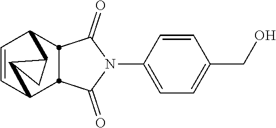 [SMILES: O=C1[C@@H]2[C@H](C(=O)N1c1ccc(CO)cc1)C1C=C[C@H]2C2C[C@@H]12]

Procedure A. Purification by flash chromatography on silica gel (gradient elution, 0→80% EtOAc in hexanes) followed by recrystallization in CH₂Cl₂/hexanes afforded 105 (80 mg, 0.27 mmol, 52%) as a pale yellow solid. IR (cm⁻¹) 3504, 3052, 3033, 2963, 2886, 1696, 1508, 1395, 1191, 1175, 1006; ¹H NMR (400 MHz, CDCl₃) δ 7.42 (d, J=8.4 Hz, 2H), 7.15 (d, J=8.4 Hz, 2H), 5.86 (dd, J=4.8, 3.6 Hz, 2H), 4.69 (s, 2H), 3.49 (m, 2H), 3.13 (dd, J=2.0, 1.6 Hz, 2H), 1.79 (br, 1H), 1.15 (m, 2H), 0.30 (m, 2H); ¹³C NMR (100 MHz, CDCl₃) δ 177.7 (2C), 141.4, 131.1, 127.8 (2C), 127.4 (2C), 126.6 (2C), 64.8, 45.3 (2C), 33.8 (2C), 9.9 (2C), 4.7; ES-API MS: m/z calcd for C₁₈H₁₈NO₃ 296.1, found 296.1 [M+H]⁺.

### 4-((3aR,4R,4aS 5aS,6S,6aS)-1,3-Dioxo-3,3a,4,4a,5,5a,6,6a-octahydro-4,6-ethenocyclopropa[f]isoindol-2(1H)-yl)-3-fluorobenzonitrile (106)

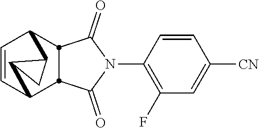 [SMILES: N#Cc1ccc(N2C(=O)[C@@H]3[C@H](C2=O)C2C=C[C@H]3C3C[C@@H]23)c(F)c1]

Procedure B (microwave). Benzonitrile 106 was obtained by crystallization in CH₂Cl₂/Et₂O/hexanes as yellow crystals (2.55 g, 8.27 mmol, 80%). IR (cm⁻¹) 3067, 2989, 2360, 1722, 1421, 1266, 1254, 1169, 1045, 899, 728; ¹H NMR (400 MHz, CDCl₃) δ 7.52 (d, J=8.4 Hz, 1H), 7.49 (d, J=8.8 Hz, 1H), 7.30 (br, 1H), 5.87 (dd, J=4.4, 4.0 Hz, 2H), 3.50 (m, 2H), 3.21 (m, 2H), 1.16 (m, 2H), 0.38-0.27 (m, 2H); ¹³C NMR (100 MHz, CDCl₃) δ 177.9 (2C), 176.2 (2C), 157.2 (d, J=256.6 Hz, 1C), 130.7, 128.7 (d, J=4.2 Hz, 1C), 128.0 (2C), 124.7 (d, J=13.3 Hz, 1C), 120.8 (d, J=23.1 Hz, 1C), 117.0 (d, J=2.6 Hz, 1C), 114.5 (d, J=9.1 Hz, 1C), 46.00, 45.96, 34.0 (2C), 10.0 (2C), 5.0; ES-API MS: m/z calcd for C₁₈H₁₄FN₂O₂ 309.1, found 309.1 [M+H]⁺.

### 4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)benzaldehyde (107)

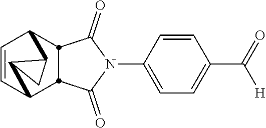 [SMILES: O=Cc1ccc(N2C(=O)[C@@H]3[C@H](C2=O)C2C=C[C@H]3C3C[C@@H]23)cc1]

To a solution of benzyl alcohol 105 (100 mg, 0.34 mmol) in anhydrous toluene was added MnO₂ (176 mg, 2.03 mmol), and the mixture was heated to 70° C. and stirred for 5 h. The reaction was cooled to rt, and filtered through a Celite pad and washed the residue with CH₂Cl₂. The solvent was removed under reduced pressure and the residue was purified by flash chromatography on silica gel (gradient elution, 0→45% EtOAc in hexanes) to afford benzaldehyde 105 (98 mg, 0.33 mmol, 98%) as an amorphous yellow solid. IR (cm⁻¹) 2999, 2954, 2824, 1773, 1704, 1700, 1601, 1386, 1186, 734, 720; ¹H NMR (400 MHz, CDCl₃) δ 10.01 (s, 1H), 7.93 (d, J=8.0 Hz, 2H), 7.40 (d, J=8.0 Hz, 2H), 5.86 (br, 2H), 3.50 (br, 2H), 3.16 (s, 2H), 1.16 (br, 2H), 0.32 (m, 2H); ¹³C NMR (100 MHz, CDCl₃) δ 191.1, 177.0 (2C), 137.0, 135.7, 130.2 (2C), 127.9 (2C), 126.9 (2C), 45.4 (2C), 33.9 (2C), 9.9 (2C), 4.7; ES-API MS: m/z calcd for C₁₈H₁₆NO₃ 294.1, found 294.1 [M+H]⁺.

### 4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)-3-fluorobenzaldehyde (108)

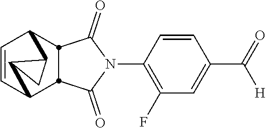 [SMILES: O=Cc1ccc(N2C(=O)[C@@H]3[C@H](C2=O)C2C=C[C@H]3C3C[C@@H]23)c(F)c1]

Benzonitrile 106 (2.46 g, 7.98 mmol) was dissolved in formic acid (16 mL, 80% in deionized H₂O) in a pressure seal-tube. Raney-Ni (1.2 g) was added in one portion to the reaction. The resulting mixture was sealed and refluxed at 110° C. for 4 h, cooled to rt, filtered through a celite pad and washed with EtOAc. The eluate was neutralized with saturated NaHCO₃, and washed with brine, dried over anhydrous Na₂SO₄, and filtered. The solvent was removed under reduced pressure and the residue was purified by flash chromatography on silica gel (gradient elution, 0→45% EtOAc in hexanes) followed by recrystallization in CH₂Cl₂/hexanes to give the desired benzaldehyde 108 as a colorless solid (1.95 g, 6.26 mmol, 79%). IR (cm⁻¹) 3069, 3045, 3015, 1780, 1698, 1588, 1512, 1391, 1253, 1181, 816, 736, 718; ¹H NMR (400 MHz, CDCl₃) δ 9.99 (s, 1H), 7.74 (d, J=8.8 Hz, 1H), 7.69 (d, J=9.6 Hz, 1H), 7.35 (br, 1H), 5.88 (dd, J=4.4, 3.6 Hz, 2H), 3.50 (m, 2H), 3.21 (m, 2H), 1.17 (m, 2H), 0.37-0.28 (m, 2H); ¹³C NMR (100 MHz, CDCl₃) δ 190.1 (d, J=1.8 Hz, 1C), 176.4 (2C), 157.9 (d, J=255.7 Hz, 1C), 138.4 (d, J=6.0 Hz, 1C), 130.4, 128.0 (2C), 126.2 (d, J=3.5 Hz, 1C), 125.4 (d, J=14.0 Hz, 1C), 116.9 (d, J=20.3 Hz, 1C), 46.04, 45.99, 34.0 (2C), 10.1 (2C), 5.0; ES-API MS: m/z calcd for C₁₈H₁₅FNO₃ 312.1, found 312.1 [M+H]⁺.

### (3aR,4R,4aR,5aS,6S,6aS)-2-(4-(Aminomethyl)phenyl)-4,4a,5,5a,6,6a-hexahydro-4,6-ethenocyclopropa[f]isoindole-1,3(2H,3aH)-dione (109)

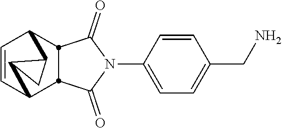 [SMILES: NCc1ccc(N2C(=O)[C@@H]3[C@H](C2=O)C2C=C[C@H]3C3C[C@@H]23)cc1]

Step 1. (3aR,4R,4a,5aS,6S,6aS)-2-(4-(Azidomethyl)phenyl)-4,4a,5,5a,6,6a-hexahydro-4,6-ethenocyclopropa[f]isoindole-1,3(2H,3aH)-dione. To a solution of benzyl alcohol 105 (50 mg, 0.17 mmol) and PPh₃ (67 mg, 0.25 mmol) in anhydrous DMF (0.85 mL) was added tetrabromomethane (84 mg, 0.25 mmol) at 0° C. The resulting yellow mixture was stirred at 0° C. until TLC showed complete conversion of the alcohol, then Na_{N}(33 mg, 0.51 mmol) was added in one portion and stirred was continued at rt for an additional 2 h. The reaction was diluted with H₂O (5 mL) and extracted with EtOAc (3×5 mL). The combined organic phase was washed with brine and dried over anhydrous Na₂SO₄, and filtered. The solvent was removed under reduced pressure and the residue was purified by flash chromatography on silica gel (gradient elution, 0→35% EtOAc in hexanes) to afford the azide (49 mg, 0.15 mmol, 91%) as a white solid. IR (cm⁻¹) 3078, 3052, 3009, 2979, 2949, 2099, 1706, 1516, 1387, 1190, 734, 714; ¹H NMR (400 MHz, CDCl₃) δ 7.36 (d, J=8.0 Hz, 2H), 7.19 (d, J=8.4 Hz, 2H), 5.84 (dd, J=4.4, 3.2 Hz, 2H), 4.33 (s, 2H), 3.47 (m, 2H), 3.12 (dd, J=1.6, 1.6 Hz, 2H), 1.15-1.11 (m, 2H), 0.33-0.24 (m, 2H); ¹³C NMR (100 MHz, CDCl₃) δ 177.5 (2C), 135.9, 131.7, 128.6 (2C), 127.8 (2C), 126.8 (2C), 54.2, 45.3 (2C), 33.8 (2C), 9.9 (2C), 4.7; ES-API MS: m/z calcd for C₁₈H₁₈N₂O₂K 333.1, found 333.1 [M+K—N₂]⁺.

Step 2: To a solution of the above azide (step 1) (1.31 g, 4.09 mmol) in THF/H₂O (v:v/10:1, 40 mL) was added PMe₃ solution (8.2 mL, 1.0 M in THF). The resulting mixture was stirred at rt for 1 h. The reaction was then diluted with H₂O (20 mL) and extracted with EtOAc (3×20 mL). The combined organic phase was washed with brine and dried over anhydrous Na₂SO₄, and filtered. The solvent was removed under reduced pressure and the residue was recrystallized in CH₂Cl₂/hexanes to afford the amine 109 (1.15 g, 0.15 mmol, 91%) as a yellow solid. IR (cm⁻¹) 3461, 3372, 3008, 2954, 2867, 1772, 1713, 1704, 1514, 1385, 1186, 734; ¹H NMR (400 MHz, CDCl₃) δ 7.36 (d, J=8.4 Hz, 2H), 7.10 (d, J=8.4 Hz, 2H), 5.83 (dd, J=4.8, 3.2 Hz, 2H), 3.85 (m, 2H), 1.76 (m, 2H), 1.14-1.10 (m, 2H), 0.32-0.23 (m, 2H); ¹³C NMR (100 MHz, CDCl₃) δ 177.7 (2C), 130.4, 128.8, 127.8 (2C), 127.7 (2C), 126.6 (2C), 126.4, 45.3 (2C), 33.8 (2C), 9.9 (2C), 4.7; ES-API MS: m/z calcd for C₁₈H₁₉N₂O₂Na 318.1, found 318.1 [M+Na+H]⁺.

### (3aR,4R,4aR,5aS,6S,6aS)-2-(4-((E)-3-(4-Melhoxyphenyl)-3-oxoprop-1-en-1-yl)phenyl)-4,4a,5,5a,6,6a-hexahydro-4,6-ethenocyclopropa[f]isoindole-1,3(2H,3aH)-dione (110)

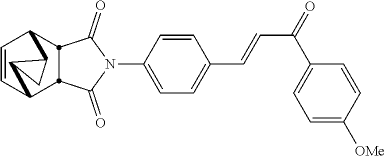 [SMILES: COc1ccc(C(=O)/C=C/c2ccc(N3C(=O)[C@@H]4[C@H](C3=O)C3C=C[C@H]4C4C[C@@H]34)cc2)cc1]

Procedure E. Purification by flash chromatography on silica gel (gradient elution, 0→50% EtOAc in hexanes) afforded enone 110 (70 mg, 0.16 mmol, 85%) as an amorphous yellow solid. IR (cm⁻¹) 3465, 3010, 2957, 2841, 1707, 1660, 1604, 1512, 1378, 1335, 1261, 1222, 1172, 1022, 819, 733; ¹H NMR (400 MHz, CDCl₃) δ 8.03 (d, J=9.2 Hz, 2H), 7.77 (d, J=15.6 Hz, 1H), 7.69 (d, J=8.4 Hz, 2H), 7.52 (d, J=15.6 Hz, 1H), 7.26 (d, J=8.4 Hz, 2H), 6.99 (d, J=8.8 Hz, 2H), 5.87 (dd, J=4.8, 3.2 Hz, 2H), 3.89 (s, 3H), 3.51 (m, 2H), 3.16 (dd, J=1.6, 1.6 Hz, 2H), 1.16 (m, 2H), 0.36-0.27 (m, 2H); ¹³C NMR (100 MHz, CDCl₃) δ 188.4, 177.4 (2C), 163.5, 142.6, 135.2, 133.3, 130.9, 130.8 (2C), 128.8 (2C), 127.8 (2C), 126.8 (2C), 122.9, 113.9 (2C), 55.5, 45.4 (2C), 33.8 (2C), 9.9 (2C), 4.7; ES-API MS: m/z calcd for C₂₇H₂₄NO₄ 426.2, found 426.0 [M+H]⁺.

### (3aR,4R,4aR,5aS,6S,6aS)-2-(4-((E)-3-(3-Chloro-4-methoxyphenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111a)

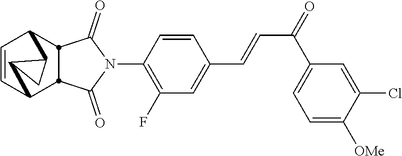 [SMILES: COc1ccc(C(=O)/C=C/c2ccc(N3C(=O)[C@@H]4[C@H](C3=O)C3C=C[C@H]4C4C[C@@H]34)c(F)c2)cc1Cl]

Procedure E. Purification by flash chromatography on silica gel (gradient elution, 0→50% EtOAc in hexanes) afforded enone 111a (263 mg, 0.55 mmol, 86%) as a pale yellow solid. IR (cm⁻¹) 3056, 3010, 2954, 1722, 1662, 1607, 1593, 1392, 1276, 734; ¹H NMR (400 MHz, CDCl₃) δ 8.04 (d, J=1.6 Hz, 1H), 7.91 (dd, J=8.4, 2.0 Hz, 1H), 7.66 (d, J=15.6 Hz, 1H), 7.44 (d, J=15.6 Hz, 1H), 7.40 (m, 1H), 7.38 (m, 1H), 7.12 (br, 1H), 6.96 (d, J=8.8 Hz, 1H), 5.84 (dd, J=4.0, 3.6 Hz, 2H), 3.93 (s, 3H), 3.45 (m, 2H), 3.16 (m, 2H), 1.12 (m, 2H), 0.32-0.24 (m, 2H); ¹³C NMR (100 MHz, CDCl₃) δ 186.9, 176.6 (2C), 158.8, 157.4 (d, J=252.9 Hz, 1C), 142.0 (d, J=2.0 Hz, 1C), 137.7 (d, J=7.6 Hz, 1C), 131.1, 130.8, 129.6, 129.1, 127.7, 124.7 (d, J=2.1 Hz, 1C), 123.3, 123.0, 121.2 (d, J=13.9 Hz, 1C), 115.6 (d, J=20.2 Hz, 1C), 111.4, 56.4, 45.7 (2C), 33.8 (2C), 9.9 (2C), 4.8; ES-API MS: m/z calcd for C₂₇H₂₂ClFNO₄ 478.1, found 478.1 [M+H]⁺.

### (3aR,4R,4aR,5aS,6S,6aS)-2-(4-((E)-3-(4-Chloro-3-methylphenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111b)

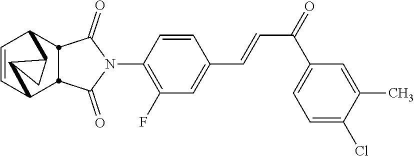 [SMILES: Cc1cc(C(=O)/C=C/c2ccc(N3C(=O)[C@@H]4[C@H](C3=O)C3C=C[C@H]4C4C[C@@H]34)c(F)c2)ccc1Cl]

Procedure E. Purification by flash chromatography on silica gel (gradient elution, 0→40% EtOAc in hexanes) afforded enone 111b (228 mg, 0.49 mmol, 77%) as a pale yellow solid. IR (cm⁻¹) 3055, 2947, 3007, 1716, 1666, 1609, 1517, 1391, 1181, 1050, 734; ¹H NMR (400 MHz, CDCl₃) δ 7.88 (d, J=1.2 Hz, 1H), 7.77 (dd, J=8.4, 2.4 Hz, 1H), 7.72 (d, J=15.6 Hz, 1H), 7.48 (d, J=2.4 Hz, 1H), 7.46-7.43 (m, 3H), 7.18 (br, 1H), 5.88 (dd, J=4.0, 4.0 Hz, 2H), 3.50 (m, 2H), 3.20 (m, 2H), 2.46 (s, 3H), 1.16 (m, 2H), 0.37-0.28 (m, 2H); ¹³C NMR (100 MHz, CDCl₃) δ 189.0, 176.8 (2C), 157.7 (d, J=253.2 Hz, 1C), 142.6 (d, J=2.3 Hz, 1C), 140.0, 137.9 (d, J=7.5 Hz, 1C), 137.0, 136.3, 131.2, 129.9, 129.7, 128.0, 127.5, 124.9 (d, J=2.9 Hz, 1C), 124.0, 123.8, 121.5 (d, J=13.9 Hz, 1C), 115.9 (d, J=20.0 Hz, 1C), 46.0, 45.9, 34.0 (2C), 20.4, 10.1 (2C), 5.0; ES-API MS: m/z calcd for C₂₇H₂₂ClFNO₃ 462.1, found 462.1 [M+H]⁺.

### (3aR,4R,4aR,5aS,6S,6aS)-2-(4-((E)-3-(3-Chloro-4-fluorophenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111c)

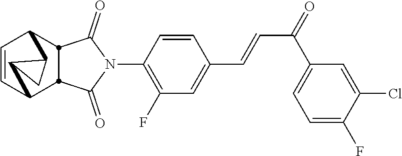 [SMILES: O=C(/C=C/c1ccc(N2C(=O)[C@@H]3[C@H](C2=O)C2C=C[C@H]3C3C[C@@H]23)c(F)c1)c1ccc(F)c(Cl)c1]

Procedure E. Purification by flash chromatography on silica gel (gradient elution, 0→40% EtOAc in hexanes) afforded enone 111c (189 mg, 0.41 mmol, 63%) as a pale yellow solid. IR (cm⁻¹) 3008, 1713, 1609, 1516, 1391, 1250, 1205, 814, 740; ¹H NMR (400 MHz, CDCl₃) δ 8.08 (dd, J=7.2, 2.0 Hz, 1H), 7.92 (m, 1H), 7.73 (d, J=15.6 Hz, 1H), 7.44 (m, 1H), 7.42 (m, 1H), 7.42 (d, J=15.6 Hz, 1H), 7.26 (dd, J=8.8, 8.4 Hz, 1H), 7.17 (br, 1H), 5.87 (dd, J=3.6, 4.0 Hz, 2H), 3.49 (m, 2H), 3.19 (m, 2H), 1.15 (m, 2H), 0.36-0.27 (m, 2H); ¹³C NMR (100 MHz, CDCl₃) δ 187.2, 176.7 (2C), 161.2 (d, J=255.9 Hz, 1C), 157.6 (d, J=253.2 Hz, 1C), 143.3 (d, J=2.4 Hz, 1C), 137.6 (d, J=7.5 Hz, 1C), 135.0 (d, J=3.6 Hz, 1C), 131.6 (d, J=0.8 Hz, 1C), 130.0, 129.1 (d, J=8.4 Hz, 1C), 127.9, 125.0 (d, J=3.1 Hz, 1C), 123.2 (2C), 122.2 (d, J=18.1 Hz, 1C), 121.7 (d, J=17.8 Hz, 1C), 117.1 (d, J=21.6 Hz, 1C), 115.9 (d, J=20.2 Hz, 1C), 45.96, 45.93, 34.0 (2C), 10.0 (2C), 5.0; ES-API MS: m/z calcd for C₂₆H₁₉ClF₂NO₃ 466.1, found 466.1 [M+H]⁺.

### (3aR,4R,4aR,5aS,6S,6aS)-2-(4-((E)-3-(4-Chloro-3-(trifluoromethyl)phenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111d)

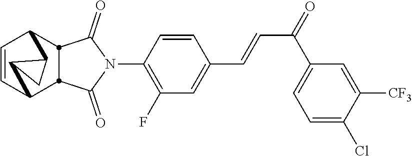 [SMILES: O=C(/C=C/c1ccc(N2C(=O)[C@@H]3[C@H](C2=O)C2C=C[C@H]3C3C[C@@H]23)c(F)c1)c1ccc(Cl)c(C(F)(F)F)c1]

Procedure E. Purification by flash chromatography on silica gel (gradient elution, 0→40% EtOAc in hexanes) afforded enone 111d (192 mg, 0.37 mmol, 58%) as a pale yellow solid. IR (cm⁻¹) 3009, 2997, 1713, 1610, 1515, 1311, 1206, 1175, 737; ¹H NMR (400 MHz, CDCl₃) δ 8.31 (s, 1H), 8.10 (dd, J=8.4, 1.2 Hz, 1H), 7.75 (d, J=15.6 Hz, 1H), 7.64 (d, J=8.4 Hz, 1H), 7.44 (d, J=15.6 Hz, 1H), 7.45 (m, 1H), 7.43 (m, 1H), 7.18 (br, 1H), 5.87 (dd, J=4.0, 3.6 Hz, 2H), 3.49 (m, 2H), 3.19 (m, 2H), 1.15 (m, 2H), 0.37-0.27 (m, 2H); ¹³C NMR (100 MHz, CDCl₃) δ 187.4, 176.7 (2C), 157.6 (d, J=253.4 Hz, 1C), 143.8 (d, J=2.2 Hz, 1C), 137.5 (q, J=1.5 Hz, 1C), 137.4 (d, J=7.5 Hz, 1C), 136.3, 132.7, 132.2, 130.0, 129.2 (q, J=31.8 Hz, 1C), 127.9, 127.8 (q, J=5.3 Hz, 1C), 125.1 (d, J=2.0 Hz, 1C), 122.9 (2C), 122.6 (q, J=272.3 Hz, 1C), 121.9 (d, J=13.8 Hz, 1C), 116.0 (d, J=20.3 Hz, 1C), 45.93, 45.87, 34.0 (2C), 10.0 (2C), 5.0; ES-API MS: m/z calcd for C₂₇H₁₉ClF₄NO₃ 516.1, found 516.1 [M+H]⁺.

### (3aR,4R,4aR,5aS,6S,6aS)-2-(4-((E)-3-(3,4-Dichlorophenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111e)

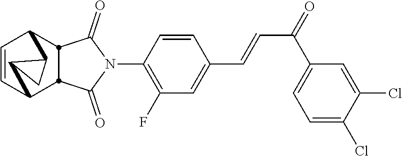 [SMILES: O=C(/C=C/c1ccc(N2C(=O)[C@@H]3[C@H](C2=O)C2C=C[C@H]3C3C[C@@H]23)c(F)c1)c1ccc(Cl)c(Cl)c1]

Procedure E. Purification by flash chromatography on silica gel (gradient elution, 0→40% EtOAc in hexanes) afforded enone 111e (175 mg, 0.36 mmol, 57%) as a pale yellow solid. IR (cm⁻¹) 2957, 1714, 1607, 1516, 1391, 1210, 1031, 739; ¹H NMR (400 MHz, CDCl₃) δ 8.08 (d, J=1.6 Hz, 1H), 7.82 (dd, J=8.4, 1.6 Hz, 1H), 7.73 (d, J=16.0 Hz, 1H), 7.57 (d, J=8.4 Hz, 1H), 7.44 (m, 1H), 7.42 (m, 1H), 7.41 (d, J=16.0 Hz, 1H), 7.17 (br, 1H), 5.87 (dd, J=4.0, 4.0 Hz, 2H), 3.49 (m, 2H), 3.19 (m, 2H), 1.15 (m, 2H), 0.36-0.27 (m, 2H); ¹³C NMR (100 MHz, CDCl₃) δ 187.6, 176.7 (2C), 157.6 (d, J=253.3 Hz, 1C), 143.5 (d, J=2.2 Hz, 1C), 137.9, 137.5 (d, J=7.5 Hz, 1C), 137.4, 133.6, 131.0, 130.7, 129.9, 128.0, 127.7, 125.0 (d, J=2.8 Hz, 1C), 123.2 (2C), 121.8 (d, J=13.8 Hz, 1C), 115.9 (d, J=20.3 Hz, 1C), 45.94, 45.90, 34.0 (2C), 10.0 (2C), 5.0; ES-API MS: m/z calcd for C₂₆H₁₉Cl₂FNO₃ 482.1, found 482.1 [M+H]⁺.

### (3aR,4R,4aR,5aS,6S,6aS)-2-(4-((E)-3-(3,5-Dichlorophenyl)-3-oxoprop-1-en-1-yl)-2-fluorophenyl)-4,4a,5,5a,6,6a-hexahydro-4,6-ethenocyclopropa[f]isoindole-1,3(2H,3aH)-dione (111f)

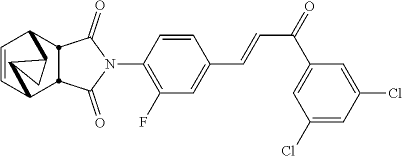 [SMILES: O=C(/C=C/c1ccc(N2C(=O)[C@@H]3[C@H](C2=O)C2C=C[C@H]3C3C[C@@H]23)c(F)c1)c1cc(Cl)cc(Cl)c1]

Procedure E. Purification by flash chromatography on silica gel (gradient elution, 0→40% EtOAc in hexanes) afforded enone 111f (271 mg, 0.56 mmol, 88%) as a pale yellow solid. IR (cm⁻¹) 3077, 3009, 2957, 1714, 1564, 1516, 1391, 1214, 1178, 738, 700; ¹H NMR (400 MHz, CDCl₃) δ 7.81 (s, 2H), 7.66 (d, J=15.6 Hz, 1H), 7.51 (m, 1H), 7.41 (m, 1H), 7.38 (m, 1H), 7.38 (d, J=15.6 Hz, 1H), 7.13 (br, 1H), 5.83 (m, 2H), 3.45 (m, 2H), 3.16 (m, 2H), 1.12 (m, 2H), 0.32-0.25 (m, 2H); ¹³C NMR (100 MHz, CDCl₃) δ 187.1, 176.6 (2C), 157.4 (d, J=253.2 Hz, 1C), 143.6 (d, J=1.8 Hz, 1C), 140.2, 137.2 (d, J=7.5 Hz, 1C), 135.7 (2C), 132.7, 129.8, 127.8, 127.0 (2C), 125.1, 122.9 (2C), 121.7 (d, J=13.9 Hz, 1C), 115.8 (d, J=20.4 Hz, 1C), 45.8 (2C), 33.8 (2C), 9.9 (2C), 4.9; ES-API MS: m/z calcd for C₂₆H₁₉Cl₂FNO₃ 482.1, found 482.0 [M+H]⁺.

### 4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)benzyl 4-methoxybenzoate (12)

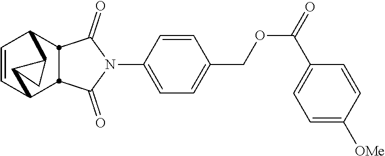 [SMILES: COc1ccc(C(=O)OCc2ccc(N3C(=O)[C@@H]4[C@@H]5C=CC([C@H]6CC65)[C@@H]4C3=O)cc2)cc1]

To a mixture of benzyl alcohol 105 (50 mg, 0.17 mmol), p-anisic acid (39 mg, 0.25 mmol) and PPh₃ (67 mg, 0.25 mmol) in anhydrous THF (1.7 mL) at 0° C. was added dropwise of DIAD (51 mg, 0.25 mmol). The resulting mixture was stirred and slowly raised to rt over 3 h. The solvent was removed under reduced pressure and the residue was purified by flash chromatography on silica gel (gradient elution, 0→45% EtOAc in hexanes) followed by recrystallization in CH₂Cl₂/hexanes to afford ester 12 (52 mg, 0.12 mmol, 71%) as a yellow solid. IR (cm⁻¹) 3450, 3056, 3008, 1707, 1606, 1512, 1377, 1274, 1257, 1169, 736; ¹H NMR (400 MHz, CDCl₃) δ 8.01 (d, J=8.4 Hz, 2H), 7.51 (d, J=8.0 Hz, 2H), 7.20 (d, J=8.4 Hz, 2H), 6.91 (d, J=8.8 Hz, 2H), 5.86 (dd, J=4.8, 3.6 Hz, 2H), 5.33 (s, 2H), 3.86 (s, 3H), 3.50 (m, 2H), 3.14 (dd, J=1.2, 1.2 Hz, 2H), 1.15 (m, 2H), 0.35-0.26 (m, 2H); ¹³C NMR (100 MHz, CDCl₃) δ 177.6 (2C), 166.0, 163.5, 136.7, 131.7 (2C), 131.6, 128.82 (2C), 127.79 (2C), 126.6 (2C), 122.3, 113.6 (2C), 65.7, 55.4, 45.3 (2C), 33.8 (2C), 9.9 (2C), 4.7; ES-API MS: m/z calcd for C₂₆H₂₄NO 430.2, found 430.1 [M+H]⁺.

### N-(4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)benzyl)-4-methoxybenzamide (17)

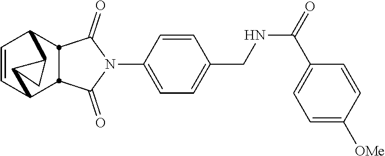 [SMILES: COc1ccc(C(=O)NCc2ccc(N3C(=O)[C@@H]4[C@@H]5C=CC([C@H]6CC65)[C@@H]4C3=O)cc2)cc1]

To a solution of benzylic amine 109 (45 mg, 0.15 mmol) in anhydrous CH₂Cl₂ (0.5 mL) was added Et₃N (18.6 mg, 0.18 mmol) and p-methoxybenzoyl chloride (27.5 mg, 0.16 mmol). The resulting mixture was stirred at rt for 2 h. The solvent was removed under reduced pressure and the residue was purified by flash chromatography on silica gel (gradient elution, 0→90% EtOAc in hexanes) followed by recrystallization in CH₂Cl₂/hexanes to afford amide 17 as a pale yellow solid (54 mg, 0.13 mmol, 83%). IR (cm⁻¹) 3338, 3010, 2956, 1704, 1607, 1505, 1387, 1255, 1181, 732; ¹H NMR (400 MHz, CDCl₃) δ 7.74 (d, J=8.8 Hz, 2H), 7.41 (d, J=8.4 Hz, 2H), 7.14 (d, J=8.4 Hz, 2H), 6.91 (d, J=8.8 Hz, 2H), 6.35 (t, J=5.6 Hz, 1H), 5.85 (dd, J=4.8, 3.2 Hz, 2H), 4.62 (d, J=5.6 Hz, 2H), 3.84 (s, 3H), 3.49 (m, 2H), 3.13 (dd, J=2.0, 2.0 Hz, 2H), 1.14 (m, 2H), 0.35-0.26 (m, 2H); ¹³C NMR (100 MHz, CDCl₃) δ 177.6 (2C), 166.8, 162.3, 138.9, 131.1, 128.77 (2C), 128.76 (2C), 127.8 (2C), 126.8 (2C), 126.4, 113.8 (2C), 55.4, 45.3 (2C), 43.6, 33.8 (2C), 9.9 (2C), 4.7; ES-API MS: m/z calcd for C₂₆H₂₅N₂O₄ 429.2, found 429.2 [M+H]⁺.

### N-(4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)benzyl)-4-methoxybenzenesulfonamide (18)

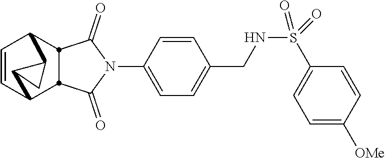 [SMILES: COc1ccc(S(=O)(=O)NCc2ccc(N3C(=O)[C@@H]4[C@@H]5C=CC([C@H]6CC65)[C@@H]4C3=O)cc2)cc1]

To a solution of p-methoxybenzenesulfonyl chloride (1.0 equiv.) in anhydrous CH₂Cl₂ (0.2 M) at 0° C. was added benzylic amine 109 (1.15 equiv.), followed by the addition of Et₃N (2.0 equiv.). The resulting yellow mixture was raised to rt and stirring at this temperature was continued for 2 h. The reaction was quenched with aq. HCl (1 N) and extracted with EtOAc. The combined organic layer was washed with sat. NaHCO₃ solution, brine and dried over anhydrous Na₂SO₄, and filtered. The solvent was removed under reduced pressure and the residue was purified by flash chromatography on silica gel (gradient elution, 0→50% EtOAc in hexanes) followed by recrystallization in CH₂Cl₂/hexanes to afford sulfonamide 18 (156 mg, 0.34 mmol, 58%) as a colorless solid. IR (cm⁻¹) 3278, 3007, 2952, 1703, 1597, 1389, 1260, 1184, 1157, 734; ¹H NMR (400 MHz, CDCl₃) δ 7.76 (d, J=9.2 Hz, 2H), 7.24 (d, J=8.4 Hz, 2H), 7.06 (d, J=8.4 Hz, 2H), 6.95 (d, J=8.8 Hz, 2H), 5.83 (dd, J=4.4, 3.2 Hz, 2H), 4.64 (t, J=6.4 Hz, 1H), 4.11 (d, J=6.0 Hz, 2H), 3.86 (s, 3H), 3.46 (m, 2H), 3.11 (dd, J=2.0, 1.6 Hz, 2H), 1.15-1.11 (m, 2H), 0.33-0.24 (m, 2H); ¹³C NMR (100 MHz, CDCl₃) δ 177.5 (2C), 163.0, 136.7, 131.4, 131.3, 129.3 (2C), 128.5 (2C), 127.8 (2C), 126.8 (2C), 114.3 (2C), 55.6, 46.7, 45.3 (2C), 33.8 (2C), 9.9 (2C), 4.7; ES-API MS: m/z calcd for C₂₅H₂₄N₂O₅SNa 487.1, found 487.1 [M+Na]⁺.

### (3aR,4R,4aR,5aS,6S,6aS)-2-(4-(3-(4-Methoxyphenyl)-3-oxopropyl)phenyl)-4,4a,5,5a,6,6a-hexahydro-4,6-ethenocyclopropa[f]isoindole-1,3(2H,3aH)-dione (19)

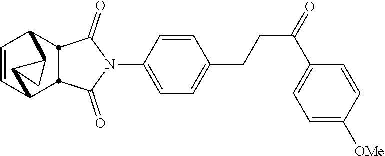 [SMILES: COc1ccc(C(=O)CCc2ccc(N3C(=O)[C@@H]4[C@@H]5C=CC([C@H]6CC65)[C@@H]4C3=O)cc2)cc1]

To a solution of enone 110 (53 mg, 0.12 mmol) and diethyl 1,4-dihydro-2,6-dimethyl-3,5-pyridinedicarboxylate (aka. Hantzsch ester) (63 mg, 0.25 mmol) in anhydrous THF (1.2 mL) was added dropwise a solution of TiCl₄ (0.18 ml, 0.18 mmol, 1 M solution in CH₂Cl₂). The resulting heterogeneous mixture slowly turned into a brown homogeneous solution. After 20 min, the reaction mixture was poured into a separatory funnel containing a biphasic layer of EtOAc (10 mL) and saturated NaHCO₃ solution (10 mL). The aqueous phase was extracted with EtOAc (2×10 mL), and the combined organic layer was washed with brine and dried over anhydrous Na₂SO₄, and filtered. The solvent was removed under reduced pressure and the residue was purified by flash chromatography on silica gel (gradient elution, 0→45% EtOAc in hexanes) followed by recrystallization in CH₂Cl₂/hexanes to give the desired compound 19 as a white solid (44 mg, 0.10 mmol, 86%). IR (cm⁻¹) 3010, 2956, 1706, 1674, 1601, 1515, 1384, 1259, 1176; ¹H NMR (400 MHz, CDCl₃) δ 7.92 (d, J=8.8 Hz, 2H), 7.31 (d, J=8.4 Hz, 2H), 7.09 (d, J=8.4 Hz, 2H), 6.92 (d, J=9.2 Hz, 2H), 5.85 (dd, J=4.8, 3.6 Hz, 2H), 3.86 (s, 3H), 3.49 (m, 2H), 3.23 (dd, J=7.6, 8.4 Hz, 2H), 3.13 (dd, J=2.0, 1.6 Hz, 2H), 3.06 (dd, J=8.0, 7.2 Hz, 2H), 1.14 (m, 2H), 0.35-0.26 (m, 2H); ¹³C NMR (100 MHz, CDCl₃) δ 197.4, 177.7 (2C), 163.5, 142.1, 130.2 (2C), 129.82, 129.79, 129.2 (2C), 127.8 (2C), 126.5 (2C), 113.7 (2C), 55.5, 45.3 (2C), 39.9, 33.8 (2C), 29.9, 9.9 (2C), 4.7; ES-API MS: m/z calcd for C₂₇H₂₆NO₄ 428.2, found 428.1 [M+H]⁺.

### (3aR,4R,4aR,5aS,6S,6aS)-2-(4-(2-(4-Methoxybenzoyl)cyclopropyl)phenyl)-4,4a,5,5a,6,6a-hexahydro-4,6-ethenocyclopropa[f]isoindole-1,3(2H,3aH)-dione (24)

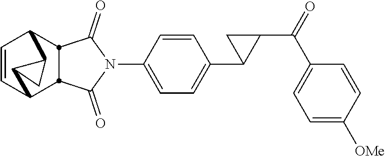 [SMILES: COc1ccc(C(=O)C2CC2c2ccc(N3C(=O)[C@@H]4[C@@H]5C=CC([C@H]6CC65)[C@@H]4C3=O)cc2)cc1]

Procedure F. Purification by flash chromatography on silica gel (gradient elution, 0→45% EtOAc in hexanes) followed by recrystallization in CH₂Cl₂/hexanes afforded 24 (70 mg, 0.16 mmol, 85%) as a pale yellow solid. IR (cm⁻¹) 3007, 2956, 1707, 1599, 1393, 1232, 1178, 1029, 734; ¹H NMR (400 MHz, CDCl₃) δ 7.97 (d, J=8.8 Hz, 2H), 7.23 (d, J=8.4 Hz, 2H), 7.10 (d, J=8.4 Hz, 2H), 6.94 (d, J=8.8 Hz, 2H), 5.86 (dd, J=4.8, 3.6 Hz, 2H), 3.87 (s, 3H), 3.50 (m, 2H), 3.14 (dd, J=1.6, 1.6 Hz, 2H), 2.83 (m, 1H), 2.65 (m, 1H), 1.90 (m, 1H), 1.49 (m, 1H), 1.15 (m, 2H), 0.35-0.26 (m, 2H); ¹³C NMR (100 MHz, CDCl₃) δ 196.5, 177.7 (2C), 163.5, 141.3, 130.6, 130.4 (2C), 130.1, 127.78, 127.76, 126.9 (2C), 126.6 (2C), 113.7 (2C), 55.5, 45.3 (2C), 33.8 (2C), 29.0, 18.7, 14.1, 9.9 (2C), 4.7; ES-API MS: m/z calcd for C₂₈H₂₆NO₄ 440.2, found 440.2 [M+H]⁺.

### (3aR,4R,4aR,5aS,6S,6aS)-2-(4-(3-(4-Methoxybenzoyl)oxiran-2-yl)phenyl)-4,4a,5,5a,6,6a-hexahydro-4,6-ethenocyclopropa[f]isoindole-1,3(2H,3aH)-dione (25)

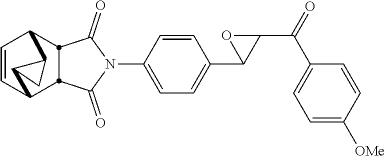 [SMILES: COc1ccc(C(=O)C2OC2c2ccc(N3C(=O)[C@@H]4[C@@H]5C=CC([C@H]6CC65)[C@@H]4C3=O)cc2)cc1]

To a solution of enone 110 (220 mg, 0.52 mmol) in MeOH/CH₂Cl₂ (5 mL, 1:1/v:v) at 0° C. was added dropwise H₂O₂ (234 mg, 2.07 mmol, 30 wt % solution) and NaOH (114 mg, 2.84 mmol, 0.5 M solution in H₂O). The resulting heterogeneous yellow mixture slowly turned into colorless mixture. The reaction was slowly raised to rt and stirred for 2 days and poured into a separatory funnel containing a biphasic layer of EtOAc (15 mL) and saturated aq. NaHCO₃ (15 mL). The aqueous phase was extracted with EtOAc (2×10 mL) and dried over anhydrous Na₂SO₄, and filtered. The solvent was removed under reduced pressure and the residue was purified by flash chromatography on silica gel (gradient elution, 0→60% EtOAc in hexanes) followed by recrystallization in CH₂Cl₂/hexanes to give the desired epoxide 25 as an amorphous white solid (200 mg, 0.45 mmol, 89%). IR (cm⁻¹) 3464, 3010, 2957, 1708, 1600, 1517, 1382, 1242, 1174, 734; ¹H NMR (400 MHz, CDCl₃) δ 7.98 (d, J=8.8 Hz, 2H), 7.44 (d, J=8.8 Hz, 2H), 7.22 (d, J=8.4 Hz, 2H), 6.95 (d, J=8.8 Hz, 2H), 5.87 (dd, J=4.0, 4.0 Hz, 2H), 4.20 (d, J=2.0 Hz, 1H), 4.08 (d, J=1.6 Hz, 1H), 3.88 (s, 3H), 3.50 (m, 2H), 3.16 (dd, J=1.6, 1.6 Hz, 2H), 1.16 (m, 2H), 0.36-0.27 (m, 2H); ¹³C NMR (100 MHz, CDCl₃) δ 190.8, 177.5 (2C), 164.3, 136.2, 132.3, 130.7 (2C), 128.5, 127.83, 127.79, 126.8 (2C), 126.4 (2C), 114.1 (2C), 60.8, 58.6, 55.6, 45.4 (2C), 33.8 (2C), 9.9 (2C), 4.7; ES-API MS: m/z calcd for C₂₇H₂₄NO₅ 442.2, found 442.1 [M+H]⁺.

### (3aR,4R,4aR,5aS,6S,6aS)-2-(4-(2-(3-Chloro-4-methoxybenzoyl)cyclopropyl)-2-fluorophenyl)-4,4a,5,5a,6,6a-hexahydro-4,6-ethenocyclopropa[f]isoindole-1,3(2H,3aH)-dione (65)

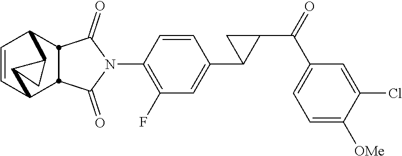 [SMILES: COc1ccc(C(=O)C2CC2c2ccc(N3C(=O)[C@@H]4[C@@H]5C=CC([C@H]6CC65)[C@@H]4C3=O)c(F)c2)cc1Cl]

Procedure F. Purification by flash chromatography on silica gel (gradient elution, 0→45% EtOAc in hexanes) followed by recrystallization in CH₂Cl₂/hexanes afforded 65 (43 mg, 0.087 mmol, 84%) as a pale yellow solid. IR (cm⁻¹) 3055, 3009, 2954, 1714, 1663, 1594, 1395, 1259, 1211, 1183, 1063, 1014, 736, 698; ¹H NMR (400 MHz, CDCl₃) δ 8.03 (d, J=2.4 Hz, 1H), 7.89 (dd, J=8.4, 2.4 Hz, 1H), 7.11 (br, 1H), 7.02 (d, J=8.0 Hz, 1H), 6.98 (d, J=8.8 Hz, 1H), 6.96 (d, J=10.8 Hz, 1H), 5.86 (dd, J=4.0, 4.0 Hz, 2H), 3.98 (s, 3H), 3.49 (m, 2H), 3.17 (m, 2H), 2.79 (m, 1H), 2.67 (m, 1H), 1.90 (m, 1H), 1.51 (m, 1H), 1.14 (m, 2H), 0.35-0.27 (m, 2H); ¹³C NMR (100 MHz, CDCl₃) δ 195.5, 177.1 (2C), 159.1, 157.6 (d, J=251.9 Hz, 1C), 144.5 (d, J=7.5 Hz, 1C), 131.1, 130.6, 129.4, 128.8, 128.0, 123.3, 122.8 (d, J=2.3 Hz, 1C), 118.1 (d, J=14.3 Hz, 1C), 114.4 (d, J=20.2 Hz, 1C), 111.5 (2C), 110.2, 56.6, 53.7, 46.0, 34.0 (2C), 29.1 (2C), 19.4, 10.1 (2C), 5.0; ES-API MS: m/z calcd for C₂₈H₂₄ClFNO₄ 492.1, found 492.2 [M+H]⁺.

### (3aR,4R,4aR,5aS,6S,6aS)-2-(4-(2-(4-chloro-3-Methylbenzoyl)cyclopropyl)-2-fluorophenyl)-4,4a,5,5a,6,6a-hexahydro-4,6-ethenocyclopropa[f]isoindole-1,3(2H,3aH)-dione (66)

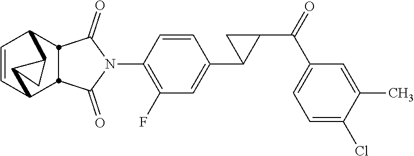 [SMILES: Cc1cc(C(=O)C2CC2c2ccc(N3C(=O)[C@@H]4[C@@H]5C=CC([C@H]6CC65)[C@@H]4C3=O)c(F)c2)ccc1Cl]

Procedure F. Purification by flash chromatography on silica gel (gradient elution, 0→40% EtOAc in hexanes) followed by recrystallization in CH₂Cl₂/hexanes afforded 66 (40 mg, 0.084 mmol, 78%) as a yellow solid. IR (cm⁻¹) 3054, 3009, 2956, 1716, 1666, 1521, 1395, 1180, 1049, 912, 735; ¹H NMR (400 MHz, CDCl₃) δ 7.84 (d, J=1.6 Hz, 1H), 7.73 (dd, J=8.4, 2.0 Hz, 1H), 7.43 (d, J=8.4 Hz, 1H), 7.11 (br, 1H), 7.02 (d, J=8.0 Hz, 1H), 6.96 (d, J=10.4 Hz, 1H), 5.86 (dd, J=4.0, 4.0 Hz, 2H), 3.49 (m, 2H), 3.17 (m, 2H), 2.82 (m, 1H), 2.68 (m, 1H), 2.44 (s, 3H), 1.92 (m, 1H), 1.53 (m, 1H), 1.15 (m, 2H), 0.36-0.27 (m, 2H); ¹³C NMR (100 MHz, CDCl₃) δ 197.1, 177.1 (2C), 157.6 (d, J=251.9 Hz, 1C), 144.4, 140.0, 136.4 (d, J=96.8 Hz, 1C), 130.7, 129.6, 127.9, 127.1, 122.8, 118.1 (d, J=13.6 Hz, 1C), 114.4 (d, J=19.3 Hz, 1C), 46.0, 45.7, 34.0 (2C), 31.8, 29.5, 29.4, 22.9, 20.3, 19.6, 14.4, 10.1 (2C), 5.0; ES-API MS: m/z calcd for C₂₈H₂₄ClFNO₃ 476.1, found 476.2 [M+H]⁺.

### (3aR,4R,4aR,5aS,6S,6aS)-2-(4-(2-(3-Chloro-4-fluorobenzoyl)cyclopropyl)-2-fluorophenyl)-4,4a,5,5a,6,6a-hexahydro-4,6-ethenocyclopropa[f]isoindole-1,3(2H,3aH)-dione (67)

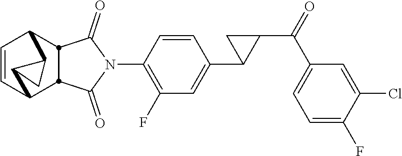 [SMILES: O=C(c1ccc(F)c(Cl)c1)C1CC1c1ccc(N2C(=O)[C@@H]3[C@@H]4C=CC([C@H]5CC54)[C@@H]3C2=O)c(F)c1]

Procedure F. Purification by flash chromatography on silica gel (gradient elution, 0→40% EtOAc in hexanes) followed by recrystallization in CH₂Cl₂/hexanes afforded 67 (18 mg, 0.038 mmol, 35%) as a white solid. IR (cm⁻¹) 3056, 3010, 2957, 1722, 1668, 1520, 1394, 1250, 1182, 1065, 736; ¹H NMR (400 MHz, CDCl₃) δ 8.05 (dd, J=6.8, 2.4 Hz, 1H), 7.88 (ddd, J=8.4, 4.4, 2.0 Hz, 1H), 7.24 (dd, J=8.8, 8.8 Hz, 1H), 7.11 (d, J=8.8 Hz, 1H), 7.02 (d, J=7.6 Hz, 1H), 6.96 (d, J=10.8 Hz, 1H), 5.86 (m, 2H), 3.49 (m, 2H), 3.17 (m, 2H), 2.79 (m, 1H), 2.70 (m, 1H), 1.93 (m, 1H), 1.56 (m, 1H), 1.15 (m, 2H), 0.36-0.27 (m, 2H); ¹³C NMR (100 MHz, CDCl₃) δ 195.5, 177.1 (2C), 161.3 (d, J=256.0 Hz, 1C), 157.6 (d, J=252.3 Hz, 1C), 144.0 (d, J=7.5 Hz, 1C), 134.7 (d, J=3.6 Hz, 1C), 131.3 (d, J=0.5 Hz, 1C), 129.5 (d, J=15.3 Hz, 1C), 128.7 (d, J=8.4 Hz, 1C), 128.2, 127.9 (d, J=14.9 Hz, 1C), 126.2 (d, J=7.3 Hz, 1C), 122.5 (d, J=39.7 Hz, 1C), 118.3 (d, J=13.7 Hz, 1C), 117.1 (d, J=21.6 Hz, 1C), 114.4 (d, J=20.0 Hz, 1C), 46.0, 45.7, 34.0, 31.8, 29.4, 22.9, 19.9, 14.4, 10.1, 5.0; ES-API MS: m/z calcd for C₂₇H₂₁ClF₂NO₃ 480.1, found 480.1 [M+H]⁺.

### (3aR,4R,4aR,5aS,6S,6aS)-2-(4-(2-(4-Chloro-3-(trifluoromethyl)benzoyl)cyclopropyl)-2-fluorophenyl)-4,4a,5,5a,6,6a-hexahydro-4,6-ethenocyclopropa[f]isoindole-1,3(2H,3aH)-dione (68)

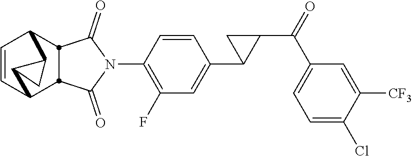 [SMILES: O=C(c1ccc(Cl)c(C(F)(F)F)c1)C1CC1c1ccc(N2C(=O)[C@@H]3[C@@H]4C=CC([C@H]5CC54)[C@@H]3C2=O)c(F)c1]

Procedure F. Purification by flash chromatography on silica gel (gradient elution, 0→40% EtOAc in hexanes) followed by recrystallization in CH₂Cl₂/hexanes afforded 68 (48 mg, 0.091 mmol, 78%) as a white solid. IR (cm⁻¹) 3055, 3010, 2957, 1715, 1674, 1603, 1520, 1394, 1315, 1036, 737; ¹H NMR (400 MHz, CDCl₃) δ 8.30 (d, J=1.6 Hz, 1H), 8.06 (dd, J=8.4, 2.0 Hz, 1H), 7.63 (d, J=8.0 Hz, 1H), 7.12 (m, 1H), 7.02 (d, J=7.6 Hz, 1H), 6.96 (d, J=10.4 Hz, 1H), 5.86 (dd, J=3.6, 3.6 Hz, 2H), 3.49 (m, 2H), 3.17 (m, 2H), 2.81 (m, 1H), 2.74 (m, 1H), 1.95 (m, 1H), 1.61 (m, 1H), 1.15 (m, 2H), 0.36-0.29 (m, 2H); ¹³C NMR (100 MHz, CDCl₃) δ 195.8, 177.1 (2C), 157.6 (d, J=252.3 Hz, 1C), 145.7, 143.8, 137.7, 135.9, 132.4, 132.2, 131.8, 129.4, 128.0, 127.5, 122.8, 118.5, 114.6, 60.6, 45.9, 45.8, 34.0 (2C), 29.8, 29.4, 20.0, 10.1 (2C), 5.0; ES-API MS: m/z calcd for C₂₈H₂₁ClF₄NO₃ 530.1, found 530.1 [M+H]⁺.

### (3aR,4R,4aR,5aS,6S,6aS)-2-(4-(2-(3,4-Dichlorobenzoyl)cyclopropyl)-2-fluorophenyl)-4,4a,5,5a,6,6a-hexahydro-4,6-ethenocyclopropa[f]isoindole-1,3(2H,3aH)-dione (69)

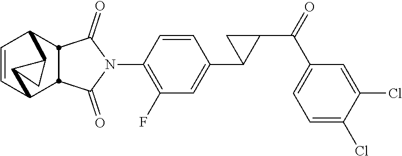 [SMILES: O=C(c1ccc(Cl)c(Cl)c1)C1CC1c1ccc(N2C(=O)[C@@H]3[C@@H]4C=CC([C@H]5CC54)[C@@H]3C2=O)c(F)c1]

Procedure F. Purification by flash chromatography on silica gel (gradient elution, 0→40% EtOAc in hexanes) followed by recrystallization in CH₂Cl₂/hexanes afforded 69 (44 mg, 0.088 mmol, 86%) as a white solid. IR (cm⁻¹) 3009, 2919, 1715, 1668, 1520, 1393, 1181, 736; ¹H NMR (400 MHz, CDCl₃) δ 8.05 (d, J=2.0 Hz, 1H), 7.79 (dd, J=8.4, 2.0 Hz, 1H), 7.56 (d, J=8.4 Hz, 1H), 7.12 (m, 1H), 7.02 (d, J=7.6 Hz, 1H), 6.96 (d, J=10.8 Hz, 1H), 5.86 (dd, J=3.6, 3.6 Hz, 2H), 3.49 (m, 2H), 3.17 (m, 2H), 2.79 (m, 1H), 2.71 (m, 1H), 1.93 (m, 1H), 1.57 (m, 1H), 1.15 (m, 2H), 0.35-0.27 (m, 2H); ¹³C NMR (100 MHz, CDCl₃) δ 195.9, 177.1 (2C), 158.9, 143.9, 138.0, 137.0, 133.7, 131.0, 130.7 (d, J=4.0 Hz, 1C), 130.3, 127.9, 127.4, 125.7, 122.7, 122.3, 46.0, 45.7, 34.0, 31.8, 29.8, 29.5, 22.9, 19.9, 14.4, 10.1, 5.0; ES-API MS: m/z calcd for C₂₇H₂₁Cl₂FNO₃ 496.1, found 496.1 [M+H]⁺.

### (3aR,4R,4aR,5aS,6S,6aS)-2-(4-(2-(3,5-Dichlorobenzoyl)cyclopropyl)-2-fluorophenyl)-4,4a,5,5a,6,6a-hexahydro-4,6-ethenocyclopropa[f]isoindole-1,3(2H,3aH)-dione (70)

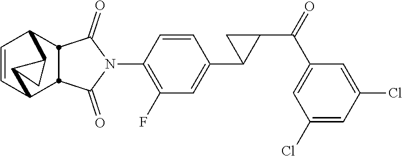 [SMILES: O=C(c1cc(Cl)cc(Cl)c1)C1CC1c1ccc(N2C(=O)[C@@H]3[C@@H]4C=CC([C@H]5CC54)[C@@H]3C2=O)c(F)c1]

Procedure F. Purification by flash chromatography on silica gel (gradient elution, 0→40% EtOAc in hexanes) followed by recrystallization in CH₂Cl₂/hexanes afforded 70 (44 mg, 0.088 mmol, 86%) as a yellow solid. IR (cm⁻¹) 3077, 3009, 2956, 1715, 1566, 1520, 1397, 1217, 1182, 736; ¹H NMR (400 MHz, CDCl₃) δ 7.80 (s, 2H), 7.53 (s, 1H), 7.09 (m, 1H), 6.99 (d, J=6.8 Hz, 1H), 6.94 (d, J=10.4 Hz, 1H), 5.84 (dd, J=3.6, 3.6 Hz, 2H), 3.46 (m, 2H), 3.15 (m, 2H), 2.77 (m, 1H), 2.70 (m, 1H), 1.89 (m, 1H), 1.55 (m, 1H), 1.13 (m, 2H), 0.33-0.25 (m, 2H); ¹³C NMR (100 MHz, CDCl₃) δ 195.6, 177.0 (2C), 157.5 (d, J=252.3 Hz, 1C), 143.7 (d, J=7.4 Hz, 1C), 139.8, 135.8 (2C), 132.8 (2C), 129.4 (d, J=17.0 Hz, 1C), 127.8 (d, J=7.5 Hz, 1C), 126.7 (2C), 122.6, 118.2 (d, J=13.7 Hz, 1C), 114.3 (d, J=20.8 Hz, 1C), 45.9, 45.6, 33.8 (2C), 29.8, 29.4, 20.2, 10.0 (2C), 4.9; ES-API MS: m/z calcd for C₂₇H₂₁Cl₂FNO₃ 496.1, found 496.1 [M+H]⁺.

### 4-((3aR,4R,4aR,5aS,6S,6aS)-1,3-Dioxo-3,3a,4,4a,5,5a,6,6a-octahydro-4,6-ethenocyclopropa[f]isoindol-2(1H)-yl)phenyl 2-(4-methoxyphenyl)acetate (13)

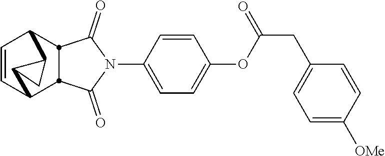 [SMILES: COc1ccc(CC(=O)Oc2ccc(N3C(=O)[C@@H]4[C@@H]5C=CC([C@H]6CC65)[C@@H]4C3=O)cc2)cc1]

To a solution of phenol 102 (50 mg, 0.18 mmol) in anhydrous THF (1.8 mL) was added NaH (17 mg, 0.71 mmol, 60% dispersion in mineral oil) and heated to 60° C. for 20 min. The mixture was cooled to rt followed by the addition of a neat 2-(4′-methoxyphenyl)-acetyl chloride (66 mg, 0.36 mmol). The cloudy mixture turned transparent and the reaction was completed in 30 min. The reaction was quenched with H₂O (5 mL) and extracted with EtOAc (2×10 mL). The combined organic phase was washed with brine and dried over anhydrous Na₂SO₄, and filtered. The solvent was removed under reduced pressure and the residue was purified by flash chromatography on silica gel (gradient elution, 0→50% EtOAc in hexanes) to afford the ester 13 as a white solid (65 mg, 0.15 mmol, 86%). IR (cm⁻¹) 3006, 2959, 1748, 1713, 1506, 1393, 1204, 1124, 1038, 812, 735; ¹H NMR (400 MHz, CDCl₃) δ 7.28 (d, J=8.4 Hz, 2H), 7.18 (d, J=8.8 Hz, 2H), 7.13 (d, J=9.2 Hz, 2H), 6.89 (d, J=8.8 Hz, 2H), 5.84 (dd, J=4.8, 3.2 Hz, 2H), 3.80 (s, 3H), 3.78 (s, 2H), 3.47 (m, 2H), 3.11 (dd, J=1.6, 1.6 Hz, 2H), 1.13 (m, 2H), 0.34-0.25 (m, 2H); ¹³C NMR (100 MHz, CDCl₃) δ 177.6 (2C), 170.0, 159.0, 150.5, 130.5 (2C), 129.4, 127.9 (2C), 127.6 (2C), 125.4, 122.2 (2C), 114.3 (2C), 55.4, 45.4 (2C), 40.6, 34.0 (2C), 10.0 (2C), 4.8; ES-API MS: m/z calcd for C₂₆H₂₃NO₅Na 452.1, found 452.1 [M+Na]⁺.

### (3aR,4R,4aR,5aS,6S,6aS)-2-(4-((E)-2-(Methoxyimino)-2-(4-methoxyphenyl)ethoxy)phenyl)-4,4a,5,5a,6,6a-hexahydro-4,6-ethenocyclopropa[f]isoindole-1,3(2H,3aH)-dione (26)

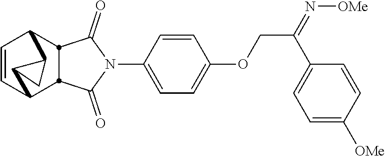 [SMILES: CO/N=C(/COc1ccc(N2C(=O)[C@@H]3[C@@H]4C=CC([C@H]5CC54)[C@@H]3C2=O)cc1)c1ccc(OC)cc1]

A mixture of ketone 1 (50 mg, 0.12 mmol), methoxyamine hydrochloride (26.3 mg, 0.31 mmol) and sodium acetate (42 mg, 0.51 mmol) was dissolved in EtOH (0.4 mL) and H₂O (1.2 mmol). The resulting suspension was heated to 70° C. and stirred for 2 h. The reaction was cooled to rt and extracted with EtOAc (3×10 mL). The combined organic layer was washed with brine and dried over anhydrous MgSO₄, and filtered. The solvent was removed under reduced pressure and the residue was purified by flash chromatography on silica gel (gradient elution, 0→40/a EtOAc in hexanes) followed by recrystallization in CH₂Cl₂/hexanes to afford 26 as a white solid (27 mg, 0.059 mmol, 49%). IR (cm⁻¹) 2938, 1707, 1608, 1512, 1250, 1180, 1048, 1030, 830, 734; ¹H NMR (400 MHz, CDCl₃) δ 7.61 (d, J=8.8 Hz, 2H), 7.05 (d, J=8.8 Hz, 2H), 6.95 (d, J=9.2 Hz, 2H), 6.86 (d, J=8.8 Hz, 2H), 5.84 (dd, J=4.4, 3.6 Hz, 2H), 5.16 (s, 2H), 4.02 (s, 3H), 3.81 (s, 3H), 3.48 (m, 2H), 3.11 (m, 2H), 1.13 (m, 2H), 0.34-0.25 (m, 2H); ¹³C NMR (100 MHz, CDCl₃) δ 178.0 (2C), 160.8, 158.2, 153.7, 128.6 (2C), 128.0 (2C), 127.9 (2C), 126.1, 125.2, 115.3 (2C), 114.0 (2C), 62.6, 60.3, 55.5, 45.5 (2C), 34.0 (2C), 10.1 (2C), 4.9; ES-API MS: m/z calcd for C₂₇H₂₇N₂O₅ 459.2, found 459.2 [M+H]⁺.

### (3aR,4R,4aR,5aS,6S,6aS)-2-(4-((E)-2-(Hydroxyiminio)-2-(4-methoxyphenyl)ethoxy)phenyl)-4,4a,5,5a,6,6a-hexahydro-4,6-ethenocyclopropa[f]isoindole-1,3(2H,3aH)-dione (27)

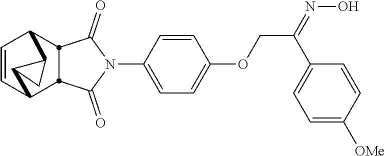 [SMILES: COc1ccc(/C(COc2ccc(N3C(=O)[C@@H]4[C@@H]5C=CC([C@H]6CC65)[C@@H]4C3=O)cc2)=N\O)cc1]

A mixture of ketone 1 (48 mg, 0.11 mmol), aminohydroxide hydrochloride (31 mg, 0.44 mmol) and sodium acetate (55 mg, 0.67 mmol) was dissolved in EtOH (0.55 mL) and H₂O (0.55 mmol). The resulting suspension was heated to 70° C. and stirred for 2 h. The reaction was cooled to rt and extracted with EtOAc (3×10 mL). The combined organic layer was washed with brine and dried over anhydrous MgSO₄, and filtered. The solvent was removed under reduced pressure and the residue was purified by flash chromatography on silica gel (gradient elution, 0→40% EtOAc in hexanes) followed by recrystallization in CH₂Cl₂/hexanes to afford 27 as a white solid (38 mg, 0.085 mmol, 78%). IR (cm⁻¹) 3370, 2838, 1704, 1513, 1250, 1181, 1029, 830, 734; ¹H NMR (400 MHz, CDCl₃) δ 8.77 (br, 1H), 7.59 (d, J=8.8 Hz, 2H), 7.05 (d, J=8.8 Hz, 2H), 6.98 (d, J=8.8 Hz, 2H), 6.88 (d, J=8.8 Hz, 2H), 5.84 (dd, J=4.4, 3.6 Hz, 2H), 5.24 (s, 2H), 3.81 (s, 3H), 3.48 (m, 2H), 3.11 (m, 2H), 1.13 (m, 2H), 0.34-0.25 (m, 2H); ¹³C NMR (100 MHz, CDCl₃) δ 178.2 (2C), 160.9, 158.2, 155.0, 128.6 (2C), 128.0 (2C), 127.9 (2C), 125.9, 125.2, 115.3 (2C), 114.1 (2C), 59.8, 55.5, 45.4 (2C), 34.0 (2C), 10.1 (2C), 4.9; ES-API MS: m/z calcd for C₂₆H₂₄NO 430.2, found 430.1 [M+H₂O—NHOH]⁺.

### (3aR,4R,4aR,5aS,6S,6aS)-2-(4-(2-Hydroxy-2-(4-methoxyphenyl)ethoxy)phenyl)-4,4a,5,5a,6,6a-hexahydro-4,6-ethenocyclopropa[f]isoindole-1,3(2H,3aH)-dione (28)

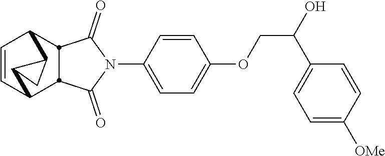

To a solution of ketone 1 (50 mg, 0.12 mmol) in MeOH/CH₂Cl₂ (v:v/1:1, 2.30 mL) was added NaBH₄ (5.7 mg, 0.15 mmol) at 0° C. The resulting mixture was stirred at this temperature for 2 h. The reaction was quenched with cold H₂O (1 mL) and extracted with EtOAc (2×5 mL). The combined organic layer was washed with brine and dried over anhydrous MgSO₄, and filtered. The solvent was removed under reduced pressure and the residue was purified by flash chromatography on silica gel (gradient elution, 0→65% EtOAc in hexanes) to give benzylic alcohol 28 as a white solid (33 mg, 0.076 mmol, 66%). IR (cm⁻¹) 3462, 2955, 1704, 1513, 1247, 1182, 734; ¹H NMR (400 MHz, CDCl₃) δ 7.36 (d, J=8.8 Hz, 2H), 7.07 (d, J=9.2 Hz, 2H), 6.95 (d, J=8.8 Hz, 2H), 6.92 (d, J=8.8 Hz, 2H), 5.85 (dd, J=4.4, 3.6 Hz, 2H), 5.05 (ddd, J=8.4, 2.8, 2.8 Hz, 1H), 4.05 (dd, J=9.6, 3.2 Hz, 1H), 3.99 (dd, J=8.8, 9.2 Hz, 1H), 3.81 (s, 3H), 3.48 (m, 2H), 3.11 (dd, J=2.0, 2.0 Hz, 2H), 2.71 (d, J=2.8 Hz, 1H), 1.14 (m, 2H), 0.34-0.25 (m, 2H); ¹³C NMR (100 MHz, CDCl₃) S 178.1 (2C), 159.8, 158.5, 131.8, 128.00 (2C), 127.97 (2C), 127.8 (2C), 125.2, 115.3 (2C), 114.2 (2C), 73.7, 72.2, 55.5, 45.5 (2C), 34.0 (2C), 10.1 (2C), 4.9; ES-API MS: m/z calcd for C₂₆H₂₄NO₄ 414.2, found 414.2 [M-OH]⁺.

### (1R,2R,4S,5S,6S,7R)-Tricyclo[3.2.2.02,4]non-8-ene-6,7-dicarboxylic acid (112)

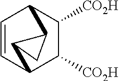 [SMILES: O=C(O)[C@@H]1[C@H](C(=O)O)[C@@H]2C=C[C@H]1C1CC12]

Anhydride 97 (4 g, 21.0 mmol) was dissolved in 40% MeOH in H₂O (105 mL) that contains NaOH (3.36 g, 84.1 mmol). The resulting mixture was heated to reflux at 90° C. and stirred overnight. A white precipitate formed after 1 h stirring. The mixture was cooled to 0° C. and neutralized with cold aq. HCl (6 N) and extracted with a mixture of CHCl₃ and i-PrOH (v:v/3:1). The remaining aqueous phase was concentrated under reduced pressure and precipitated NaCl salt was removed via filtration. The precipitated salt was washed several times with CHCl₃. The solvent was removed under reduced pressure and the residue was purified by flash chromatography on silica gel (gradient elution, 0→15% MeOH in CH₂Cl₂) followed by recrystallization from MeOH/CH₂Cl₂/hexanes to afford the acid 112 (2.7 g, 13.0 mmol, 61%) as a white solid. IR (cm⁻¹) 2998, 2737, 1705, 1416, 1230, 943, 702; ¹H NMR (400 MHz, DMSO-d₆) δ 11.8 (s, 2H), 5.72 (dd, J=4.8, 3.6 Hz, 2H), 2.99 (m, 2H), 2.94 (m, 2H), 0.96 (m, 2H), 0.08-−0.02 (m, 2H); ¹³C NMR (100 MHz, DMSO-d₆) δ 173.9 (2C), 127.2 (2C), 47.8 (2C), 33.7 (2C), 9.2 (2C), 2.6 (2C); ES-API MS: m/z calcd for C₁₁H₁₂O₄ 208.1, found 208.1 [M]⁺.

### (1R,3aS,4S,5S,5aS,6aR,6bR,7R)-4-Bromo-2-oxooctahydro-2H-1,5-methanocyclopropa[e]benzofuran-7-carboxylic acid (113)

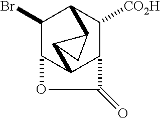 [SMILES: O=C(O)[C@@H]1[C@@H]2C(=O)O[C@@H]3[C@@H](Br)[C@H]1C1CC1[C@@H]32]

To a mixture of acid 112 (500 mg, 2.40 mmol) and NaHCO₃ (504 mg, 6.00 mmol) was added H₂O (24 mL). Noted that immediate frothing occurred. The mixture was stirred and heated to 50° C. for 10 min to ensure complete formation of the dicarboxylate salt. The mixture was cooled to 0° C. and a solution of bromine (575 mg, 3.6 mmol) was added dropwise until no further decolorization occurred. The resulting mixture was raised to rt and stirred for an additional 6 h. The mixture was extracted with CHCl₃ (3×). The combined organic layer was washed with 15% aq. Na₂S₂O₅, brine and dried over anhydrous MgSO₄, and filtered. The solvent was removed under reduced pressure and the residue was purified by flash chromatography on silica gel (gradient elution, 0→5% MeOH in CH₂Cl₂) followed by recrystallization in EtOAc/hexanes to afford bromolactone 113 (380 mg, 1.32 mmol, 55%) as a colorless solid. IR (cm⁻¹) 3022, 1773, 1717, 1164, 981; ¹H NMR (400 MHz, CDCl₃/CD₃OD) δ 4.58 (ddd, J=3.2, 1.2, 0.8 Hz, 1H), 4.49 (ddd, J=4.8, 1.2, 0.8 Hz, 1H), 3.09 (ddd, 1=9.2, 4.8, 4.8 Hz, 1H), 3.00 (dd, J=10.0, 1.2 Hz, 1H), 2.82-2.77 (m, 2H), 1.15 (ddd, J=6.8, 4.0, 3.6 Hz, 1H), 1.12-1.03 (m, 2H), 0.68 (ddd, J=8.0, 8.0, 6.8 Hz, 1H); ¹³C NMR (100 MHz, CDCl₃/CD₃OD) δ 177.5, 173.5, 84.3, 46.4, 46.1, 41.9, 39.6, 35.7, 11.5, 8.0, 6.2; ES-API MS: m/z calcd for C₁₁H₁₂BrO₄ 287.0 (⁷⁹Br isotope), found 286.9 [M+H]⁺.

### (1R,3aS,4S,5S,5aS,6aR,6bR,7R)-4-Bromo-N-(4-(2-(4-methoxyphenyl)-2-oxoethoxy)phenyl)-2-oxooctahydro-2H-1,5-methanocyclopropa[e]benzofuran-?-carboxamide (114)

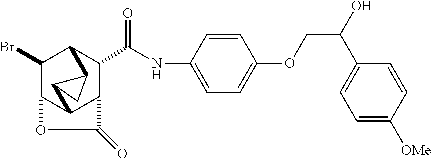 [SMILES: COc1ccc(C(O)COc2ccc(NC(=O)[C@@H]3[C@@H]4C(=O)O[C@@H]5[C@@H](Br)[C@H]3C3CC3[C@@H]54)cc2)cc1]

To a solution of bromo-lactone 113 (30 mg, 0.10 mmol) and HATU (40 mg, 0.10 mmol) in anhydrous DMF (0.2 mL) was added DIPEA (13.5 mg, 0.10 mmol). The reaction mixture was stirred at rt for 15 min until it became homogeneous. A solution of 2-(4-aminophenoxy)-1-(4-methoxyphenyl)ethan-1-one (26 mg, 0.10 mmol) in anhydrous DMF (0.2 mL) was added to the reaction mixture via a syringe, and the mixture was stirred at rt until TLC indicated complete consumption of the aniline. The mixture was quenched with aq. HCl solution (1 N, 5 mL) and extracted with EtOAc (2×5 mL). The combined organic layer was washed with brine and dried over anhydrous Na₂SO₄, and filtered. The solvent was removed under reduced pressure and the residue was purified by flash chromatography on silica gel (gradient elution, 0→55% EtOAc in hexanes) to obtain the amide 114 (33 mg, 0.062 mmol, 63%) as a white amorphous solid. IR (cm⁻¹) 3446, 2934, 1704, 1694, 1601, 1513, 1234, 1172, 834, 729; ¹H NMR (400 MHz, CDCl₃) δ 7.98 (d, J=8.8 Hz, 2H), 7.21 (d, J=8.8 Hz, 2H), 7.00 (d, J=9.2 Hz, 2H), 6.96 (d, J=9.2 Hz, 2H), 5.20 (s, 2H), 4.06 (dd, J=6.4, 3.2 Hz, 1H), 3.88 (s, 3H), 3.76 (m, 1H), 3.26 (dd, J=9.6, 4.0 Hz, 1H), 3.13 (dd, J=6.4, 3.2 Hz, 1H), 3.01 (dd, J=10.0, 3.2 Hz, 1H), 2.90 (dd, J=6.8, 3.2 Hz, 1H), 2.68 (d, J=2.8 Hz, 1H), 1.39 (m, 1H), 1.19-1.09 (m, 2H), 0.74 (m, 1H); ¹³C NMR (100 MHz, CDCl₃) δ 192.9, 178.5, 177.2, 164.3, 158.3, 130.8 (2C), 128.2 (2C), 127.7, 125.5, 115.6 (2C), 114.3 (2C), 76.3, 71.1, 55.8, 51.7, 47.4, 41.2, 38.0, 37.4, 13.7, 11.2, 8.6; ES-API MS: m/z calcd for C₂₆H₂₆BrNO₇ 527.1 (⁸¹Br isotope), found 527.1 [M]⁺.

### (3aR,4R,4aR,5aS,6S,6aS,7S,8S)-7,8-Dihydroxy-2-(4-(2-(4-methoxyphenyl)-2-oxoethoxy)phenyl)hexahydro-4,6-ethanocyclopropa[f]isoindole-1,3(2H,3aH)-dione (6)

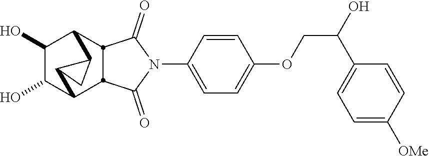

To a solution of lactone 114 (31 mg, 0.060 mmol) in anhydrous DMF (0.6 mL) was added NaH (2.6 mg, 0.065 mmol, 60% in dispersion oil). The resulting mixture was sealed in a pressure seal-tube and heated to 100° C. for 3 h. The mixture was cooled to rt and quenched with H₂O (5 mL) and extracted with EtOAc (2×10 mL). The combined organic layer was washed with brine and dried over anhydrous Na₂SO₄, and filtered. The solvent was removed under reduced pressure and the residue was purified by preparative TLC (70% EtOAc in hexanes) to obtain the diol 6 (18 mg, 0.038 mmol, 67%) as a white solid. IR (cm⁻¹) 3343, 2921, 2851, 1714, 1694, 1682, 1506, 1259, 1221, 1171, 802; ¹H NMR (400 MHz, CDCl₃) δ 7.99 (d, J=9.2 Hz, 2H), 7.18 (d, J=8.8 Hz, 2H), 6.99 (d, J=8.8 Hz, 2H), 6.96 (d, J=8.8 Hz, 2H), 5.19 (s, 2H), 4.16 (m, 1H), 3.88 (s, 3H), 3.14 (m, 1H), 3.00 (d, J=4.8 Hz, 1H), 2.84 (ddd, J=8.8, 3.2, 1.6 Hz, 1H), 1.89 (dd, J=8.8, 1.2 Hz, 1H), 1.80 (d, J=1.6 Hz, 1H), 1.58 (br, 2H), 1.18 (m, 2H), 0.58 (m, 1H), 0.49 (m, 1H); ¹³C NMR (100 MHz, CDCl₃) δ 193.0, 176.2, 175.5, 164.3, 157.9, 130.8 (2C), 128.3 (2C), 127.7, 126.2, 115.5 (2C), 114.3 (2C), 71.9, 71.2, 55.8, 50.2, 40.6, 38.7, 36.1, 27.8, 14.9, 9.6, 8.0; ES-API MS: m/z calcd for C₂₆H₂₅NO₇ 463.2, found 463.2 [M]⁺.

(4aS)-2-(4-(Hydroxy(4-methoxyphenyl)methyl)phenyl)-4,4a,5,5a,6,6a-hexahydro-4,6-ethenocyclopropa[f]isoindole-1,3(2H,3aH)-dione (14). To a solution of aldehyde 107 (67 mg, 0.23 mmol) in anhydrous THF (0.77 mL) was added 4-methoxyphenylmagnesium bromide (0.7 mL, 0.34 mmol, 0.5 M solution in THF) at 0° C. The mixture was slowly raised to rt and stirring overnight. Another 1.5 equiv. of 4-methoxyphenylmagnesium bromide was added and the mixture was heated to 65° C. After cooling to rt, the reaction mixture was poured into a separatory funnel containing EtOAc and saturated aq. NH₄Cl. The aqueous phase was removed and the organic layer was neutralized with saturated aq. NaHCO₃ and dried over Na₂SO₄. The crude material was filtered, concentrated in vacuo, and purified by flash chromatography (gradient elution, 0-50% EtOAc/hexanes) to afford compound 14 (45 mg, 0.11 mmol, 49%) to give an amorphous yellow solid. ¹H NMR (400 MHz, CDCl₃) δ 7.43 (d, J=8.4 Hz, 2H), 7.26 (d, J=8.4 Hz, 2H), 7.13 (d, J=8.4 Hz, 2H), 6.85 (d, J=8.8 Hz, 2H), 5.84 (dd, J=4.4, 3.6 Hz, 2H), 5.80 (br, 1H), 3.79 (s, 3H), 3.48 (m, 2H), 3.12 (dd, J=1.6, 1.6 Hz, 2H), 2.21 (d, J=2.8 Hz, 1H), 1.14 (m, 2H), 0.34-0.25 (m, 2H); ¹³C NMR (100 MHz, CDCl₃) δ 177.7 (2C), 159.2, 144.3, 135.6, 130.8, 128.0 (2C), 127.8 (2C), 127.0 (2C), 126.4 (2C), 113.9 (2C), 75.4, 55.3, 45.3 (2C), 33.8 (2C), 9.9 (2C), 4.7; ES-API MS: m/z calcd for C₂₅H₂₂NO₃ 384.2 found 384.1 [M-OH]⁺.

(4aS)-2-(4-(1-Hydroxy-2-phenylethyl)phenyl)-4,4a,5,5a,6,6a-hexahydro-4,6-ethenocyclopropa[f]isoindole-1,3(2H,3aH)-dione (21). To a solution of aldehyde 107 (50 mg, 0.17 mmol) in anhydrous THF (0.6 mL) was added benzylmagnesium chloride (0.17 mL, 0.25 mmol, 2 M solution in THF) at 0° C. The mixture was slowly raised to rt and stirring overnight. The reaction mixture was poured into a separatory funnel containing EtOAc and saturated aq. NH₄Cl. The aqueous phase was removed and the organic layer was neutralized with saturated aq. NaHCO₃, and dried over Na₂SO₄. The crude material was filtered, concentrated in vacuo, and purified with flash chromatography (gradient elution, 0-45% EtOAc/hexanes) to afford compound 21 (40 mg, 0.10 mmol, 62%) as an amorphous yellow solid. ¹H NMR (400 MHz, CDCl₃) δ 7.44 (d, J=8.8 Hz, 2H), 7.32 (dd, J=7.6, 6.8 Hz, 2H), 7.23 (m, 3H), 7.16 (d, J=8.4 Hz, 2H), 5.87 (dd, J=4.4, 3.6 Hz, 2H), 4.92 (m, 1H), 3.50 (m, 2H), 3.14 (dd, J=1.6, 1.6 Hz, 2H), 3.03 (dd, J=13.6, 4.4 Hz, 1H), 2.93 (dd, J=13.6, 8.8 Hz, 1H), 1.95 (d, J=2.8 Hz, 1H), 1.15 (m, 2H), 0.36-0.27 (m, 2H); ¹³C NMR (100 MHz, CDCl₃) δ 177.7 (2C), 144.3, 137.8, 131.0, 129.5 (2C), 128.6 (2C), 127.8 (2C), 126.7, 126.50 (2C), 126.46 (2C), 74.8, 46.1, 45.3 (2C), 33.8 (2C), 9.9 (2C), 4.7; ES-API MS: m/z calcd for C₂₅H₂₄NO₃ 386.2 found 386.1 [M+H]⁺.

(4aS)-2-(4-(1-Hydroxy-3-phenylpropyl)phenyl)-4,4a,5,5a,6,6a-hexahydro-4,6-ethenocyclopropa[f]isoindole-1,3(2H,3aH)-dione (22). To a solution of aldehyde 107 (50 mg, 0.17 mmol) in anhydrous THF (0.6 mL) was added phenethylmagnesium chloride (0.34 mL, 0.34 mmol, 1 M solution in THF) at 0° C. The mixture was slowly raised to rt and stirring was continued overnight. The reaction mixture was poured into a separatory funnel containing EtOAc and saturated aq. NH₄Cl. The aqueous phase was removed and the organic layer was neutralized with saturated aq. NaHCO₃, and dried over Na₂SO₄. The crude material was filtered, concentrated in vacuo, and purified by flash chromatography (gradient elution, 0-50% EtOAc/hexanes) to afford compound 22 (48 mg, 0.12 mmol, 71%) as an amorphous yellow solid. ¹H NMR (400 MHz, CDCl₃) δ 7.41 (d, J=8.4 Hz, 2H), 7.27 (d, J=8.4 Hz, 2H), 7.20-7.15 (m, 5H), 5.86 (dd, J=4.8, 3.6 Hz, 2H), 4.70 (m, 1H), 3.49 (m, 2H), 3.14 (dd, J=1.6, 1.6 Hz, 2H), 2.73 (m, 2H), 2.10 (m, 1H), 2.00 (m, 1H), 1.89 (d, J=3.2 Hz, 1H), 1.15 (m, 2H), 0.35-0.26 (m, 2H); ¹³C NMR (100 MHz, CDCl₃) δ 177.7 (2C), 145.0, 141.5, 131.0, 128.42 (2C), 128.40 (2C), 127.8 (2C), 126.6 (2C), 126.5 (2C), 125.9, 73.3, 45.3 (2C), 40.3, 33.8 (2C), 31.9, 9.9 (2C), 4.7, ES-API MS: m/z calcd for C₂₆H₂₄NO₂ 382.2, found 382.2 [M-OH]⁺.

### (3aR,4R,4aR,5aS,6S,6aS)-2-(4-Ethynylphenyl)-4,4a,5,5a,6,6a-hexahydro-4,6-ethenocyclopropa[f]isoindole-1,3(2H,3aH)-dione (115)

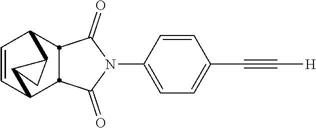

Procedure A. Purification by flash chromatography on silica gel (gradient elution, 0→35% EtOAc in hexanes) followed by recrystallization in CH₂Cl₂/hexanes afforded alkyne 115 (1.42 g, 4.9 mmol, 47%) as an orange solid. IR (cm⁻¹) 3278, 1703, 1509, 1391, 1194; ¹H NMR (400 MHz, CDCl₃) δ 7.52 (d, J=8.4 Hz, 2H), 7.15 (d, J=8.8 Hz, 2H), 5.83 (dd, J=4.8, 3.2 Hz, 2H), 3.47 (m, 2H), 3.12 (dd, J=1.6, 1.6 Hz, 2H), 3.08 (s, 1H), 1.13 (m, 2H), 0.33-0.24 (m, 2H); ¹³C NMR (100 MHz, CDCl₃) δ 177.3 (2C), 132.7 (2C), 132.0, 127.8 (2C), 126.3 (2C), 122.3, 82.7, 78.2, 45.3 (2C), 33.8 (2C), 9.9 (2C), 4.7; ES-API MS: m/z calcd for C₁₉H₁₆NO₂ 290.1, found 290.1 [M+H]⁺.

### (3aR,4R,4aR,5aS,6S,6aS)-2-(4-(3-(4-Methoxyphenyl)-3-oxoprop-1-yn-1-yl)phenyl)-4,4a,5,5a,6,6a-hexahydro-4,6-ethenocyclopropa[f]isoindole-1,3(2H,3aH)-dione (23)

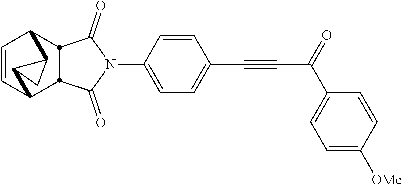

A flamed-dried round-bottom flask was charged with PdCl₂(PPh₃)₂ (2.4 mg, 0.003 mmol), Cut (1.3 mg, 0.007 mmol) and anhydrous THF (0.6 mL). The mixture was degassed and purged with argon (×3). A degassed solution of Et₃N (0.025 mL, 0.18 mmol) was added to the reaction mixture, followed by addition of 4-methoxybenzoyl chloride (31 mg, 0.18 mmol) and a solution of alkyne 115 (50 mg, 0.17 mmol) in anhydrous THF (0.5 mL) via syringe. The resulting mixture was stirred at rt for 2 h. Note that the mixture turned from yellow/orange to a greenish precipitate. The mixture was poured into a separatory funnel containing saturated aq. NaHCO₃ (5 mL) and EtOAc (10 mL). The organic phase was separated and washed with brine and dried over anhydrous Na₂SO₄, and filtered. The solvent was removed under reduced pressure and the residue was purified by flash chromatography on silica gel (gradient elution, 0→30% EtOAc in hexanes) followed by recrystallization in CH₂Cl₂/hexanes to afford ynone 23 (52 g, 0.12 mmol, 71%) as a yellow solid. IR (cm⁻¹) 2201, 1711, 1633, 1597, 1509, 1377, 1292, 1261, 1163, 736, ¹H NMR (400 MHz, CDCl₃) δ 8.17 (d, J=8.8 Hz, 2H), 7.72 (d, J=8.4 Hz, 2H), 7.29 (d, J=8.4 Hz, 2H), 6.99 (d, J=9.2 Hz, 2H), 5.87 (dd, J=4.8, 3.6 Hz, 2H), 3.90 (s, 3H), 3.51 (m, 2H), 3.16 (dd, J=1.6, 1.6 Hz, 2H), 1.16 (m, 2H), 0.37-0.27 (m, 2H); ¹³C NMR (100 MHz, CDCl₃) δ 177.1 (2C), 176.5, 164.6, 133.6, 133.5 (2C), 132.0 (2C), 130.2, 127.8 (2C), 126.5 (2C), 120.4, 113.9 (2C), 91.0, 87.4, 55.6, 45.4 (2C), 33.9 (2C), 9.9 (2C), 4.7; ES-API MS: m/z calcd for C₂₇H₂₂NO₄ 424.2, found 424.1 [M+H]⁺.

### (1aS,2S,2aS,9aR,10R,10aR)-5-Methoxy-1a,2,2a,9a,10,10a-hexahydro-2,10-ethenobenzo[4,5]imidazo[2,1-a]cyclopropa[f]isoindol-9(1H)-one (116)

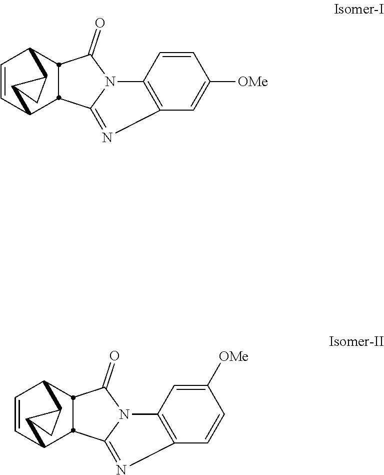

Procedure B (conventional heating). Purification by flash chromatography on silica gel (gradient elution, 0→70% EtOAc in hexanes) afforded an inseparable mixture of regioisomers (2:1 ratio in favor of desired isomer 116) (131 mg, 0.45 mmol, 85%) as a pale yellow solid. IR (cm⁻¹) 3458, 3364, 3007, 2955, 1768, 1630, 1515, 1212, 1188, 735; ¹H NMR (400 MHz, CDCl₃) 116: δ 6.83 (d, J=8.4 Hz, 1H), 6.37 (dd, J=8.4, 2.8 Hz, 1H), 6.29 (d, J=2.8 Hz, 1H), 5.94 (dd, J=4.4, 3.6 Hz, 2H), 3.75 (s, 3H), 3.50 (m, 2H), 3.16 (m, 2H), 1.15 (m, 2H), 0.37-0.23 (m, 2H); regioisomer: δ 6.71 (d, J=8.8 Hz, 1H), 6.37 (dd, J=8.8, 2.4 Hz, 1H), 6.33 (d, J=2.4 Hz, 1H), 5.87 (dd, J=4.0, 3.6 Hz, 2H), 3.62 (m, 2H), 3.53 (s, 3H), 3.13 (m, 2H), 1.15 (m, 2H), 0.37-0.23 (m, 2H); ¹³C NMR (100 MHz, CDCl₃) 116: δ 177.7, 161.1, 143.8, 129.6, 128.6 (2C), 127.8, 110.8, 104.9, 102.78, 101.91, 55.3, 45.7, 33.9, 9.8, 4.8; regioisomer: δ 178.0, 161.1, 143.6, 129.2, 128.7, 127.8, 111.8, 110.0, 105.3, 102.80, 101.91, 55.30, 45.2, 33.8, 9.9, 4.6; ES-API MS: m/z calcd for C₁₈H₁₇N₂O₂ 293.1, found 293.1 [M+H]⁺.

### (1aR,2R,2aR,9aS,10S,10aS)-5-Hydroxy-1a,2,2a,9a,10,10a-hexahydro-2,10-ethenobenzo[4,5]imidazo[2,1-a]cyclopropa[f]isoindol-9(1H)-one (117)

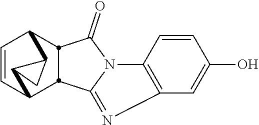

To a solution of the above mixture of regioisomeric methyl ethers 116 (873 mg, 3.00 mmol) in CH₂Cl₂ at −78° C. was added dropwise BBr₃ (5 mL, 4.5 mmol, 1 M solution in CH₂Cl₂). The resulting mixture was stirred for 12 h while allowing the temperature to slowly raise to rt. The reaction was quenched by adding MeOH (5 mL) and H₂O (2 mL) at 0° C., and extracting the mixture with EtOAc (4×10 mL). The combined organic layer was washed with brine and dried over anhydrous Na₂SO₄, and filtered. The solvent was removed under reduced pressure and the residue was purified by flash chromatography on silica gel (gradient elution, 0→80% EtOAc in hexanes) to give a mixture of regioisomeric phenols 117 (680 mg, 2.44 mmol, 82%) as a white amorphous solid. IR (cm⁻¹) 3360, 1694, 1514, 1180; ¹H NMR (400 MHz, CD₃OD) δ 6.70 (d, J=8.4 Hz, 1H), 6.26 (m, 1H), 6.18 (dd, J=8.8, 2.8 Hz, 1H), 5.92 (dd, J=4.8, 3.6 Hz, 2H), 3.37 (m, 2H), 3.20 (dd, J=2.0, 2.0 Hz, 2H), 1.20 (m, 2H), 0.35-0.22 (m, 2H); ¹³C NMR (100 MHz, CD₃OD) δ 179.6, 158.7, 145.6, 128.9, 128.3, 127.5, 109.7, 105.3, 104.7, 102.3, 45.6, 45.3, 33.7, 9.3, 9.2, 3.8, 3.5.

### (1aR,2R,2aR,9aS,10S,10aS)—S-(2-(4-Methoxyphenyl)-2-oxoethoxy)-1a,2,2a,9a,10,10a-hexahydro-2,10-ethenobenzo[4,5]imidazo[2,1-a]cyclopropa[f]isoindol-9(1H)-one (87)

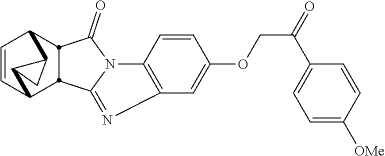

To a solution of the regioisomeric mixture of the above phenols 117 (50 mg, 0.18 mmol) in ethanol (1.8 mL) was added Cs₂CO₃ (176 mg, 0.54 mmol) and 2-bromo-4′-OMe-acetophenone (82 mg, 0.36 mmol). The resulting mixture was heated to 50° C. for 4 h. The solvent was removed under reduced pressure and the residue was purified by flash chromatography on silica gel (gradient elution, 0→45% EtOAc in hexanes) followed by purification with HPLC (reverse phase, MeCN/H₂O/0.1% TFA) and recrystallization in CH₂Cl₂/hexanes to afford the desired product 87 as a single regioisomer (23 mg, 0.05 mmol, 30%) as a white solid. IR (cm⁻¹) 3460, 3365, 3051, 3009, 2956, 1704, 1601, 1514, 1393, 1308, 1241, 1173, 735; ¹H NMR (400 MHz, CDCl₃) δ 7.98 (d, J=8.8 Hz, 2H), 6.95 (d, J=8.8 Hz, 2H), 6.82 (d, J=8.4 Hz, 1H), 6.39 (d, J=1.6 Hz, 1H), 6.36 (dd, J=6.8, 2.4 Hz, 1H), 5.94-5.85 (m, 2H), 5.11 (s, 2H), 3.88 (s, 3H), 3.50 (m, 2H), 3.16-3.13 (m, 2H), 1.15 (m, 2H), 0.36-0.23 (m, 2H); ¹³C NMR (100 MHz, CDCl₃) δ 193.0, 177.6, 164.0, 159.5, 144.0, 130.7 (2C), 129.7, 128.6 (2C), 127.8, 127.6, 114.0 (2C), 111.6, 105.4, 102.9, 71.0, 55.5, 45.7, 45.2, 33.8, 9.9, 9.8, 4.8, 4.6; ES-API MS: m/z calcd for C₂₆H₂₃N₂O₄ 427.2, found 427.1 [M+H]⁺.

### (5-Aminobenzofuran-2-yl)(4-methoxyphenyl)methanone (118)

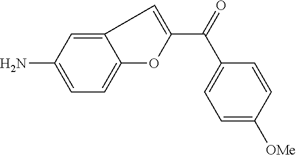 [SMILES: COc1ccc(C(=O)c2cc3cc(N)ccc3o2)cc1]

A mixture of N-(3-formyl-4-hydroxyphenyl)acetamide (200 mg, 1.12 mmol), K₂CO₃ (308 mg, 2.23 mmol) and 4′-MeO-2-bromoacetophenone (511 mg, 2.23 mmol) was dissolved in anhydrous DMF (3.72 mL). The resulting mixture was heated to 90° C. and stirred for 4 h. The solvent was removed under reduced pressure and the residue was purified by flash chromatography on silica gel (gradient elution, 0→55% EtOAc in CH₂Cl₂) to give an intermediate acetamide (200 mg, 0.65 mmol, 58%) as a pale yellow solid. The acetamide (200 mg, 0.65 mmol) was then dissolved in EtOH (6.5 mL) and aq. HCl (3 N, 3.2 mL) was added and the mixture was refluxed overnight. The solvent was removed under reduced pressure, and the residue was diluted with EtOAc and neutralized with saturated aq. NaHCO₃. The organic layer was washed with brine and dried over anhydrous MgSO₄, and filtered. The solvent was removed under reduced pressure and the residue was purified by flash chromatography on silica gel (100% EtOAc) to give the free aniline 118 (168 mg, 0.63 mmol, 97%) as a bright yellow solid. IR (cm⁻¹) 3445, 3360, 1634, 1601, 1573, 1548, 1510, 1329, 1261, 1164, 975, 765; ¹H NMR (400 MHz, CDCl₃) δ 8.08 (d, J=8.8 Hz, 2H), 7.40 (d, J=8.8 Hz, 1H), 7.35 (s, 1H), 7.00 (d, J=9.2 Hz, 2H), 6.91 (d, J=2.0 Hz, 1H), 6.87 (dd, J=8.4, 2.4 Hz, 1H), 3.89 (s, 3H), 3.71 (br, 2H); ¹³C NMR (100 MHz, CDCl₃) δ 183.0, 163.6, 153.3, 150.7, 143.2, 132.1 (2C), 130.1, 128.1, 118.3, 115.3, 113.9 (2C), 113.0, 106.5, 55.7; ES-API MS: m/z calcd for C₁₆H₁₄NO₃ 268.1, found 268.1 [M+H]⁺.

### (3aR,4R,4aR,5aS,6S,6aS)-2-(2-(4-Methoxybenzoyl)benzofuran-5-yl)-4,4a,5,5a,6,6a-hexahydro-4,6-ethenocyclopropa[f]isoindole-1,3(2H,3aH)-dione (88)

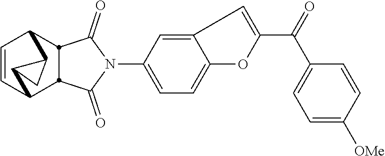

A mixture of aniline 118 (175 mg, 0.92 mmol) and maleic anhydride 97 (175 mg, 0.92 mmol) was dissolved in acetic acid (3.0 mL), heated to 100° C. and stirred for 3 h. The mixture was cooled to rt and diluted with EtOAc, and quenched with saturated aq. NaHCO₃. The organic layer was washed with brine and dried over anhydrous MgSO₄, and filtered. The solvent was removed under reduced pressure and the residue was purified by flash chromatography on silica gel (gradient elution, 0→60% EtOAc in hexanes) followed by recrystallization in CH₂Cl₂/hexanes to give the product 88 (227 mg, 0.52 mmol, 56%) as a yellow-orange solid. IR (cm⁻¹) 2952, 1714, 1644, 1601, 1558, 1392, 1281, 1259, 1175, 884, 735; ¹H NMR (400 MHz, CDCl₃) δ 8.09 (d, J=8.8 Hz, 2H), 7.68 (d, J=8.8 Hz, 1H), 7.54 (d, J=2.0 Hz, 1H), 7.50 (s, 1H), 7.26 (dd, J=8.8, 2.0 Hz, 1H), 7.02 (d, J=8.8 Hz, 2H), 5.90 (dd, J=4.8, 3.6 Hz, 2H), 3.91 (s, 3H), 3.52 (m, 2H), 3.18 (dd, J=1.6, 1.6 Hz, 2H), 1.17 (m, 2H), 0.37-0.28 (m, 2H); ¹³C NMR (100 MHz, CDCl₃) δ 182.8, 178.0 (2C), 164.0, 155.2, 153.9, 132.2 (2C), 129.8, 128.1 (2C), 128.0, 127.7, 126.7, 121.7, 115.3, 114.1 (2C), 113.4, 55.8, 45.5 (2C), 34.1 (2C), 10.1 (2C), 4.9; ES-API MS: m/z calcd for C₂₇H₂₂NO 440.1, found 440.1 [M+H]⁺.

### (3aR,4R,4aR,5aS,6S,6aS)-2-(4-((E)-3-(2-Bromo-4-methoxyphenyl)-3-oxoprop-1-en-1-yl)phenyl)-4,4a,5,5a,6,6a-hexahydro-4,6-ethenocyclopropa[f]isoindole-1,3(2H,3aH)-dione (119)

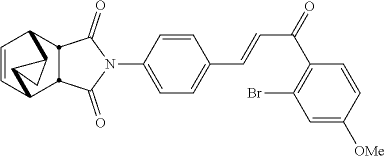

Procedure E. Purification by flash chromatography on silica gel (gradient elution, 0→50% EtOAc in hexanes) followed by recrystallization in CH₂Cl₂/hexanes afforded enone 119 (333 mg, 0.66 mmol, 71%) as a pale yellow solid. IR (cm⁻¹) 3008, 2956, 1709, 1599, 1378, 1293, 1233, 1181, 1032, 732; ¹H NMR (400 MHz, CDCl₃) δ 7.63 (d, J=8.8 Hz, 2H), 7.50 (d, J=16.0 Hz, 1H), 7.48 (d, J=8.8 Hz, 1H), 7.25 (d, J=8.8 Hz, 2H), 7.18 (d, J=2.4 Hz, 1H), 7.17 (d, J=16.0 Hz, 1H), 6.93 (dd, J=8.4, 2.4 Hz, 1H), 5.86 (dd, J=4.8, 3.6 Hz, 2H), 3.86 (s, 3H), 3.50 (m, 2H), 3.15 (dd, J=1.6, 2.0 Hz, 2H), 1.16 (m, 2H), 0.36-0.26 (m, 2H); ¹³C NMR (100 MHz, CDCl₃) δ 193.1, 177.5 (2C), 161.9, 143.6, 135.0, 133.8, 133.4, 131.5, 129.2 (2C), 128.0 (2C), 127.2, 127.0 (2C), 121.4, 119.2, 113.5, 56.0, 45.6 (2C), 34.1 (2C), 10.1 (2C), 4.9; ES-API MS: m/z calcd for C₂₇H₂₂BrNO₄Na 528.0 (⁸¹Br isotope), found 528.0 [M+Na]⁺.

### (3aR,4R,4aR,5aS,6S,6aS)-2-(4-(5-Methoxy-1-oxo-J H-inden-3-yl)phenyl)-4,4a,5,5a,6,6a-hexahydro-4,6-ethenocyclopropa[f]isoindole-1,3(2H,3aH)-dione (89)

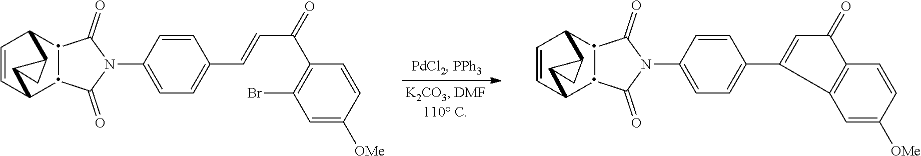

A mixture of enone 119 (20 mg, 0.040 mmol), PdCl₂ (3.5 mg, 0.02 mmol), PPh₃ (16 mg, 0.06 mmol) and K₂CO₃ (14 mg, 0.10 mmol) in DMF (0.5 mL) was heated to 110° C. and stirred for 3 h. The reaction was cooled to rt and filtered through a Celite pad and washed with EtOAc. The solvent was removed under reduced pressure and the residue was purified by flash chromatography on silica gel (gradient elution, 0→45% EtOAc in hexanes) to obtain benzofuranone 89 (5.0 mg, 0.012 mmol, 31%) as a yellow solid. IR (cm⁻¹) 3006, 2925, 2855, 1711, 1601, 1384, 1180, 734; ¹H NMR (400 MHz, CDCl₃) δ 7.68 (d, J=8.4 Hz, 2H), 7.49 (d, J=8.0 Hz, 1H), 7.35 (d, J=8.4 Hz, 2H), 6.88 (d, J=2.0 Hz, 1H), 6.69 (dd, J=8.0, 1.6 Hz, 1H), 6.01 (s, 1H), 5.89 (dd, J=4.4, 4.0 Hz, 2H), 3.85 (s, 3H), 3.53 (m, 2H), 3.19 (m, 2H), 1.18 (m, 2H), 0.38-0.28 (m, 2H); ¹³C NMR (100 MHz, CDCl₃) δ 195.6, 177.4, 164.0, 159.5, 146.3, 133.1, 128.0 (2C), 127.9 (2C), 126.9 (2C), 125.3, 124.8, 124.7, 110.9, 110.6, 55.8, 45.4 (2C), 33.9 (2C), 29.7, 9.9 (2C), 4.7; ES-API MS: m/z calcd for C₂₇H₂₂NO₄ 424.2, found 424.1 [M+H]⁺.

### (3aR,4R,4aR,5aS,6S,6aS)-2-(4-((6-Methoxybenzo[d]isoxazol-3-yl)methoxy)phenyl)-4,4a,5,5a,6,6a-hexahydro-4,6-ethenocyclopropa[f]isoindole-1,3(2H,3aH)-dione (90)

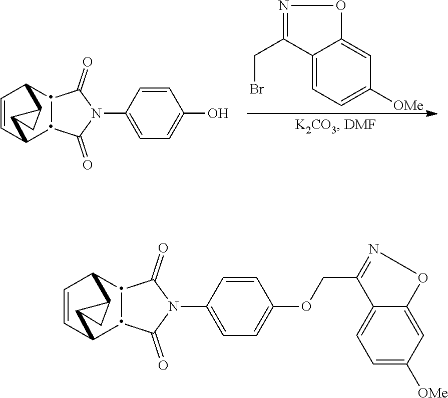

A mixture of phenol 102 (45 mg, 0.16 mmol) and K₂CO₃ (33 mg, 0.24 mmol) in anhydrous DMF (0.30 mL) was heated at 50° C. for 30 min and cooled to rt before adding 3-(bromomethyl)-6-methoxy-benzoisoxazole (120, 77 mg, 0.32 mmol). The resulting mixture was heated to 65° C. and stirred overnight. The solvent was removed under reduced pressure and the residue was purified by flash chromatography on silica gel (gradient elution, 0→40% EtOAc in hexanes) followed by recrystallization in CH₂Cl₂/hexanes to deliver benzoisoxazole analog 90 (55 mg, 0.14 mmol, 77%) as a white solid. IR (cm⁻¹) 3054, 3006, 2960, 1705, 1616, 1511, 1140, 831; ¹H NMR (400 MHz, CDCl₃) δ 7.65 (dd, J=8.8, 0.4 Hz, 1H), 7.09 (s, 4H), 7.00 (d, J=2.0 Hz, 1H), 6.92 (dd, J=8.8, 2.0 Hz, 1H), 5.84 (dd, J=4.8, 3.6 Hz, 2H), 5.42 (s, 2H), 3.88 (s, 3H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 178.0 (2C), 165.7, 162.7, 158.0, 154.9, 128.1 (2C), 128.0 (2C), 125.6, 122.5, 115.4 (2C), 115.1, 114.2, 92.8, 62.3, 56.0, 45.5 (2C), 34.0 (2C), 10.1 (2C), 4.9, ES-API MS: m/z calcd for C₂₆H₂₃N₂O₅ 443.2, found 443.2 [M+H]⁺.

### (3aR,4R,4aR,5aS,6S,6aS)-2-(3-Hydroxyphenyl)-4,4a,5,5a,6,6a-hexahydro-4,6-ethenocyclopropa[f]isoindole-1,3(2H,3aH)-dione (121)

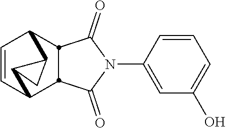

Procedure B. Purification by flash chromatography on silica gel (gradient elution, 0→50% EtOAc in hexanes) followed by recrystallization in CH₂Cl₂/hexanes afforded phenol 121 (90 mg, 0.32 mmol, 61%) as colorless crystals. IR (cm⁻¹) 3310, 1773, 1686, 1595, 1389, 1272, 1179, 733, 714; ¹H NMR (400 MHz, CDCl₃) δ 7.26 (dd, J=8.4, 7.6 Hz, 1H), 6.79 (dd, J=8.4, 2.4 Hz, 1H), 6.72 (dd, J=7.6, 0.8 Hz, 1H), 6.62 (dd, J=2.0, 2.0 Hz, 1H), 5.85 (dd, J=4.8, 3.6 Hz, 2H), 5.63 (br, 1H), 3.49 (m, 2H), 3.14 (dd, J=2.0, 1.6 Hz, 2H), 1.15 (m, 2H), 0.35-0.26 (m, 2H); ¹³C NMR (100 MHz, CDCl₃) δ 177.9 (2C), 156.2, 132.6, 130.0, 127.8 (2C), 118.6, 116.0, 113.8, 45.3 (2C), 33.8 (2C), 9.9 (2C), 4.6; ES-API MS: m/z calcd for C₁₇H₁₆NO₃ 282.1, found 282.1 [M+H]⁺.

### (3aR,4R,4aR,5aS,6S,6aS)-2-(3-(2-(4-Methoxyphenyl)-2-oxoethoxy)phenyl)-4, 4a,5,5a,6,6a-hexahydro-4,6-ethenocyclopropa[f]isoindole-1,3(2H,3aH)-dione (91)

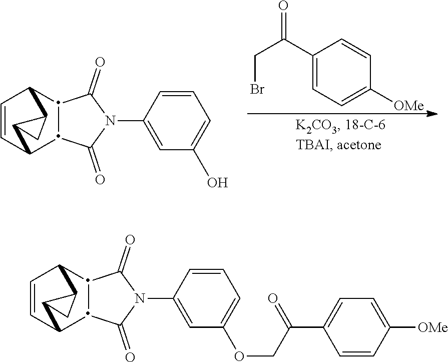

To a solution of phenol 121 (37 mg, 0.13 mmol) in reagent grade acetone (1.3 mL) was added K₂CO₃ (54 mg, 0.39 mmol), 18-crown-6 (17 mg, 0.07 mmol), tetrabutylammonium iodide (24 mg, 0.07 mmol) and 2-bromo-4′-OMe-acetophenone (60 mg, 0.26 mmol). The resulting mixture was refluxed at 65-70° C. for 48 h (Note that the product has the same Rf as the starting material). The solvent was removed under reduced pressure and the residue was purified by flash chromatography on silica gel (gradient elution, 0→50% EtOAc in hexanes) followed by recrystallization in CH₂Cl₂/hexanes to afford the product 91 (40 mg, 0.09 mmol, 71%) as a pale yellow solid. IR (cm⁻¹) 3082, 3051, 3007, 2841, 1772, 1709, 1602, 1493, 1384, 1262, 1234, 1181, 971, 839, 733; ¹H NMR (400 MHz, CDCl₃) δ 7.98 (d, J=8.8 Hz, 2H), 7.32 (dd, J=8.0, 8.0 Hz, 1H), 6.96 (d, J=8.8 Hz, 2H), 6.93 (m, 1H), 6.80 (m, 2H), 5.84 (dd, J=4.8, 3.6 Hz, 2H), 5.17 (s, 2H), 3.88 (s, 3H), 3.48 (m, 2H), 3.12 (dd, J=1.6, 1.6 Hz, 2H), 1.14 (m, 2H), 0.34-0.25 (m, 2H); ¹³C NMR (100 MHz, CDCl₃) δ 192.6, 177.4 (2C), 164.1, 158.5, 132.9, 130.6 (2C), 129.9, 127.8 (2C), 127.5, 119.7, 114.8, 114.0 (2C), 113.6, 70.9, 55.5, 45.3 (2C), 33.8 (2C), 9.9 (2C), 4.7; ES-API MS: m/z calcd for C₂₆H₂₄NO₅ 430.2, found 430.1 [M+H]⁺.

### In Vivo Studies

Chronic, once daily oral dosing of the analogs of Tables 1-9 is well tolerated by mice. Body weights were evaluated in ICR SCID mice treated once per day P.O. with vehicle or 5, 20 or 60 mg/kg doses of analog for 14 consecutive days. n=5 per arm. Complete blood counts, including total hemoglobin (Hb) and platelets (PLT) were evaluated at Day 14. Serum chemistry analyses were also evaluated at Day 14. Alanine (ALT) and aspartate (AST) aminotransferases were used markers of liver damage. Creatinine was used as a marker of kidney function. Triglycerides were used as a systemic biomarker for cholesterol synthesis inhibition. Plasma levels of analog were evaluated at Day 14.

Results from these in vivo animal studies demonstrate that the analogs of Tables 1-9 are bioavailable and brain penetrant, do not lead to body weight loss, do not cause hematologic, hepatic, or renal toxicity, and do not increase but actually decrease triglyceride levels in mice during chronic treatment.

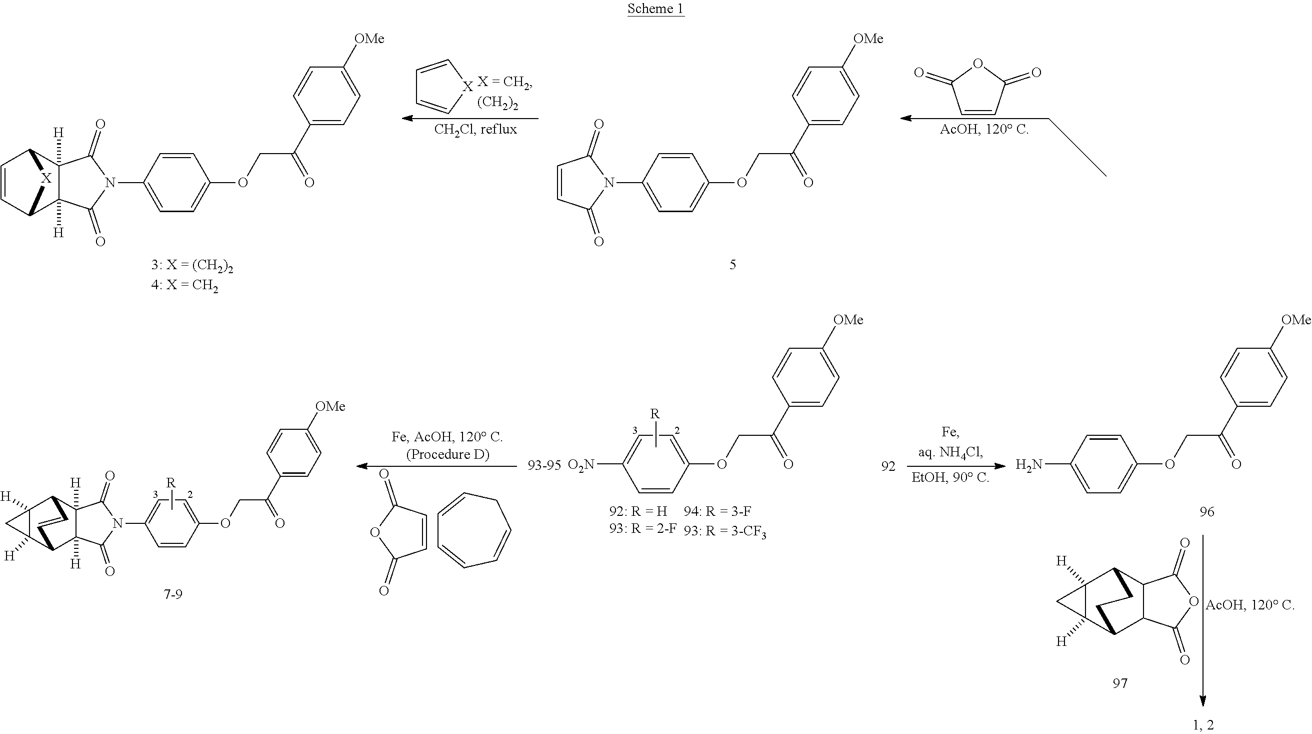

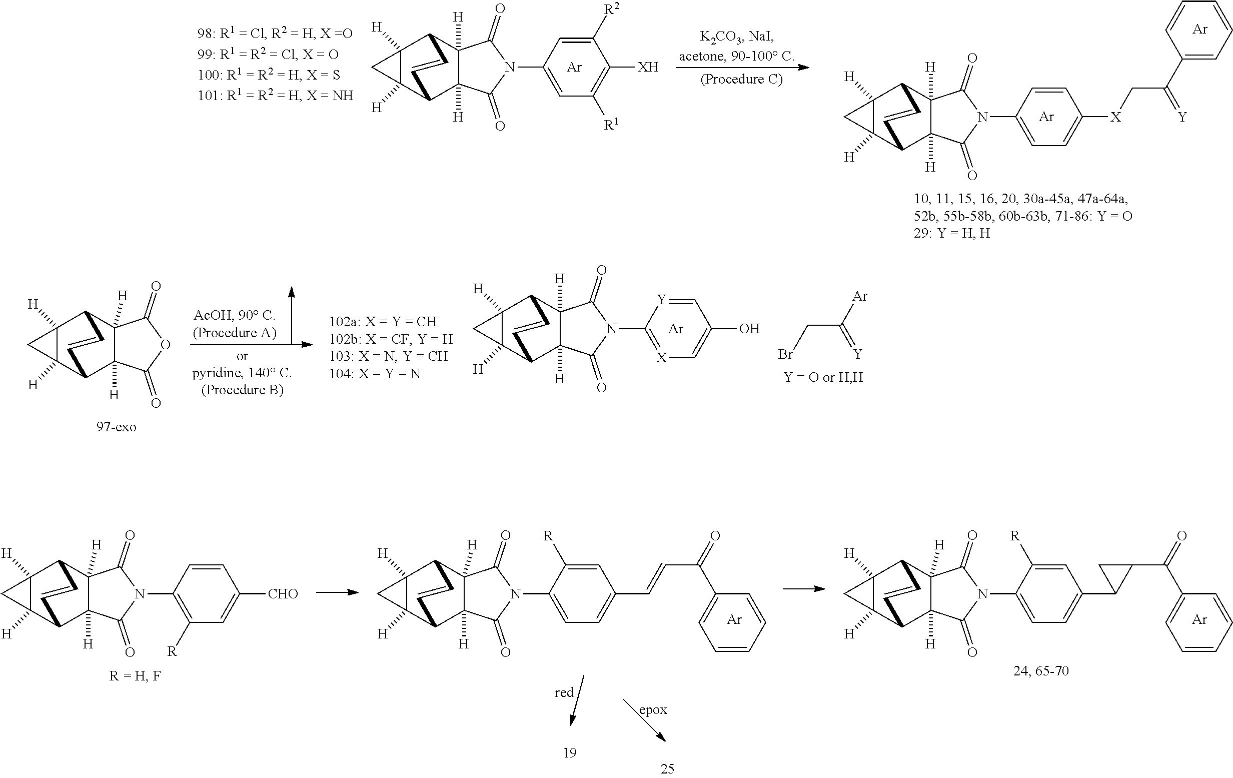

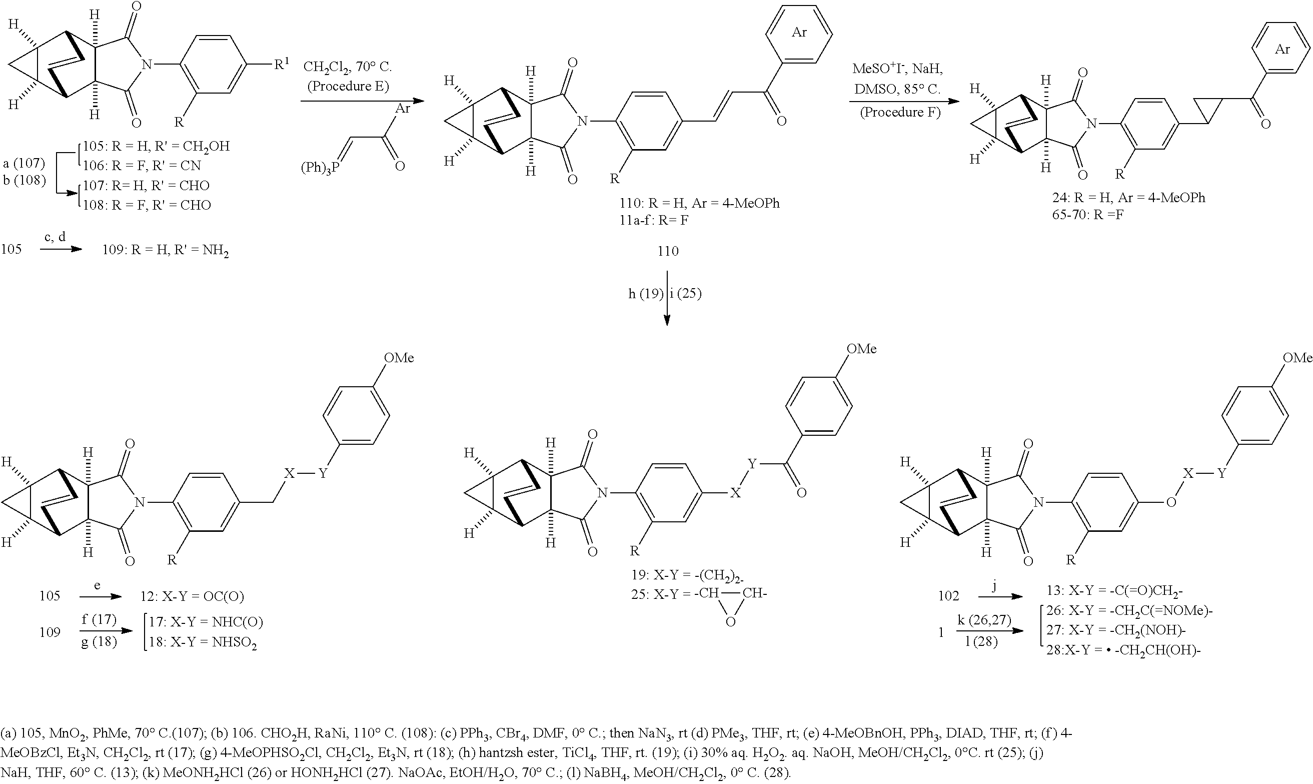

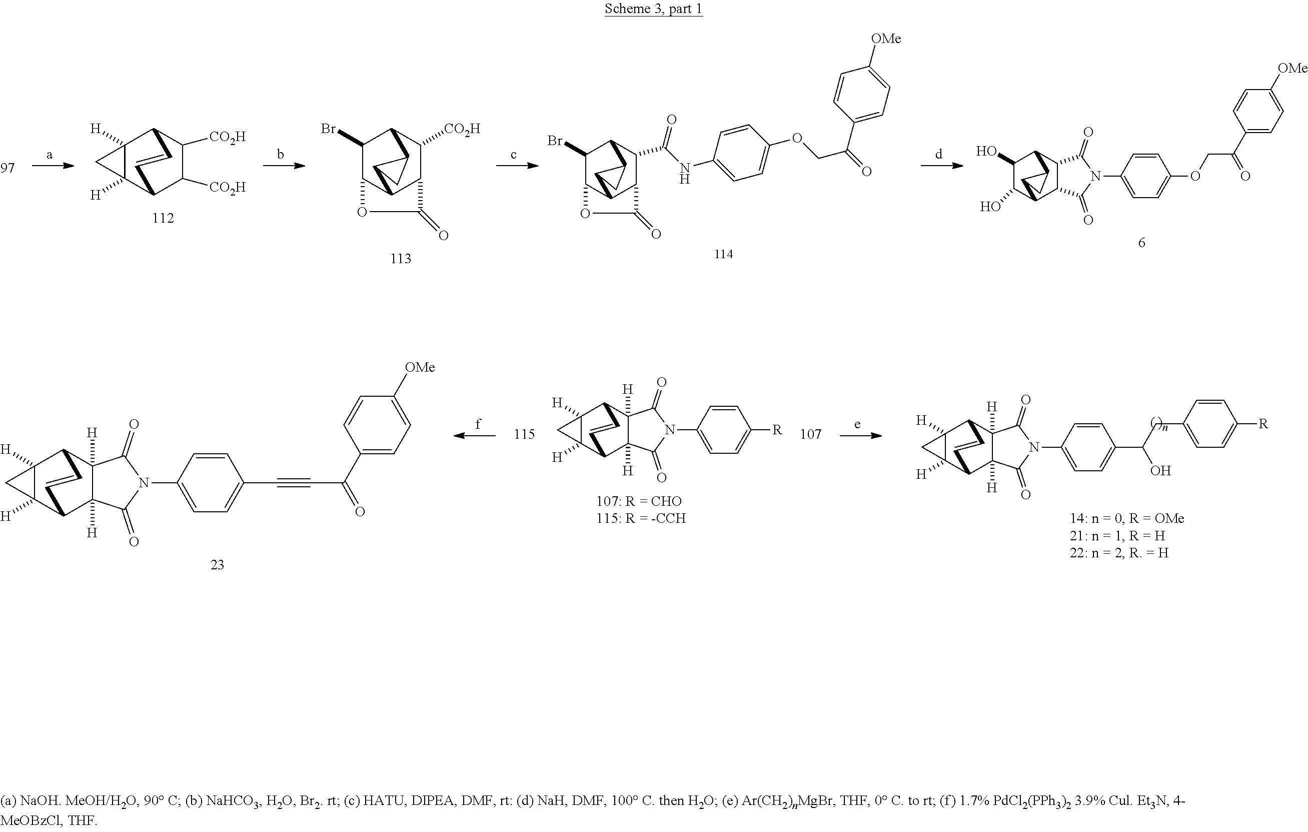

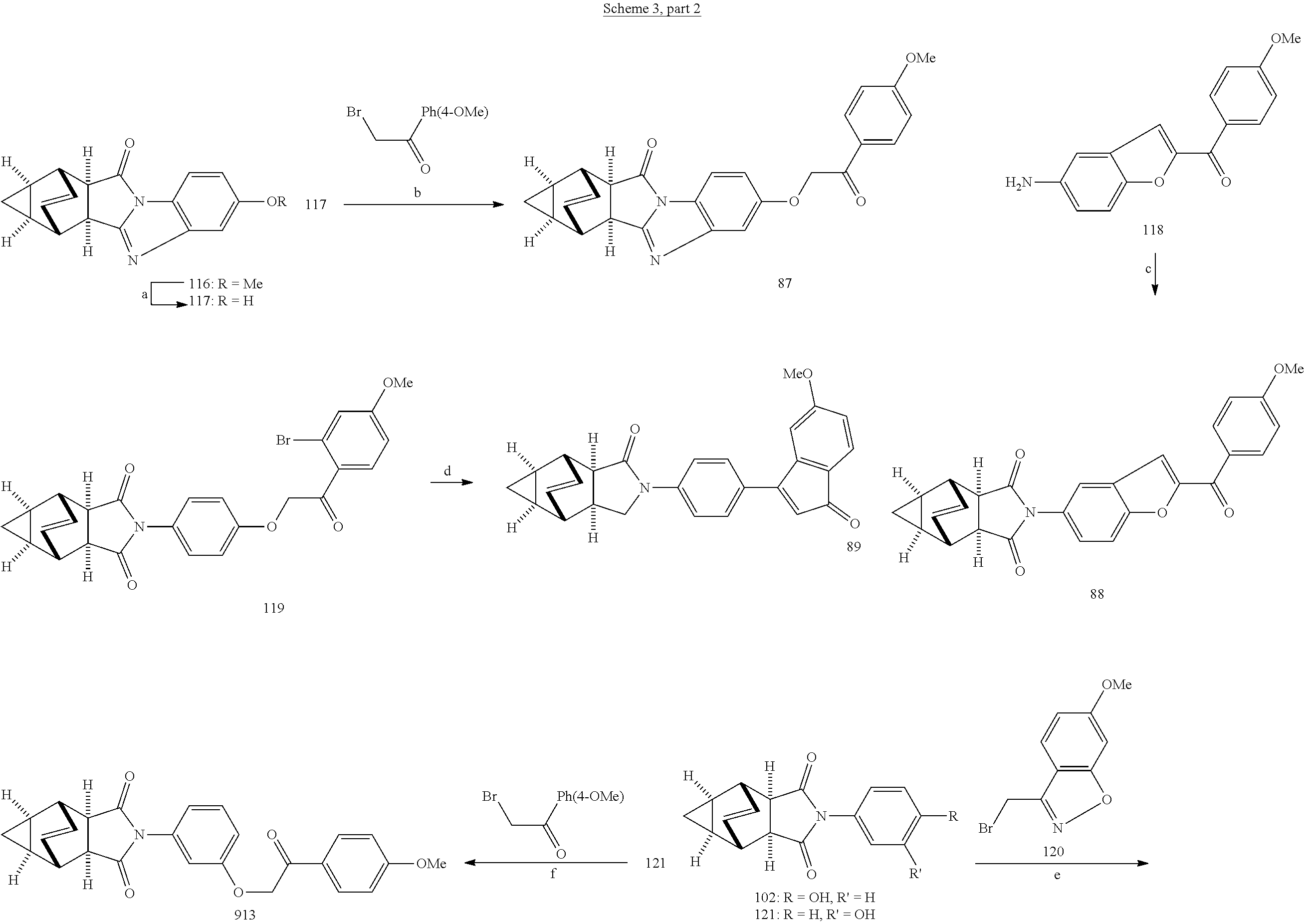

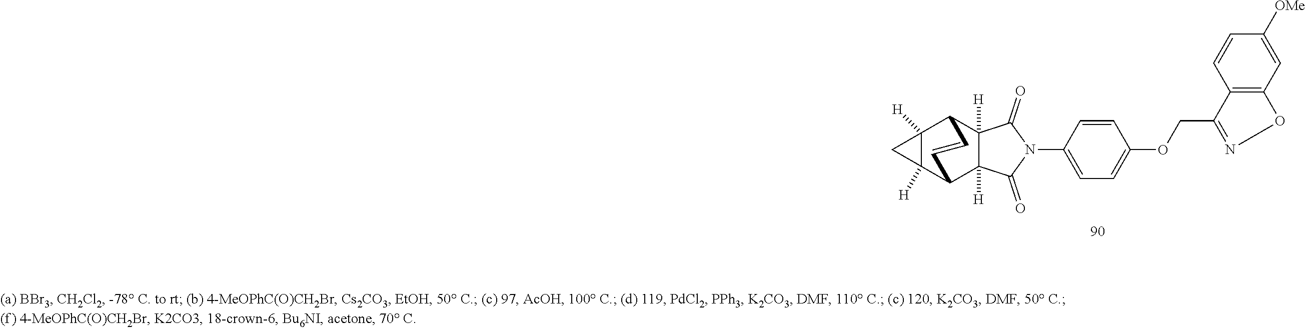 [SMILES: COc1ccc2c(COc3ccc(N4C(=O)[C@@H]5[C@@H]6C=C[C@@H]([C@H]7C[C@@H]67)[C@@H]5C4=O)cc3)noc2c1]

TABLE 1
SAR of the dicarboximide moiety

| cpd | R₂N | Mut6 IC₅₀ (μM) | S9 T_{1/2} (min) |
|---|---|---|---|
| 1 (endo) | 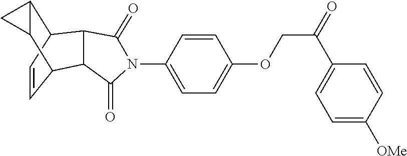 | 0.036 ± 0.004 | 14 |
| 2 (exo) | 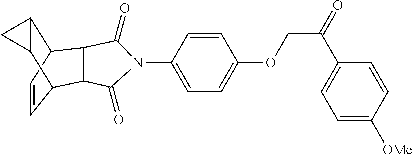 | 0.169 ± 0.010 | 14 |
| 3 | 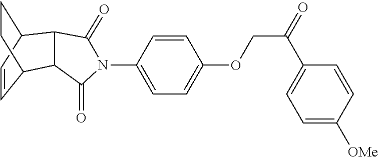 | 0.128 ± 0.005 | 21 |
| 4 | 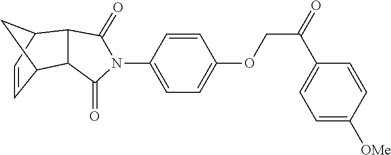 | 1.661 ± 0.054 | NT |
| 5 | 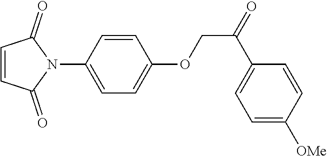 | 13.684 ± 1.285 | NT |
| 6 | 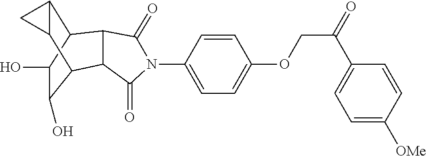 | 7.927 ± 0.534 | NT |

TABLE 2
SAR of the central phenyl ring

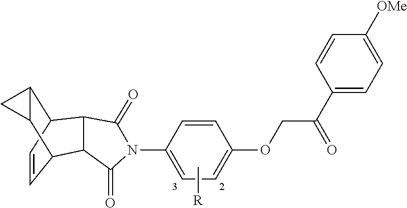

| cpd | R | Mut6 IC₅₀ (μM) | S9 T_{1/2} (min) |
|---|---|---|---|
| 7 | 3-F | 0.928 ± 0.066 | >240 |
| 8 | 2-F | 0.113 ± 0.011 | 6.78 |
| 9 | 3-CF₃ | 33.866 ± 4.278 | NT |
| 10 | 2-Cl | 0.100 ± 0.015 | NT |
| 11 | 2,6-Cl₂ | 0.210 ± 0.020 | NT |

TABLE 3
SAR of the linker

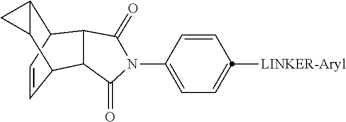

| cpd | LINKER-Aryl | Mut6 IC₅₀ (μM) | S9 T_{1/2} (min) |
|---|---|---|---|
| 1 | 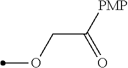 | 0.036 ± | 14 |
| 12 | 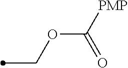 | 1.075 ± 0.115 | 16.9 |
| 13 | 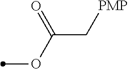 | 41.165 ± 5.775 | NT |
| 14 | 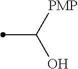 | 3.394 ± 0.207 | NT |
| 15 | 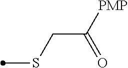 | 0.098 ± 0.008 | NT |
| 16 | 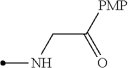 | 0.372 ± 0.023 | NT |
| 17 | 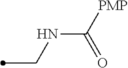 | 3.758 ± 0.342 | NT |
| 18 | 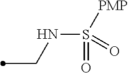 | 0.244 ± 0.012 | NT |
| 19 | 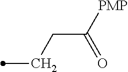 | 0.032 ± 0.003 | 17.2 |
| 20 | 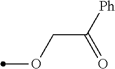 | 0.462 ± 0.026 | NT |
| 21 | 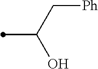 | 3.895 ± 0.291 | 10.9 |
| 22 | 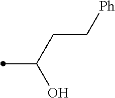 | 0.362 ± 0.027 | NT |
| 23 | 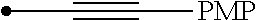 | 1.525 ± 0.111 | NT |
| 24 | 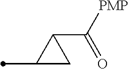 | 0.090 ± 0.004 | 92.4 |
| 25 | 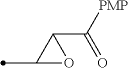 | 0.160 ± 0.006 | 6.0 |
| 26 | 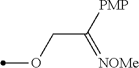 | 1.523 ± 0.137 | NT |
| 27 | 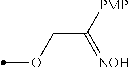 | 1.025 ± 0.068 | NT |
| 28 | 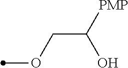 | 1.171 ± 0.124 | 119.5 |
| 29 | 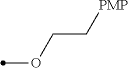 | 0.136 ± 0.013 | 57.8 |

TABLE 4
SAR of the benzoyl moiety

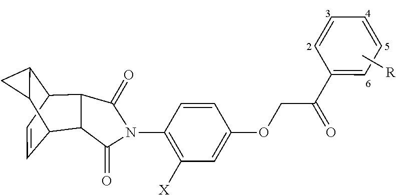

a: X = H; b: X = F

| cpd | R | Mut6 IC₅₀ (μM) | S9 T_{1/2} (min) |
|---|---|---|---|
| 20 | H | 0.462 ± 0.026 | NT |
| 1 | 4-OMe | 0.036 | 14 |
| 30a | 3-OMe | 0.031 ± 0.002 | 6.1 |
| 31a | 2-OMe | 4.153 ± 0.355 | 18.8 |
| 32a | 4-OEt | 0.139 ± 0.011 | NT |
| 33a | 4-OCH₂CCH | 0.131 ± 0.009 | 13.6 |
| 34a | 4-OBu | 0.868 ± 0.073 | NT |
| 35a | 4-F | 0.084 ± 0.005 | NT |
| 36a | 4-Cl | 0.013 ± 0.001 | 8.2 |
| 37a | 4-Br | 0.015 ± 0.001 | 9.2 |
| 38a | 4-Me | 0.042 ± 0.002 | NT |
| 39a | 4-CF₃ | 0.0196 ± 0.0005 | 29.1 |
| 40a | 4-OCF₃ | 0.060 ± 0.003 | 59.7 |
| 41a | 4-OH | 3.373 ± 0.330 | NT |
| 42a | 4-OAc | 3.496 ± 0.202 | NT |
| 43a | 4-OCO₂Me | 1.311 ± 0.135 | NT |
| 44a | 4-NHCO₂Me | 0.951 ± 0.077 | NT |
| 45a | 4-CO₂Me | 1.593 ± 0.103 | NT |
| 46a | 4-C(O)Ph | 3.115 ± 0.312 | NT |
| 47a | 3-Cl | 0.076 ± 0.006 | 7.3 |
| 48a | 2,3-Cl₂ | 1.276 ± 0.141 | NT |
| 49a | 2,5-Cl₂ | 0.852 ± 0.062 | NT |
| 50a | 2,6-Cl₂ | 2.886 ± 0.208 | NT |
| 51a | 2,3,4-Cl₃ | 0.359 ± 0.022 | NT |
| 52a | 3,5-Cl₂ | 0.063 ± 0.003 | >240 |
| 52b | 3,5-Cl₂ | 0.472 ± 0.023 | 23.9 |
| 53a | 3-Cl, 5-F | 0.110 ± 0.008 | 8.0 |
| 54a | 3-CF3, 5-Cl | 0.107 ± 0.006 | 51.0 |
| 55a | 3,4-(OMe)₂ | 0.010 ± 0.001 | 54.1 |
| 55b | 3,4-(OMe)₂ | 0.076 ± 0.008 | 58.2 |
| 56a | 3,4-(OCH₂O) | 0.104 ± 0.011 | 24.8 |
| 56b | 3,4-(OCH₂O) | 0.750 ± 0.076 | 19.1 |
| 57a | 3,4-Mez | 0.0047 ± 0.0004 | 20.0 |
| 57b | 3,4-Me₂ | 0.033 ± 0.003 | 19.9 |
| 58a | 3,4-Cl₂ | 0.00151 ± | 11.6 |
| | | [symbol] | |
| 58b | 3,4-Cl₂ | 0.019 ± 0.001 | 11.1 |
| 59a | 3-F, 4-OMe | 0.027 ± 0.001 | 27.9 |
| 60a | 3-Cl, 4-OMe | 0.0035 ± 0.0003 | 18.6 |
| 60b | 3-Cl, 4-OMe | 0.024 ± 0.002 | 16.7 |
| 61a | 3-Me, 4-Cl | 0.0012 ± 0.0001 | 9.2 |
| 61b | 3-Me, 4-Cl | 0.029 ± 0.002 | 7.6 |
| 62a | 3-Cl, 4-F | 0.011 ± 0.001 | 5.2 |
| 62b | 3-Cl, 4-F | 0.055 ± 0.004 | 8.5 |
| 63a | 3-CF₃, 4-Cl | 0.018 ± 0.001 | 29.1 |
| 63b | 3-CF₃, 4-Cl | 0.165 ± 0.010 | 14.4 |
| 64a | 3,4-(CHCHCHCH) | 0.0085 ± 0.0006 | NT |

[symbol]

indicates data missing or illegible when filed

TABLE 5
SAR of trans-benzoylcyclopropane analogs

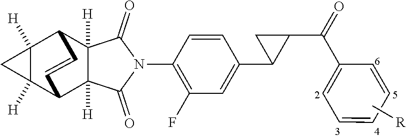

| cpd | R | Mut6 IC₅₀ (μM) | S9 T_{1/2} (min) |
|---|---|---|---|
| 65 | 3-Cl, 4-OMe | 0.030 ± 0.002 | 157.5 |
| 66 | 3-Me, 4-Cl | 0.027 ± 0.002 | 192.5 |
| 67 | 3-Cl, 4-F | 0.100 ± 0.008 | 56.8 |
| 68 | 3-CF₃, 4-Cl | 0.115 ± 0.013 | 169 |
| 69 | 3,4-Cl₂ | 0.034 ± 0.003 | 47.1 |
| 70 | 3,5-Cl₂ | 0.199 ± 0.011 | 18.3 |

TABLE 6
SAR of benzoylcyclopropane analogs

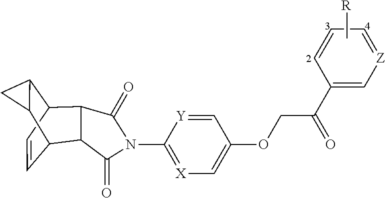

| | | | | S9 T_{1/2} |
|---|---|---|---|---|
| cpd | X/Y/Z | R | Mut6 IC₅₀ (μM) | (min) |
| 1 | CH/CH/CH | 4-OMe | 0.036 ± 0.004 | 14 |
| 71 | N/CH/CH | 4-OMe | 0.914 ± 0.080 | 13.7 |
| 72 | CH/CH/N | 4-OMe | 0.434 ± 0.027 | 7.5 |
| 73 | N/CH/N | 4-OMe | 3.324 ± 0.288 | 9.4 |
| 74 | N/N/CH | 4-OMe | 3.637 ± 0.346 | 2.0 |
| 75 | N/CH/CH | 3-Cl, 4-OMe | 0.040 ± 0.003 | 13.4 |
| 76 | N/CH/CH | 3-Me, 4-Cl | 0.013 ± 0.001 | 8.1 |
| 77 | N/CH/CH | 3-Cl, 4-F | 0.100 ± 0.005 | 3.2 |
| 78 | N/CH/CH | 3-CF₃, 4-Cl | 0.162 ± 0.005 | 9.6 |
| 79 | N/CH/CH | 3,4-Cl₂ | 0.014 ± 0.001 | >240 |
| 80 | N/CH/CH | 3,5-Cl₂ | 0.391 ± 0.047 | >240 |
| 81 | CH/CH/N | 3,4-Cl₂ | 0.095 ± 0.005 | 5.3 |
| 82 | N/N/CH | 3-Cl, 4-OCH₃ | 0.194 ± 0.014 | 3.3 |
| 83 | N/N/CH | 3-CH₃, 4-Cl | 0.086 ± 0.004 | 2.3 |
| 84 | N/N/CH | 3-Cl, 4-F | 0.418 ± 0.030 | 3.9 |
| 85 | N/N/CH | 3,4-Cl₂ | 0.100 ± 0.010 | >240 |
| 86 | N/N/CH | 3,5-Cl₂ | 2.185 ± 0.152 | >240 |

TABLE 7
Series II compounds were synthetized consistent with synthetic
schemes 1-3, supra; anti-GBM activity was confirmed between 1-200 nm
in the survival screen supra, using Mut 6, a murine GBM stem-like
cell (GSC) line, from a genetically engineered mouse model of GBM
wherein autochthonous tumor formation is driven by GFAP-Cre-mediated
silencing of the tumor suppressors Trp53, Pten, and Nf1 in murine astrocytes.
Animal studies confirm that these analogs are bioavailable and brain
penetrant, do not lead to body weight loss, do not cause hematologic,
hepatic, or renal toxicity, and do not increase but actually decrease
triglyceride levels in mice during chronic treatment.

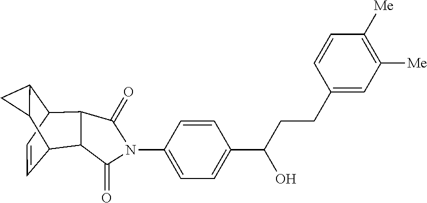 [SMILES: Cc1ccc(CCC(O)c2ccc(N3C(=O)C4C5C=CC(C6CC56)C4C3=O)cc2)cc1C]

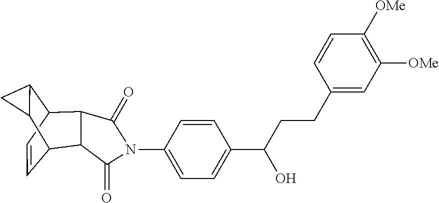 [SMILES: COc1ccc(CCC(O)c2ccc(N3C(=O)C4C5C=CC(C6CC56)C4C3=O)cc2)cc1OC]

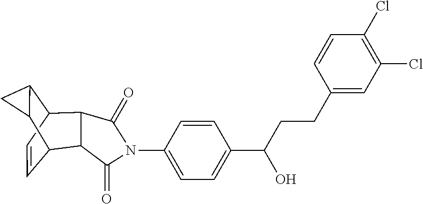 [SMILES: O=C1C2C3C=CC(C4CC34)C2C(=O)N1c1ccc(C(O)CCc2ccc(Cl)c(Cl)c2)cc1]

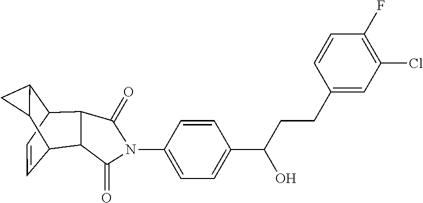 [SMILES: O=C1C2C3C=CC(C4CC34)C2C(=O)N1c1ccc(C(O)CCc2ccc(F)c(Cl)c2)cc1]

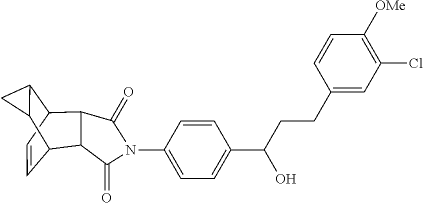 [SMILES: COc1ccc(CCC(O)c2ccc(N3C(=O)C4C5C=CC(C6CC56)C4C3=O)cc2)cc1Cl]

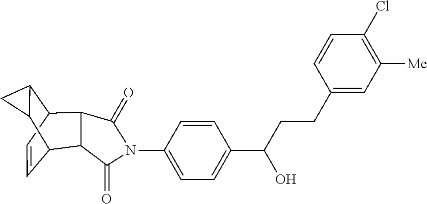 [SMILES: Cc1cc(CCC(O)c2ccc(N3C(=O)C4C5C=CC(C6CC56)C4C3=O)cc2)ccc1Cl]

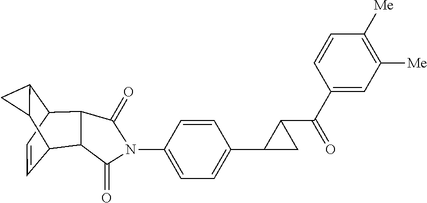 [SMILES: Cc1ccc(C(=O)C2CC2c2ccc(N3C(=O)C4C5C=CC(C6CC56)C4C3=O)cc2)cc1C]

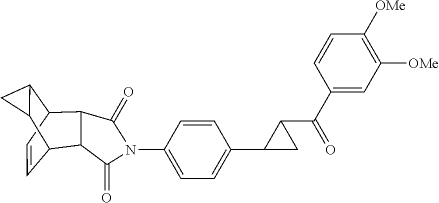 [SMILES: COc1ccc(C(=O)C2CC2c2ccc(N3C(=O)C4C5C=CC(C6CC56)C4C3=O)cc2)cc1OC]

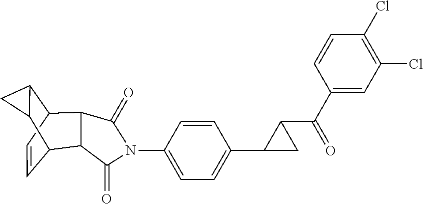 [SMILES: O=C(c1ccc(Cl)c(Cl)c1)C1CC1c1ccc(N2C(=O)C3C4C=CC(C5CC45)C3C2=O)cc1]

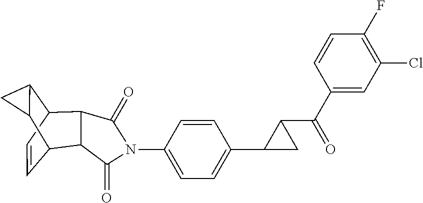 [SMILES: O=C(c1ccc(F)c(Cl)c1)C1CC1c1ccc(N2C(=O)C3C4C=CC(C5CC45)C3C2=O)cc1]

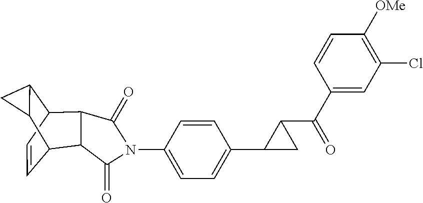 [SMILES: COc1ccc(C(=O)C2CC2c2ccc(N3C(=O)C4C5C=CC(C6CC56)C4C3=O)cc2)cc1Cl]

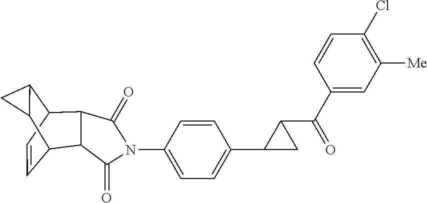 [SMILES: Cc1cc(C(=O)C2CC2c2ccc(N3C(=O)C4C5C=CC(C6CC56)C4C3=O)cc2)ccc1Cl]

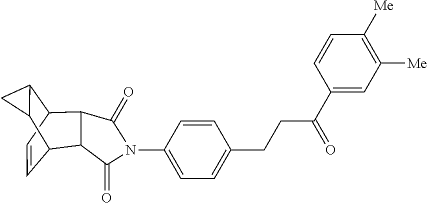 [SMILES: Cc1ccc(C(=O)CCc2ccc(N3C(=O)C4C5C=CC(C6CC56)C4C3=O)cc2)cc1C]

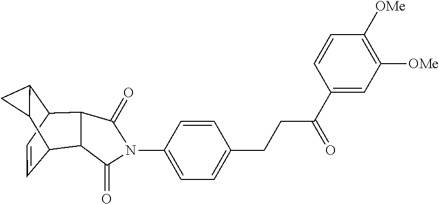 [SMILES: COc1ccc(C(=O)CCc2ccc(N3C(=O)C4C5C=CC(C6CC56)C4C3=O)cc2)cc1OC]

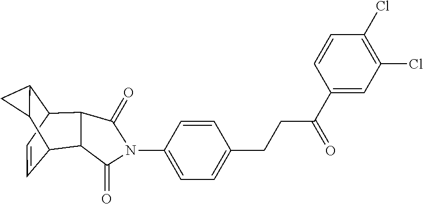 [SMILES: O=C(CCc1ccc(N2C(=O)C3C4C=CC(C5CC45)C3C2=O)cc1)c1ccc(Cl)c(Cl)c1]

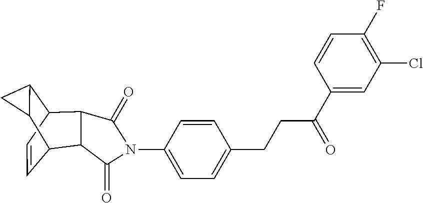 [SMILES: O=C(CCc1ccc(N2C(=O)C3C4C=CC(C5CC45)C3C2=O)cc1)c1ccc(F)c(Cl)c1]

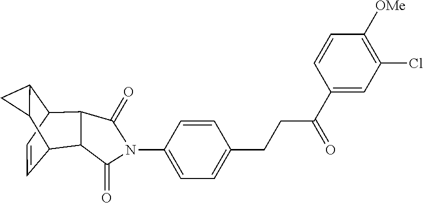 [SMILES: COc1ccc(C(=O)CCc2ccc(N3C(=O)C4C5C=CC(C6CC56)C4C3=O)cc2)cc1Cl]

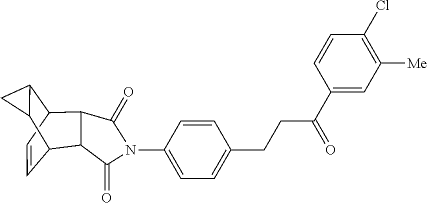 [SMILES: Cc1cc(C(=O)CCc2ccc(N3C(=O)C4C5C=CC(C6CC56)C4C3=O)cc2)ccc1Cl]

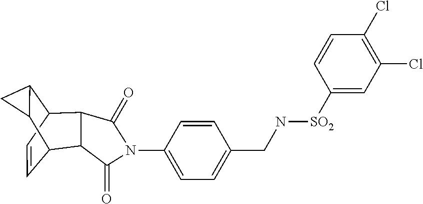 [SMILES: O=C1C2C3C=CC(C4CC34)C2C(=O)N1c1ccc(CNS(=O)(=O)c2ccc(Cl)c(Cl)c2)cc1]

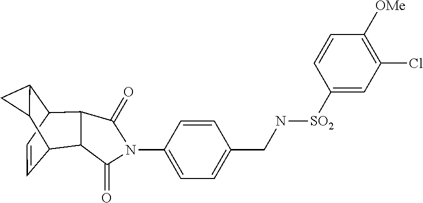 [SMILES: COc1ccc(S(=O)(=O)NCc2ccc(N3C(=O)C4C5C=CC(C6CC56)C4C3=O)cc2)cc1Cl]

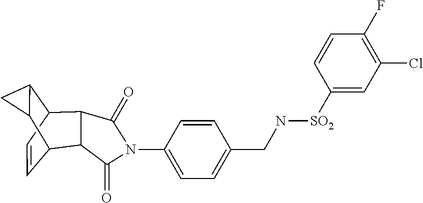 [SMILES: O=C1C2C3C=CC(C4CC34)C2C(=O)N1c1ccc(CNS(=O)(=O)c2ccc(F)c(Cl)c2)cc1]

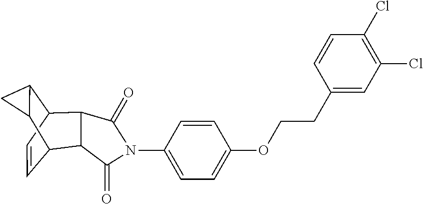 [SMILES: O=C1C2C3C=CC(C4CC34)C2C(=O)N1c1ccc(OCCc2ccc(Cl)c(Cl)c2)cc1]

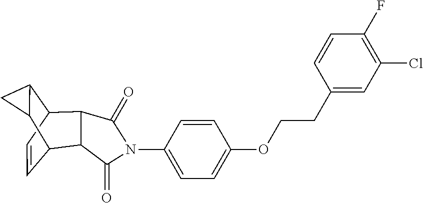 [SMILES: O=C1C2C3C=CC(C4CC34)C2C(=O)N1c1ccc(OCCc2ccc(F)c(Cl)c2)cc1]

TABLE 8
Series III compounds were synthetized consistent with synthetic schemes
1-3, supra; anti-GBM activity was confirmed between 1-200 nm in the
survival screen supra, using Mut 6, a murine GBM stem-like cell (GSC)
line, from a genetically engineered mouse model of GBM wherein
autochthonous tumor formation is driven by GFAP-Cre-mediated
silencing of the tumor suppressors Trp53, Pten, and Nf1 in murine
astrocytes. Animal studies confirm that these analogs are
bioavailable and brain penetrant, do not lead to body weight loss, do not
cause hematologic, hepatic, or renal toxicity, and do not increase but
actually decrease triglyceride levels in mice during chronic treatment.

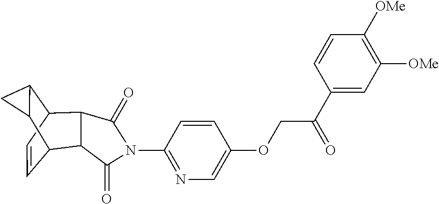 [SMILES: COc1ccc(C(=O)COc2ccc(N3C(=O)C4C5C=CC(C6CC56)C4C3=O)nc2)cc1OC]

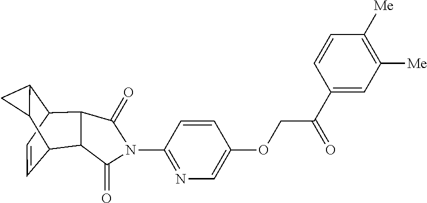 [SMILES: Cc1ccc(C(=O)COc2ccc(N3C(=O)C4C5C=CC(C6CC56)C4C3=O)nc2)cc1C]

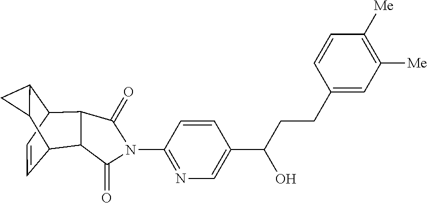 [SMILES: Cc1ccc(CCC(O)c2ccc(N3C(=O)C4C5C=CC(C6CC56)C4C3=O)nc2)cc1C]

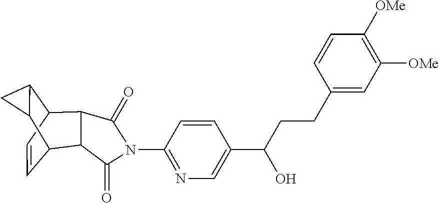 [SMILES: COc1ccc(CCC(O)c2ccc(N3C(=O)C4C5C=CC(C6CC56)C4C3=O)nc2)cc1OC]

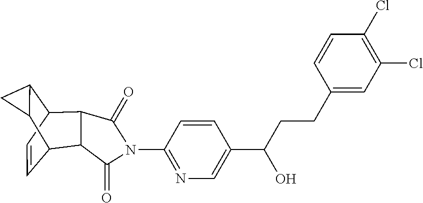 [SMILES: O=C1C2C3C=CC(C4CC34)C2C(=O)N1c1ccc(C(O)CCc2ccc(Cl)c(Cl)c2)cn1]

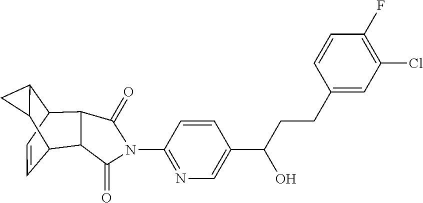 [SMILES: O=C1C2C3C=CC(C4CC34)C2C(=O)N1c1ccc(C(O)CCc2ccc(F)c(Cl)c2)cn1]

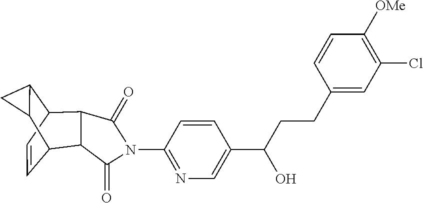 [SMILES: COc1ccc(CCC(O)c2ccc(N3C(=O)C4C5C=CC(C6CC56)C4C3=O)nc2)cc1Cl]

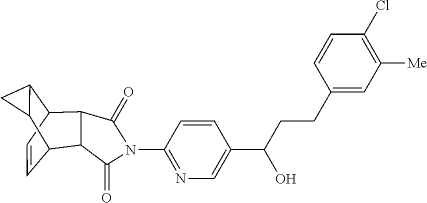 [SMILES: Cc1cc(CCC(O)c2ccc(N3C(=O)C4C5C=CC(C6CC56)C4C3=O)nc2)ccc1Cl]

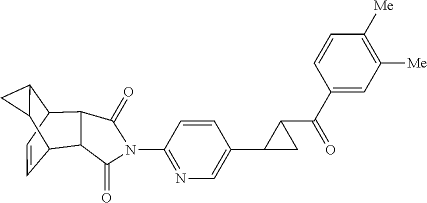 [SMILES: Cc1ccc(C(=O)C2CC2c2ccc(N3C(=O)C4C5C=CC(C6CC56)C4C3=O)nc2)cc1C]

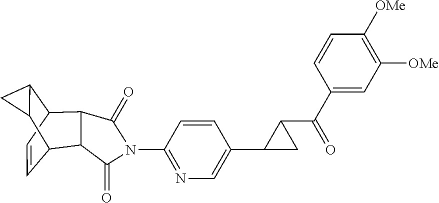 [SMILES: COc1ccc(C(=O)C2CC2c2ccc(N3C(=O)C4C5C=CC(C6CC56)C4C3=O)nc2)cc1OC]

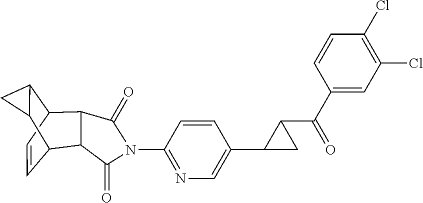 [SMILES: O=C(c1ccc(Cl)c(Cl)c1)C1CC1c1ccc(N2C(=O)C3C4C=CC(C5CC45)C3C2=O)nc1]

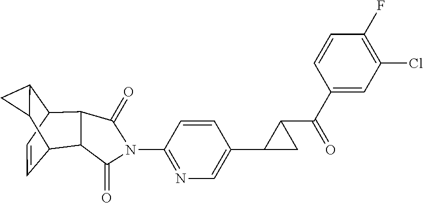 [SMILES: O=C(c1ccc(F)c(Cl)c1)C1CC1c1ccc(N2C(=O)C3C4C=CC(C5CC45)C3C2=O)nc1]

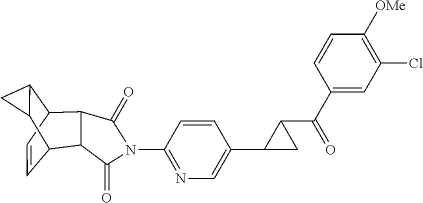 [SMILES: COc1ccc(C(=O)C2CC2c2ccc(N3C(=O)C4C5C=CC(C6CC56)C4C3=O)nc2)cc1Cl]

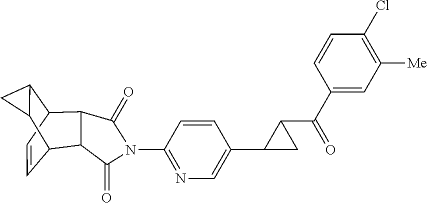 [SMILES: Cc1cc(C(=O)C2CC2c2ccc(N3C(=O)C4C5C=CC(C6CC56)C4C3=O)nc2)ccc1Cl]

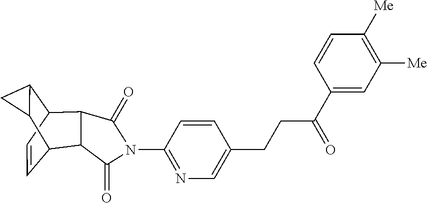 [SMILES: Cc1ccc(C(=O)CCc2ccc(N3C(=O)C4C5C=CC(C6CC56)C4C3=O)nc2)cc1C]

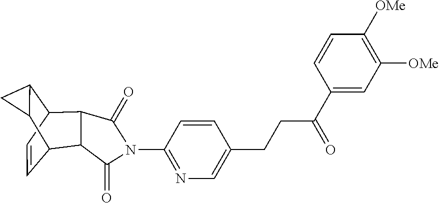 [SMILES: COc1ccc(C(=O)CCc2ccc(N3C(=O)C4C5C=CC(C6CC56)C4C3=O)nc2)cc1OC]

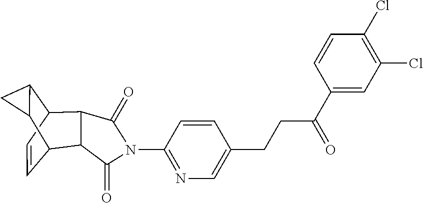 [SMILES: O=C(CCc1ccc(N2C(=O)C3C4C=CC(C5CC45)C3C2=O)nc1)c1ccc(Cl)c(Cl)c1]

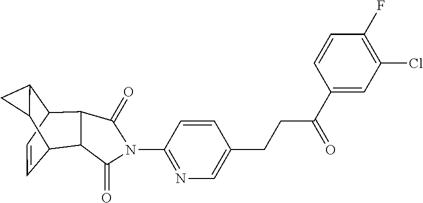 [SMILES: O=C(CCc1ccc(N2C(=O)C3C4C=CC(C5CC45)C3C2=O)nc1)c1ccc(F)c(Cl)c1]

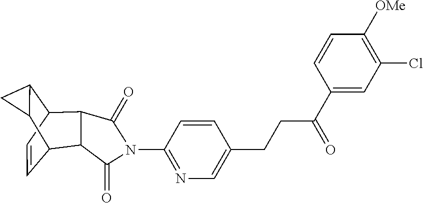 [SMILES: COc1ccc(C(=O)CCc2ccc(N3C(=O)C4C5C=CC(C6CC56)C4C3=O)nc2)cc1Cl]

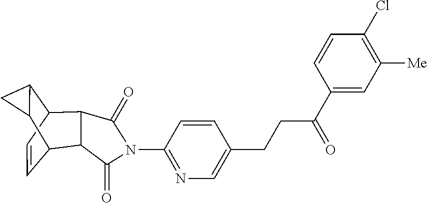 [SMILES: Cc1cc(C(=O)CCc2ccc(N3C(=O)C4C5C=CC(C6CC56)C4C3=O)nc2)ccc1Cl]

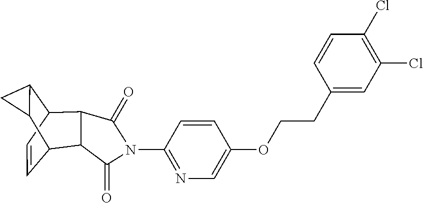 [SMILES: O=C1C2C3C=CC(C4CC34)C2C(=O)N1c1ccc(OCCc2ccc(Cl)c(Cl)c2)cn1]

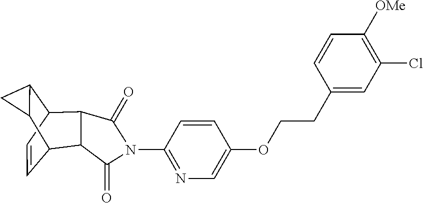 [SMILES: COc1ccc(CCOc2ccc(N3C(=O)C4C5C=CC(C6CC56)C4C3=O)nc2)cc1Cl]

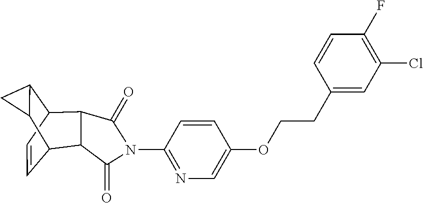 [SMILES: O=C1C2C3C=CC(C4CC34)C2C(=O)N1c1ccc(OCCc2ccc(F)c(Cl)c2)cn1]

TABLE 9
Series IV compounds were synthetized consistent with synthetic schemes
1-3, supra; anti-GBM activity was confirmed between 1-200 nm in the
survival screen supra, using Mut 6, a murine GBM stem-like cell (GSC)
line, from a genetically engineered mouse model of GBM wherein
autochthonous tumor formation is driven by GFAP-Cre-mediated
silencing of the tumor suppressors Trp53, Pten, and Nf1 in murine
astrocytes. Animal studies confirm that these analogs are bioavailable and
brain penetrant, do not lead to body weight loss, do not cause hematologic,
hepatic, or renal toxicity, and do not increase but actually decrease
triglyceride levels in mice during chronic treatment.

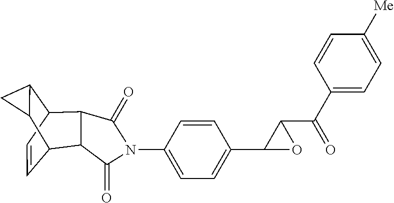 [SMILES: Cc1ccc(C(=O)C2OC2c2ccc(N3C(=O)C4C5C=CC(C6CC56)C4C3=O)cc2)cc1]

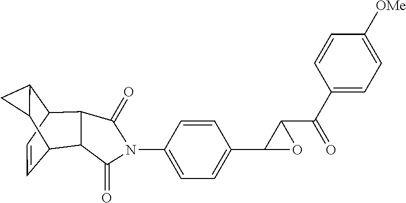 [SMILES: COc1ccc(C(=O)C2OC2c2ccc(N3C(=O)C4C5C=CC(C6CC56)C4C3=O)cc2)cc1]

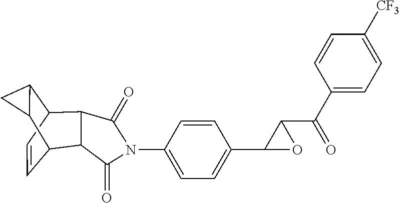 [SMILES: O=C(c1ccc(C(F)(F)F)cc1)C1OC1c1ccc(N2C(=O)C3C4C=CC(C5CC45)C3C2=O)cc1]

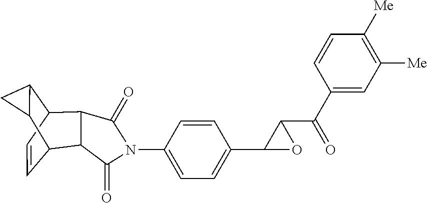 [SMILES: Cc1ccc(C(=O)C2OC2c2ccc(N3C(=O)C4C5C=CC(C6CC56)C4C3=O)cc2)cc1C]

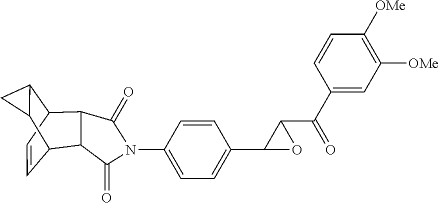 [SMILES: COc1ccc(C(=O)C2OC2c2ccc(N3C(=O)C4C5C=CC(C6CC56)C4C3=O)cc2)cc1OC]

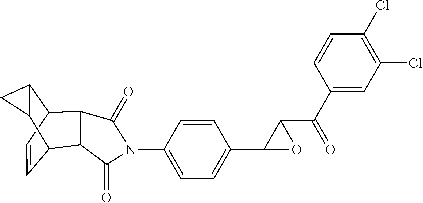 [SMILES: O=C(c1ccc(Cl)c(Cl)c1)C1OC1c1ccc(N2C(=O)C3C4C=CC(C5CC45)C3C2=O)cc1]

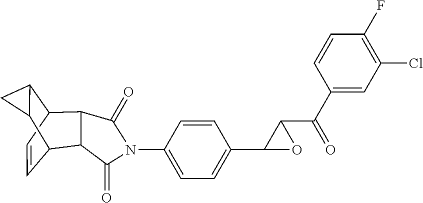 [SMILES: O=C(c1ccc(F)c(Cl)c1)C1OC1c1ccc(N2C(=O)C3C4C=CC(C5CC45)C3C2=O)cc1]

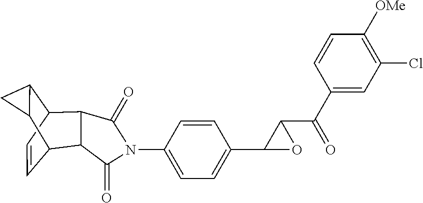 [SMILES: COc1ccc(C(=O)C2OC2c2ccc(N3C(=O)C4C5C=CC(C6CC56)C4C3=O)cc2)cc1Cl]

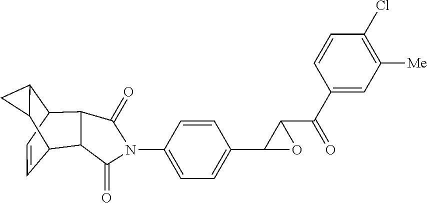 [SMILES: Cc1cc(C(=O)C2OC2c2ccc(N3C(=O)C4C5C=CC(C6CC56)C4C3=O)cc2)ccc1Cl]

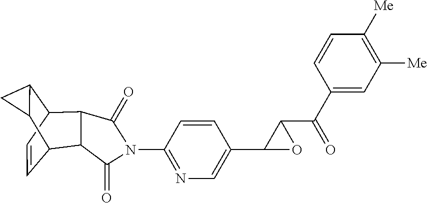 [SMILES: Cc1ccc(C(=O)C2OC2c2ccc(N3C(=O)C4C5C=CC(C6CC56)C4C3=O)nc2)cc1C]

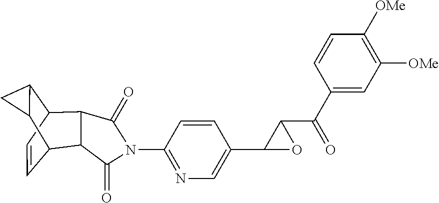 [SMILES: COc1ccc(C(=O)C2OC2c2ccc(N3C(=O)C4C5C=CC(C6CC56)C4C3=O)nc2)cc1OC]

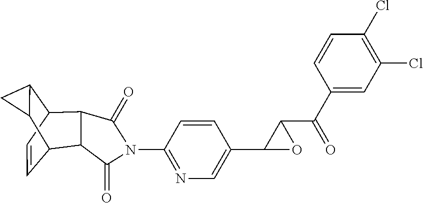 [SMILES: O=C(c1ccc(Cl)c(Cl)c1)C1OC1c1ccc(N2C(=O)C3C4C=CC(C5CC45)C3C2=O)nc1]

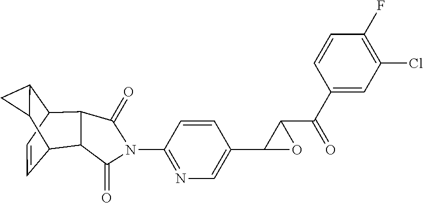 [SMILES: O=C(c1ccc(F)c(Cl)c1)C1OC1c1ccc(N2C(=O)C3C4C=CC(C5CC45)C3C2=O)nc1]

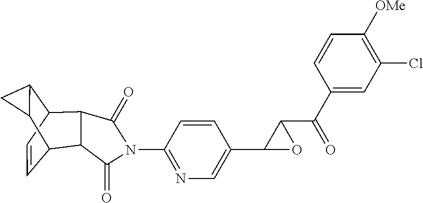 [SMILES: COc1ccc(C(=O)C2OC2c2ccc(N3C(=O)C4C5C=CC(C6CC56)C4C3=O)nc2)cc1Cl]

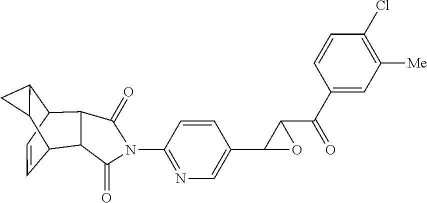 [SMILES: Cc1cc(C(=O)C2OC2c2ccc(N3C(=O)C4C5C=CC(C6CC56)C4C3=O)nc2)ccc1Cl]

TABLE 10
Pharmacokinetic parameters

| | | | | | |
|---|---|---|---|---|---|
| cpd | 24 | 52a | 65 | 79 | 85 |

In Vitro Properties

| | | | | | |
|---|---|---|---|---|---|
| S9 T_{1/2} | 92.4/ | >240/ | 157.5/ | >240/ | >240/ |
| (min)/buffer | 115 | 116 | 109 | 101 | 101 |
| stability (%) | | | | | |
| Phase I T_{1/2} | 70.7/ | >240/ | NT | NT | NT |
| (min)/buffer | 102 | 87 | | | |
| stability (%) | | | | | |
| Phase I + | 83.5/ | >240/ | NT | NT | NT |
| II T_{1/2} (min)/ | 88 | 90 | | | |
| buffer | | | | | |
| stability (%) | | | | | |
| Plasma | >24 | >24 | >24 | NT | >1440 |
| stability (h) | | | | | |

Mouse Pharmacokinetic Profile (IP)

| | | | | | |
|---|---|---|---|---|---|
| Plasma AUC | 260768 | 289859.53 | 440322 | 897663.20 | 286807.33 |
| (min*ng/mL) | | | | | |
| Plasma Cₘₐₓ | 887 | 1303.33 | 1970 | 7016.67 | 3440 |
| (ng/mL) | | | | | |
| Plasma T_{1/2} | 212 | 234.05 | 335 | 118.24 | 74,51 |
| (min) | | | | | |
| Brain AUC | 294638 | 720502.77 | 220405 | 147368.94 | 83822.58 |
| (min*ng/g) | | | | | |
| Brain Cₘₐₓ | 992 | 2525.99 | 1113 | 1044.17 | 978.2 |
| (ng/g) | | | | | |
| Brain T_{1/2} | 194 | 182.33 | 272 | 62.42 | 121.61 |
| (min) | | | | | |
| BBB (ratio)^{d} | 1.13 | 2.49 | 0.5 | 0.16 | 0.29 |

^{b}clogP, clogD, PSA and pKa were calculated using MarvinSketch software.
^{d}BBB was calculated as AUC brain/AUC plasma.

1. A compound of formula I: 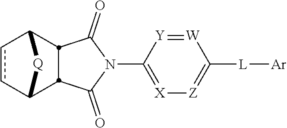
or a salt, hydrate or stereoisomer thereof, wherein:
Q is selected from cyclopropane-1,2-diyl, CH2, (CH2)2, O;
X, Y, Z and W are each independently selected from CR and N; R=H, halogen (e.g. F, Cl, Br); CH3, optionally fluorinated (e.g. CF3), optionally deuterated (e.g. CD3);
L is selected from: —OCHRC(O)—[R=H, Me, CF3, CHF2, CH2F], —NHCH2C(O)—, —NHCHMeC(O)—, —NMeCH2C(O)—, —NMeCHMeC(O)—, —SCH2C(O)—, —SCHMeC(O)—, —CH2OC(O)—, —CHMeOC(O)—, —CH2NHC(O)—, CHMeNHC(O)—, —OCH2NMeC(O)—, —CHMeNMeC(O)—, —CH2NHSO2-, —CHMeNHSO2-, —CH2NMeSO2-, —CMeNMeSO2-, —O(CH2)n- [n=0-2], —OCHMe-, —OCHMeCH2-, —OCH2CHR—[R=Me, OH, OMe, OCF3, OCHF2, OCH2F], —OCHMeCHR—[R=OH, OMe, OCF3, OCHF2, OCH2F], —(CH2)nC(O)—[n=0-2], —CHRCH2C(O)—[R=Me, OH, OMe, OCF3, OCHF2, OCH2F], —CH2CHRC(O)—[R=Me, OH, OMe, OCF3, OCHF2, OCH2F], —CHORCH2n- [n=0-2; R=H, Me, CF3, CHF2, CH2F], —(CH2)nCHOR— [n=1-2; R=H, Me, CF3, CHF2, CH2F], —CHORCH2CHOR′—[R, R′ independently selected from H, Me, CF3, CHF2, CH2F], —CHORCHMeCHOR′—[R, R′ independently selected from H, Me, CF3, CHF2, CH2F], —CHORCMeCH2- [R=H, Me, CF3, CHF2, CH2F], —CH(OR)CH═CH—[R=H, Me, CF3, CHF2, CH2F], —(CH2)n- [n=0-3], —CH2NR—[R=H, Me]-, —CH═CHC(O)—, CH═CHCHR—[R=OH, OMe, OCF3, OCHF2, OCH2F], —CMe=CHC(O)—, CH═CMeC(O)—, CMe=CHCHOR—[R=H, Me, CF3, CHF2, CH2F], CH═CMeCHOR [R=H, Me, CF3, CHF2, CH2F], —C≡CC(O)—, —C≡CCHR [R=H, Me, OH, OMe, OCF3, OCHF2, OCH2F], -(cyclopropane-1,2-diyl)C(O)—, -(cyclopropane-1,2-diyl)CHR—[R=H, Me, OH, OMe, OCF3, OCHF2, OCH2F], -(oxiran-2,3-diyl)C(O)—, -(oxiran-2,3-diyl)CHR—[R=H, Me, OH, OMe, OCF3, OCHF2, OCH2F], —C(O)(cyclopropane-1,2-diyl)-, —CHR(cyclopropane-1,2-diyl)- [R=H, Me, OH, OMe, OCF3, OCHF2, OCH2F], —C(O)(oxiran-2,3-diyl)-, —CHR(oxiran-2,3-diyl)- [R=H, Me, OH, OMe, OCF3, OCHF2, OCH2F]; and
Ar is selected from 2-, or 3- or, 4-mono- or, di- or trisubstituted phenyls (with preferred substituents selected from halogen (e.g. F, Cl, Br), Me or OMe, cycloPr, CN, —NHCHO, each optionally fluorinated and optionally deuterated (e.g. OCF3, CF3, CD3); and optionally substituted phenyls and heteroaryls), mono- and fused bicyclic heteroaryl, fused heteroaryl [e.g. pyridine; pyrimide, pyrazine, pyridazine, oxazole, benzoxazole, pyrrazole, quinoline, quinoxaline, isoquinoline], biaryls and fused aryls (naphtyls); each optionally fluorinated and optionally deuterated (e.g. OCF3, CF3, CD3); with optional substituents selected from halogen (e.g. F, Cl, Br), CF3, Me or OMe, cPr, CN, aryl, and heteroaryl, each optionally fluorinated and optionally deuterated (e.g. OCF3, CF3, CD3).

2. The compound of claim 1, wherein:
Q is cyclopropane-1,2-diyl.

3. The compound of claim 1, wherein:
X is N and Y, Z and W are each CH; or X, Y, Z and W are each CH.

4. The compound of claim 1, wherein:
L is selected from: —OCHC(O)—, —NHCH2C(O)—, —NHCHMeC(O)—, —NMeCH2C(O)—, —NMeCHMeC(O)—, —CH2OC(O)—, —CHMeOC(O)—, —CH2NHC(O)—, CHMeNHC(O)—, —OCH2NMeC(O)—, —CHMeNMeC(O)—, —OCHMe-, —OCHMeCH2-, —OCH2CHMe-, —OCHMeCHOH—, —(CH2)C(O)—, —CHMeCH2C(O)—, —CH2CHMeC(O)—, —CHOHCH2-, —CH2CHOH—, —CHOHCH2CHOH′—, —CHOHCHMeCHOH—, —CHOHCMeCH2-, —CH(OH)CH═CH2-, —CH2NH—, -(cyclopropane-1,2-diyl)C(O)—, -(cyclopropane-1,2-diyl)CH2-, -(oxiran-2,3-diyl)C(O)—, -(oxiran-2,3-diyl)CH2-, —C(O)(cyclopropane-1,2-diyl)-, —CH2(cyclopropane-1,2-diyl)-, —C(O)(oxiran-2,3-diyl)-, —CH2(oxiran-2,3-diyl)-.

5. The compound of claim 1, wherein:
Ar is 2-, or 3- or, 4-mono- or, di- or trisubstituted phenyls, with substituents selected from halogen, Me or OMe, cycloPr, CN, and —NHCHO, each optionally fluorinated and optionally deuterated.

6. The compound of claim 1, wherein:
Q is cyclopropane-1,2-diyl;
X is N and Y, Z and W are each CH; or X, Y, Z and W are each CH; and
Ar is 2-, or 3- or, 4-mono- or, di- or trisubstituted phenyls, with substituents selected from halogen, Me or OMe, cycloPr, CN, and —NHCHO, each optionally fluorinated and optionally deuterated.

7. The compound of claim 1, wherein:
Q is cyclopropane-1,2-diyl;
X is N and Y, Z and W are each CH; or X, Y, Z and W are each CH;
L is selected from: —OCHC(O)—, —NHCH2C(O)—, —NHCHMeC(O)—, —NMeCH2C(O)—, —NMeCHMeC(O)—, —CH2OC(O)—, —CHMeOC(O)—, —CH2NHC(O)—, CHMeNHC(O)—, —OCH2NMeC(O)—, —CHMeNMeC(O)—, —OCHMe-, —OCHMeCH2-, —OCH2CHMe-, —OCHMeCHOH—, —(CH2)C(O)—, —CHMeCH2C(O)—, —CH2CHMeC(O)—, —CHOHCH2-, —CH2CHOH—, —CHOHCH2CHOH′—, —CHOHCHMeCHOH—, —CHOHCMeCH2-, —CH(OH)CH═CH2-, —CH2NH—, -(cyclopropane-1,2-diyl)C(O)—, -(cyclopropane-1,2-diyl)CH2-, -(oxiran-2,3-diyl)C(O)—, -(oxiran-2,3-diyl)CH2-, —C(O)(cyclopropane-1,2-diyl)-, —CH2(cyclopropane-1,2-diyl)-, —C(Oxoxiran-2,3-diyl)-, —CH2(oxiran-2,3-diyl)-; and
Ar is 2-, or 3- or, 4-mono- or, di- or trisubstituted phenyls, with substituents selected from halogen, Me or OMe, cycloPr, CN, and —NHCHO, each optionally fluorinated and optionally deuterated.

8. A compound of claim 1, having a structure of Table 1, herein.

9. A compound of claim 1, having a structure of Table 2, herein.

10. A compound of claim 1, having a structure of Table 3, herein.

11. A compound of claim 1, having a structure of Table 4, herein.

12. A compound of claim 1, having a structure of Table 5, herein.

13. A compound of claim 1, having a structure of Table 6, herein.

14. A compound of claim 1, having a structure of Table 7, herein.

15. A compound of claim 1, having a structure of Table 8, herein.

16. A compound of claim 1, having a structure of Table 9, herein.

17. A compound of claim 1, which inhibits lanosterol synthase (LSS), is orally bioavailable, and crosses the blood-brain barrier.

18. A pharmaceutical composition comprising a compound of claim 1, or a pharmaceutically acceptable salt thereof, and a pharmaceutically acceptable carrier or excipient.

19. A method of inhibiting lanosterol synthase, or of upregulating 24,25-epoxycholesterol (EPC), or for treating a neurological disease or condition, the method comprising:
administering to a person in need thereof a small molecule lanosterol synthase inhibitor of claim 1,
optionally wherein the neurological disease or condition is glioblastoma (GBM) or a neurodegenerative disease, such as amyotrophic lateral sclerosis, multiple sclerosis, Parkinson's disease, Alzheimer's disease, Huntington's disease; and
optionally further comprising the antecedent step of detecting or diagnosing the disease or condition and/or the subsequent step of detecting a resultant improvement or delay of progression of the disease or condition.

20. A method of screening for candidate therapeutics for treating a neurological disease or condition, the method comprising identifying inhibitors of lanosterol synthase (p75).
